(19) United States
(12) Patent Application Publication
(10) Pub. No.: US 2026/0258418 A1
(43) Pub. Date: Sep. 3, 2026

(54) METHODS AND COMPOSITIONS FOR TARGETED TRANS-SPLICING

(71) Applicant: Amber Bio Inc., South San Francisco, CA (US)

(72) Inventors: Jacob BORRAJO, South San Francisco, CA (US); Basem AL-SHAYEB, South San Francisco, CA (US)

(21) Appl. No.: 19/673,407

(22) Filed: May 11, 2026

Related U.S. Application Data
(63) Continuation of application No. 18/938,195, filed on Nov. 5, 2024, now Pat. No. 12,668,800.
Application No. 18/938,195 is a continuation of application No. PCT/US2023/081868, filed on Nov. 30, 2023.
(60) Provisional application No. 63/429,031, filed on Nov. 30, 2022.

(51) Int. Cl. C12N 15/113; C12N 15/85

(52) U.S. Cl. CPC C12N 15/113; C12N 15/85; C12N 2310/14; C12N 2310/531

(57) ABSTRACT

The present disclosure provides compositions and methods of use thereof for targeting trans-splicing of a pre-mRNA in a cell.

### CROSS-REFERENCE TO RELATED APPLICATIONS

This application claims priority to U.S. Provisional Application No. 63/429,031, filed on Nov. 30, 2022, the entire contents of which are incorporated herein.

### FIELD

The disclosure relates to a nucleic acid composition for targeting trans-splicing of a pre-mRNA in a cell, and related methods.

### SEQUENCE LISTING SUBMISSION VIA EFS-WEB

The instant application contains a sequence listing, which has been submitted in XML format via EFS-Web. The contents of the XML copy named “AMR-014PC/134241-5014_Sequence Listing”, which was created on Nov. 30, 2023 and is 513,000 bytes in size, the contents of which are incorporated herein by reference in their entirety.

### BACKGROUND

Gene editing is widely recognized as a promising approach to treat numerous diseases associated with viral infection, enzymatic deficiency, and hereditary myopathies. For example, gene-editing using a CRISPR/Cas system can introduce a double-stranded break in a gene of interest that is repaired by endogenous DNA repair pathways to introduce a gene knockout or a correction of a mutation. Appropriate gene edits can function to eliminate a mutation in, decrease expression of, or alter function or activity of an encoded protein in order to provide desirable therapeutic outcomes. However, despite significant progress, gene editing approaches remain problematic due to the risk of introducing deleterious off-target edits to the genome and packaging constraints for delivery of system components. An alternative approach to introduce genetic information to a cell that avoids the risk of introducing permanent changes to the genome is by regulating splicing of endogenous nucleic acids (e.g., RNA transcripts).

Splicing is a reaction that occurs in the nucleus of eukaryotic cells and is catalyzed by the spliceosome, a large ribonucleoprotein (RNP) complex. Splicing removes noncoding sequences (introns) from RNA transcripts (pre-mRNA) and ligates coding sequences (exons) together. The spliceosome typically mediates cis-splicing of endogenous RNA transcripts, in which a lariat is formed in an intron and then excised in order to join two exons in the same RNA transcript (see, e.g., Matera, et al (2014) Nat Rev Mol Cell Biol 15(2):108-21; Wilkinson, et al (2020) Annu Rev Biochem 89:359). The spliceosome can also perform trans-splicing, in which exons from two different primary RNA transcripts are joined end-to-end and ligated (Lasda, et al (2011) Wiley Interdiscip Rev RNA 2:417-34). Trans-splicing yields a chimeric molecule comprising one or more exonic regions from the first RNA molecule and one or more exonic regions from the second RNA molecule.

Trans-splicing using an exogenous nucleic acid encoding desired genetic information is a promising avenue for therapeutic nucleic acid editing and other biotechnology applications. For example, it has been demonstrated that introducing an artificial RNA introduced to a cell can undergo trans-splicing with an endogenous pre-mRNA (see, e.g., Puttaraju, et al (1999) Nat Biotech 17:246). Such trans-splicing efforts have focused on spliceosome-mediated RNA trans-splicing (SMaRT), where activity of a pre-mRNA trans-splicing molecule (PTM) is achieved by RNA-RNA interactions between a binding domain that hybridizes to a target pre-mRNA (Puttaraju, 1999). While some groups have been able to demonstrate in vitro and in vivo activity with SMaRT technology (Mansfield, et al (2000) Gene therapy 7: 1885-1895; Liu, et al (2002) Nat. Biotechnol. 20:47), it is a relatively inefficient process that has not yet progressed to the clinic 20 (Berger, et al (2016) Wiley Interdisciplinary Reviews: RNA 7:487-98).

Thus, new approaches are needed to enable targeted and efficient trans-splicing to introduce desired genetic information to a cell.

### SUMMARY OF THE DISCLOSURE

In some aspects, the disclosure provides a nucleic acid for targeting trans-splicing of a pre-mRNA in a cell, the nucleic acid comprising a nucleotide sequence comprising (a) at least one intronic sequence comprising (i) one or more binding domain sequences of about 4 to about 300 nucleotides each with complementarity to a pre-mRNA target sequence; and (ii) a non-coding RNA (ncRNA) sequence of about 7 to about 300 nucleotides in length which forms a secondary structure and/or comprises a sequence motif to direct the one or more binding domains to the pre-mRNA target sequence; (b) a splice acceptor and/or splice donor sequence; and (c) at least one exonic sequence.

In some embodiments, the one or more binding domain sequences is at least about 5 to about 10, about 5 to about 15, about 5 to about 20, about 10 to about 15, about 10 to about 20, about 15 to about 20, or about 20, 19, 18, 17, 16, 15, 14, 13, 12, 11, 10, 9, 8, 7, 6, or 5 nucleotides in length. In some embodiments, the one or more binding domain sequences is less than about 250 to about 300, about 200 to about 300, about 150 to about 300, about 100 to about 300, about 50 to about 300, about 100 to about 250, about 100 to about 200, about 100 to about 150, about 50 to about 250, about 50 to about 200, about 50 to about 150, about 50 to about 100, or about 300, 250, 200, 150, 100, or 50 nucleotides in length. In some embodiments, the one or more binding domain sequences is about 5 to about 20, about 5 to about 30, about 5 to about 40, about 5 to about 50, about 10 to about 50, about 10 to about 100, about 20 to about 100, about 30 to about 100, about 40 to about 100, about 50 to about 100, about 50 to about 150, about 50 to about 200, about 50 to about 250, about 100 to about 150, about 100 to about 200, about 100 to about 250, or about 100 to about 300 nucleotides in length.

In some embodiments, the at least one intronic sequence comprises one binding domain sequence. In some embodiments, the at least one intronic sequence comprises at least two binding domain sequences. In some embodiments, the at least one intronic sequence comprises 3, 4, 5, 6, 7, 8, 9, or 10 binding domain sequences.

In some embodiments, when the nucleic acid is introduced to the cell an exon in the pre-mRNA is targeted for trans-splicing. In some embodiments, the target sequence is positioned in a region of the pre-mRNA comprising the exon targeted for trans-splicing. In some embodiments, the target sequence is positioned proximal to a splice site. In some embodiments, the target sequence is positioned proximal to a splice donor or a splice acceptor.

In some embodiments, the ncRNA sequence is selected from an snRNA, a snoRNA, a lncRNA, an rRNA, a ribozyme, an sRNA, a scaRNA, and a vault RNA. In some embodiments, the ncRNA sequence is an snRNA. In some embodiments, the snRNA is selected from a U1 snRNA, a U2 snRNA, a U4 snRNA, a U4atac snRNA, a U5 snRNA, a U6 snRNA, a U6atac snRNA, a U11 snRNA, a U12 snRNA, and a U7 snRNA. In some embodiments, the ncRNA sequence is a snoRNA. In some embodiments, the snoRNA comprises an H/ACA box or C/D box.

In some embodiments, the ncRNA sequence assembles into an RNP. In some embodiments, the ncRNA sequence comprises a sequence motif that assembles into an RNP. In some embodiments, the ncRNA sequence comprises a secondary structure that assembles into an RNP. In some embodiments, the ncRNA sequence comprises a sequence motif and a secondary structure that assembles into an RNP. In some embodiments, the secondary structure comprises one or more stem loops. In some embodiments, the RNP is selected from a small nuclear RNP (snRNP), a small nucleolar RNP (snoRNP), a small cajal body RNP (scaRNP), and a combination thereof. In some embodiments, the RNP is a snRNP. In some embodiments, the RNP is selected from U1, U2, U4, U4atac, U5, U6, U6atac, U7, U11, and U12. In some embodiments, the RNP is a snoRNP. In some embodiments, the RNP is selected from a C/D box snoRNP and a H/ACA box snoRNP.

In some embodiments, the ncRNA comprises an Sm sequence motif. In some embodiments, the Sm sequence motif assembles with an Sm or Lsm protein into an RNP. In some embodiments, the Sm or Lsm proteins are selected from a B/B′, D3, D2, D1, E, F, G, LSm5, LSm7, LSm4, LSm8, LSm2, LSm3, LSm6 and LSm10 proteins.

In some embodiments, the at least one intronic sequence comprises a splice acceptor. In some embodiments, the at least one intronic sequence comprises a splice donor. In some embodiments, the at least one intronic sequences comprises one or more splicing signals. In some embodiments, the one or more splicing signals are selected from an exonic splicing enhancer (ESE), an intronic splicing enhancer (ISE), an exonic splicing silencer (ESS), intronic splicing silencer (ISS), a polypyrimidine tract, a branch point, and a combination thereof. In some embodiments, the at least one intronic sequences comprises a branch point and a polypyrimidine tract. In some embodiments, the nucleic acid comprising a nucleotide sequence comprises 1, 2, 3, 4, 5, 6, 7, 8, 9, or 10 exons.

In some aspects, the disclosure provides a nucleic acid for targeting trans-splicing of a pre-mRNA in a cell, the nucleic acid comprising a nucleotide sequence comprising from 5′ to 3′: (a) at least one intronic sequences comprising (i) one or more binding domain sequences of about 4 to about 300 nucleotides each with complementarity to a pre-mRNA target sequence; (ii) a non-coding RNA (ncRNA) sequence of about 7 to about 300 nucleotides in length which forms a secondary structure and/or comprises a sequence motif to direct the one or more binding domains to the pre-mRNA target sequence; and (iii) one or more splicing signals; (b) a splice acceptor; and (c) at least one exonic sequences. In some embodiments, when the nucleic acid is introduced to the cell an exon in the pre-mRNA is targeted for trans-splicing. In some embodiments, the target sequence is positioned upstream the exon in the pre-mRNA targeted for trans-splicing. In some embodiments, the target sequence is positioned proximal to a splice site (e.g., a splice acceptor or splice donor). In some embodiments, the target sequence is positioned proximal to a splice acceptor. In some embodiments, the target sequence is positioned proximal to a splice donor. In some embodiments, trans-splicing occurs between a splice donor upstream the exon in the pre-mRNA and the splice acceptor of the nucleic acid. In some embodiments, trans-splicing results in ligation of the 3′end of an exon upstream the splice donor in the pre-mRNA with the 5′end of the at least one exonic sequence of the nucleic acid. In some embodiments, the one or more splicing signals comprises a branch point and a polypyrimidine tract.

In some aspects, the disclosure provides a nucleic acid for targeting trans-splicing a pre-mRNA in a cell, the nucleic acid comprising a nucleotide sequence comprising from 5′ to 3′: (a) at least one exonic sequence; (b) a splice donor; (c) at least one intronic sequence comprising (i) a non-coding RNA (ncRNA) sequence of about 7 to about 300 nucleotides in length, and (ii) one or more binding domain sequences of about 4 to about 300 nucleotides each with complementarity to a pre-mRNA target sequence, wherein the ncRNA forms a secondary structure and/or comprises a sequence motif to direct the one or more binding domains to the pre-mRNA target sequence. In some embodiments, when the nucleic acid is introduced to the cell an exon in the pre-mRNA is targeted for trans-splicing. In some embodiments, the target sequence is positioned downstream the exon in the pre-mRNA. In some embodiments, the target sequence is positioned proximal to a splice site (e.g., a splice donor or splice acceptor). In some embodiments, the target sequence is positioned proximal to a splice donor. In some embodiments, the target sequence is positioned proximal to a splice acceptor. In some embodiments, trans-splicing occurs between the splice donor of the nucleic acid and a splice acceptor downstream the exon in the pre-mRNA. In some embodiments, trans-splicing results in ligation of the 3′end of the at least one exonic sequence of the nucleic acid with the 5′end of an exon downstream the splice acceptor in the pre-mRNA.

In some embodiments, the ncRNA sequence is an snRNA. In some embodiments, the snRNA is selected from a U1 snRNA, a U2 snRNA, a U4 snRNA, a U4atac snRNA, a U5 snRNA, a U6 snRNA, a U6atac snRNA, a U11 snRNA, a U12 snRNA, and a U7 snRNA. In some embodiments, the snRNA assembles into an snRNP. In some embodiments, the snRNA is a U1 snRNA. In some embodiments, the U1 snRNA assembles into a U1 RNP. In some embodiments, the snRNA is a U11 snRNA. In some embodiments, the U11 snRNA assembles into a U11 RNP. In some embodiments, the snRNA is a U7 snRNA. In some embodiments, the U7 snRNA assembles into a U7 RNP. In some embodiments, the ncRNA sequence comprises an Sm sequence motif In some embodiments, the ncRNA sequence comprises an Sm sequence motif and a U7 snRNA. In some embodiments, the Sm sequence motif comprises a sequence set forth in SEQ ID NOs: 3 and 4. In some embodiments, the Sm sequence motif assembles with an Sm protein into an RNP. In some embodiments, the Sm protein is selected from a B/B′, D3, D2, D1, E, F, and G Sm protein.

In some embodiments, the ncRNA sequence comprises a sequence having at least 80% sequence identity to a sequence selected from SEQ ID NOs: 9-589 or a portion thereof (e.g., a contiguous portion thereof). In some embodiments, the ncRNA sequence comprises a region of about 7 to about 40 nucleotides in length, wherein the region comprises an Sm sequence motif In some embodiments, the ncRNA sequence comprises a region of about 40 to about 300 nucleotides in length, wherein the region comprises a secondary structure and/or an Sm sequence motif In some embodiments, the Sm sequence motif comprises a sequence selected from SEQ ID NOs: 209-399.

In some embodiments, the at least one intronic sequences comprises one binding domain sequence. In some embodiments, the one binding domain sequence is about 5 to about 20, about 5 to about 30, about 5 to about 40, about 5 to about 50, about 10 to about 50, about 10 to about 100, about 20 to about 100, about 30 to about 100, about 40 to about 100, about 50 to about 100, about 50 to about 150, about 50 to about 200, about 50 to about 250, about 100 to about 150, about 100 to about 200, about 100 to about 250, or about 100 to about 300 nucleotides in length.

In some embodiments, the at least one intronic sequences more than one binding domain sequence. In some embodiments, more than one binding domain sequences are each about 5 to about 20, about 5 to about 30, about 5 to about 40, about 5 to about 50, about 10 to about 50, about 10 to about 100, about 20 to about 100, about 30 to about 100, about 40 to about 100, about 50 to about 100, about 50 to about 150, about 50 to about 200, about 50 to about 250, about 100 to about 150, about 100 to about 200, about 100 to about 250, or about 100 to about 300 nucleotides in length.

In some aspects, the disclosure provides a nucleic acid for targeting trans-splicing of a pre-mRNA in a cell, the nucleic acid comprising a nucleotide sequence comprising from 5′ to 3′ (a) at least one intronic sequence comprising (i) a ncRNA sequence comprising an H/ACA box or a C/D box and one or more binding domain sequences of about 4 to about 30 nucleotides each with complementarity to a pre-mRNA target sequence; and (ii) one or more splicing signals; (b) a splice acceptor; and (c) at least one exonic sequence. In some embodiments, when the nucleic acid is introduced to the cell an exon in the pre-mRNA is targeted for trans-splicing. In some embodiments, the target sequence is positioned upstream the exon in the pre-mRNA. In some embodiments, the target sequence is positioned proximal to a splice site. In some embodiments, the target sequence is positioned proximal to a splice donor or splice acceptor. In some embodiments, trans-splicing occurs between a splice donor upstream the exon in the pre-mRNA and the splice acceptor of the nucleic acid. In some embodiments, trans-splicing results in ligation of the 3′ end of an exon upstream the splice donor in the pre-mRNA with the 5′end of the at least one exonic sequence of the nucleic acid. In some embodiments, the one or more splicing signals comprises a branch point and a polypyrimidine tract.

In some aspects, the disclosure provides a nucleic acid for targeting trans-splicing of a pre-mRNA in a cell, the nucleic acid comprising a nucleotide sequence comprising from 5′ to 3′ (a) at least one exonic sequence; (b) a splice donor; and (c) at least one intronic sequence comprising a ncRNA sequence comprising an H/ACA box or a C/D box and one or more binding domain sequences of about 4 to about 30 nucleotides each with complementarity to a pre-mRNA target sequence. In some embodiments, when the nucleic acid is introduced to the cell an exon in the pre-mRNA is targeted for trans-splicing. In some embodiments, the target sequence is positioned downstream the exon in the pre-mRNA. In some embodiments, the target sequence is positioned proximal to a splice site. In some embodiments, the target sequence is positioned proximal to a splice donor or a splice acceptor. In some embodiments, trans-splicing occurs between the splice donor of the nucleic acid and a splice acceptor downstream the exon in the pre-mRNA. In some embodiments, trans-splicing results in ligation of the 3′end of the at least one exonic sequence of the nucleic acid with the 5′end of an exon downstream the splice acceptor in the pre-mRNA.

In some embodiments, the ncRNA sequence comprises an H/ACA box comprising 5′ to 3′ an H consensus sequence and an ACA consensus sequence. In some embodiments, the ncRNA sequence comprises the at least one binding domain sequence positioned (i) upstream the H consensus sequence; (ii) downstream the ACA consensus sequence; (iii) between the H consensus sequence and the ACA consensus sequence; or (iv) a combination of (i)-(iii).

In some embodiments, the ncRNA sequence comprises C/D box comprising 5′ to 3′ a C consensus sequence, a D′ consensus sequence, a C′ consensus sequence, and a D consensus sequence. In some embodiments, the ncRNA sequence comprises the at least one binding domain positioned (i) upstream the C consensus sequence; (ii) between the C consensus sequence and the D′ consensus sequence; (iii) between the C′ consensus sequence and the D consensus sequence; (iv) downstream the D consensus sequence; or (iv) a combination of (i)-(iii).

In some embodiments, the ncRNA sequence comprises a sequence having at least 80% sequence identity to a sequence selected from SEQ ID NOs: 590-657 or a portion thereof (e.g., a contiguous portion thereof). In some embodiments, the ncRNA sequence comprises a region of about 40 to about 300 nucleotides in length and comprising an H consensus sequence and an ACA consensus sequence.

In some embodiments, the ncRNA sequence comprises one binding domain sequence. In some embodiments, the ncRNA sequence comprises more than one binding domain sequence.

In some embodiments, the at least one intronic sequence comprises at least one binding domain sequence with full complementarity to the pre-mRNA target sequence. In some embodiments, the at least one intronic sequence comprises at least one binding domain sequence with partial complementarity to the pre-mRNA target sequence. In some embodiments, the at least one binding domain sequence comprises one or more mismatches relative to the pre-mRNA target sequence. In some embodiments, the at least one binding domain sequence has at least 95% complementarity to the pre-mRNA target sequence.

In some embodiments, the nucleic acid comprises a sequence up to about 20,000 nucleotides in length. In some embodiments, the nucleic acid comprises a sequence of about 50 to about 500, about 50 to about 1000, about 100 to about 500, about 100 to about 1000, about 500 to about 1000, about 500 to about 2000, about 500 to about 3,000, about 500 to about 4,000, about 500 to about 5,000, about 1,000 to about 5,000, about 1,000 to about 10,000, about 5,000 to about 15,000, or about 5,000 to about 20,000 nucleotides in length.

In some embodiments, the nucleic acid is introduced to the cell as an RNA. In some embodiments, the nucleic acid is introduced to the cell as a DNA. In some embodiments, the nucleic acid is introduced to the cell by a viral vector. In some embodiments, the viral vector is an AAV. In some embodiments, the nucleic acid is introduced to the cell by a non-viral vector.

In some embodiments, introduction of the nucleic acid to the cell results in an efficiency of trans-splicing that is greater than a nucleic acid lacking the ncRNA sequence. In some embodiments, the efficiency of trans-splicing is greater than about 10%, about 20%, about 30%, about 40%, about 50%, about 60%, about 70%, about 80%, about 90%, or about 99%.

In some embodiments, the nucleic acid is formulated as a lipid nanoparticle.

In some aspects, the disclosure provides a viral vector comprising a nucleic acid described herein.

In some aspects, the disclosure provides a lipid nanoparticle comprising a nucleic acid described herein.

In some aspects, the disclosure provides a cell comprising a nucleic acid described herein, a viral vector described herein, or a lipid nanoparticle described herein.

In some aspects, the disclosure provides a pharmaceutical composition comprising a nucleic acid described herein, a viral vector described herein, or a lipid nanoparticle described herein, and a pharmaceutically acceptable carrier.

In some aspects, the disclosure provides a pharmaceutical composition comprising a cell described herein, and a pharmaceutically acceptable carrier.

In some aspects, the disclosure provides a method of targeting trans-splicing of a pre-mRNA in a cell, the method comprising contacting the cell with a nucleic acid described herein, a viral vector described herein, a lipid nanoparticle described herein, or a pharmaceutical composition described herein, wherein when the nucleic acid, the viral vector, the lipid nanoparticle, or the pharmaceutical composition contacts the cell, the one or more binding domain sequences bind to the pre-mRNA, thereby targeting the pre-mRNA for trans-splicing.

In some aspects, the disclosure provides a method of correcting a mutation in a pre-mRNA in a cell, the method comprising contacting the cell with a nucleic acid described herein, a viral vector described herein, a lipid nanoparticle described herein, or a pharmaceutical composition described herein, wherein when the nucleic acid, the viral vector, the lipid nanoparticle, or the pharmaceutical composition contacts the cell, the one or more binding domain sequences bind to the pre-mRNA at a location proximal to the mutation, and wherein trans-splicing replaces one or more exons in the pre-mRNA comprising the mutation, thereby correcting the mutation.

In some aspects, the disclosure provides a method of treating a patient with a disease or disorder associated with a mutation in a pre-mRNA, the method comprising administering to the patient an effective amount of a nucleic acid described herein, a viral vector described herein, a lipid nanoparticle described herein, or a pharmaceutical composition described herein, wherein when the nucleic acid, the viral vector, the lipid nanoparticle, or the pharmaceutical composition is administered, the one or more binding domain sequences bind to the pre-mRNA at a location proximal to the mutation, and wherein trans-splicing replaces one or more exons in the pre-mRNA comprising the mutation, thereby correcting the mutation. In some embodiments, the trans-splicing results in an mRNA that alleviates the disease or does not cause or contribute to the disease.

In some aspects, the disclosure provides a nucleic acid of any one of the embodiments disclosed herein, the viral vector of any one of the embodiments disclosed herein, the lipid nanoparticle of any one of the embodiments disclosed herein, or the pharmaceutical composition of any one of the embodiments disclosed herein for use in treating a patient with a disease or disorder associated with a mutation in a pre-mRNA, the treatment comprising administering to the patient the nucleic acid, the viral vector, the lipid nanoparticle, or the pharmaceutical composition, wherein when the nucleic acid, the viral vector, the lipid nanoparticle, or the pharmaceutical composition is administered, the one or more binding domain sequences bind to the pre-mRNA at a location proximal to the mutation, and wherein trans-splicing replaces one or more exons in the pre-mRNA comprising the mutation, thereby correcting the mutation.

In some aspects, the disclosure provides a nucleic acid of any one of the embodiments disclosed herein, the viral vector of any one of the embodiments disclosed herein, the lipid nanoparticle of any one of the embodiments disclosed herein, or the pharmaceutical composition of any one of the embodiments disclosed herein for the manufacture of a medicament for use in treating a patient with a disease or disorder associated with a mutation in a pre-mRNA, the treatment comprising administering to the patient the medicament, wherein when the medicament is administered, the one or more binding domain sequences of the nucleic acid binds to the pre-mRNA at a location proximal to the mutation, and wherein trans-splicing replaces one or more exons in the pre-mRNA comprising the mutation, thereby correcting the mutation.

In some aspects, the disclosure provides a kit comprising a container comprising a nucleic acid described herein, a viral vector described herein, a lipid nanoparticle described herein, or a pharmaceutical composition described herein, with instructions for use in correcting a mutation in a pre-mRNA.

### BRIEF DESCRIPTION OF THE FIGURES

FIG. 1A and FIG. 1B provide schematics, without wishing to be bound by theory, depicting cis-splicing of a pre-mRNA (FIG. 1A) and trans-splicing between two pre-mRNA molecules (FIG. 1B). “SD” refers to a splice donor. “SA” refers to a splice acceptor.

FIG. 1C provides a schematic, without wishing to be bound by theory, depicting an exemplary splice editor nucleic acid molecule of the disclosure for targeted trans-splicing of a pre-mRNA. Labeling of the splice editor indicates the segments corresponding to an RNA binding domain, a non-coding RNA (ncRNA), SA, and exon. Labeling of the pre-mRNA indicates segments of the pre-mRNA corresponding to the 5′exon, SD, SA, and 3′exon.

FIG. 1D provides a schematic, without wishing to be bound by theory, depicting a predicted secondary structure of an exemplary splice editor nucleic acid molecule of the disclosure and pre-mRNA and the interactions between the splice editor and pre-mRNA that yield a trans-splicing mRNA product.

FIG. 1E provides a graph showing the proportion of ncRNAs identified as containing a sequence motif (Sm sequence motif, H/ACA box, and/or C/D box).

FIG. 1F provides a graph showing the length in number of nucleotides for exemplary ncRNAs of the disclosure.

FIG. 2A, FIG. 2B, and FIG. 2C provide a schematic, without wishing to be bound by theory, depicting the extended and folded secondary structure of an exemplary splice editor nucleic acid molecule of the disclosure having 5′ to 3′ an RNA binding domain, U1 snRNA, intron, SA, and exon (FIG. 2A) and the interaction of the exemplary splice editor with a target pre-mRNA that initiates a trans-splicing event between the SD of the pre-mRNA and the SA of the exemplary splice editor (FIG. 2A, FIG. 2B).

FIG. 3A, FIG. 3B, and FIG. 3C provide a schematic, without wishing to be bound by theory, depicting the extended and folded secondary structure of an exemplary splice editor nucleic acid molecule of the disclosure having 5′ to 3′ an RNA binding domain, U11 snRNA, intron, SA, and exon (FIG. 3A) and the interaction of the exemplary splice editor with a target pre-mRNA that initiates a trans-splicing event between the SD of the pre-mRNA and the SA of the exemplary splice editor (FIG. 3A, FIG. 3B).

FIG. 4A, FIG. 4B, and FIG. 4C provide a schematic, without wishing to be bound by theory, depicting the extended and folded secondary structure of an exemplary splice editor nucleic acid molecule of the disclosure having 5′ to 3′ an RNA binding domain, an ncRNA having an Sm sequence motif and a U7 snRNA, intron, SA, and exon (FIG. 4A) and the interaction of the exemplary splice editor with a target pre-mRNA that initiates a trans-splicing event between the SD of the pre-mRNA and the SA of the exemplary splice editor (FIG. 4A, FIG. 4B, and FIG. 4C).

FIG. 5A, FIG. 5B, and FIG. 5C provides a schematic, without wishing to be bound by theory, depicting the extended and folded secondary structure of an exemplary splice editor nucleic acid molecule of the disclosure having 5′ to 3′ an RNA binding domain, an ncRNA having an Sm sequence motif, intron, SA, and exon (FIG. 5A) and the interaction of the exemplary splice editor with a target pre-mRNA that initiates a trans-splicing event between the SD of the pre-mRNA and the SA of the exemplary splice editor (FIG. 5B, and FIG. 5C).

FIG. 6A, FIG. 6B, and FIG. 6C provides a schematic, without wishing to be bound by theory, depicting the extended and folded secondary structure of an exemplary splice editor nucleic acid molecule of the disclosure having 5′ to 3′ a snoRNA having an insertion of two RNA binding domains, intron, SA, and exon (FIG. 6A) and the interaction of the exemplary splice editor with a target pre-mRNA that initiates a trans-splicing event between the SD of the pre-mRNA and the SA of the exemplary splice editor (FIG. 6B, and FIG. 6C).

FIG. 7A is an image showing, without wishing to be bound by theory, the design of snoRNA guide constructs for exon skipping.

FIG. 7B is a graph showing exon skipping results for four splice acceptor targeting 25 snoRNAs (i.e., labeled “snord45a_11_acceptor”, “snord45a_7_acceptor”, “aca46_acceptor_29”, and “aca44_2_acceptor_35”, and sequences shown in Table 4).

FIG. 8 is a graph showing GFP production as a result of exon skipping from four different splice acceptor targeting snoRNAs (i.e., labeled “V5_snord45a_11_acceptor”, “V4_snord45a_7_acceptor”, “V1_aca46_acceptor_29”, and “V2_aca44_2_acceptor_35”, and sequences shown in Table 4).

FIG. 9 is a graph showing exon skipping results for four splice acceptor targeting snoRNAs (i.e., labeled “snord45a_11_acceptor”, “snord45a_7_acceptor”, “aca46_acceptor_29”, and “aca44_2_acceptor_35”) compared to the four randomized guides of FIG. 8.

FIG. 10 is an image showing, without wishing to be bound by theory, the design of U7 guide constructs for trans-splicing.

FIG. 11 is a graph showing the binding of the U7 guide constructs to different target elements and the effects of trans-splicing.

FIG. 12 is a graph showing independent validation of the results from FIG. 11. The first four bars from the left side show U7 constructs with specific targeting elements for trans-splicing, and the remaining bars on the graph show U7 constructs with non-targeting elements for trans-splicing.

FIG. 13 is a graph showing U7 targeting piggybac-integrated USH2A (773; hybridizing region and hairpin intact) compared to non-targeting guides (774 and 776), and mutated U7 hairpins (775; intact hybridizing region, but hairpin region mutated).

### DETAILED DESCRIPTION

The present disclosure provides nucleic acid molecules for targeting trans-splicing of a target RNA (e.g., a pre-mRNA) in a cell. In some embodiments, the nucleic acid molecules are engineered to comprise a nucleotide sequence comprising (i) at least one noncoding sequence (an intronic sequence) comprising an RNA-guided domain that binds to one or more target sequences in the target RNA (e.g., the pre-mRNA), (ii) a splice acceptor and/or splice donor, and (iii) at least one coding sequence (an exonic sequence). The nucleic acid molecules of the disclosure are referred to herein as “splice editor nucleic acids” or “splice editor nucleic acid molecules.” Without being bound by theory, the binding event brings the splice editor nucleic acid into proximity of a region of the target RNA (e.g., pre-mRNA) selected for trans-splicing and recruits the spliceosome to the target RNA (e.g., pre-mRNA) such that efficient trans-splicing occurs. In some embodiments, the target RNA is a pre-mRNA. In some embodiments, the pre-mRNA comprises a nucleotide sequence comprising a disease-causing mutation. In some embodiments, the trans-splicing generates a mRNA comprising a desired alteration compared to an mRNA generated by cis-splicing of the pre-mRNA. For example, in some embodiments, the desired alteration is correction of a disease-causing mutation in the pre-mRNA.

In some embodiments, the RNA-guided domain comprises (i) one or more binding domains each having complementarity to a target sequence in the target RNA (e.g., pre-mRNA), and (ii) a non-coding RNA (ncRNA) sequence. In some embodiments, the ncRNA sequence comprises a secondary structure and/or a sequence motif that assembles into a ribonucleoprotein (RNP). Without being bound by theory, assembly of the ncRNA to form an RNP endows the trans-splicing nucleic acid molecule with one or more desirable properties that enable efficient trans-splicing. For example, in some embodiments, the RNP functions to (i) stabilize RNA secondary structures present in the splice editor nucleic acid molecule, the pre-mRNA, or both; (ii) stabilize RNA-RNA interactions formed between the splice editor nucleic acid molecule and the pre-mRNA; (iii) protect the splice editor nucleic acid molecule and/or the pre-mRNA from degradation; (iv) localize the splice editor nucleic acid molecule to a subcellular compartment where pre-mRNA is present; and (v) a combination of (i)-(iv).

In some embodiments, the disclosure provides methods of targeting trans-splicing of a target RNA (e.g., pre-mRNA) in a cell, comprising introducing to the cell a splice editor nucleic acid molecule described herein. In some embodiments, the disclosure provides methods of correcting a mutation in at target RNA (e.g., a pre-mRNA) in a cell, comprising introducing to the cell a splice editor nucleic acid molecule described herein. In some aspects, the introducing is performed in vivo. In some embodiments, the introducing is performed ex vivo. In some embodiments, the methods described herein are used to introduce a desired edit to a target nucleic acid edit in a manner that avoids certain disadvantages of gene-editing, e.g., gene-editing performed using a CRISPR/Cas system. Whereas gene-editing is associated with a risk of introducing a permanent and disease-causing off-target edit to the genome, the present disclosure provides methods of trans-splicing that avoid altering genomic DNA and enable transient editing. Thus, and without being bound by theory, the methods of the disclosure are used to introduce edits to nucleic acids in a cell in a manner that is safer than gene-editing. Additionally, in some embodiments, the methods of the disclosure are used to inactivate an undesirable off-target gene edit introduced to the genome, thereby preventing or ameliorating deleterious phenotypes associated with gene editing approaches.

In some embodiments, the disclosure provides methods for treating a disease or disorder in a subject in need thereof, the disease or disorder associated with (i) one or more genetic mutations, and/or (ii) an aberrant expression level and/or activity of a gene, or a transcriptional or translational product thereof, comprising administering to a subject one or more splice editor nucleic acid molecules described herein.

In some embodiments, the disclosure provides methods and compositions for delivery of the splice editor nucleic acid molecule to a cell or a subject. In some embodiments, the splice editor nucleic acid molecule is delivered as a DNA. In some embodiments, the splice editor nucleic acid molecule is delivered as an RNA. In some aspects, the delivery comprises administering a recombinant expression vector (e.g., a viral vector, e.g., an AAV) comprising the splice editor nucleic acid molecule. In some aspects, the delivery comprises administering a non-viral vector (e.g., a lipid particle) comprising the splice editor nucleic acid molecule.

### Splice Editor Nucleic Acid Molecules for Targeted Trans-Splicing

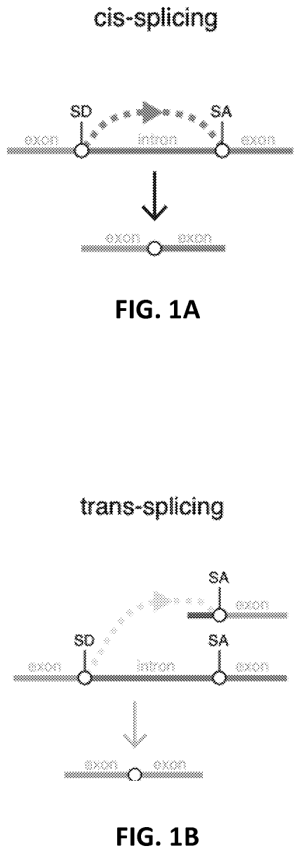

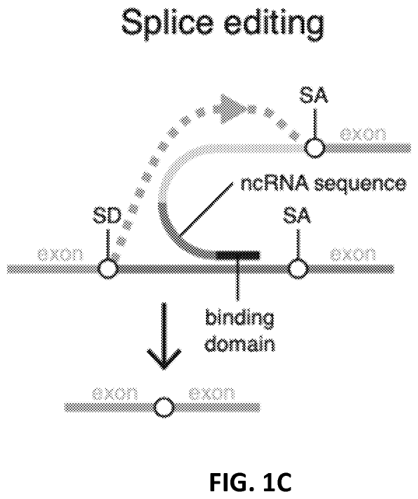

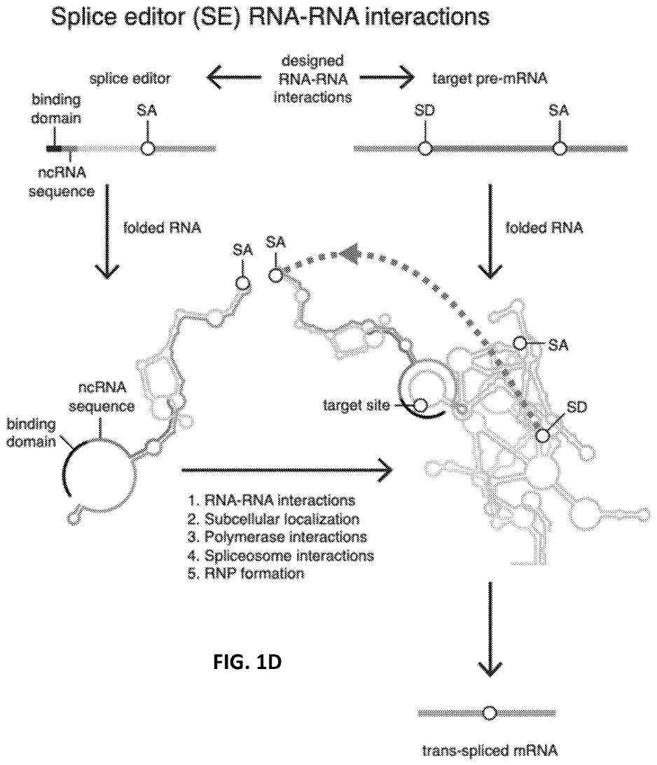

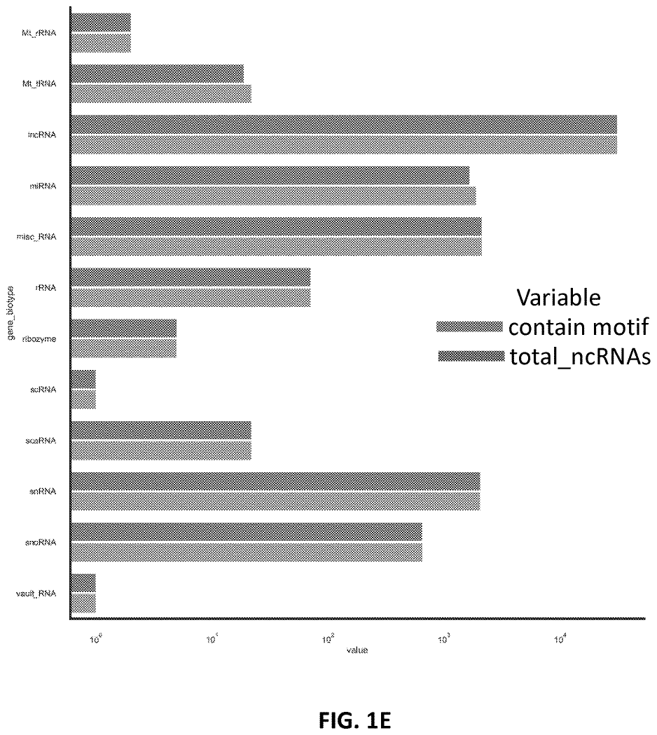

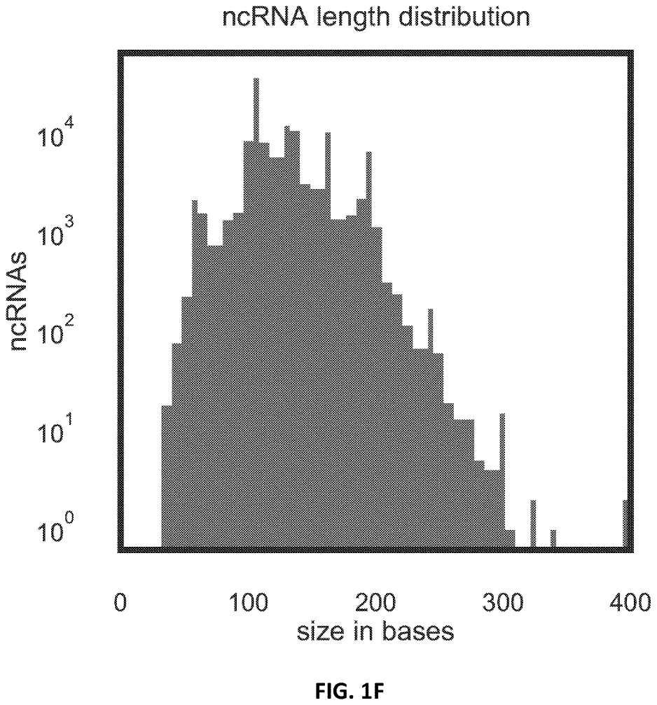

Accurate pre-mRNA splicing is critical for proper protein expression. Nuclear pre-mRNA splicing is catalyzed by the spliceosome. Vertebrate gene architecture often consists of relatively long introns and short internal exons. The exon-intron boundaries are defined by a splice donor (the 5′ splice site or splice site at the 3′end of an exon) and a splice acceptor (the 3′splice site or splice site at the 5′end of an exon). In addition to recognizing splice sites, the spliceosome relies on various splicing signals to mediate a splicing event, including a branch point sequence and a polypyrimidine tract. Typically, the branch point sequence comprises an adenosine situated within a consensus sequence and is situated about 18-40 nucleotides upstream of the 3′ splice site. The polypyrimidine tract comprises a repetitive sequence of uracils and is proximal the 3′splice site. Alternative signals can enhance or decrease splicing activity, including exonic splicing enhancers (ESEs), exonic splicing silencers (ESSs), intronic splicing enhancers (ISEs), and intronic splicing silencers (ISSs). Splicing in cis (“cis-splicing”) occurs when the 2′OH group of the branch adenosine of the intron carries out a nucleophilic attack on the 5′splice site (splice donor). This results in cleavage at this site and ligation of the 5′end of the intron to the branch adenosine, forming a lariat structure. The 3′splice site (splice acceptor) is attacked by the 3′OH of the 5′exon, resulting in ligation of the 5′ and 3′ exons to form the mRNA and release of the intron lariat (see, e.g., FIG. 1A).

In contrast, splicing in trans (“trans-splicing”) occurs between two different RNA molecules, wherein the 3′splice site (splice acceptor) of a second RNA is attacked by the 3′OH of the 5′exon of a first RNA, resulting in ligation of the 5′exon of the first RNA and the 3′exon of the second RNA, thereby forming a chimeric RNA (see, e.g., FIG. 1B).

The present disclosure provides splice editor nucleic acid molecules for targeting trans-splicing of a target RNA (e.g., pre-mRNA) in a cell, the splice editor nucleic acid molecule comprising a nucleotide sequence comprising (i) at least one intronic sequence comprising an RNA-guided domain; (ii) one or more splice sites (e.g., a splice acceptor and/or splice donor); and (iii) at least one exonic sequence. In some embodiments, the RNA-guided domain is designed to bind to a specific region of a target RNA (e.g., pre-mRNA), thereby enabling splicing between the one or more splice sites of the splice editor nucleic acid molecule and one or more splice sites of the target RNA (e.g., pre-mRNA). In some embodiments, the trans-splicing results in a chimeric mRNA comprising the at least one exonic sequence of the splice editor nucleic acid and one or more exons of the target RNA (e.g., pre-mRNA).

In humans, exon definition is determined by splice sites paired across an exon (e.g., a splice acceptor (3′splice site) at the 5′end of the exon and a splice donor (5′splice site) at the 3′end of the exon). Other splicing signals (e.g., branch point sequences, polypyrimidine tracts, exonic (or intronic) splicing enhancers and silencers) contribute to proper splicing together of exons to form a mature mRNA. During pre-mRNA splicing, the spliceosome searches for a pair of closely spaced splice sites. Without being bound by theory, the splice editor nucleic acid molecules described herein mediate efficient trans-splicing by bringing a splice site of the target RNA (e.g., pre-mRNA, e.g., a splice acceptor or splice donor of the target pre-mRNA) into close proximity with a splice site of the splice editor nucleic acid molecule (e.g., a splice acceptor or splice donor of the splice editor nucleic acid molecule), such that the spliceosome mediates splicing between the splice site of the target pre-mRNA and the splice site of the splice editor nucleic acid molecule.

In some embodiments, the splice editor nucleic acid molecule comprises a nucleotide sequence comprising from 5′ to 3′ (i) at least one intronic sequence comprising an RNA-guided domain; (ii) a splice acceptor; and (iii) at least one exonic sequence.

In some embodiments, the splice editor nucleic acid molecule comprises a nucleotide sequence comprising from 5′ to 3′ (i) at least one exonic sequence; (ii) a splice donor; and (iii) at least one intronic sequence comprising an RNA-guided domain.

In some embodiments, the at least one intronic sequences comprises one or more splicing signals (e.g., a branch point sequence, a polypyrimidine tract, an ISE, and/or an ISS). In some embodiments, the at least one exonic sequences comprises one or more splicing signals (e.g., an ESE and/or an ESS).

### RNA Guided Domain

In some embodiments, the RNA guided domain comprises a nucleotide sequence comprising (i) one or more binding domains, each having complementarity to a target sequence in the target RNA (e.g., pre-mRNA); and (ii) a ncRNA. In some embodiments, the one or more binding domains mediate binding of the trans-splicing nucleic acid molecules to a target RNA (e.g., pre-mRNA) in a cell. In some embodiments, the ncRNA mediates assembly into an RNP.

In some embodiments, the RNA guided domain comprises a nucleotide sequence having from 5′ to 3′: (i) one or more binding domains, each having complementarity to a target sequence in the target RNA (e.g., pre-mRNA); and (ii) a ncRNA.

In some embodiments, the RNA guided domain comprises a nucleotide sequence having from 5′ to 3′: (i) a ncRNA; and (ii) one or more binding domains, each having complementarity to a target sequence in the target RNA (e.g., pre-mRNA).

In some embodiments, the RNA guided domain comprises a nucleotide sequence having a ncRNA, wherein the one or more binding domains are inserted into the ncRNA or exchanged for contiguous nucleotides of the ncRNA.

### Target Sequence

In some embodiments, the one or more binding domains of the RNA guided domain are each complementary to a target sequence in a target RNA (e.g., pre-mRNA) targeted for trans-splicing.

As used herein, the term “target sequence” refers to a sequence of contiguous nucleotides present in a target RNA (e.g., pre-mRNA) targeted for trans-splicing. As used herein, the term “contiguous nucleotides” refers to a string of nucleotides that are covalently linked and immediately adjacent to one another. In some embodiments, the target sequence is at least about 4, 5, 6, 7, 8, 9, 10, 11, 12, 13, 14, 15, 16, 17, 18, 19, or 20 nucleotides in length. In some embodiments, the target sequence is less than about 300, 250, 200, 100, 150, or 50 nucleotides in length. In some embodiments, the target sequence is about 5-10, about 5-15, about 5-20, about 10-20, about 10-30, about 10-40, about 10-50, about 10-60, about 10-70, about 10-80, about 10-90, about 10-100, about 50-100, about 50-150, about 50-200, about 50-250, about 50-300, about 100-200, about 100-300, or about 200-300 nucleotides in length.

In some embodiments, the target sequence is in a region comprising a splice site in the target RNA (e.g., pre-mRNA) targeted for trans-splicing. As used herein, “a splice site in the target RNA (e.g., pre-mRNA) targeted for trans-splicing” refers to a splice site in the target RNA (e.g., pre-mRNA) selected for trans-splicing, wherein upon introducing a splice editor nucleic acid molecule described herein to a cell comprising the target RNA (e.g., pre-mRNA), a trans-splicing event mediates ligation between the splice site of the target RNA (e.g., pre-mRNA) and a splice site of the splice editor nucleic acid molecule. In some embodiments, the target sequence is upstream the splice site in the target RNA (e.g., pre-mRNA) targeted for trans-splicing. In some embodiments, the target sequence is downstream the splice site in the target RNA (e.g., pre-mRNA) targeted for trans-splicing. In some embodiments, the target sequence is in a region comprising the splice site in the target RNA (e.g., pre-mRNA) targeted for trans-splicing, wherein the region spans at least about 50, about 100, about 150, about 200, about 300, about 400, about 500, about 1,000, about 2,000, about 3,000, about 4,000, about 5,000 nucleotides.

In some embodiments, the target sequence is proximal to the splice site in the target RNA (e.g., pre-mRNA) targeted for trans-splicing. As used herein, the term “proximal to the splice site” refers to a region of less than about 500 nucleotides extending upstream and/or downstream of the splice site in the target RNA (e.g., pre-mRNA) targeted for trans-splicing.

In some embodiments, the target sequence is proximal to a splice acceptor targeted for trans-splicing. In some embodiments, the target sequence is upstream a splice acceptor targeted for trans-splicing. In some embodiments, the target sequence is downstream of a splice acceptor targeted for trans-splicing. In some embodiments, the target sequence overlaps a splice acceptor targeted for trans-splicing.

In some embodiments, the target sequence is proximal to a splice donor targeted for trans-splicing. In some embodiments, the target sequence is upstream a splice donor targeted for trans-splicing. In some embodiments, the target sequence is downstream of a splice donor targeted for trans-splicing. In some embodiments, the target sequence overlaps a splice donor targeted for trans-splicing.

In some embodiments, the target sequence is in a region of the target RNA (e.g., pre-mRNA) comprising an exon targeted for trans-splicing. As used herein, an “exon targeted for trans-splicing” refers to an exon in the target RNA that is selected for removal following trans-splicing between the target RNA and a splice editor nucleic acid described herein, wherein the trans-splicing results in ligation between one or more exons of the target RNA (e.g., pre-mRNA) and the at least one exonic sequence of the splice editor nucleic acid to form a chimeric RNA molecule, and wherein the exon targeted for trans-splicing is present in the target RNA, but absent in the chimeric RNA molecule formed by the trans-splicing event.

In some embodiments, the target sequence is upstream the exon targeted for trans-splicing. In some embodiments, the target sequence is downstream the exon targeted for trans-splicing. In some embodiments, the target sequence is within the exon targeted for trans-splicing.

In some embodiments, the target sequence is proximal to a splice acceptor of the exon targeted for trans-splicing. In some embodiments, the target sequence is upstream the splice acceptor of the exon targeted for trans-splicing. In some embodiments, the target sequence is downstream the splice acceptor of the exon targeted for trans-splicing. In some embodiments, the target sequence overlaps the splice acceptor of the exon targeted for trans-splicing.

In some embodiments, the target sequence is proximal to a splice donor of the exon targeted for trans-splicing. In some embodiments, the target sequence is upstream the splice donor of the exon targeted for trans-splicing. In some embodiments, the target sequence is downstream the splice donor of the exon targeted for trans-splicing. In some embodiments, the target sequence overlaps the splice donor of the exon targeted for trans-splicing.

### RNA Binding Domain

In some embodiments, the binding domain complementary to a target sequence in the target RNA (e.g., pre-mRNA) is at least 4 nucleotides in length. In some embodiments, the binding domain is less than about 300 nucleotides in length. In some embodiments, the binding domain is at least about 5, about 6, about 7, about 8, about 9, about 10, about 11, about 12, about 13, about 14, about 15, about 16, about 17, about 18, about 19 or about 20 nucleotides in length. In some embodiments, the binding domain about 250 to about 300, about 200 to about 300, about 150 to about 300, about 100 to about 300, about 50 to about 300, about 100 to about 250, about 100 to about 200, about 100 to about 150, about 50 to about 250, about 50 to about 200, about 50 to about 150, about 50 to about 100, or about 300, 250, 200, 150, 100, or 50 nucleotides in length.

In some embodiments, the binding domain is about 5 to about 20, about 5 to about 30, about 5 to about 40, about 5 to about 50, about 10 to about 50, about 10 to about 100, about 20 to about 100, about 30 to about 100, about 40 to about 100, about 50 to about 100, about 50 to about 150, about 50 to about 200, about 50 to about 250, about 100 to about 150, about 100 to about 200, about 100 to about 250, or about 100 to about 300 nucleotides in length.

In some embodiments, the binding domain is 10-50 nucleotides in length, e.g., 10-45, 10-40, 10-35, 10-30, 10-20, 11-45, 11-40, 11-35, 11-30, 11-20, 12-45, 12-40, 12-35, 12-30, 12-25, 12-20, 13-45, 13-40, 13-35, 13-30, 13-25, 13-20, 14-45, 14-40, 14-35, 14-30, 14-25, 14-20, 15-45, 15-40, 15-35, 15-30, 15-25, 15-20, 16-45, 16-40, 16-35, 16-30, 16-25, 16-20, 17-45, 17-40, 17-35, 17-30, 17-25, 17-20, 18-45, 18-40, 18-35, 18-30, 18-25, 18-20, 19-45, 19-40, 19-35, 19-30, 19-25, 19-20, e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In some embodiments, the RNA-guided domain comprises one binding domain. In some embodiments, the RNA-guided domain comprises more than one binding domain. In some embodiments, the RNA-guided domain comprises 2, 3, 4, 5, 6, 7, 8, 9, or 10 binding domains. In some embodiments, the more than one binding domains are immediately adjacent to one another. In some embodiments, the more than one binding domains are linked by an intervening nucleotide spacer sequence.

In some embodiments, the one or more binding domains each comprise a sequence that is sufficiently complementary to its target sequence to enable the splice editor nucleic acid molecule to specifically bind to the target sequence by forming base pairs. As used herein, the term “base pair” refers to two nucleobases on opposite complementary nucleic acid strands that interact by formation of specific hydrogen bonding (e.g., Watson-Crick, Hoogsteen, or reversed Hoogsteen hydrogen bonding). In some embodiments, the base pair is formed by Watson-Crick base pairing. As understood by the skilled artisan, Watson-Crick base pairing refers to the set of base pairing rules wherein a purine nucleobase binds to a pyrimidine nucleobase to form a complementary base pair. The nature of the hydrogen bonding depends upon the particular base pair. For example, a guanosine-cytosine base pair is formed by three hydrogen bonds and the adenine-thymine or adenine-uracil base pair is formed by two hydrogen bonds. It is understood that analogs or derivatives of canonical nucleobases will form base pair interactions via Watson Crick base pairing or non-canonical base pairing.

A binding domain that “specifically binds to” a target sequence in a target RNA (e.g., pre-mRNA) refers to one that will not appreciably bind to a reference sequence, e.g., a nucleic acid lacking the target sequence. For example, a splice editor nucleic acid molecule comprising a binding domain that specifically binds a target sequence will exhibit substantially higher binding affinity for a target RNA (e.g., pre-mRNA) comprising a nucleotide sequence comprising the target sequence compared to a target RNA (e.g., pre-mRNA) lacking the target sequence. As is understood by the skilled artisan, the binding affinity between a first nucleic acid strand and a second nucleic acid strand is measured as the melting temperature (Tm), which is the temperature at which half the first nucleic acid strand is duplexed to the second nucleic acid strand.

In some embodiments, a binding domain is complementary to a target sequence in the target RNA (e.g., pre-mRNA) if it base-pairs to the target sequence under conditions suitable for modulating trans-splicing. Such conditions can be stringent conditions, e.g., combination of the target RNA (e.g., pre-mRNA) and splice editor nucleic acid molecule in buffer comprising 400 mM NaCl, 40 mM PIPES pH 6.4, 1 mM EDTA at a temperature of 50° C.-70° C. for 12-16 hours, followed by washing (see, e.g., “Molecular Cloning: A Laboratory Manual, Sambrook, et al. (1989) Cold Spring Harbor Laboratory Press). Other conditions include physiologically relevant conditions as can be encountered inside an organism. The skilled person will be able to determine the set of conditions most appropriate for a test of complementarity of two sequences in accordance with the ultimate application of the hybridized nucleotides.

### Non-Coding RNAs

In some embodiments, the RNA-guided domain comprises a ncRNA. As used herein, a “ncRNA” refers to an RNA sequence that does not encode a protein and functions in one or more cellular regulatory processes (e.g., RNA splicing, histone modification, translation, RNA pseudouridylation, RNA methylation, RNA cleavage, RNA processing and RNA modification).

For example, certain ncRNAs function in RNA-guided systems that have evolved to (i) stabilize RNA secondary structures and RNA-RNA interactions (Rossi 1996 Journal of Biological Chemistry 271.39 (1996): 23985-23991, Sabath, et al (2013) RNA 19:1726-1744; Skrajna, et al (2017) RNA 23:938-951); (ii) assemble into ribonucleoproteins (RNPs) to envelope and protect RNA from degradation (Darzacq 2006); and (iii) localize to relevant subcellular compartments (Roithovi, et al (2018) Nucleic acids research 46:3774-3790). In some embodiments, the ncRNA is identified according to a method described herein.

In some embodiments, the method comprises identifying a ncRNA sequence from a database. Databases listing ncRNA sequences are known in the art. For example, in some embodiments, the database is RNAcentral (see, e.g., Nucleic Acids Res 45:D128 (2017). RNAcentral is a searchable database that provides ncRNA sequences annotated with unique identifiers and information regarding the one or more species in which the RNA sequence has been observed.

In some embodiments, the method comprises identifying a ncRNA expressed by a cell or organism. Methods to identify ncRNAs are known in the art (see, e.g., Huttenhofer, et al (2006) Nucleic Acids Res 34:635). In some embodiments, cellular RNA is extracted from a cell or organism, separated by PAGE and elution from the gel, and ncRNAs are identified by sequence analysis (e.g., via 2D RNA fingerprinting or enzymatic or chemical RNA sequencing). In some embodiments, a cDNA library is generated by reverse transcription of ncRNAs obtained from a cell or organism through a selection process based on size or antibody-binding that is then subjected to sequence analysis. In some embodiments, total RNA is harvested from a cell or organism and microarray hybridization is used to detect ncRNAs. In some embodiments, genomic SELEX is used to identify ncRNAs obtained from a cell or organism. In some embodiments, the ncRNA sequence is identified from any known organism. In some embodiments, the organism is a bacteria. In some embodiments, the organism is a archaebacteria. In some embodiments, the organism is a metazoan. In some embodiments, the organism is a vertebrate. In some embodiments, the organism is a mammal, amphibian, reptile, fish, or bird. In some embodiments, the organism is a human.

In some embodiments, ncRNA functions to modify, alter, inhibit, or promote RNP formation and/or canonical processing. In some embodiments, the ncRNA assembles into an RNP. In some embodiments, the RNP functions to stabilize the RNA secondary structure of the splice editor nucleic acid. In some embodiments, the RNP functions in to stabilize RNA-RNA interactions within the splice editor nucleic acid and/or with a target RNA (e.g., pre-mRNA). In some embodiments, the RNP functions in to protect the splice editor nucleic acid from degradation. In some embodiments, the RNP functions in to localize the splice editor nucleic acid to a subcellular compartment comprising a target pre-mRNA. Methods to measure assembly of one or more nucleic acids (e.g., RNA or DNA) and one or more proteins to form an RNP are known in the art. Such methods include, but are not limited to, electrophoretic mobility shift assay (EMSA), DNA or RNA pull-down assays, oligonucleotide-targeted RNase H protection assays, fluorescent in situ hybridization co-localization, co-immunoprecipitation assays, and RNA sequencing and cross-linking methods such as high throughput sequencing crosslinking immunoprecipitation (HITS-CLIP).

In some embodiments, a ncRNA sequence identified according to a method described herein is incorporated into a splice editor nucleic acid of the disclosure. In some embodiments, the entire ncRNA sequence is incorporated into the splice editor nucleic acid. In some embodiments, a portion of the ncRNA sequence is incorporated into the splice editor nucleic acid.

In some embodiments, a splice editor of the disclosure comprises an ncRNA sequence or portion thereof, wherein the ncRNA is selected from an snRNA, a snoRNA, a lncRNA, a an rRNA, a ribozyme, an sRNA, a scaRNA, a vault RNA, and a combination thereof.

In some embodiments, the ncRNA sequence or portion thereof is less than about 500 nucleotides in length. In some embodiments, the ncRNA sequence or portion thereof is less than about 400 nucleotides in length. In some embodiments, the ncRNA sequence or portion thereof is less than about 300 nucleotides in length. In some embodiments, the ncRNA sequence or portion thereof is about 250 to about 300, about 200 to about 300, about 150 to about 300, about 100 to about 300, about 50 to about 300, about 100 to about 250, about 100 to about 200, about 100 to about 150, about 50 to about 250, about 50 to about 200, about 50 to about 150, about 50 to about 100, or about 300, 250, 200, 150, 100, or 50 nucleotides in length.

In some embodiments, the ncRNA sequence or portion thereof is at least about 5, about 6, about 7, about 8, about 9, about 10, about 11, about 12, about 13, about 14, about 15, about 16, about 17, about 18, about 19 or about 20 nucleotides in length.

In some embodiments, the ncRNA sequence or portion thereof is about 5 to about 20, about 5 to about 30, about 5 to about 40, about 5 to about 50, about 10 to about 50, about 10 to about 100, about 20 to about 100, about 30 to about 100, about 40 to about 100, about 50 to about 100, about 50 to about 150, about 50 to about 200, about 50 to about 250, about 100 to about 150, about 100 to about 200, about 100 to about 250, or about 100 to about 300 nucleotides in length.

In some embodiments, the ncRNA sequence or portion thereof comprises one or more RNA secondary structures that assembles into an RNP. Methods to determine the secondary structure formed by an RNA sequence are known in the art. In some embodiments, the method comprises an experimental assay, e.g., nuclear magnetic resonance, cryo-electron microscopy, or X-ray crystal structure analysis. In some embodiments, the method comprises a computational prediction, e.g., based on a thermodynamic model such as Turner's nearest-neighbor model (Schroeder, et al, Methods Enzymol. 468, 371-387 (2009); Turner, et al Nucleic Acids Res. 38, D280-2 (2010)) or the Zuker algorithm (Zuker, et al Nucleic Acids Res. 9, 133-148 (1981); Zuker, et al Nucleic Acids Res. 31, 3406-3415 (2003); Markham, et al Methods Mol. Biol. 453, 3-31 (2008); Hofacker, et al Nucleic Acids Res. 31, 3429-3431 (2003); Lorenz, Algorithms Mol. Biol. 6, 26 (2011); Matthews, et al Molecular Modeling of Nucleic Acids. Vol. 682 of ACS Symposium Series. 246-257; Reuther, et al BMC Bioinform. 11, 129 (2010)); a machine learning technique such as CONTRAfold (Do, et al Bioinformatics 22, e90-8 (2006); Foo, et al Advances in Neural Information Processing Systems 20, 377-384), ContextFold (Zakov, et al J. Comput. Biol. 18, 1525-1542 (2011)); a probabilistic generative model such as stochastic context-free grammars (Rivas, et al RNA 18, 193-212 (2012)); a hybrid model such as SimFold (Andronescu, et al Bioinformatics 23, i19-28 (2007); Andronescu et al RNA 16, 2304-2318 (2010)) or MXfold (Akiyama, et al J. Bioinform. Comput. Biol. 16, 1840025 (2018)); a deep learning approach such as SPOT-RNA (Singh, et al Nat. Commun. 10, 5407 (2019)) or E2Efold (Chen et al Proceedings of the 8th International Conference on Learning Representations; arXiv:2002.05810 (2020)).

In some embodiments, the one or more RNA secondary structures comprises a single-stranded RNA sequence, a double-stranded RNA sequence, or a combination thereof. In some embodiments, the one or more RNA secondary structure comprises a duplex structure, a stem-loop, a pseudoknot, an internal loop, a multi-branch loop, a bulge loop, an external loop, or a combination thereof. In some embodiments, the ncRNA sequence or portion thereof comprises a sequence motif that assembles into an RNP. In some embodiments, the sequence motif comprises a single-stranded RNA sequence that assembles into an RNP. In some embodiments, the ncRNA sequence or portion thereof comprises a sequence motif and one or more RNA secondary structures that assemble into an RNP. In some embodiments, the secondary structure and/or a sequence motif assembles with one or more proteins in a human cell to form an RNP.

In some embodiments, the ncRNA sequence or portion thereof comprises one or more RNA secondary structures. In some embodiments, the ncRNA sequence or portion thereof comprises one or more sequence motifs. In some embodiments, the ncRNA sequence or portion thereof comprises one or more RNA secondary structures and one or more sequence motifs. In some embodiments, the sequence motif comprises a sequence selected from Table 1. In some embodiments, the sequence motif comprises an H consensus sequence comprising or consisting of a sequence set forth in Table 1. In some embodiments, the H consensus sequence comprises or consists of SEQ ID NO: 1. In some embodiments, the sequence motif comprises an ACA consensus sequence comprising or consisting of a sequence set forth in Table 1. In some embodiments, the ACA consensus sequence comprises or consists of SEQ ID NO: 2. In some embodiments, the sequence motif comprises an H/ACA box, wherein the H/ACA box comprises a sequence comprising an H consensus sequence and an ACA consensus sequence, each comprising a sequence set forth in Table 1. In some embodiments, the H/ACA box comprises a sequence comprising SEQ ID NO: 1 and SEQ ID NO: 2. In some embodiments, the sequence motif comprises a C consensus sequence comprising or consisting of a sequence set forth in Table 1. In some embodiments, the C consensus sequence comprises or consists of SEQ ID NO: 5. In some embodiments, the sequence motif comprises a D consensus sequence comprising or consisting of a sequence set forth in Table 1. In some embodiments, the D consensus sequence comprises or consists of SEQ ID NO: 6. In some embodiments, the sequence motif comprises a C/D box, wherein the C/D box comprises a C consensus sequence and a D consensus sequence, each comprising a sequence set forth in Table 1. In some embodiments, the C/D box comprises SEQ ID NO: 5 and SEQ ID NO: 6. In some embodiments, the sequence motif comprises an Sm motif comprising a sequence set forth in Table 1. In some embodiments, the Sm motif comprises SEQ ID NO: 3. In some embodiments, the Sm motif comprises SEQ ID NO: 4.

TABLE 1
ncRNA Sequence Motifs

| Name/Identifier | RNA Sequence | SEQ ID NO |
|---|---|---|
| H box | ANANNA | 1 |
| | N = A, C, G, or U | |
| ACA box | ACA | 2 |
| Sm motif | AAUUUUUGG | 3 |
| Sm U7 motif | AAUUUGUCU | 4 |
| C box | RUGAUGA | 5 |
| | R = A or G | |
| D box | CUGA | 6 |
| C′ box | RUGAUGA | 7 |
| D′ box | CUGA | 8 |

In some embodiments, a splice editor of the disclosure comprises a ncRNA sequence having at least about 60%, about 65%, about 70%, about 75%, about 80%, about 85%, about 90%, about 91%, about 92%, about 93%, about 94%, about 95%, about 96%, about 97%, about 98%, about 99% identity to a sequence selected from SEQ ID NOs: 9-657 or portion thereof. In some embodiments, a splice editor of the disclosure comprises a ncRNA sequence selected from SEQ ID NOs: 9-657 or a portion thereof.

In some embodiments, the ncRNA sequence or portion thereof comprises a contiguous nucleotide sequence of at least about 5, about 6, about 7, about 8, about 9, about 10, about 11, about 12, about 13, about 14, about 15, about 16, about 17, about 18, about 19 or about 20 nucleotides in length, wherein the contiguous nucleotide sequence comprises a Sm sequence motif (e.g., an Sm sequence motif set forth in Table 1).

In some embodiments, the ncRNA sequence or portion thereof comprises a contiguous nucleotide sequence of about 5 to about 20, about 5 to about 30, about 5 to about 40, about 5 to about 50, about 10 to about 50, about 20 to about 50, about 30 to about 50, or about 40 to about 50 nucleotides in length, wherein the contiguous nucleotide sequence comprises a Sm sequence motif (e.g., an Sm sequence motif set forth in Table 1).

In some embodiments, the ncRNA sequence or portion thereof comprises a contiguous nucleotide sequence of about 30 to about 100, about 40 to about 100, about 50 to about 100, about 50 to about 150, about 50 to about 200, about 50 to about 250, about 100 to about 150, about 100 to about 200, about 100 to about 250, or about 100 to about 300 nucleotides in length, wherein the contiguous nucleotide sequence comprises a Sm sequence motif (e.g., an Sm sequence motif set forth in Table 1).

In some embodiments, the ncRNA sequence or portion thereof comprises a contiguous nucleotide sequence of about 30 to about 100, about 40 to about 100, about 50 to about 100, about 50 to about 150, about 50 to about 200, about 50 to about 250, about 100 to about 150, about 100 to about 200, about 100 to about 250, or about 100 to about 300 nucleotides in length, wherein the contiguous nucleotide sequence comprises a (i) H consensus sequence (e.g., a H consensus sequence set forth in Table 1); (ii) ACA consensus sequence (e.g., an ACA consensus sequence set forth in Table 1); or (iii) combination of (i)-(ii).

In some embodiments, the ncRNA sequence or portion thereof comprises a contiguous nucleotide sequence of about 30 to about 100, about 40 to about 100, about 50 to about 100, about 50 to about 150, about 50 to about 200, about 50 to about 250, about 100 to about 150, about 100 to about 200, about 100 to about 250, or about 100 to about 300 nucleotides in length, wherein the contiguous nucleotide sequence comprises a (i)C-box motif described herein (e.g., a C-box motif set forth in Table 1), (ii) C′-box motif described herein (e.g., a C′-box motif set forth in Table 1), (iii) D-box motif described herein (e.g., a D-box motif set forth in Table 1), (iv) a D′-box motif described herein (e.g., a D′-box motif set forth in Table 1), or (v) a combination of (i)-(iv).

In some embodiments, the ncRNA sequence or portion thereof comprises a nucleotide sequence having at least about 60%, about 65%, about 70%, about 75%, about 80%, about 85%, about 90%, about 91%, about 92%, about 93%, about 94%, about 95%, about 96%, about 97%, about 98%, about 99% identity to a sequence selected from SEQ ID NOs: 9-657, wherein the nucleotide sequence comprises a region of at least about 5, about 6, about 7, about 8, about 9, about 10, about 11, about 12, about 13, about 14, about 15, about 16, about 17, about 18, about 19 or about 20 nucleotides in length, wherein the region comprises one or more Sm sequence motifs (e.g., one or more Sm sequence motifs set forth in Table 1).

In some embodiments, the ncRNA sequence or portion thereof comprises a nucleotide sequence having at least about 60%, about 65%, about 70%, about 75%, about 80%, about 85%, about 90%, about 91%, about 92%, about 93%, about 94%, about 95%, about 96%, about 97%, about 98%, about 99% identity to a sequence selected from SEQ ID NOs: 9-657, wherein the nucleotide sequence comprises a region of at least about 5 to about 20, about 5 to about 30, about 5 to about 40, about 5 to about 50, about 10 to about 50, about 20 to about 50, about 30 to about 50, or about 40 to about 50 nucleotides in length, wherein the region comprises one or more Sm sequence motifs (e.g., one or more Sm sequence motifs set forth in Table 1).

In some embodiments, the ncRNA sequence or portion thereof comprises a nucleotide sequence having at least about 60%, about 65%, about 70%, about 75%, about 80%, about 85%, about 90%, about 91%, about 92%, about 93%, about 94%, about 95%, about 96%, about 97%, about 98%, about 99% identity to a sequence selected from SEQ ID NOs: 9-657, wherein the nucleotide sequence comprises a region of at least about 30 to about 100, about 40 to about 100, about 50 to about 100, about 50 to about 150, about 50 to about 200, about 50 to about 250, about 100 to about 150, about 100 to about 200, about 100 to about 250, or about 100 to about 300 nucleotides in length, wherein the region comprises one or more Sm sequence motifs (e.g., one or more Sm sequence motifs set forth in Table 1).

In some embodiments, the ncRNA sequence or portion thereof comprises a nucleotide sequence having at least about 60%, about 65%, about 70%, about 75%, about 80%, about 85%, about 90%, about 91%, about 92%, about 93%, about 94%, about 95%, about 96%, about 97%, about 98%, about 99% identity to a sequence selected from SEQ ID NOs: 9-657, wherein the nucleotide sequence comprises a region of at least about 30 to about 100, about 40 to about 100, about 50 to about 100, about 50 to about 150, about 50 to about 200, about 50 to about 250, about 100 to about 150, about 100 to about 200, about 100 to about 250, or about 100 to about 300 nucleotides in length, wherein the region comprises (i) an H consensus sequence (e.g., an H consensus sequence set forth in Table 1); (ii) an ACA consensus sequence (e.g., an ACA consensus sequence set forth in Table 1); or (iii) a combination of (i)-(ii).

In some embodiments, the ncRNA sequence or portion thereof comprises a nucleotide sequence having at least about 60%, about 65%, about 70%, about 75%, about 80%, about 85%, about 90%, about 91%, about 92%, about 93%, about 94%, about 95%, about 96%, about 97%, about 98%, about 99% identity to a sequence selected from SEQ ID NOs: 9-657, wherein the nucleotide sequence comprises a region of at least about 30 to about 100, about 40 to about 100, about 50 to about 100, about 50 to about 150, about 50 to about 200, about 50 to about 250, about 100 to about 150, about 100 to about 200, about 100 to about 250, or about 100 to about 300 nucleotides in length, (i) a C-box motif described herein (e.g., a C-box motif set forth in Table 1), (ii) a C′-box motif described herein (e.g., a C′-box motif set forth in Table 1), (iii) a D-box motif described herein (e.g., a D-box motif set forth in Table 1), (iv) a D′-box motif described herein (e.g., a D′-box motif set forth in Table 1), or (v) a combination of (i)-(iv).

In some embodiments, the ncRNA is an snRNA. In some embodiments, a splice editor nucleic acid molecule of the disclosure comprises a full-length snRNA or portion thereof comprising a nucleotide sequence comprising one or more sequence motifs described herein (e.g., one or more sequence motifs set forth in Table 1). In some embodiments, an snRNA sequence described herein or identified according to a method described herein is incorporated into a splice editor nucleic acid of the disclosure. In some embodiments, a full-length snRNA sequence described herein or identified according to a method described herein is incorporated into a splice editor nucleic acid of the disclosure. In some embodiments, a portion of a snRNA sequence described herein or identified according to a method described herein (e.g., a region of contiguous nucleotides in the snRNA) is incorporated into a splice editor nucleic acid of the disclosure. In some embodiments, the full-length snRNA or portion of the snRNA assembles into a small nuclear RNP (snRNP). In some embodiments, the full-length snRNA or the portion of the snRNA comprises one or more secondary RNA structures that assembles to form an snRNP. In some embodiments, the full-length snRNA or the portion of the snRNA comprises one or more sequence motifs that assembles to form an snRNP. In some embodiments, the full-length snRNA or the portion of the snRNA comprises (i) one or more one or more secondary RNA structures, and (ii) one or more sequence motifs, wherein (i), (ii), or both assemble to form an snRNP.

Exemplary metazoan snRNA systems include U1 and U11 snRNAs, which are snRNAs that guide spliceosome RNPs to splice sites (Black, et al (1985) Cell 42: 737-750; Kolossova, et al (1997) RNA 3: 227). Other snRNAs include U2, U4, U4atac, U5, U6, U6atac, and U12, which also form RNPs in the major and minor spliceosome (Turunen, et al (2013) RNA 4:61-76; Nguyen, et al (2015), Nature 523:47-52; Charenton, et al (2019) Science 364:362-367). U7 RNAs are responsible for histone pre-mRNA cleavage and polyadenylation (Strub, et al (1984) EMBO journal 3:2801-2807; Soldati, et al (1988), Molecular and Cellular Biology 8:1518-1524; Cotton, et al (1988) The EMBO Journal 7:801-808).

In some embodiments, a splice editor nucleic acid molecule of the disclosure comprises a full-length snRNA or portion thereof comprising a nucleotide sequence comprising one or more sequence motifs described herein (e.g., one or more sequence motifs set forth in Table 1), and wherein the snRNA is selected from RNU1-1, RNU1-100P, RNU1-101P, RNU1-103P, RNU1-104P, RNU1-105P, RNU1-107P, RNU1-108P, RNU1-109P, RNU1-112P, RNU1-114P, RNU1-115P, RNU1-116P, RNU1-117P, RNU1-119P, RNU1-11P, RNU1-123P, RNU1-124P, RNU1-125P, RNU1-128P, RNU1-129P, RNU1-130P, RNU1-131P, RNU1-132P, RNU1-133P, RNU1-134P, RNU1-136P, RNU1-138P, RNU1-139P, RNU1-140P, RNU1-141P, RNU1-142P, RNU1-143P, RNU1-146P, RNU1-148P, RNU1-149P, RNU1-14P, RNU1-150P, RNU1-151P, RNU1-153P, RNU1-154P, RNU1-155P, RNU1-15P, RNU1-16P, RNU1-17P, RNU1-18P, RNU1-19P, RNU1-2, RNU1-20P, RNU1-21P, RNU1-22P, RNU1-23P, RNU1-24P, RNU1-27P, RNU1-28P, RNU1-29P, RNU1-3, RNU1-30P, RNU1-31P, RNU1-32P, RNU1-33P, RNU1-34P, RNU1-35P, RNU1-36P, RNU1-38P, RNU1-39P, RNU1-4, RNU1-40P, RNU1-41P, RNU1-42P, RNU1-43P, RNU1-44P, RNU1-45P, RNU1-46P, RNU1-47P, RNU1-48P, RNU1-49P, RNU1-51P, RNU1-52P, RNU1-54P, RNU1-55P, RNU1-56P, RNU1-57P, RNU1-58P, RNU1-5P, RNU1-61P, RNU1-62P, RNU1-63P, RNU1-64P, RNU1-65P, RNU1-67P, RNU1-68P, RNU1-6P, RNU1-70P, RNU1-72P, RNU1-73P, RNU1-74P, RNU1-75P, RNU1-76P, RNU1-77P, RNU1-78P, RNU1-79P, RNU1-7P, RNU1-80P, RNU1-82P, RNU1-83P, RNU1-84P, RNU1-86P, RNU1-88P, RNU1-89P, RNU1-8P, RNU1-91P, RNU1-93P, RNU1-94P, RNU1-95P, RNU1-96P, RNU1-97P, RNU1-98P, RNU11, RNU11-2P, RNU11-3P, RNU11-4P, RNU11-5P, RNU11-6P, RNU12, RNU12-2P, RNU2-12P, RNU2-13P, RNU2-16P, RNU2-18P, RNU2-19P, RNU2-24P, RNU2-27P, RNU2-30P, RNU2-31P, RNU2-34P, RNU2-35P, RNU2-37P, RNU2-38P, RNU2-41P, RNU2-42P, RNU2-46P, RNU2-50P, RNU2-53P, RNU2-55P, RNU2-60P, RNU2-66P, RNU2-69P, RNU2-70P, RNU2-72P, RNU2-7P, RNU2-9P, RNU4-1, RNU4-10P, RNU4-11P, RNU4-12P, RNU4-13P, RNU4-14P, RNU4-15P, RNU4-16P, RNU4-17P, RNU4-18P, RNU4-2, RNU4-20P, RNU4-21P, RNU4-22P, RNU4-23P, RNU4-24P, RNU4-26P, RNU4-27P, RNU4-28P, RNU4-29P, RNU4-30P, RNU4-31P, RNU4-32P, RNU4-33P, RNU4-34P, RNU4-35P, RNU4-36P, RNU4-37P, RNU4-38P, RNU4-39P, RNU4-40P, RNU4-41P, RNU4-42P, RNU4-43P, RNU4-44P, RNU4-45P, RNU4-46P, RNU4-47P, RNU4-49P, RNU4-4P, RNU4-50P, RNU4-51P, RNU4-52P, RNU4-53P, RNU4-54P, RNU4-55P, RNU4-56P, RNU4-57P, RNU4-58P, RNU4-59P, RNU4-5P, RNU4-60P, RNU4-61P, RNU4-62P, RNU4-63P, RNU4-64P, RNU4-65P, RNU4-66P, RNU4-67P, RNU4-68P, RNU4-69P, RNU4-6P, RNU4-70P, RNU4-71P, RNU4-72P, RNU4-73P, RNU4-74P, RNU4-75P, RNU4-76P, RNU4-77P, RNU4-78P, RNU4-79P, RNU4-7P, RNU4-80P, RNU4-81P, RNU4-82P, RNU4-83P, RNU4-84P, RNU4-85P, RNU4-87P, RNU4-88P, RNU4-89P, RNU4-8P, RNU4-90P, RNU4-91P, RNU4-92P, RNU4-9P, RNU4ATAC, RNU4ATAC1OP, RNU4ATAC 11P, RNU4ATAC12P, RNU4ATAC13P, RNU4ATAC14P, RNU4ATAC15P, RNU4ATAC16P, RNU4ATAC17P, RNU4ATAC18P, RNU4ATAC2P, RNU4ATAC3P, RNU4ATAC4P, RNU4ATAC5P, RNU4ATAC6P, RNU4ATAC7P, RNU4ATAC8P, RNU4ATAC9P, RNU5A-1, RNU5A-2P, RNU5A-3P, RNU5A-4P, RNU5A-5P, RNU5A-6P, RNU5A-7P, RNU5A-8P, RNU5B-1, RNU5B-2P, RNU5B-3P, RNU5B-4P, RNU5B-6P, RNU5D-1, RNU5D-2P, RNU5E-1, RNU5E-1OP, RNU5E-3P, RNU5E-4P, RNU5E-5P, RNU5E-6P, RNU5E-7P, RNU5E-8P, RNU5E-9P, RNU5F-1, RNU5F-2P, RNU5F-3P, RNU5F-4P, RNU5F-6P, RNU5F-7P, RNU5F-8P, RNU6-1, RNU6-1000P, RNU6-1001P, RNU6-1003P, RNU6-1004P, RNU6-1005P, RNU6-1006P, RNU6-1007P, RNU6-1008P, RNU6-1009P, RNU6-100P, RNU6-1010P, RNU6-1011P, RNU6-1012P, RNU6-1013P, RNU6-1014P, RNU6-1015P, RNU6-1016P, RNU6-1017P, RNU6-1018P, RNU6-1019P, RNU6-101P, RNU6-1020P, RNU6-1021P, RNU6-1022P, RNU6-1023P, RNU6-1024P, RNU6-1025P, RNU6-1026P, RNU6-1027P, RNU6-1028P, RNU6-1029P, RNU6-102P, RNU6-1031P, RNU6-1032P, RNU6-1034P, RNU6-1035P, RNU6-1036P, RNU6-1037P, RNU6-1038P, RNU6-1039P, RNU6-103P, RNU6-1040P, RNU6-1041P, RNU6-1042P, RNU6-1043P, RNU6-1044P, RNU6-1045P, RNU6-1046P, RNU6-1047P, RNU6-1048P, RNU6-1049P, RNU6-104P, RNU6-1050P, RNU6-1051P, RNU6-1052P, RNU6-1053P, RNU6-1054P, RNU6-1055P, RNU6-1056P, RNU6-1057P, RNU6-1059P, RNU6-105P, RNU6-1060P, RNU6-1061P, RNU6-1062P, RNU6-1064P, RNU6-1065P, RNU6-1066P, RNU6-1067P, RNU6-1068P, RNU6-1069P, RNU6-106P, RNU6-1071P, RNU6-1072P, RNU6-1073P, RNU6-1074P, RNU6-1075P, RNU6-1076P, RNU6-1077P, RNU6-1078P, RNU6-1079P, RNU6-107P, RNU6-1080P, RNU6-1081P, RNU6-1082P, RNU6-1083P, RNU6-1084P, RNU6-1085P, RNU6-1086P, RNU6-1087P, RNU6-1088P, RNU6-1089P, RNU6-108P, RNU6-1090P, RNU6-1091P, RNU6-1092P, RNU6-1093P, RNU6-1094P, RNU6-1095P, RNU6-1096P, RNU6-1097P, RNU6-1098P, RNU6-1099P, RNU6-109P, RNU6-10P, RNU6-1100P, RNU6-1101P, RNU6-1102P, RNU6-1103P, RNU6-1104P, RNU6-1105P, RNU6-1106P, RNU6-1107P, RNU6-1108P, RNU6-1109P, RNU6-110P, RNU6-1110P, RNU6-1111P, RNU6-1112P, RNU6-1113P, RNU6-1114P, RNU6-1115P, RNU6-1116P, RNU6-1117P, RNU6-1118P, RNU6-1119P, RNU6-111P, RNU6-1120P, RNU6-1121P, RNU6-1122P, RNU6-1123P, RNU6-1124P, RNU6-1125P, RNU6-1126P, RNU6-1127P, RNU6-1128P, RNU6-1129P, RNU6-112P, RNU6-1130P, RNU6-1131P, RNU6-1132P, RNU6-1133P, RNU6-1134P, RNU6-1135P, RNU6-1136P, RNU6-1137P, RNU6-1138P, RNU6-113P, RNU6-1140P, RNU6-1141P, RNU6-1143P, RNU6-1144P, RNU6-1145P, RNU6-1146P, RNU6-1147P, RNU6-1148P, RNU6-1149P, RNU6-114P, RNU6-1150P, RNU6-1151P, RNU6-1152P, RNU6-1153P, RNU6-1154P, RNU6-1155P, RNU6-1156P, RNU6-1157P, RNU6-1158P, RNU6-1159P, RNU6-115P, RNU6-1160P, RNU6-1161P, RNU6-1162P, RNU6-1163P, RNU6-1164P, RNU6-1165P, RNU6-1167P, RNU6-1168P, RNU6-1169P, RNU6-116P, RNU6-1170P, RNU6-1171P, RNU6-1172P, RNU6-1174P, RNU6-1175P, RNU6-1176P, RNU6-1177P, RNU6-1178P, RNU6-1179P, RNU6-117P, RNU6-1180P, RNU6-1181P, RNU6-1183P, RNU6-1184P, RNU6-1186P, RNU6-1187P, RNU6-1188P, RNU6-1189P, RNU6-118P, RNU6-1190P, RNU6-1191P, RNU6-1192P, RNU6-1193P, RNU6-1194P, RNU6-1195P, RNU6-1196P, RNU6-1197P, RNU6-1198P, RNU6-1199P, RNU6-119P, RNU6-11P, RNU6-1200P, RNU6-1201P, RNU6-1203P, RNU6-1204P, RNU6-1205P, RNU6-1206P, RNU6-1207P, RNU6-1208P, RNU6-1209P, RNU6-120P, RNU6-1210P, RNU6-1211P, RNU6-1212P, RNU6-1213P, RNU6-1214P, RNU6-1215P, RNU6-1216P, RNU6-1217P, RNU6-1218P, RNU6-1219P, RNU6-121P, RNU6-1220P, RNU6-1222P, RNU6-1223P, RNU6-1224P, RNU6-1225P, RNU6-1226P, RNU6-1227P, RNU6-1228P, RNU6-1229P, RNU6-122P, RNU6-1230P, RNU6-1231P, RNU6-1232P, RNU6-1233P, RNU6-1234P, RNU6-1235P, RNU6-1236P, RNU6-1237P, RNU6-1238P, RNU6-1239P, RNU6-123P, RNU6-1240P, RNU6-1241P, RNU6-1242P, RNU6-1243P, RNU6-1244P, RNU6-1245P, RNU6-1246P, RNU6-1247P, RNU6-1248P, RNU6-1249P, RNU6-1250P, RNU6-1251P, RNU6-1252P, RNU6-1254P, RNU6-1255P, RNU6-1256P, RNU6-1257P, RNU6-1258P, RNU6-125P, RNU6-1260P, RNU6-1261P, RNU6-1262P, RNU6-1263P, RNU6-1264P, RNU6-1265P, RNU6-1266P, RNU6-1267P, RNU6-1268P, RNU6-1269P, RNU6-126P, RNU6-1270P, RNU6-1271P, RNU6-1272P, RNU6-1273P, RNU6-1274P, RNU6-1275P, RNU6-1276P, RNU6-1277P, RNU6-1278P, RNU6-1279P, RNU6-127P, RNU6-1280P, RNU6-1281P, RNU6-1282P, RNU6-1283P, RNU6-1284P, RNU6-1285P, RNU6-1286P, RNU6-1287P, RNU6-1288P, RNU6-1289P, RNU6-128P, RNU6-1290P, RNU6-1291P, RNU6-1292P, RNU6-1293P, RNU6-1294P, RNU6-1296P, RNU6-1297P, RNU6-1298P, RNU6-1299P, RNU6-129P, RNU6-12P, RNU6-1300P, RNU6-1301P, RNU6-1303P, RNU6-1304P, RNU6-1305P, RNU6-1306P, RNU6-1307P, RNU6-1308P, RNU6-1309P, RNU6-130P, RNU6-1310P, RNU6-1311P, RNU6-1312P, RNU6-1313P, RNU6-1314P, RNU6-1315P, RNU6-1316P, RNU6-1317P, RNU6-1318P, RNU6-1319P, RNU6-131P, RNU6-1320P, RNU6-1321P, RNU6-1322P, RNU6-1323P, RNU6-1324P, RNU6-1325P, RNU6-1326P, RNU6-1327P, RNU6-1328P, RNU6-1329P, RNU6-132P, RNU6-1330P, RNU6-1331P, RNU6-1332P, RNU6-1333P, RNU6-1334P, RNU6-1335P, RNU6-1336P, RNU6-1337P, RNU6-1338P, RNU6-1339P, RNU6-133P, RNU6-1340P, RNU6-135P, RNU6-136P, RNU6-137P, RNU6-138P, RNU6-139P, RNU6-13P, RNU6-140P, RNU6-141P, RNU6-142P, RNU6-143P, RNU6-144P, RNU6-145P, RNU6-146P, RNU6-147P, RNU6-148P, RNU6-14P, RNU6-150P, RNU6-151P, RNU6-152P, RNU6-153P, RNU6-154P, RNU6-155P, RNU6-156P, RNU6-157P, RNU6-158P, RNU6-159P, RNU6-15P, RNU6-160P, RNU6-161P, RNU6-162P, RNU6-163P, RNU6-164P, RNU6-165P, RNU6-166P, RNU6-167P, RNU6-168P, RNU6-169P, RNU6-16P, RNU6-170P, RNU6-171P, RNU6-172P, RNU6-173P, RNU6-174P, RNU6-175P, RNU6-176P, RNU6-177P, RNU6-178P, RNU6-179P, RNU6-17P, RNU6-180P, RNU6-181P, RNU6-182P, RNU6-183P, RNU6-184P, RNU6-185P, RNU6-187P, RNU6-188P, RNU6-189P, RNU6-18P, RNU6-190P, RNU6-191P, RNU6-192P, RNU6-193P, RNU6-194P, RNU6-195P, RNU6-196P, RNU6-197P, RNU6-198P, RNU6-199P, RNU6-19P, RNU6-2, RNU6-200P, RNU6-201P, RNU6-202P, RNU6-203P, RNU6-204P, RNU6-205P, RNU6-206P, RNU6-207P, RNU6-208P, RNU6-209P, RNU6-20P, RNU6-210P, RNU6-211P, RNU6-212P, RNU6-213P, RNU6-214P, RNU6-215P, RNU6-216P, RNU6-217P, RNU6-218P, RNU6-219P, RNU6-21P, RNU6-220P, RNU6-221P, RNU6-222P, RNU6-223P, RNU6-224P, RNU6-225P, RNU6-226P, RNU6-227P, RNU6-228P, RNU6-229P, RNU6-22P, RNU6-230P, RNU6-231P, RNU6-232P, RNU6-233P, RNU6-234P, RNU6-235P, RNU6-236P, RNU6-237P, RNU6-238P, RNU6-239P, RNU6-23P, RNU6-240P, RNU6-241P, RNU6-242P, RNU6-243P, RNU6-244P, RNU6-245P, RNU6-246P, RNU6-247P, RNU6-248P, RNU6-249P, RNU6-24P, RNU6-250P, RNU6-251P, RNU6-252P, RNU6-253P, RNU6-254P, RNU6-255P, RNU6-256P, RNU6-257P, RNU6-258P, RNU6-259P, RNU6-25P, RNU6-260P, RNU6-261P, RNU6-262P, RNU6-263P, RNU6-264P, RNU6-266P, RNU6-267P, RNU6-268P, RNU6-269P, RNU6-26P, RNU6-270P, RNU6-271P, RNU6-272P, RNU6-273P, RNU6-274P, RNU6-275P, RNU6-276P, RNU6-277P, RNU6-278P, RNU6-279P, RNU6-27P, RNU6-280P, RNU6-281P, RNU6-282P, RNU6-283P, RNU6-284P, RNU6-285P, RNU6-286P, RNU6-287P, RNU6-288P, RNU6-289P, RNU6-28P, RNU6-290P, RNU6-291P, RNU6-293P, RNU6-294P, RNU6-295P, RNU6-296P, RNU6-297P, RNU6-298P, RNU6-299P, RNU6-29P, RNU6-300P, RNU6-301P, RNU6-302P, RNU6-303P, RNU6-304P, RNU6-306P, RNU6-307P, RNU6-308P, RNU6-309P, RNU6-30P, RNU6-310P, RNU6-311P, RNU6-312P, RNU6-313P, RNU6-314P, RNU6-315P, RNU6-316P, RNU6-317P, RNU6-318P, RNU6-319P, RNU6-31P, RNU6-320P, RNU6-321P, RNU6-322P, RNU6-323P, RNU6-324P, RNU6-325P, RNU6-326P, RNU6-327P, RNU6-328P, RNU6-329P, RNU6-32P, RNU6-330P, RNU6-331P, RNU6-332P, RNU6-333P, RNU6-334P, RNU6-335P, RNU6-336P, RNU6-337P, RNU6-338P, RNU6-339P, RNU6-33P, RNU6-340P, RNU6-341P, RNU6-342P, RNU6-343P, RNU6-344P, RNU6-345P, RNU6-346P, RNU6-347P, RNU6-348P, RNU6-349P, RNU6-34P, RNU6-351P, RNU6-352P, RNU6-353P, RNU6-354P, RNU6-355P, RNU6-356P, RNU6-358P, RNU6-359P, RNU6-35P, RNU6-360P, RNU6-361P, RNU6-362P, RNU6-363P, RNU6-364P, RNU6-365P, RNU6-366P, RNU6-367P, RNU6-368P, RNU6-369P, RNU6-36P, RNU6-370P, RNU6-371P, RNU6-373P, RNU6-374P, RNU6-375P, RNU6-376P, RNU6-377P, RNU6-378P, RNU6-379P, RNU6-37P, RNU6-380P, RNU6-381P, RNU6-382P, RNU6-383P, RNU6-384P, RNU6-386P, RNU6-387P, RNU6-388P, RNU6-389P, RNU6-38P, RNU6-390P, RNU6-391P, RNU6-392P, RNU6-393P, RNU6-394P, RNU6-395P, RNU6-396P, RNU6-397P, RNU6-398P, RNU6-399P, RNU6-39P, RNU6-3P, RNU6-400P, RNU6-401P, RNU6-402P, RNU6-403P, RNU6-405P, RNU6-406P, RNU6-407P, RNU6-408P, RNU6-409P, RNU6-40P, RNU6-410P, RNU6-411P, RNU6-412P, RNU6-413P, RNU6-414P, RNU6-415P, RNU6-416P, RNU6-417P, RNU6-418P, RNU6-419P, RNU6-41P, RNU6-420P, RNU6-421P, RNU6-422P, RNU6-424P, RNU6-425P, RNU6-426P, RNU6-428P, RNU6-429P, RNU6-42P, RNU6-430P, RNU6-431P, RNU6-432P, RNU6-433P, RNU6-434P, RNU6-435P, RNU6-436P, RNU6-437P, RNU6-438P, RNU6-439P, RNU6-43P, RNU6-440P, RNU6-441P, RNU6-442P, RNU6-444P, RNU6-445P, RNU6-446P, RNU6-447P, RNU6-448P, RNU6-449P, RNU6-44P, RNU6-450P, RNU6-451P, RNU6-452P, RNU6-453P, RNU6-454P, RNU6-455P, RNU6-456P, RNU6-457P, RNU6-458P, RNU6-45P, RNU6-460P, RNU6-461P, RNU6-462P, RNU6-463P, RNU6-464P, RNU6-465P, RNU6-466P, RNU6-467P, RNU6-468P, RNU6-469P, RNU6-46P, RNU6-470P, RNU6-471P, RNU6-472P, RNU6-473P, RNU6-474P, RNU6-475P, RNU6-476P, RNU6-477P, RNU6-478P, RNU6-479P, RNU6-47P, RNU6-480P, RNU6-481P, RNU6-482P, RNU6-483P, RNU6-484P, RNU6-485P, RNU6-486P, RNU6-487P, RNU6-488P, RNU6-489P, RNU6-48P, RNU6-490P, RNU6-491P, RNU6-492P, RNU6-493P, RNU6-494P, RNU6-495P, RNU6-496P, RNU6-497P, RNU6-498P, RNU6-499P, RNU6-49P, RNU6-4P, RNU6-500P, RNU6-501P, RNU6-502P, RNU6-503P, RNU6-504P, RNU6-505P, RNU6-506P, RNU6-507P, RNU6-508P, RNU6-509P, RNU6-50P, RNU6-510P, RNU6-511P, RNU6-512P, RNU6-513P, RNU6-514P, RNU6-516P, RNU6-517P, RNU6-518P, RNU6-519P, RNU6-520P, RNU6-521P, RNU6-522P, RNU6-523P, RNU6-524P, RNU6-525P, RNU6-526P, RNU6-527P, RNU6-528P, RNU6-529P, RNU6-530P, RNU6-531P, RNU6-532P, RNU6-533P, RNU6-534P, RNU6-535P, RNU6-536P, RNU6-537P, RNU6-538P, RNU6-539P, RNU6-53P, RNU6-540P, RNU6-541P, RNU6-542P, RNU6-543P, RNU6-544P, RNU6-545P, RNU6-546P, RNU6-547P, RNU6-548P, RNU6-549P, RNU6-54P, RNU6-550P, RNU6-551P, RNU6-552P, RNU6-553P, RNU6-554P, RNU6-555P, RNU6-556P, RNU6-557P, RNU6-558P, RNU6-559P, RNU6-55P, RNU6-560P, RNU6-561P, RNU6-562P, RNU6-563P, RNU6-564P, RNU6-565P, RNU6-566P, RNU6-567P, RNU6-56P, RNU6-570P, RNU6-571P, RNU6-572P, RNU6-573P, RNU6-574P, RNU6-575P, RNU6-576P, RNU6-577P, RNU6-578P, RNU6-579P, RNU6-57P, RNU6-580P, RNU6-581P, RNU6-582P, RNU6-583P, RNU6-584P, RNU6-586P, RNU6-587P, RNU6-588P, RNU6-589P, RNU6-58P, RNU6-590P, RNU6-591P, RNU6-592P, RNU6-593P, RNU6-595P, RNU6-596P, RNU6-597P, RNU6-598P, RNU6-599P, RNU6-59P, RNU6-5P, RNU6-600P, RNU6-601P, RNU6-602P, RNU6-603P, RNU6-604P, RNU6-605P, RNU6-606P, RNU6-607P, RNU6-608P, RNU6-609P, RNU6-60P, RNU6-610P, RNU6-611P, RNU6-612P, RNU6-613P, RNU6-614P, RNU6-615P, RNU6-616P, RNU6-617P, RNU6-618P, RNU6-619P, RNU6-61P, RNU6-620P, RNU6-621P, RNU6-622P, RNU6-623P, RNU6-624P, RNU6-625P, RNU6-626P, RNU6-627P, RNU6-628P, RNU6-629P, RNU6-62P, RNU6-630P, RNU6-631P, RNU6-632P, RNU6-633P, RNU6-634P, RNU6-635P, RNU6-636P, RNU6-637P, RNU6-638P, RNU6-639P, RNU6-63P, RNU6-640P, RNU6-641P, RNU6-642P, RNU6-643P, RNU6-644P, RNU6-645P, RNU6-646P, RNU6-647P, RNU6-648P, RNU6-649P, RNU6-64P, RNU6-650P, RNU6-651P, RNU6-652P, RNU6-653P, RNU6-654P, RNU6-655P, RNU6-656P, RNU6-657P, RNU6-658P, RNU6-659P, RNU6-65P, RNU6-660P, RNU6-661P, RNU6-662P, RNU6-663P, RNU6-664P, RNU6-665P, RNU6-666P, RNU6-667P, RNU6-668P, RNU6-669P, RNU6-66P, RNU6-670P, RNU6-672P, RNU6-673P, RNU6-674P, RNU6-675P, RNU6-677P, RNU6-678P, RNU6-679P, RNU6-67P, RNU6-680P, RNU6-681P, RNU6-682P, RNU6-684P, RNU6-685P, RNU6-686P, RNU6-687P, RNU6-689P, RNU6-68P, RNU6-690P, RNU6-692P, RNU6-693P, RNU6-694P, RNU6-695P, RNU6-696P, RNU6-697P, RNU6-698P, RNU6-699P, RNU6-6P, RNU6-7, RNU6-700P, RNU6-701P, RNU6-702P, RNU6-703P, RNU6-704P, RNU6-705P, RNU6-706P, RNU6-707P, RNU6-708P, RNU6-709P, RNU6-70P, RNU6-710P, RNU6-711P, RNU6-712P, RNU6-713P, RNU6-714P, RNU6-715P, RNU6-716P, RNU6-717P, RNU6-718P, RNU6-719P, RNU6-71P, RNU6-720P, RNU6-721P, RNU6-722P, RNU6-723P, RNU6-724P, RNU6-725P, RNU6-726P, RNU6-727P, RNU6-728P, RNU6-729P, RNU6-72P, RNU6-730P, RNU6-731P, RNU6-732P, RNU6-733P, RNU6-735P, RNU6-737P, RNU6-738P, RNU6-739P, RNU6-73P, RNU6-740P, RNU6-741P, RNU6-742P, RNU6-743P, RNU6-744P, RNU6-745P, RNU6-746P, RNU6-747P, RNU6-748P, RNU6-749P, RNU6-74P, RNU6-750P, RNU6-751P, RNU6-752P, RNU6-753P, RNU6-754P, RNU6-755P, RNU6-756P, RNU6-757P, RNU6-758P, RNU6-759P, RNU6-75P, RNU6-760P, RNU6-761P, RNU6-762P, RNU6-763P, RNU6-764P, RNU6-765P, RNU6-766P, RNU6-767P, RNU6-768P, RNU6-769P, RNU6-76P, RNU6-770P, RNU6-771P, RNU6-772P, RNU6-774P, RNU6-775P, RNU6-776P, RNU6-777P, RNU6-778P, RNU6-77P, RNU6-780P, RNU6-781P, RNU6-782P, RNU6-783P, RNU6-784P, RNU6-785P, RNU6-786P, RNU6-787P, RNU6-788P, RNU6-789P, RNU6-78P, RNU6-790P, RNU6-791P, RNU6-792P, RNU6-793P, RNU6-794P, RNU6-795P, RNU6-796P, RNU6-797P, RNU6-798P, RNU6-799P, RNU6-79P, RNU6-8, RNU6-800P, RNU6-801P, RNU6-803P, RNU6-804P, RNU6-805P, RNU6-806P, RNU6-807P, RNU6-808P, RNU6-809P, RNU6-80P, RNU6-810P, RNU6-811P, RNU6-812P, RNU6-813P, RNU6-815P, RNU6-816P, RNU6-817P, RNU6-818P, RNU6-819P, RNU6-81P, RNU6-820P, RNU6-821P, RNU6-822P, RNU6-823P, RNU6-824P, RNU6-826P, RNU6-827P, RNU6-828P, RNU6-829P, RNU6-82P, RNU6-830P, RNU6-831P, RNU6-832P, RNU6-833P, RNU6-834P, RNU6-835P, RNU6-836P, RNU6-837P, RNU6-838P, RNU6-839P, RNU6-83P, RNU6-840P, RNU6-841P, RNU6-842P, RNU6-843P, RNU6-844P, RNU6-845P, RNU6-847P, RNU6-848P, RNU6-849P, RNU6-84P, RNU6-850P, RNU6-851P, RNU6-853P, RNU6-854P, RNU6-855P, RNU6-856P, RNU6-857P, RNU6-858P, RNU6-859P, RNU6-85P, RNU6-860P, RNU6-861P, RNU6-862P, RNU6-863P, RNU6-864P, RNU6-865P, RNU6-866P, RNU6-867P, RNU6-869P, RNU6-86P, RNU6-871P, RNU6-873P, RNU6-874P, RNU6-875P, RNU6-876P, RNU6-877P, RNU6-878P, RNU6-879P, RNU6-87P, RNU6-880P, RNU6-881P, RNU6-882P, RNU6-883P, RNU6-884P, RNU6-885P, RNU6-886P, RNU6-887P, RNU6-888P, RNU6-889P, RNU6-88P, RNU6-890P, RNU6-891P, RNU6-892P, RNU6-893P, RNU6-894P, RNU6-895P, RNU6-896P, RNU6-897P, RNU6-898P, RNU6-899P, RNU6-89P, RNU6-9, RNU6-900P, RNU6-901P, RNU6-902P, RNU6-903P, RNU6-904P, RNU6-905P, RNU6-906P, RNU6-907P, RNU6-908P, RNU6-909P, RNU6-90P, RNU6-910P, RNU6-911P, RNU6-912P, RNU6-913P, RNU6-914P, RNU6-915P, RNU6-916P, RNU6-917P, RNU6-918P, RNU6-919P, RNU6-91P, RNU6-920P, RNU6-921P, RNU6-922P, RNU6-923P, RNU6-924P, RNU6-925P, RNU6-926P, RNU6-927P, RNU6-928P, RNU6-929P, RNU6-92P, RNU6-930P, RNU6-931P, RNU6-932P, RNU6-933P, RNU6-934P, RNU6-935P, RNU6-936P, RNU6-937P, RNU6-938P, RNU6-939P, RNU6-940P, RNU6-941P, RNU6-942P, RNU6-943P, RNU6-944P, RNU6-945P, RNU6-946P, RNU6-947P, RNU6-948P, RNU6-949P, RNU6-94P, RNU6-950P, RNU6-951P, RNU6-952P, RNU6-953P, RNU6-954P, RNU6-955P, RNU6-956P, RNU6-957P, RNU6-958P, RNU6-959P, RNU6-95P, RNU6-960P, RNU6-961P, RNU6-964P, RNU6-965P, RNU6-966P, RNU6-967P, RNU6-968P, RNU6-969P, RNU6-970P, RNU6-971P, RNU6-972P, RNU6-973P, RNU6-974P, RNU6-975P, RNU6-976P, RNU6-977P, RNU6-978P, RNU6-979P, RNU6-97P, RNU6-980P, RNU6-982P, RNU6-983P, RNU6-984P, RNU6-985P, RNU6-986P, RNU6-987P, RNU6-988P, RNU6-989P, RNU6-98P, RNU6-990P, RNU6-991P, RNU6-992P, RNU6-993P, RNU6-994P, RNU6-995P, RNU6-996P, RNU6-997P, RNU6-998P, RNU6-999P, RNU6-99P, RNU6ATAC, RNU6ATAC10P, RNU6ATAC1IP, RNU6ATAC12P, RNU6ATAC13P, RNU6ATAC14P, RNU6ATAC15P, RNU6ATAC16P, RNU6ATAC17P, RNU6ATAC18P, RNU6ATAC19P, RNU6ATAC20P, RNU6ATAC21P, RNU6ATAC22P, RNU6ATAC23P, RNU6ATAC24P, RNU6ATAC25P, RNU6ATAC26P, RNU6ATAC27P, RNU6ATAC28P, RNU6ATAC29P, RNU6ATAC2P, RNU6ATAC30P, RNU6ATAC31P, RNU6ATAC32P, RNU6ATAC33P, RNU6ATAC34P, RNU6ATAC36P, RNU6ATAC37P, RNU6ATAC38P, RNU6ATAC39P, RNU6ATAC3P, RNU6ATAC40P, RNU6ATAC41P, RNU6ATAC42P, RNU6ATAC4P, RNU6ATAC5P, RNU6ATAC6P, RNU6ATAC7P, RNU6ATAC8P, RNU6ATAC9P, RNU6V, RNU7-1, RNU7-102P, RNU7-103P, RNU7-104P, RNU7-105P, RNU7-106P, RNU7-107P, RNU7-1OP, RNU7-110P, RNU7-111P, RNU7-113P, RNU7-115P, RNU7-116P, RNU7-119P, RNU7-11P, RNU7-120P, RNU7-121P, RNU7-123P, RNU7-124P, RNU7-125P, RNU7-126P, RNU7-127P, RNU7-128P, RNU7-129P, RNU7-12P, RNU7-130P, RNU7-133P, RNU7-134P, RNU7-136P, RNU7-137P, RNU7-138P, RNU7-13P, RNU7-140P, RNU7-141P, RNU7-143P, RNU7-144P, RNU7-147P, RNU7-148P, RNU7-149P, RNU7-14P, RNU7-151P, RNU7-152P, RNU7-153P, RNU7-154P, RNU7-155P, RNU7-156P, RNU7-157P, RNU7-159P, RNU7-160P, RNU7-161P, RNU7-164P, RNU7-165P, RNU7-167P, RNU7-169P, RNU7-170P, RNU7-171P, RNU7-172P, RNU7-173P, RNU7-174P, RNU7-175P, RNU7-176P, RNU7-179P, RNU7-180P, RNU7-181P, RNU7-182P, RNU7-183P, RNU7-185P, RNU7-186P, RNU7-187P, RNU7-188P, RNU7-18P, RNU7-190P, RNU7-192P, RNU7-193P, RNU7-194P, RNU7-195P, RNU7-196P, RNU7-197P, RNU7-19P, RNU7-200P, RNU7-20P, RNU7-21P, RNU7-22P, RNU7-23P, RNU7-24P, RNU7-25P, RNU7-26P, RNU7-27P, RNU7-28P, RNU7-29P, RNU7-2P, RNU7-30P, RNU7-34P, RNU7-35P, RNU7-37P, RNU7-38P, RNU7-3P, RNU7-40P, RNU7-41P, RNU7-43P, RNU7-45P, RNU7-46P, RNU7-47P, RNU7-48P, RNU7-49P, RNU7-4P, RNU7-50P, RNU7-51P, RNU7-52P, RNU7-53P, RNU7-54P, RNU7-55P, RNU7-56P, RNU7-57P, RNU7-59P, RNU7-60P, RNU7-61P, RNU7-62P, RNU7-63P, RNU7-65P, RNU7-66P, RNU7-67P, RNU7-69P, RNU7-6P, RNU7-70P, RNU7-71P, RNU7-73P, RNU7-74P, RNU7-75P, RNU7-77P, RNU7-79P, RNU7-7P, RNU7-80P, RNU7-81P, RNU7-82P, RNU7-84P, RNU7-85P, RNU7-87P, RNU7-88P, RNU7-8P, RNU7-90P, RNU7-92P, RNU7-93P, RNU7-94P, RNU7-95P, RNU7-96P, RNU7-97P, RNU7-99P, RNU7-9P, RNVU1-1, RNVU1-14, RNVU1-15, RNVU1-17, RNVU1-18, RNVU1-19, RNVU1-2, RNVU1-21, RNVU1-22, RNVU1-23, RNVU1-24, RNVU1-25, RNVU1-26, RNVU1-27, RNVU1-28, RNVU1-29, RNVU1-2A, RNVU1-3, RNVU1-30, RNVU1-31, RNVU1-32, RNVU1-33, RNVU1-34, RNVU1-4, RNVU1-6, RNVU1-7, RNVU1-8, U1, U2, U4, U6, U7.

In some embodiments, a splice editor nucleic acid molecule of the disclosure comprises a full-length snRNA or portion thereof comprising a nucleotide sequence comprising one or more sequence motifs described herein (e.g., one or more sequence motifs set forth in Table 1), and wherein the snRNA is selected from a U1 snRNA, a U2 snRNA, a U4 snRNA, a U4atac snRNA, a U5 snRNA, a U6 snRNA, a U6atac snRNA, a U11 snRNA, a U12 snRNA, and a U7 snRNA.

In some embodiments, the snRNA is a U1 snRNA. In some embodiments, the U1 snRNA assembles into a U1 RNP. In some embodiments, the snRNA is a U2 snRNA. In some embodiments, the U2 snRNA assembles into a U2 RNP. In some embodiments, the snRNA is a U4 snRNA. In some embodiments, the U1 snRNA assembles into a U4 RNP. In some embodiments, the snRNA is a U4atac snRNA. In some embodiments, the U1 snRNA assembles into a U4atac RNP. In some embodiments, the snRNA is a U5 snRNA. In some embodiments, the U1 snRNA assembles into a U5 RNP. In some embodiments, the snRNA is a U6 snRNA. In some embodiments, the U1 snRNA assembles into a U6 RNP. In some embodiments, the snRNA is a U6atac snRNA. In some embodiments, the U1 snRNA assembles into a U6atac RNP. In some embodiments, the snRNA is a U7 snRNA. In some embodiments, the U1 snRNA assembles into a U7 RNP. In some embodiments, the snRNA is a U11 snRNA. In some embodiments, the U1 snRNA assembles into a U11 RNP. In some embodiments, the snRNA is a U12 snRNA. In some embodiments, the U1 snRNA assembles into a U12 RNP.

In some embodiments, the ncRNA comprises a Sm sequence motif. In some embodiments, the Sm sequence motif assembles with an Sm protein to form an RNP. In some embodiments, the Sm protein is B/B′, D3, D2, D1, E, F, and G Sm proteins.

In some embodiments, a splice editor nucleic acid molecule of the disclosure comprises a full-length snoRNA or portion thereof comprising a nucleotide sequence comprising one or more sequence motifs described herein (e.g., one or more sequence motifs set forth in Table 1). In some embodiments, a snoRNA sequence described herein or identified according to a method described herein is incorporated into a splice editor nucleic acid of the disclosure. In some embodiments, a full-length snoRNA sequence described herein or identified according to a method described herein is incorporated into a splice editor nucleic acid of the disclosure. In some embodiments, a portion of a snoRNA sequence described herein or identified according to a method described herein (e.g., a region of contiguous nucleotides in the snRNA) is incorporated into a splice editor nucleic acid of the disclosure.

In some embodiments, the full-length snoRNA or portion thereof assembles into a small nucleolar RNP (snoRNP). snoRNAs are responsible for RNA methylation and RNA pseudouridylation (Bachellerie 2002, Kiss 2004). There are two classes of snoRNAs, namely (i) H/ACA box snoRNAs which are responsible for pseudouridylation and (ii) C/D box snoRNAs which are responsible for 2′-O-ribose methylation (Jorjani 2016, Kufel 2019). snoRNAs can also form RNPs termed snoRNPs (Khanna 2006) and hybridize to their RNA targets via Watson-Crick base pairing (Jin 2007).

In some embodiments, the full-length snoRNA or portion thereof comprises an H/ACA box. In some embodiments, the H/ACA box comprises a nucleotide sequence comprising from 5′ to 3′ an H consensus sequence (e.g., an H consensus sequence comprising the sequence set forth in SEQ ID NO: 1) and an ACA consensus sequence (e.g., an ACA consensus sequence comprising the sequence set forth in SEQ ID NO: 2). In some embodiments, the H/ACA box snoRNA assembles to form an H/ACA snoRNP. In some embodiments, the full-length snoRNA or portion thereof comprises a C/D box. In some embodiment, the C/D box comprises a nucleotide sequence comprising from 5′ to 3′ a C consensus sequence (e.g., a C consensus sequence comprising the sequence set forth in SEQ ID NO: 5), a D′ consensus sequence (e.g., a D′ consensus sequence comprising the sequence set forth in SEQ ID NO: 8), a C′ consensus sequence (e.g., a C′ consensus sequence comprising the sequence set forth in SEQ ID NO: 7), and a D consensus sequence (e.g., a D consensus sequence comprising the sequence set forth in SEQ ID NO: 6). In some embodiments, the C/D box snoRNP assembles to form a C/D snoRNP.

In some embodiments, a splice editor nucleic acid molecule of the disclosure comprises a full-length snoRNA or portion thereof comprising a nucleotide sequence comprising one or more sequence motifs described herein (e.g., one or more sequence motifs set forth in Table 1), and wherein the snoRNA is selected from SCARNA18, SCARNA18B, SNORA1, SNORA10, SNORA108, SNORA10B, SNORA11, SNORA11B, SNORA11C, SNORA11D, SNORA11E, SNORA11F, SNORA11G, SNORA12, SNORA13, SNORA14A, SNORA14B, SNORA15, SNORA15B-1, SNORA15B-2, SNORA16A, SNORA16B, SNORA17A, SNORA17B, SNORA18, SNORA19, SNORA1B, SNORA20, SNORA20B, SNORA21, SNORA21B, SNORA22, SNORA22B, SNORA22C, SNORA24, SNORA24B, SNORA25, SNORA25B, SNORA26, SNORA27, SNORA28, SNORA29, SNORA2A, SNORA2B, SNORA2C, SNORA30, SNORA30B, SNORA31, SNORA3i1, SNORA32, SNORA33, SNORA35, SNORA35B, SNORA36A, SNORA36B, SNORA36C, SNORA37, SNORA38, SNORA38B, SNORA3A, SNORA3B, SNORA3C, SNORA4, SNORA40, SNORA40B, SNORA40C, SNORA41, SNORA41B, SNORA44, SNORA46, SNORA47, SNORA48, SNORA48B, SNORA49, SNORA50A, SNORA50B, SNORA50C, SNORA50D, SNORA51, SNORA52, SNORA54, SNORA55, SNORA56, SNORA57, SNORA58, SNORA58B, SNORA59A, SNORA5A, SNORA5B, SNORA5C, SNORA6, SNORA60, SNORA61, SNORA62, SNORA63, SNORA63B, SNORA63C, SNORA63D, SNORA63E, SNORA64, SNORA65, SNORA66, SNORA67, SNORA68, SNORA68B, SNORA69, SNORA70, SNORA70B, SNORA70C, SNORA70D, SNORA70E, SNORA70F, SNORA70G, SNORA70H, SNORA70I, SNORA70J, SNORA71, SNORA71A, SNORA71C, SNORA71D, SNORA71E, SNORA72, SNORA73, SNORA74, SNORA74D, SNORA75, SNORA75B, SNORA77, SNORA77B, SNORA78, SNORA79, SNORA79B, SNORA7A, SNORA7B, SNORA8, SNORA80A, SNORA80B, SNORA80C, SNORA80D, SNORA80E, SNORA81, SNORA84, SNORA9, SNORA9B, SNORD10, SNORD100, SNORD101, SNORD102, SNORD104, SNORD105, SNORD105B, SNORD107, SNORD108, SNORD109A, SNORD109B, SNORD11, SNORD110, SNORD111, SNORD111B, SNORD112, SNORD113-1, SNORD113-2, SNORD113-3, SNORD113-4, SNORD113-5, SNORD113-6, SNORD113-7, SNORD113-8, SNORD113-9, SNORD114-1, SNORD114-10, SNORD114-11, SNORD114-12, SNORD114-13, SNORD114-14, SNORD114-15, SNORD114-16, SNORD114-17, SNORD114-18, SNORD114-19, SNORD114-2, SNORD114-20, SNORD114-21, SNORD114-22, SNORD114-23, SNORD114-24, SNORD114-25, SNORD114-26, SNORD114-27, SNORD114-28, SNORD114-29, SNORD114-3, SNORD114-30, SNORD114-31, SNORD114-4, SNORD114-5, SNORD114-6, SNORD114-7, SNORD114-9, SNORD115, SNORD115-1, SNORD115-10, SNORD115-11, SNORD115-12, SNORD115-13, SNORD115-14, SNORD115-15, SNORD115-16, SNORD115-17, SNORD115-18, SNORD115-19, SNORD115-2, SNORD115-20, SNORD115-21, SNORD115-22, SNORD115-23, SNORD115-24, SNORD115-25, SNORD115-26, SNORD115-27, SNORD115-28, SNORD115-29, SNORD115-3, SNORD115-30, SNORD115-31, SNORD115-32, SNORD115-33, SNORD115-34, SNORD115-35, SNORD115-36, SNORD115-37, SNORD115-38, SNORD115-39, SNORD115-4, SNORD115-40, SNORD115-41, SNORD115-42, SNORD115-43, SNORD115-44, SNORD115-45, SNORD115-46, SNORD115-47, SNORD115-48, SNORD115-5, SNORD115-6, SNORD115-7, SNORD115-8, SNORD115-9, SNORD116, SNORD116-1, SNORD116-10, SNORD116-11, SNORD116-12, SNORD116-13, SNORD116-14, SNORD116-15, SNORD116-16, SNORD116-17, SNORD116-18, SNORD116-19, SNORD116-2, SNORD116-20, SNORD116-21, SNORD116-22, SNORD116-23, SNORD116-24, SNORD116-25, SNORD116-26, SNORD116-27, SNORD116-28, SNORD116-29, SNORD116-3, SNORD116-30, SNORD116-4, SNORD116-5, SNORD116-6, SNORD116-7, SNORD116-8, SNORD116-9, SNORD117, SNORD118, SNORD11B, SNORD12, SNORD121A, SNORD121B, SNORD123, SNORD124, SNORD125, SNORD126, SNORD127, SNORD12B, SNORD12C, SNORD13, SNORD13D, SNORD13E, SNORD13P1, SNORD13P3, SNORD14, SNORD14A, SNORD14B, SNORD14C, SNORD14D, SNORD14E, SNORD15A, SNORD15B, SNORD16, SNORD18, SNORD18A, SNORD18B, SNORD18C, SNORD19, SNORD19B, SNORD19C, SNORD1A, SNORD1B, SNORD1C, SNORD2, SNORD20, SNORD21, SNORD22, SNORD23, SNORD24, SNORD25, SNORD26, SNORD27, SNORD28, SNORD28B, SNORD29, SNORD30, SNORD31B, SNORD32A, SNORD32B, SNORD33, SNORD34, SNORD35A, SNORD35B, SNORD36, SNORD36A, SNORD36B, SNORD36C, SNORD37, SNORD38A, SNORD38B, SNORD38C, SNORD38D, SNORD39, SNORD41, SNORD42, SNORD42A, SNORD42B, SNORD43, SNORD45A, SNORD45B, SNORD45C, SNORD46, SNORD48, SNORD49A, SNORD49B, SNORD4A, SNORD4B, SNORD5, SNORD50B, SNORD51, SNORD52, SNORD53, SNORD53B, SNORD54, SNORD55, SNORD56, SNORD56B, SNORD57, SNORD58, SNORD58A, SNORD58B, SNORD58C, SNORD59A, SNORD6, SNORD60, SNORD61, SNORD62, SNORD62A, SNORD62B, SNORD63, SNORD63B, SNORD64, SNORD65, SNORD65B, SNORD65C, SNORD66, SNORD67, SNORD68, SNORD69, SNORD7, SNORD70, SNORD70B, SNORD71, SNORD72, SNORD73A, SNORD73B, SNORD74B, SNORD77B, SNORD79, SNORD8, SNORD81, SNORD82, SNORD83, SNORD83A, SNORD83B, SNORD84, SNORD86, SNORD87, SNORD88A, SNORD88B, SNORD88C, SNORD89, SNORD9, SNORD90, SNORD92, SNORD93, SNORD94, SNORD95, SNORD96A, SNORD96B, SNORD97, SNORD98, SNORD99, U8, snoZ196.

In some embodiments, the ncRNA is a scaRNA. In some embodiments, a splice editor nucleic acid molecule of the disclosure comprises a full-length scaRNA or portion thereof comprising a nucleotide sequence comprising one or more sequence motifs described herein (e.g., one or more sequence motifs set forth in Table 1). In some embodiments, a scaRNA sequence described herein or identified according to a method described herein is incorporated into a splice editor nucleic acid of the disclosure. In some embodiments, a full-length scaRNA sequence described herein or identified according to a method described herein is incorporated into a splice editor nucleic acid of the disclosure. In some embodiments, a portion of a scaRNA sequence described herein or identified according to a method described herein (e.g., a region of contiguous nucleotides in the scaRNA) is incorporated into a splice editor nucleic acid of the disclosure. In some embodiments, the full-length scaRNA or portion thereof assembles into a small cajal body RNP (scaRNP). In some embodiments, the full-length scaRNA or the portion thereof comprises one or more secondary RNA structures that assembles to form a scaRNP. In some embodiments, the full-length scaRNA or the portion thereof comprises one or more sequence motifs that assembles to form a scaRNP. In some embodiments, the full-length scaRNA or the portion thereof comprises (i) one or more one or more secondary RNA structures, and (ii) one or more sequence motifs, wherein (i), (ii), or both assemble to form a scaRNP. In some embodiments, the full-length scaRNA or the portion thereof comprises an H/ACA box, wherein the H/ACA box comprises a nucleotide sequence comprising from 5′ to 3′ an H consensus sequence (e.g., an H consensus sequence comprising the sequence set forth in SEQ ID NO: 1) and an ACA consensus sequence (e.g., an ACA consensus sequence comprising the sequence set forth in SEQ ID NO: 2).

In some embodiments, a splice editor nucleic acid molecule of the disclosure comprises a full-length scaRNA or portion thereof comprising a nucleotide sequence comprising one or more sequence motifs described herein (e.g., one or more sequence motifs set forth in Table 1), wherein the scaRNA is selected from SCARNA1, SCARNA11, SCARNA14, SCARNA15, SCARNA17, SCARNA20, SCARNA21, SCARNA21B, SCARNA22, SCARNA23, SCARNA3, SCARNA4, SCARNA8.

In some embodiments, the ncRNA is a lncRNA. In some embodiments, a splice editor nucleic acid molecule of the disclosure comprises a full-length lncRNA or portion thereof comprising a nucleotide sequence comprising one or more sequence motifs described herein (e.g., one or more sequence motifs set forth in Table 1). In some embodiments, a lncRNA sequence described herein or identified according to a method described herein is incorporated into a splice editor nucleic acid of the disclosure. In some embodiments, a full-length lncRNA sequence described herein or identified according to a method described herein is incorporated into a splice editor nucleic acid of the disclosure. In some embodiments, a portion of a lncRNA sequence described herein or identified according to a method described herein (e.g., a region of contiguous nucleotides in the lncRNA) is incorporated into a splice editor nucleic acid of the disclosure. In some embodiments, the full-length lncRNA or portion thereof assembles into an RNP. In some embodiments, the full-length lncRNA or the portion thereof comprises one or more secondary RNA structures that assembles to form an RNP. In some embodiments, the full-length lncRNA or the portion thereof comprises one or more sequence motifs that assembles to form an RNP. In some embodiments, the full-length lncRNA or the portion thereof comprises (i) one or more one or more secondary RNA structures, and (ii) one or more sequence motifs, wherein (i), (ii), or both assemble to form an RNP.

In some embodiments, a splice editor nucleic acid molecule of the disclosure comprises a full-length lncRNA or portion thereof comprising a nucleotide sequence comprising one or more sequence motifs described herein (e.g., one or more sequence motifs set forth in Table 1), wherein the lncRNA is selected from AADACL2-AS1, ARHGEF26-AS1, ARMC2-AS1, BCAR3-AS1, C4B, CABIN1, CAPN15, CARS1-AS1, CASC19, CELF2-AS2, CPB2-AS1, EPHA5-AS1, ETV7-AS1, F 11-AS1, FLG-AS1, GATA6-AS1, GLYCTK-AS1, HCG17, HCG27, HCG9, HHATL-AS1, HOTAIRM1, KIFC1, LINC00511, LINC00824, LINC01060, LINC01358, LINC01378, LINC01409, LINC01606, LINC01676, LINC01943, LINC02276, LINC02301, LINC02690, LINC02695, LINC02790, LINC02805, LRIG3-DT, LY6E-DT, MALATI, MAP3K14, MAPK4, MEIOB, OR12D3, PCDH9-AS2, PIF1, PSMB1, SLC8A1-AS1, SNHG25, SPRY4-AS1, TEX41, TTTY17A, TTTY17B, UST-AS2, ZEB2-AS1, hsa-mir-1253, hsa-mir-423.

In some embodiments, the ncRNA is a miscRNA. In some embodiments, a splice editor nucleic acid molecule of the disclosure comprises a full-length miscRNA or portion thereof comprising a nucleotide sequence comprising one or more sequence motifs described herein (e.g., one or more sequence motifs set forth in Table 1). In some embodiments, a miscRNA sequence described herein or identified according to a method described herein is incorporated into a splice editor nucleic acid of the disclosure. In some embodiments, a full-length miscRNA sequence described herein or identified according to a method described herein is incorporated into a splice editor nucleic acid of the disclosure. In some embodiments, a portion of a miscRNA sequence described herein or identified according to a method described herein (e.g., a region of contiguous nucleotides in the miscRNA) is incorporated into a splice editor nucleic acid of the disclosure. In some embodiments, the full-length miscRNA or portion thereof assembles into an RNP. In some embodiments, the full-length miscRNA or the portion thereof comprises one or more secondary RNA structures that assembles to form an RNP. In some embodiments, the full-length miscRNA or the portion thereof comprises one or more sequence motifs that assembles to form an RNP. In some embodiments, the full-length miscRNA or the portion thereof comprises (i) one or more one or more secondary RNA structures, and (ii) one or more sequence motifs, wherein (i), (ii), or both assemble to form an RNP.

In some embodiments, a splice editor nucleic acid molecule of the disclosure comprises a full-length miscRNA or portion thereof comprising a nucleotide sequence comprising one or more sequence motifs described herein (e.g., one or more sequence motifs set forth in Table 1), wherein the miscRNA is selected from RN7SKP12, RN7SKP223, RN7SKP233, RN7SKP260, RN7SKP295, RN7SKP298, RN7SKP35, RN7SKP83, RN7SKP98, RNY1, RNY1P1, RNY1P10, RNY1P11, RNY1P12, RNY1P13, RNY1P14, RNY1P15, RNY1P16, RNY1P2, RNY1P3, RNY1P4, RNY1P5, RNY1P6, RNY1P7, RNY1P8, RNY1P9, RNY3, RNY3P1, RNY3P10, RNY3P11, RNY3P12, RNY3P13, RNY3P14, RNY3P15, RNY3P16, RNY3P2, RNY3P3, RNY3P4, RNY3P5, RNY3P7, RNY3P8, RNY3P9, RNY4, RNY4P10, RNY4P13, RNY4P14, RNY4P16, RNY4P17, RNY4P18, RNY4P19, RNY4P20, RNY4P23, RNY4P24, RNY4P25, RNY4P27, RNY4P28, RNY4P29, RNY4P3, RNY4P30, RNY4P34, RNY4P36, RNY4P37, RNY4P6, RNY4P7, RNY4P9, VTRNA1-1, VTRNA1-2, VTRNA1-3, VTRNA2-2P, VTRNA3-1P, and Vault, Y_RNA.

In some embodiments, the ncRNA is a Mt tRNA. In some embodiments, a splice editor nucleic acid molecule of the disclosure comprises a full-length Mt tRNA or portion thereof comprising a nucleotide sequence comprising one or more sequence motifs described herein (e.g., one or more sequence motifs set forth in Table 1). In some embodiments, a Mt tRNA sequence described herein or identified according to a method described herein is incorporated into a splice editor nucleic acid of the disclosure. In some embodiments, a full-length Mt tRNA sequence described herein or identified according to a method described herein is incorporated into a splice editor nucleic acid of the disclosure. In some embodiments, a portion of a Mt tRNA sequence described herein or identified according to a method described herein (e.g., a region of contiguous nucleotides in the Mt tRNA) is incorporated into a splice editor nucleic acid of the disclosure. In some embodiments, the full-length Mt tRNA or portion thereof assembles into an RNP. In some embodiments, the full-length Mt tRNA or the portion thereof comprises one or more secondary RNA structures that assembles to form an RNP. In some embodiments, the full-length Mt tRNA or the portion thereof comprises one or more sequence motifs that assembles to form an RNP. In some embodiments, the full-length Mt tRNA or the portion thereof comprises (i) one or more one or more secondary RNA structures, and (ii) one or more sequence motifs, wherein (i), (ii), or both assemble to form an RNP.

In some embodiments, a splice editor nucleic acid molecule of the disclosure comprises a full-length Mt RNA or portion thereof comprising a nucleotide sequence comprising one or more sequence motifs described herein (e.g., one or more sequence motifs set forth in Table 1), wherein the Mt tRNAs is selected from MT-TA, MT-TC, MT-TD, MT-TE, MT-TF, MT-TG, MT-TH, MT-TI, MT-TK, MT-TL1, MT-TL2, MT-TM, MT-TN, MT-TP, MT-TQ, MT-TR, MT-TS1, MT-TS2, MT-TT, MT-TV, MT-TW, and MT-TY.

In some embodiments, the ncRNA is an rRNA. In some embodiments, a splice editor nucleic acid molecule of the disclosure comprises a full-length rRNA or portion thereof comprising a nucleotide sequence comprising one or more sequence motifs described herein (e.g., one or more sequence motifs set forth in Table 1). In some embodiments, a rRNA sequence described herein or identified according to a method described herein is incorporated into a splice editor nucleic acid of the disclosure. In some embodiments, a full-length rRNA sequence described herein or identified according to a method described herein is incorporated into a splice editor nucleic acid of the disclosure. In some embodiments, a portion of a rRNA sequence described herein or identified according to a method described herein (e.g., a region of contiguous nucleotides in the rRNA) is incorporated into a splice editor nucleic acid of the disclosure. In some embodiments, the full-length rRNA or portion thereof assembles into an RNP. In some embodiments, the full-length rRNA or the portion thereof comprises one or more secondary RNA structures that assembles to form an RNP. In some embodiments, the full-length rRNA or the portion thereof comprises one or more sequence motifs that assembles to form an RNP. In some embodiments, the full-length rRNA or the portion thereof comprises (i) one or more one or more secondary RNA structures, and (ii) one or more sequence motifs, wherein (i), (ii), or both assemble to form an RNP.

In some embodiments, a splice editor nucleic acid molecule of the disclosure comprises a full-length rRNA or portion thereof comprising a nucleotide sequence comprising one or more sequence motifs described herein (e.g., one or more sequence motifs set forth in Table 1), wherein the rRNAs is selected from RNA5S1, RNA5S2, RNA5S3, RNA5S4, RNA5S5, RNA5S6, RNA5S7, RNA5S8, RNA5S9, RNA5S10, RNA5S11, RNA5S12, RNA5S13, RNA5S14, RNA5S15, RNA5S16, RNA5S17, RNR1, RNR2, RNR3, RNR4, RNR5, RNA18SN1, RNA18SN2, RNA18SN3, RNA18SN4, RNA18SN5, RNA28SN1, RNA28SN2, RNA28SN3, RNA28SN4, RNA28SN5, RNA45SN1, RNA45SN2, RNA45SN3, RNA45SN4, RNA45SN5, RNA5-8SN1, RNA5-8SN2, RNA5-8SN3, RNA5-8SN4, and RNA5-8SN5.

In some embodiments, the ncRNA is a vault RNA. In some embodiments, a splice editor nucleic acid molecule of the disclosure comprises a full-length vault RNA or portion thereof comprising a nucleotide sequence comprising one or more sequence motifs described herein (e.g., one or more sequence motifs set forth in Table 1). In some embodiments, a vault RNA sequence described herein or identified according to a method described herein is incorporated into a splice editor nucleic acid of the disclosure. In some embodiments, a full-length vault RNA sequence described herein or identified according to a method described herein is incorporated into a splice editor nucleic acid of the disclosure. In some embodiments, a portion of a vault RNA sequence described herein or identified according to a method described herein (e.g., a region of contiguous nucleotides in the vault RNA) is incorporated into a splice editor nucleic acid of the disclosure. In some embodiments, the full-length vault RNA or portion thereof assembles into an RNP. In some embodiments, the full-length vault RNA or the portion thereof comprises one or more secondary RNA structures that assembles to form an RNP. In some embodiments, the full-length vault RNA or the portion thereof comprises one or more sequence motifs that assembles to form an RNP. In some embodiments, the full-length vault RNA or the portion thereof comprises (i) one or more one or more secondary RNA structures, and (ii) one or more sequence motifs, wherein (i), (ii), or both assemble to form an RNP.

In some embodiments, a splice editor nucleic acid molecule of the disclosure comprises a vault RNA or portion thereof, wherein the vault RNA is VTRNA2-1.

### Methods to Engineer a Splice Editor Nucleic Acid of the Disclosure

The present disclosure provides methods to engineer a splice editor nucleic acid described herein. In some embodiments, the method comprises (A) identifying one or more candidate ncRNAs; (B) obtaining a ncRNA sequence from the one or more candidate ncRNAs; and (C) producing a splice editor nucleic acid comprising a nucleotide sequence comprising (i) an intronic sequence comprising (a) the ncRNA sequence, and (b) one or more binding domains described herein; (ii) a splice acceptor and/or splice donor; and (iii) one or more exonic sequences, thereby providing a splice editor nucleic acid for targeting trans-splicing of the target RNA (e.g., target pre-mRNA). In some embodiments, the method comprises introducing the splice editor nucleic acid to a cell or population of cells and determining the efficiency of trans-splicing of the target RNA (e.g., target pre-mRNA) according to a method described herein. In some embodiments, the efficiency of trans-splicing of the splice editor nucleic acid is compared to that of a control nucleic acid. In some embodiments, the control nucleic acid comprises (i), (ii), and (iii) and lacks the ncRNA sequence.

Methods of Identifying Candidate ncRNA Sequences

In some embodiments, identifying one or more candidate ncRNA sequences comprises (i) obtaining one or more ncRNA sequences from a database and/or by experimental analysis of RNA expressed by a cell or organism as described herein; (ii) predicting the secondary structure formed by the one or more ncRNA sequences according to a method described herein; (iii) comparing the predicted secondary structure of (ii) to a reference secondary structure (e.g., a secondary structure present in a ncRNA known in the art); and (iv) selecting one or more candidate ncRNA sequences with a predicted secondary structure having substantial similarity to the reference secondary structure. Computational methods for predicting the secondary structures formed by a ncRNA sequence are known in the art (see, e.g., Lorenz, et al. ViennaRNA Package 2.0 Algorithms for Molecular Biology, 6:1 26, 2011). Methods for performing RNA sequence analysis by comparing predicted secondary structures to a reference secondary structure are also known in the art (see, e.g., Eddy, et al (1994) Nucleic Acids Res 22:2079).

In some embodiments, identifying one or more candidate ncRNA sequences comprises (i) obtaining one or more ncRNA sequences from a database and/or by experimental analysis of RNA expressed by a cell or organism as described herein; (ii) predicting the secondary structure formed by the one or more ncRNA sequences according to a method described herein; (iii) comparing the predicted secondary structure of (ii) to a reference secondary structure (e.g., a secondary structure present in a ncRNA known in the art); and (iv) selecting one or more candidate ncRNA sequences with a predicted secondary structure having substantial similarity to the reference secondary structure and comprising a sequence motif described herein (e.g., a sequence motif comprises one or more sequences set forth in Table 1).

In some embodiments, the one or more candidate ncRNA sequences are selected from any one or any combination of sequences set forth in SEQ ID NOs: 9-657

Method of Obtaining a ncRNA Sequence

In some embodiments, obtaining a ncRNA sequence for inclusion in a splice editor nucleic acid of the disclosure comprises (i) identifying one or more candidate ncRNA sequences as described herein; and (ii) selecting a ncRNA sequence from the one or more candidate ncRNA sequences.

In some embodiments, obtaining the ncRNA sequence comprises selecting a ncRNA sequence from the one or more candidate ncRNA sequences (e.g., one or more candidate ncRNA sequences selected from any one or any combination of sequences set forth in SEQ ID NOs: 9-657), wherein the ncRNA sequence is at least about 7, about 8, about 9, about 10, about 11, about 12, about 13, about 14, or about 15 nucleotides in length and comprises a Sm motif described herein (e.g., a Sm motif set forth in Table 1). In some embodiments, the ncRNA sequence is about 7 nucleotides in length and comprises a Sm motif described herein (e.g., a Sm motif set forth in Table 1). In some embodiments, the ncRNA sequence is about 8 nucleotides in length and comprises a Sm motif described herein (e.g., a Sm motif set forth in Table 1). In some embodiments, the ncRNA sequence is about 9 nucleotides in length and comprises a Sm motif described herein (e.g., a Sm motif set forth in Table 1). In some embodiments, the ncRNA sequence is about 10 nucleotides in length and comprises a Sm motif described herein (e.g., a Sm motif set forth in Table 1). In some embodiments, the ncRNA sequence is about 11 nucleotides in length and comprises a Sm motif described herein (e.g., a Sm motif set forth in Table 1). In some embodiments, the ncRNA sequence is about 12 nucleotides in length and comprises a Sm motif described herein (e.g., a Sm motif set forth in Table 1).

In some embodiments, obtaining the ncRNA sequence comprises selecting a ncRNA sequence from the one or more candidate ncRNA sequences (e.g., one or more candidate ncRNA sequences selected from any one or any combination of sequences set forth in SEQ ID NOs: 9-657), wherein the ncRNA sequence is at least about 40, about 45, about 50, about 55, about 60, about 65, about 70, about 75, about 80, about 85, about 90, about 95, or about 100 nucleotides in length and comprises a Sm motif described herein (e.g., a Sm motif set forth in Table 1).

In some embodiments, obtaining the ncRNA sequence comprises selecting a ncRNA sequence having at least about 60%, about 65%, about 70%, about 75%, about 80%, about 85%, about 90%, about 91%, about 92%, about 93%, about 94%, about 95%, about 96%, about 97%, about 98%, about 99% to one or more candidate ncRNA sequences or a portion thereof (e.g., one or more candidate ncRNA sequences selected from any one or any combination of sequences set forth in SEQ ID NOs: 9-657), wherein the ncRNA sequence is at least about 40, about 45, about 50, about 55, about 60, about 65, about 70, about 75, about 80, about 85, about 90, about 95, or about 100 nucleotides in length and comprises a Sm motif described herein (e.g., a Sm motif set forth in Table 1).

In some embodiments, obtaining the ncRNA sequence comprises selecting a ncRNA sequence from the one or more candidate ncRNA sequences (e.g., one or more candidate ncRNA sequences selected from any one or any combination of sequences set forth in SEQ ID NOs: 9-657), wherein the ncRNA sequence is at least about 40, about 45, about 50, about 55, about 60, about 65, about 70, about 75, about 80, about 85, about 90, about 95, or about 100 nucleotides in length and comprises a H-box motif described herein (e.g., a H-box motif set forth in Table 1).

In some embodiments, obtaining the ncRNA sequence comprises selecting a ncRNA sequence having at least about 60%, about 65%, about 70%, about 75%, about 80%, about 85%, about 90%, about 91%, about 92%, about 93%, about 94%, about 95%, about 96%, about 97%, about 98%, about 99% to one or more candidate ncRNA sequences or a portion thereof (e.g., one or more candidate ncRNA sequences selected from any one or any combination of sequences set forth in SEQ ID NOs: 9-657 or a portion thereof), wherein the ncRNA sequence is at least about 40, about 45, about 50, about 55, about 60, about 65, about 70, about 75, about 80, about 85, about 90, about 95, or about 100 nucleotides in length and comprises a H-box motif described herein (e.g., a H-box motif set forth in Table 1).

In some embodiments, obtaining the ncRNA sequence comprises selecting a ncRNA sequence from the one or more candidate ncRNA sequences (e.g., one or more candidate ncRNA sequences selected from any one or any combination of sequences set forth in SEQ ID NOs: 9-657), wherein the ncRNA sequence is at least about 40, about 45, about 50, about 55, about 60, about 65, about 70, about 75, about 80, about 85, about 90, about 95, or about 100 nucleotides in length and comprises an ACA-box motif described herein (e.g., an ACA-box motif set forth in Table 1).

In some embodiments, obtaining the ncRNA sequence comprises selecting a ncRNA sequence having at least about 60%, about 65%, about 70%, about 75%, about 80%, about 85%, about 90%, about 91%, about 92%, about 93%, about 94%, about 95%, about 96%, about 97%, about 98%, about 99% to one or more candidate ncRNA sequences or a portion thereof (e.g., one or more candidate ncRNA sequences selected from any one or any combination of sequences set forth in SEQ ID NOs: 9-657 or a portion thereof), wherein the ncRNA sequence is at least about 40, about 45, about 50, about 55, about 60, about 65, about 70, about 75, about 80, about 85, about 90, about 95, or about 100 nucleotides in length and comprises a ACA-box motif described herein (e.g., a ACA-box motif set forth in Table 1).

In some embodiments, obtaining the ncRNA sequence comprises selecting a ncRNA sequence from the one or more candidate ncRNA sequences (e.g., one or more candidate ncRNA sequences selected from any one or any combination of sequences set forth in SEQ ID NOs: 9-657), wherein the ncRNA sequence is at least about 40, about 45, about 50, about 55, about 60, about 65, about 70, about 75, about 80, about 85, about 90, about 95, or about 100 nucleotides in length and comprises an H-box and an ACA-box motif described herein (e.g., a H-box and a ACA-box motif set forth in Table 1).

In some embodiments, obtaining the ncRNA sequence comprises selecting a ncRNA sequence having at least about 60%, about 65%, about 70%, about 75%, about 80%, about 85%, about 90%, about 91%, about 92%, about 93%, about 94%, about 95%, about 96%, about 97%, about 98%, about 99% to one or more candidate ncRNA sequences or a portion thereof (e.g., one or more candidate ncRNA sequences selected from any one or any combination of sequences set forth in SEQ ID NOs: 9-657 or a portion thereof), wherein the ncRNA sequence is at least about 40, about 45, about 50, about 55, about 60, about 65, about 70, about 75, about 80, about 85, about 90, about 95, or about 100 nucleotides in length and comprises an H-box and an ACA-box motif described herein (e.g., a H-box and a ACA-box motif set forth in Table 1).

In some embodiments, obtaining the ncRNA sequence comprises selecting a ncRNA sequence from the one or more candidate ncRNA sequences (e.g., one or more candidate ncRNA sequences selected from any one or any combination of sequences set forth in SEQ ID NOs: 9-657), wherein the ncRNA sequence is at least about 40, about 45, about 50, about 55, about 60, about 65, about 70, about 75, about 80, about 85, about 90, about 95, or about 100 nucleotides in length and comprises a (i)C-box motif described herein (e.g., a C-box motif set forth in Table 1), (ii) C′-box motif described herein (e.g., a C′-box motif set forth in Table 1), (iii) D-box motif described herein (e.g., a D-box motif set forth in Table 1), (iv) a D′-box motif described herein (e.g., a D′-box motif set forth in Table 1), or (v) a combination of (i)-(iv).

In some embodiments, obtaining the ncRNA sequence comprises selecting a ncRNA sequence having at least about 60%, about 65%, about 70%, about 75%, about 80%, about 85%, about 90%, about 91%, about 92%, about 93%, about 94%, about 95%, about 96%, about 97%, about 98%, about 99% to one or more candidate ncRNA sequences or a portion thereof (e.g., one or more candidate ncRNA sequences selected from any one or any combination of sequences set forth in SEQ ID NOs: 9-657 or a portion thereof), wherein the sequence is at least about 40, about 45, about 50, about 55, about 60, about 65, about 70, about 75, about 80, about 85, about 90, about 95, or about 100 nucleotides in length and comprises a (i)C-box motif described herein (e.g., a C-box motif set forth in Table 1), (ii) C′-box motif described herein (e.g., a C′-box motif set forth in Table 1), (iii) D-box motif described herein (e.g., a D-box motif set forth in Table 1), (iv) a D′-box motif described herein (e.g., a D′-box motif set forth in Table 1), or (v) a combination of (i)-(iv).

### Methods of Producing the Splice Editor Nucleic Acids

The splice editor nucleic acids provided by the disclosure are produced by suitable nucleic acid synthesis method or means known in the art. In some embodiments, the splice editor nucleic acid is produced as an RNA. In some embodiments, the splice editor nucleic acid is produced as a DNA. The present disclosure further provides delivery systems comprising the splice editor nucleic acid, e.g., a vector comprising the splice editor nucleic acid, a lipid particle comprising the splice editor nucleic acid.

Methods of producing the splice editor nucleic acids include, but are not limited to, in vitro transcription (IVT), synthetic and/or chemical synthesis methods, or a combination thereof. In some embodiments, enzymatic (e.g., IVT), solid-phase, liquid-phase, combined synthetic methods, small region synthesis, and ligation methods are utilized.

In some embodiments, the disclosure provides splice editor nucleic acids produced using IVT enzymatic synthesis methods. Methods of making polynucleotides by IVT are known in the art and are described in International Application PCT/US2013/30062.

In some embodiments, the disclosure provides splice editor nucleic acids chemically synthesized by any means described in the art. In some embodiments, the splice editor nucleic acids are produced by oligonucleotide synthesis. Oligonucleotide synthesis is the chemical synthesis of relatively short fragments or strands of single-stranded nucleic acids with a defined chemical structure (sequence). Methods of oligonucleotide synthesis are known in the art (see e.g., Reese (2005) Organic & Biomolecular Chemistry 3(21):3851). While chemical synthetic procedures are continually expanding, purifications of such nucleic acids by procedures such as high performance liquid chromatography (HPLC, which avoids the use of gels such as PAGE) tends to become more challenging as polynucleotide lengths increase significantly beyond a hundred or so nucleotides. One approach used for generating nucleic acids of greater length is to produce two or more molecules that are ligated together.

### Methods of Determining Splice Editor Nucleic Acid Tran Splicing Efficiency

In some embodiments, the disclosure provides methods to determine the efficiency of trans-splicing of a target RNA (e.g., pre-mRNA) using a splice editor nucleic acid molecule described herein.

In some embodiments, the method comprises use of a fluorescence-based splicing reporter assay. In some embodiments, the assay comprises contacting a reporter cell or population of cells with the splice editor nucleic acid molecule according to a method described herein (e.g., via transfection with a viral vector encoding the splice editor nucleic acid molecule), wherein the splice editor nucleic acid molecule comprises at least one exon encoding a reporter molecule, wherein a trans-splicing event is indicated by the presence of a fluorescent signal from the reporter molecule that is detected using a method known in the art. For example, in some embodiments, the reporter molecule is a fluorescent protein detected using fluorescence-activated cell sorting (FACS). For example, in some embodiments, the splice editor nucleic acid molecule comprises a nucleotide sequence encoding a first portion of a fluorescent protein and the target RNA comprises a nucleotide sequence encoding a second portion of a fluorescent protein, wherein the trans-splicing generates an RNA comprising a nucleotide sequence encoding the full-length fluorescent protein, and wherein the trans-splicing event is detected using a method of fluorescent measurement (e.g., FACS).

In some embodiments, the splice editor nucleic acid is introduced to a cell using a method described herein (e.g., via a viral or non-viral vector) for a duration, whereupon RNA from the cell is extracted and trans-splicing products are detected. For example, an mRNA spliced from a target RNA (e.g., a target pre-mRNA) is analyzed by a suitable method known in the art (e.g., end-point or quantitative RT-PCR or RNA sequencing). In some embodiments, a cell or population of cells is contacted with the splice editor nucleic acid molecule, wherein next-generating sequencing (NGS) techniques are used to determine the extent of trans-splicing. For example, in some embodiments, mRNA extracted from cells treated or contacted with a splice editor nucleic acid provided by the disclosure is enzymatically converted into cDNA, which is further by analyzed by NGS analysis to determine the extent of mRNA molecule comprising exonic sequence incorporated from the splice editor nucleic acid.

In some embodiments, trans-splicing is determined by protein sequence analysis of a polypeptide translated from an mRNA spliced from the pre-mRNA. In some embodiments, an RNA-guided molecule corrects a mutation by the incorporation of a corrected exon, wherein translation of the mRNA resulting from trans-splicing of the pre-mRNA and the splice editor nucleic acid generates a polypeptide comprising an amino acid sequence encoded by the corrected exon. The protein sequence analysis is performed using techniques including, but not limited to, Sanger sequencing, mass spectrometry, functional assays that measure an enzymatic activity of the polypeptide, or immunoblotting using an antibody reactive to the corrected amino acid sequence.

In some embodiments, trans-splicing is determined by measuring the activity of a protein translated from an mRNA spliced from the pre-mRNA. For example, in some embodiments, the protein is an enzyme and the method of measuring trans-splicing comprises measuring enzymatic activity using a functional ELISA.

In some embodiments, a method for measuring the efficiency of trans-splicing using a splice editor nucleic acid of the disclosure is described in U.S. application Ser. No. 16/994,230, incorporated herein by reference.

In some embodiments, a method for measuring the efficiency of trans-splicing using a splice editor nucleic acid of the disclosure is any one described in Chen, et al (2009) Gene Ther 16:211; Rindt, et al (2012) Cell Mol Life Sci 69:4191; Monjaret, et al (2014) Mol Ther 22:1176; Berger, et al (2015) Mol Ther 23:918.

In some embodiments, a method described herein is used to measure the efficiency of trans-splicing of a pre-mRNA using a splice editor nucleic acid molecule described herein.

In some embodiments, a splice editor nucleic acid molecule described herein comprising a nucleotide sequence comprising (i) at least one intronic sequence comprising one or more binding domains each having complementarity to a target sequence in the pre-mRNA and a ncRNA; (ii) one or more splice sites (e.g., a splice acceptor and/or splice donor); and (iii) at least one exonic sequence results in an efficiency of trans-splicing that is greater than the splice editor nucleic acid molecule without the ncRNA, as measured using a method described herein. In some embodiments, the splice editor nucleic acid molecule described herein results in an efficiency of trans-splicing that is increased by at least about 1.5-fold, about 2-fold, about 3-fold, about 4-fold, about 5-fold, about 10-fold, or about 20-fold compared to the efficiency of trans-splicing of a splice editor nucleic acid molecule without the ncRNA.

### Exemplary Splice Editor Nucleic Acids

In some embodiments, the disclosure provides a nucleic acid of Subgroup I for targeting trans-splicing of a target RNA (e.g., pre-mRNA) in a cell, the nucleic acid comprising a nucleotide sequence comprising from 5′ to 3′: (a) at least one intronic sequence comprising (i) one or more binding domain sequences each with complementarity to a target sequence in the target RNA (e.g., pre-mRNA); (ii) a ncRNA sequence that forms a secondary structure and/or comprises a sequence motif to direct the one or more binding domain sequences to the target RNA (e.g., pre-mRNA); and (iii) one or more splicing signals; (b) a splice acceptor; and (c) at least one exonic sequence.

In some embodiments, the nucleic acid of Subgroup I comprises a nucleotide sequence comprising from 5′ to 3′: (a) at least one intronic sequence comprising (i) one or more binding domain sequences each with complementarity to a target sequence in the target RNA (e.g., pre-mRNA); (ii) a ncRNA sequence that forms a secondary structure and/or comprises a sequence motif to direct the one or more binding domain sequences to the target RNA (e.g., pre-mRNA); and (iii) one or more splicing signals, wherein the ncRNA is an snRNA; (b) a splice acceptor; and (c) at least one exonic sequence. In some embodiments, the snRNA is selected from a U1 snRNA, a U2 snRNA, a U4 snRNA, a U4atac snRNA, a U5 snRNA, a U6 snRNA, a U6atac snRNA, a U11 snRNA, a U12 snRNA, and a U7 snRNA.

In some embodiments, the nucleic acid of Subgroup I comprises a nucleotide sequence comprising from 5′ to 3′: (a) at least one intronic sequence comprising (i) one or more binding domain sequences each with complementarity to a target sequence in the target RNA (e.g., pre-mRNA); (ii) a ncRNA comprising a sequence motif to direct the one or more binding domain sequences to the target RNA (e.g., pre-mRNA), wherein the sequence motif selected from a Sm sequence motif and a Lsm sequence motif, and (iii) one or more splicing signals; (b) a splice acceptor; and (c) at least one exonic sequence.

In some embodiments, the nucleic acid of Subgroup I comprises a nucleotide sequence comprising from 5′ to 3′: (a) at least one intronic sequence comprising (i) one or more binding domain sequences each with complementarity to a target sequence in the target RNA (e.g., pre-mRNA); (ii) a ncRNA sequence that forms a secondary structure and comprises a sequence motif to direct the one or more binding domain sequences to the target RNA (e.g., pre-mRNA), wherein the sequence motif selected from a Sm sequence motif and a Lsm sequence motif, and (iii) one or more splicing signals; (b) a splice acceptor; and (c) at least one exonic sequence.

In some embodiments, the nucleic acid of Subgroup I comprises a nucleotide sequence comprising from 5′ to 3′: (a) at least one intronic sequence comprising (i) one or more binding domain sequences each with complementarity to a target sequence in the target RNA (e.g., pre-mRNA); (ii) a ncRNA sequence that forms a secondary structure and/or comprises a sequence motif that assembles to form an RNP that directs the binding domain to the target RNA (e.g., pre-mRNA); and (iii) one or more splicing signals; (b) a splice acceptor; and (c) at least one exonic sequence.

In some embodiments, the nucleic acid of Subgroup I comprises a nucleotide sequence comprising from 5′ to 3′: (a) at least one intronic sequence comprising (i) one or more binding domain sequences each with complementarity to a target sequence in the target RNA (e.g., pre-mRNA); (ii) a ncRNA sequence that forms a secondary structure and/or comprises a sequence motif that assembles to form an RNP that directs the one or more binding domains to the target RNA (e.g., pre-mRNA), wherein the ncRNA is an snRNA; and (iii) one or more splicing signals; (b) a splice acceptor; and (c) at least one exonic sequence. In some embodiments, the snRNA is selected from a U1 snRNA, a U2 snRNA, a U4 snRNA, a U4atac snRNA, a U5 snRNA, a U6 snRNA, a U6atac snRNA, a U11 snRNA, a U12 snRNA, and a U7 snRNA.

In some embodiments, the nucleic acid of Subgroup I comprises a nucleotide sequence comprising from 5′ to 3′: (a) at least one intronic sequence comprising (i) one or more binding domain sequences each with complementarity to a target sequence in the target RNA (e.g., pre-mRNA); (ii) a ncRNA sequence comprising a sequence motif that assembles to form an RNP that directs the binding domain to the target RNA (e.g., pre-mRNA), wherein the sequence motif selected from a Sm sequence motif and a Lsm sequence motif, and (iii) one or more splicing signals; (b) a splice acceptor; and (c) at least one exonic sequence.

In some embodiments, the nucleic acid of Subgroup I comprises a nucleotide sequence comprising from 5′ to 3′: (a) at least one intronic sequence comprising (i) one or more binding domain sequences each with complementarity to a target sequence in the target RNA (e.g., pre-mRNA); (ii) a ncRNA sequence that forms a secondary structure and comprises a sequence motif that assembles to form an RNP that directs the binding domain to the target RNA (e.g., pre-mRNA), wherein the sequence motif selected from a Sm sequence motif and a Lsm sequence motif, and (iii) one or more splicing signals; (b) a splice acceptor; and (c) at least one exonic sequence.

In some embodiments, the nucleic acid of Subgroup I comprises a nucleotide sequence comprising from 5′ to 3′: (a) at least one intronic sequence comprising (i) one or more binding domain sequences of about 4 to about 300 nucleotides each with complementarity to a target sequence in the target RNA (e.g., pre-mRNA); (ii) a ncRNA sequence of about 7 to about 300 nucleotides in length that forms a secondary structure and/or comprises a sequence motif to direct the one or more binding domain sequences to the target RNA (e.g., pre-mRNA); and (iii) one or more splicing signals; (b) a splice acceptor; and (c) at least one exonic sequence.

In some embodiments, the nucleic acid of Subgroup I comprises a nucleotide sequence comprising from 5′ to 3′: (a) at least one intronic sequence comprising (i) one or more binding domain sequences of about 4 to about 300 nucleotides each with complementarity to a target sequence in the target RNA (e.g., pre-mRNA); (ii) a ncRNA sequence of about 7 to about 300 nucleotides in length that forms a secondary structure and/or comprises a sequence motif to direct the one or more binding domain sequences to the target RNA (e.g., pre-mRNA), wherein the ncRNA is an snRNA; and (iii) one or more splicing signals; (b) a splice acceptor; and (c) at least one exonic sequence. In some embodiments, the snRNA is selected from a U1 snRNA, a U2 snRNA, a U4 snRNA, a U4atac snRNA, a U5 snRNA, a U6 snRNA, a U6atac snRNA, a U11 snRNA, a U12 snRNA, and a U7 snRNA.

In some embodiments, the nucleic acid of Subgroup I comprises a nucleotide sequence comprising from 5′ to 3′: (a) at least one intronic sequence comprising (i) one or more binding domain sequences of about 4 to about 300 nucleotides each with complementarity to a target sequence in the target RNA (e.g., pre-mRNA); (ii) a ncRNA sequence of about 7 to about 300 nucleotides in length comprising a sequence motif to direct the one or more binding domain sequences to the target RNA (e.g., pre-mRNA), wherein the sequence motif selected from a Sm sequence motif and a Lsm sequence motif, and (iii) one or more splicing signals; (b) a splice acceptor; and (c) at least one exonic sequence.

In some embodiments, the nucleic acid of Subgroup I comprises a nucleotide sequence comprising from 5′ to 3′: (a) at least one intronic sequence comprising (i) one or more binding domain sequences of about 4 to about 300 nucleotides each with complementarity to a target sequence in the target RNA (e.g., pre-mRNA); (ii) a ncRNA sequence of about 7 to about 300 nucleotides in length that forms a secondary structure and comprises a sequence motif to direct the one or more binding domain sequences to the target RNA (e.g., pre-mRNA), wherein the sequence motif selected from a Sm sequence motif and a Lsm sequence motif, and (iii) one or more splicing signals; (b) a splice acceptor; and (c) at least one exonic sequence.

In some embodiments, the nucleic acid of Subgroup I comprises a nucleotide sequence comprising from 5′ to 3′: (a) at least one intronic sequence comprising (i) one or more binding domain sequences of about 4 to about 300 nucleotides each with complementarity to a target sequence in the target RNA (e.g., pre-mRNA); (ii) a ncRNA sequence of about 7 to about 300 nucleotides in length which forms a secondary structure and/or comprises a sequence motif that assembles to form an RNP that directs the binding domain to the target RNA (e.g., pre-mRNA); and (iii) one or more splicing signals; (b) a splice acceptor; and (c) at least one exonic sequence.

In some embodiments, the nucleic acid of Subgroup I comprises a nucleotide sequence comprising from 5′ to 3′: (a) at least one intronic sequence comprising (i) one or more binding domain sequences of about 4 to about 300 nucleotides each with complementarity to a target sequence in the target RNA (e.g., pre-mRNA); (ii) a ncRNA sequence of about 7 to about 300 nucleotides in length which forms a secondary structure and/or comprises a sequence motif that assembles to form an RNP that directs the binding domain to the target RNA (e.g., pre-mRNA), wherein the ncRNA is an snRNA; and (iii) one or more splicing signals; (b) a splice acceptor; and (c) at least one exonic sequence. In some embodiments, the snRNA is selected from a U1 snRNA, a U2 snRNA, a U4 snRNA, a U4atac snRNA, a U5 snRNA, a U6 snRNA, a U6atac snRNA, a U11 snRNA, a U12 snRNA, and a U7 snRNA.

In some embodiments, the nucleic acid of Subgroup I comprises a nucleotide sequence comprising from 5′ to 3′: (a) at least one intronic sequence comprising (i) one or more binding domain sequences of about 4 to about 300 nucleotides each with complementarity to a target sequence in the target RNA (e.g., pre-mRNA); (ii) a ncRNA sequence of about 7 to about 300 nucleotides in comprising a sequence motif that assembles to form an RNP that directs the binding domain to the target RNA (e.g., pre-mRNA), wherein the sequence motif selected from a Sm sequence motif and a Lsm sequence motif, and (iii) one or more splicing signals; (b) a splice acceptor; and (c) at least one exonic sequence.

In some embodiments, the nucleic acid of Subgroup I comprises a nucleotide sequence comprising from 5′ to 3′: (a) at least one intronic sequence comprising (i) one or more binding domain sequences of about 4 to about 300 nucleotides each with complementarity to a target sequence in the target RNA (e.g., pre-mRNA); (ii) a ncRNA sequence of about 7 to about 300 nucleotides in length which forms a secondary structure and comprises a sequence motif that assembles to form an RNP that directs the binding domain to the target RNA (e.g., pre-mRNA), wherein the sequence motif selected from a Sm sequence motif and a Lsm sequence motif, and (iii) one or more splicing signals; (b) a splice acceptor; and (c) at least one exonic sequence.

In some embodiments, the nucleic acid of Subgroup I comprises one binding domain. In some embodiments, the nucleic acid of Subgroup I comprises two binding domains. In some embodiments, the nucleic acid of Subgroup I comprises three binding domains. In some embodiments, the nucleic acid of Subgroup I comprises four binding domains. In some embodiments, the nucleic acid of Subgroup I comprises five binding domains.

In some embodiments, the target RNA is a pre-mRNA. In some embodiments, the pre-mRNA comprises from 5′ to3′: a 5′ exon, a splice donor, an intron, a splice acceptor, and a 3′ exon, wherein the 3′ exon comprises a mutation. In some embodiments, each of the one or more binding domains of the nucleic acid of Subgroup I is complementary to a target sequence in the target RNA (e.g., pre-mRNA), wherein the target sequence is positioned in the 5′ exon in the pre-mRNA.

In some embodiments, the target sequence is proximal to the splice donor of the pre-mRNA. In some embodiments, the target sequence is within the intron of the pre-mRNA. In some embodiments, the target sequence is proximal to the splice acceptor of the pre-mRNA. In some embodiments, the target sequence is positioned in the 3′ exon of the pre-mRNA. In some embodiments, trans-splicing occurs between the splice donor of the pre-mRNA and the splice acceptor of the nucleic acid of Subgroup I. In some embodiments, the trans-splicing results in ligation of the 3′end of the 5′ exon of the pre-mRNA and the 5′end of the at least one exonic sequence of the nucleic acid of Subgroup I.

In some embodiments, the one or more splicing signals of the nucleic acid of Subgroup I comprises a branch point. In some embodiments, the one or more splicing signals of the nucleic acid of Subgroup I comprises a polypyrimidine tract. In some embodiments, the one or more splicing signals of the nucleic acid of Subgroup I comprises a branch point and polypyrimidine tract. In some embodiments, the one or more splicing signals further comprises a ISE. In some embodiments, the one or more splicing signals further comprises a ISS.

In some embodiments, the at least one exonic sequence of the nucleic acid of Subgroup I comprises an ESE. In some embodiments, the at least one exonic sequence of the nucleic acid of Subgroup I comprises an ESS.

In some embodiments, the disclosure provides a nucleic acid for targeting trans-splicing of a target RNA (e.g., pre-mRNA) in a cell, the nucleic acid comprising a nucleotide sequence comprising from 5′ to 3′: (a) at least one exonic sequence; (b) a splice donor; (c) at least one intronic sequence comprising (i) a ncRNA sequence, and (ii) one or more binding domain sequences each with complementarity to a target sequence in the target RNA (e.g., pre-mRNA), wherein the ncRNA forms a secondary structure and/or comprises a sequence motif to direct the one or more binding domain to the target RNA.

In some embodiments, the disclosure provides a nucleic acid of Subgroup II for targeting trans-splicing of a target RNA (e.g., pre-mRNA) in a cell, the nucleic acid comprising a nucleotide sequence comprising from 5′ to 3′: (a) at least one exonic sequence; (b) a splice donor; (c) at least one intronic sequence comprising (i) a ncRNA sequence, and (ii) one or more binding domain sequences each with complementarity to a target sequence in the target RNA (e.g., pre-mRNA), wherein the ncRNA forms a secondary structure and/or comprises a sequence motif to direct the one or more binding domain to the target RNA, and wherein the ncRNA is an snRNA. In some embodiments, the snRNA is selected from a U1 snRNA, a U2 snRNA, a U4 snRNA, a U4atac snRNA, a U5 snRNA, a U6 snRNA, a U6atac snRNA, a U11 snRNA, a U12 snRNA, and a U7 snRNA.

In some embodiments, the nucleic acid of Subgroup II comprises a nucleotide sequence comprising from 5′ to 3′: (a) at least one exonic sequence; (b) a splice donor; (c) at least one intronic sequence comprising (i) a ncRNA sequence, and (ii) one or more binding domain sequences each with complementarity to a target sequence in the target RNA (e.g., pre-mRNA), wherein the ncRNA sequence comprises a sequence motif to direct the one or more binding domain to the target RNA, and wherein the sequence motif selected from a Sm sequence motif and a Lsm sequence motif.

In some embodiments, the nucleic acid of Subgroup II comprises a nucleotide sequence comprising from 5′ to 3′: (a) at least one exonic sequence; (b) a splice donor; (c) at least one intronic sequence comprising (i) a ncRNA sequence, and (ii) one or more binding domain sequences each with complementarity to a target sequence in the target RNA (e.g., pre-mRNA), wherein the ncRNA sequence forms a secondary structure and comprises a sequence motif to direct the one or more binding domain to the target RNA, and wherein the sequence motif selected from a Sm sequence motif and a Lsm sequence motif.

In some embodiments, the nucleic acid of Subgroup II comprises a nucleotide sequence comprising from 5′ to 3′: (a) at least one exonic sequence; (b) a splice donor; (c) at least one intronic sequence comprising (i) a ncRNA sequence, and (ii) one or more binding domain sequences each with complementarity to a target sequence in the target RNA (e.g., pre-mRNA), wherein the ncRNA forms a secondary structure and/or comprises a sequence motif that assembles to form an RNP to direct the one or more binding domain to the target RNA.

In some embodiments, the nucleic acid of Subgroup II comprises a nucleotide sequence comprising from 5′ to 3′: (a) at least one exonic sequence; (b) a splice donor; (c) at least one intronic sequence comprising (i) a ncRNA sequence, and (ii) one or more binding domain sequences each with complementarity to a target sequence in the target RNA (e.g., pre-mRNA), wherein the ncRNA forms a secondary structure and/or comprises a sequence motif that assembles to form an RNP to direct the one or more binding domain to the target RNA, and wherein the ncRNA is an snRNA. In some embodiments, the snRNA is selected from a U1 snRNA, a U2 snRNA, a U4 snRNA, a U4atac snRNA, a U5 snRNA, a U6 snRNA, a U6atac snRNA, a U11 snRNA, a U12 snRNA, and a U7 snRNA.

In some embodiments, the nucleic acid of Subgroup II comprises a nucleotide sequence comprising from 5′ to 3′: (a) at least one exonic sequence; (b) a splice donor; (c) at least one intronic sequence comprising (i) a ncRNA sequence, and (ii) one or more binding domain sequences each with complementarity to a target sequence in the target RNA (e.g., pre-mRNA), wherein the ncRNA sequence comprises a sequence motif that assembles to form an RNP to direct the one or more binding domain to the target RNA, and wherein the sequence motif selected from a Sm sequence motif and a Lsm sequence motif.

In some embodiments, the nucleic acid of Subgroup II comprises a nucleotide sequence comprising from 5′ to 3′: (a) at least one exonic sequence; (b) a splice donor; (c) at least one intronic sequence comprising (i) a ncRNA sequence, and (ii) one or more binding domain sequences each with complementarity to a target sequence in the target RNA (e.g., pre-mRNA), wherein the ncRNA sequence forms a secondary structure and comprises a sequence motif that assembles to form an RNP to direct the one or more binding domain to the target RNA, and wherein the sequence motif selected from a Sm sequence motif and a Lsm sequence motif.

In some embodiments, the nucleic acid of Subgroup II comprises a nucleotide sequence comprising from 5′ to 3′: (a) at least one exonic sequence; (b) a splice donor; (c) at least one intronic sequence comprising (i) a ncRNA sequence of about 7 to about 300 nucleotides in length, and (ii) one or more binding domain sequences of about 4 to about 300 nucleotides each with complementarity to a target sequence in the target RNA (e.g., pre-mRNA), wherein the ncRNA forms a secondary structure and/or comprises a sequence motif to direct the one or more binding domain to the target RNA.

In some embodiments, the nucleic acid of Subgroup II comprises a nucleotide sequence comprising from 5′ to 3′: (a) at least one exonic sequence; (b) a splice donor; (c) at least one intronic sequence comprising (i) a ncRNA sequence of about 7 to about 300 nucleotides in length, and (ii) one or more binding domain sequences of about 4 to about 300 nucleotides each with complementarity to a target sequence in the target RNA (e.g., pre-mRNA), wherein the ncRNA forms a secondary structure and/or comprises a sequence motif to direct the one or more binding domain to the target RNA, and wherein the ncRNA is an snRNA. In some embodiments, the snRNA is selected from a U1 snRNA, a U2 snRNA, a U4 snRNA, a U4atac snRNA, a U5 snRNA, a U6 snRNA, a U6atac snRNA, a U11 snRNA, a U12 snRNA, and a U7 snRNA.

In some embodiments, the nucleic acid of Subgroup II comprises a nucleotide sequence comprising from 5′ to 3′: (a) at least one exonic sequence; (b) a splice donor; (c) at least one intronic sequence comprising (i) a ncRNA sequence of about 7 to about 300 nucleotides in length, and (ii) one or more binding domain sequences of about 4 to about 300 nucleotides each with complementarity to a target sequence in the target RNA (e.g., pre-mRNA), wherein the ncRNA sequence comprises a sequence motif to direct the one or more binding domain to the target RNA, and wherein the sequence motif selected from a Sm sequence motif and a Lsm sequence motif.

In some embodiments, the nucleic acid of Subgroup II comprises a nucleotide sequence comprising from 5′ to 3′: (a) at least one exonic sequence; (b) a splice donor; (c) at least one intronic sequence comprising (i) a ncRNA sequence of about 7 to about 300 nucleotides in length, and (ii) one or more binding domain sequences of about 4 to about 300 nucleotides each with complementarity to a target sequence in the target RNA (e.g., pre-mRNA), wherein the ncRNA sequence forms a secondary structure and comprises a sequence motif to direct the one or more binding domain to the target RNA, and wherein the sequence motif selected from a Sm sequence motif and a Lsm sequence motif.

In some embodiments, the nucleic acid of Subgroup II comprises a nucleotide sequence comprising from 5′ to 3′: (a) at least one exonic sequence; (b) a splice donor; (c) at least one intronic sequence comprising (i) a ncRNA sequence of about 7 to about 300 nucleotides in length, and (ii) one or more binding domain sequences of about 4 to about 300 nucleotides each with complementarity to a target sequence in the target RNA (e.g., pre-mRNA), wherein the ncRNA forms a secondary structure and/or comprises a sequence motif that assembles to form an RNP to direct the one or more binding domain to the target RNA.

In some embodiments, the nucleic acid of Subgroup II comprises a nucleotide sequence comprising from 5′ to 3′: (a) at least one exonic sequence; (b) a splice donor; (c) at least one intronic sequence comprising (i) a ncRNA sequence of about 7 to about 300 nucleotides in length, and (ii) one or more binding domain sequences of about 4 to about 300 nucleotides each with complementarity to a target sequence in the target RNA (e.g., pre-mRNA), wherein the ncRNA forms a secondary structure and/or comprises a sequence motif that assembles to form an RNP to direct the one or more binding domain to the target RNA, and wherein the ncRNA is an snRNA. In some embodiments, the snRNA is selected from a U1 snRNA, a U2 snRNA, a U4 snRNA, a U4atac snRNA, a U5 snRNA, a U6 snRNA, a U6atac snRNA, a U11 snRNA, a U12 snRNA, and a U7 snRNA.

In some embodiments, the nucleic acid of Subgroup II comprises a nucleotide sequence comprising from 5′ to 3′: (a) at least one exonic sequence; (b) a splice donor; (c) at least one intronic sequence comprising (i) a ncRNA sequence of about 7 to about 300 nucleotides in length, and (ii) one or more binding domain sequences of about 4 to about 300 nucleotides each with complementarity to a target sequence in the target RNA (e.g., pre-mRNA), wherein the ncRNA sequence comprises a sequence motif that assembles to form an RNP to direct the one or more binding domain to the target RNA, and wherein the sequence motif selected from a Sm sequence motif and a Lsm sequence motif.

In some embodiments, the nucleic acid of Subgroup II comprises a nucleotide sequence comprising from 5′ to 3′: (a) at least one exonic sequence; (b) a splice donor; (c) at least one intronic sequence comprising (i) a ncRNA sequence of about 7 to about 300 nucleotides in length, and (ii) one or more binding domain sequences of about 4 to about 300 nucleotides each with complementarity to a target sequence in the target RNA (e.g., pre-mRNA), wherein the ncRNA sequence forms a secondary structure and comprises a sequence motif that assembles to form an RNP to direct the one or more binding domain to the target RNA, and wherein the sequence motif selected from a Sm sequence motif and a Lsm sequence motif.

In some embodiments, the nucleic acid of Subgroup II comprises one binding domain. In some embodiments, the nucleic acid of Subgroup II comprises two binding domains. In some embodiments, the nucleic acid of Subgroup II comprises three binding domains. In some embodiments, the nucleic acid of Subgroup II comprises four binding domains. In some embodiments, the nucleic acid of Subgroup II comprises five binding domains.

In some embodiments, the target RNA is a pre-mRNA. In some embodiments, the pre-mRNA comprises from 5′ to 3′: a 5′ exon, a splice donor, an intron, a splice acceptor, and a 3′ exon, wherein the 5′ exon comprises a mutation. In some embodiments, each of the one or more binding domains of the nucleic acid of Subgroup II is complementary to a target sequence in the target RNA (e.g., pre-mRNA), wherein the target sequence is positioned in the 5′ exon in the pre-mRNA. In some embodiments, the target sequence is proximal to the splice donor of the pre-mRNA. In some embodiments, the target sequence is within the intron of the pre-mRNA. In some embodiments, the target sequence is proximal to the splice acceptor of the pre-mRNA. In some embodiments, the target sequence is positioned in the 3′ exon of the pre-mRNA. In some embodiments, trans-splicing occurs between the splice donor of the nucleic acid of Subgroup II and the splice acceptor of the pre-mRNA. In some embodiments, the trans-splicing results in ligation of the 3′end of the 5′ exon of the at least one exonic sequence of the nucleic acid of Subgroup II and the 5′end of the 3′exon of the pre-mRNA.

In some embodiments, the at least one exonic sequence of the nucleic acid of Subgroup II comprises an ESE. In some embodiments, the at least one exonic sequence of the nucleic acid of Subgroup II comprises an ESS.

In some embodiments, the disclosure provides a nucleic acid of Subgroup III for targeting trans-splicing of a target RNA (e.g., pre-mRNA) in a cell, the nucleic acid comprising a nucleotide sequence comprising from 5′ to 3′ (a) at least one intronic sequence comprising (i) a snoRNA sequence comprising an H/ACA box or a C/D box and one or more binding domain sequences each with complementarity to a pre-mRNA target sequence; and (ii) one or more splicing signals; (b) a splice acceptor; and (c) at least one exonic sequence.

In some embodiments, the disclosure provides a nucleic acid of Subgroup III for targeting trans-splicing of a target RNA (e.g., pre-mRNA) in a cell, the nucleic acid comprising a nucleotide sequence comprising from 5′ to 3′ (a) at least one intronic sequence comprising (i) a snoRNA sequence comprising an H/ACA box or a C/D box to direct the one or more binding domain sequences to the target RNA (e.g., pre-mRNA) and one or more binding domain sequences each with complementarity to a pre-mRNA target sequence; and (ii) one or more splicing signals; (b) a splice acceptor; and (c) at least one exonic sequence.

In some embodiments, the nucleic acid of Subgroup III comprises a nucleotide sequence comprising from 5′ to 3′ (a) at least one intronic sequence comprising (i) a snoRNA sequence comprising an H/ACA box or a C/D box that assembles to form an RNP to direct the one or more binding domain sequences to the target RNA (e.g., pre-mRNA) and one or more binding domain sequences each with complementarity to a pre-mRNA target sequence; and (ii) one or more splicing signals; (b) a splice acceptor; and (c) at least one exonic sequence.

In some embodiments, the nucleic acid of Subgroup III comprises a nucleotide sequence comprising from 5′ to 3′ (a) at least one intronic sequence comprising (i) a snoRNA sequence comprising an H/ACA box or a C/D box that directs the one or more binding domain sequences to the target RNA (e.g., pre-mRNA) and one or more binding domain sequences of about 4 to about 30 nucleotides in length, each with complementarity to a pre-mRNA target sequence; and (ii) one or more splicing signals; (b) a splice acceptor; and (c) at least one exonic sequence.

In some embodiments, the nucleic acid of Subgroup III comprises a nucleotide sequence comprising from 5′ to 3′ (a) at least one intronic sequence comprising (i) a snoRNA sequence comprising an H/ACA box or a C/D box that assembles to form an RNP to direct the one or more binding domain sequences to the target RNA (e.g., pre-mRNA) and one or more binding domain sequences of about 4 to about 30 nucleotides in length, each with complementarity to a pre-mRNA target sequence; and (ii) one or more splicing signals; (b) a splice acceptor; and (c) at least one exonic sequence.

In some embodiments, the target RNA is a pre-mRNA. In some embodiments, the pre-mRNA comprises from 5′ to 3′: a 5′ exon, a splice donor, an intron, a splice acceptor, and a 3′ exon, wherein the 3′ exon comprises a mutation. In some embodiments, each of the one or more binding domains of the nucleic acid of Subgroup III is complementary to a target sequence in the target RNA (e.g., pre-mRNA), wherein the target sequence is positioned in the 5′ exon in the pre-mRNA. In some embodiments, the target sequence is proximal to the splice donor of the pre-mRNA. In some embodiments, the target sequence is within the intron of the pre-mRNA. In some embodiments, the target sequence is proximal to the splice acceptor of the pre-mRNA. In some embodiments, the target sequence is positioned in the 3′ exon of the pre-mRNA. In some embodiments, trans-splicing occurs between the splice donor of the pre-mRNA and the splice acceptor of the nucleic acid of Subgroup III. In some embodiments, the trans-splicing results in ligation of the 3′end of the 5′ exon of the pre-mRNA and the 5′end of the at least one exonic sequence of the nucleic acid of Subgroup III.

In some embodiments, the one or more splicing signals of the nucleic acid of Subgroup III comprises a branch point. In some embodiments, the one or more splicing signals of the nucleic acid of Subgroup III comprises a polypyrimidine tract. In some embodiments, the one or more splicing signals of the nucleic acid of Subgroup III comprises a branch point and polypyrimidine tract. In some embodiments, the one or more splicing signals further comprises a ISE. In some embodiments, the one or more splicing signals further comprises a ISS.

In some embodiments, the at least one exonic sequence of the nucleic acid of Subgroup III comprises an ESE. In some embodiments, the at least one exonic sequence of the nucleic acid of Subgroup III comprises an ESS.

In some embodiments, the disclosure provides a nucleic acid of Subgroup IV for targeting trans-splicing of a target RNA (e.g., pre-mRNA) in a cell, the nucleic acid comprising a nucleotide sequence comprising from 5′ to 3′ (a) at least one exonic sequence; (b) a splice donor; and (c) at least one intronic sequence comprising a snoRNA sequence comprising an H/ACA box or a C/D box and one or more binding domain sequences each with complementarity to a pre-mRNA target sequence.

In some embodiments, the nucleic acid of Subgroup IV comprises a nucleotide sequence comprising from 5′ to 3′ (a) at least one exonic sequence; (b) a splice donor; and (c) at least one intronic sequence comprising a snoRNA sequence comprising an H/ACA box or a C/D box to direct the one or more binding domain sequences to the target RNA (e.g., pre-mRNA) and one or more binding domain sequences each with complementarity to a pre-mRNA target sequence.

In some embodiments, the nucleic acid of Subgroup IV comprises a nucleotide sequence comprising from 5′ to 3′ (a) at least one exonic sequence; (b) a splice donor; and (c) at least one intronic sequence comprising a snoRNA sequence comprising an H/ACA box or a C/D box that assembles to form an RNP to direct the one or more binding domain sequences to the target RNA (e.g., pre-mRNA) and one or more binding domain sequences, each with complementarity to a pre-mRNA target sequence.

In some embodiments, the nucleic acid of Subgroup IV comprises a nucleotide sequence comprising from 5′ to 3′ (a) at least one exonic sequence; (b) a splice donor; and (c) at least one intronic sequence comprising a snoRNA sequence comprising an H/ACA box or a C/D box to direct the one or more binding domain sequences to the target RNA (e.g., pre-mRNA) and one or more binding domain sequences of about 4 to about 30 nucleotides in length, each with complementarity to a pre-mRNA target sequence.

In some embodiments, the nucleic acid of Subgroup IV comprises a nucleotide sequence comprising from 5′ to 3′ (a) at least one exonic sequence; (b) a splice donor; and (c) at least one intronic sequence comprising a snoRNA sequence comprising an H/ACA box or a C/D box that assembles to form an RNP to direct the one or more binding domain sequences to the target RNA (e.g., pre-mRNA) and one or more binding domain sequences of about 4 to about 30 nucleotides in length, each with complementarity to a pre-mRNA target sequence.

In some embodiments, the target RNA is a pre-mRNA. In some embodiments, the pre-mRNA comprises from 5′ to 3′: a 5′ exon, a splice donor, an intron, a splice acceptor, and a 3′ exon, wherein the 5′ exon comprises a mutation. In some embodiments, each of the one or more binding domains of the nucleic acid of Subgroup IV is complementary to a target sequence in the target RNA (e.g., pre-mRNA), wherein the target sequence is positioned in the 5′ exon in the pre-mRNA. In some embodiments, the target sequence is proximal to the splice donor of the pre-mRNA. In some embodiments, the target sequence is within the intron of the pre-mRNA. In some embodiments, the target sequence is proximal to the splice acceptor of the pre-mRNA. In some embodiments, the target sequence is positioned in the 3′ exon of the pre-mRNA. In some embodiments, trans-splicing occurs between the splice donor of the nucleic acid of Subgroup IV and the splice acceptor of the pre-mRNA. In some embodiments, the trans-splicing results in ligation of the 3′end of the 5′ exon of the at least one exonic sequence of the nucleic acid of Subgroup IV and the 5′end of the 3′exon of the pre-mRNA.

In some embodiments, the at least one exonic sequence of the nucleic acid of Subgroup IV comprises an ESE. In some embodiments, the at least one exonic sequence of the nucleic acid of Subgroup IV comprises an ESS.

In some embodiments, the nucleic acid of Subgroup III or Subgroup IV comprises an H/ACA box, wherein the H/ACA box comprises a nucleotide sequence having from 5′ to 3′ an H consensus sequence and an ACA consensus sequence. In some embodiments, the one or more binding domain sequences of the nucleic acid of Subgroup III or Subgroup IV is positioned upstream the H consensus sequence. In some embodiments, the one or more binding domain sequences of the nucleic acid of Subgroup III or Subgroup IV is positioned downstream the ACA consensus sequence. In some embodiments, the one or more binding domain sequences of the nucleic acid of Subgroup III or Subgroup IV is positioned between the H consensus sequence and the ACA consensus sequence.

In some embodiments, the nucleic acid of Subgroup III or Subgroup IV comprises an H/ACA box comprising a nucleotide sequence having from 5′ to 3′ an H consensus sequence and an ACA consensus sequence; and one binding domain. In some embodiments, the one binding domain sequence is positioned upstream the H consensus sequence. In some embodiments, the one binding domain is positioned downstream the ACA consensus sequence. In some embodiments, the one binding domain sequence is positioned between the H consensus sequence and the ACA consensus sequence.

In some embodiments, the nucleic acid of Subgroup III or Subgroup IV comprises an H/ACA box comprising a nucleotide sequence having from 5′ to 3′ an H consensus sequence and an ACA consensus sequence, a first binding domain, and second binding domain. In some embodiments, the first binding domain sequence and the second binding domain sequence are each positioned upstream the H consensus sequence. In some embodiments, the first binding domain sequence and the second binding domain sequence are each positioned downstream the ACA consensus sequence. In some embodiments, the first binding domain sequence and the second binding domain sequence are each positioned between the H consensus sequence and the ACA consensus sequence. In some embodiments, the first binding domain sequence is positioned upstream the H consensus sequence and the second binding domain sequence is positioned between the H consensus sequence and the ACA consensus sequence or downstream the ACA consensus sequence. In some embodiments, the first binding domain sequence is positioned upstream the H consensus sequence or between the H consensus sequence and the ACA consensus sequence and the second binding domain sequence is positioned between the H consensus sequence and the ACA consensus sequence or downstream the ACA consensus sequence.

In some embodiments, the nucleic acid of Subgroup III or Subgroup IV comprises an H/ACA box and one binding domain, wherein the H/ACA box comprises a nucleotide sequence having from 5′ to 3′ an H consensus sequence and an ACA consensus sequence, and wherein the one binding domain is positioned upstream the H consensus sequence; downstream the ACA consensus sequence; or between the H consensus sequence and the ACA consensus sequence.

In some embodiments, the nucleic acid of Subgroup III or Subgroup IV comprises an H/ACA box and two binding domains, wherein the H/ACA box comprises a nucleotide sequence having from 5′ to 3′ an H consensus sequence and an ACA consensus sequence, and wherein the two binding domains are each independently positioned upstream the H consensus sequence; downstream the ACA consensus sequence; and/or between the H consensus sequence and the ACA consensus sequence.

In some embodiments, the nucleic acid of Subgroup III or Subgroup IV comprises an H/ACA box and three binding domains, wherein the H/ACA box comprises a nucleotide sequence having from 5′ to 3′ an H consensus sequence and an ACA consensus sequence, and wherein the three binding domains are each independently positioned upstream the H consensus sequence; downstream the ACA consensus sequence; and/or between the H consensus sequence and the ACA consensus sequence.

In some embodiments, the nucleic acid of Subgroup III or Subgroup IV comprises an H/ACA box and four binding domains, wherein the H/ACA box comprises a nucleotide sequence having from 5′ to 3′ an H consensus sequence and an ACA consensus sequence, and wherein the four binding domains are each independently positioned upstream the H consensus sequence; downstream the ACA consensus sequence; and/or between the H consensus sequence and the ACA consensus sequence.

In some embodiments, the nucleic acid of Subgroup III or Subgroup IV comprises an H/ACA box and five binding domains, wherein the H/ACA box comprises a nucleotide sequence having from 5′ to 3′ an H consensus sequence and an ACA consensus sequence, and wherein the five binding domains are each independently positioned upstream the H consensus sequence; downstream the ACA consensus sequence; and/or between the H consensus sequence and the ACA consensus sequence.

In some embodiments, the nucleic acid of Subgroup III or Subgroup IV comprises a C/D box, wherein the C/D box comprises a nucleotide sequence having from 5′ to 3′ a C consensus sequence, a D′ consensus sequence, a C′ consensus sequence, and a D consensus sequence. In some embodiments, the one or more binding domain sequences of the nucleic acid of Subgroup III or Subgroup IV is positioned upstream the C consensus sequence. In some embodiments, the one or more binding domain sequences of the nucleic acid of Subgroup III or Subgroup IV is positioned between the C consensus sequence and the D′ consensus sequence. In some embodiments, the one or more binding domain sequences of the nucleic acid of Subgroup III or Subgroup IV is positioned between the C′ consensus sequence and the D consensus sequence. In some embodiments, the one or more binding domain sequences of the nucleic acid of Subgroup III or Subgroup IV is positioned downstream the D consensus sequence.

In some embodiments, the nucleic acid of Subgroup III or Subgroup IV comprises a C/D box and one binding domain, wherein the C/D box comprises a nucleotide sequence having from 5′ to 3′ a C consensus sequence, a D′ consensus sequence, a C′ consensus sequence, and a D consensus sequence; and wherein the one binding domain is positioned upstream the C consensus sequence; between the C consensus sequence and the D′ consensus sequence; between the C′ consensus sequence and the D consensus sequence; or downstream the D consensus sequence.

In some embodiments, the nucleic acid of Subgroup III or Subgroup IV comprises a C/D box and two binding domains, wherein the C/D box comprises a nucleotide sequence having from 5′ to 3′ a C consensus sequence, a D′ consensus sequence, a C′ consensus sequence, and a D consensus sequence; and wherein the two binding domains are each independently positioned upstream the C consensus sequence; between the C consensus sequence and the D′ consensus sequence; between the C′ consensus sequence and the D consensus sequence; and/or downstream the D consensus sequence.

In some embodiments, the nucleic acid of Subgroup III or Subgroup IV comprises a C/D box and three binding domains, wherein the C/D box comprises a nucleotide sequence having from 5′ to 3′ a C consensus sequence, a D′ consensus sequence, a C′ consensus sequence, and a D consensus sequence; and wherein the three binding domains are each independently positioned upstream the C consensus sequence; between the C consensus sequence and the D′ consensus sequence; between the C′ consensus sequence and the D consensus sequence; and/or downstream the D consensus sequence.

In some embodiments, the nucleic acid of Subgroup III or Subgroup IV comprises a C/D box and four binding domains, wherein the C/D box comprises a nucleotide sequence having from 5′ to 3′ a C consensus sequence, a D′ consensus sequence, a C′ consensus sequence, and a D consensus sequence; and wherein the four binding domains are each independently positioned upstream the C consensus sequence; between the C consensus sequence and the D′ consensus sequence; between the C′ consensus sequence and the D consensus sequence; and/or downstream the D consensus sequence.

In some embodiments, the nucleic acid of Subgroup III or Subgroup IV comprises a C/D box and five binding domains, wherein the C/D box comprises a nucleotide sequence having from 5′ to 3′ a C consensus sequence, a D′ consensus sequence, a C′ consensus sequence, and a D consensus sequence; and wherein the five binding domains are each independently positioned upstream the C consensus sequence; between the C consensus sequence and the D′ consensus sequence; between the C′ consensus sequence and the D consensus sequence; and/or downstream the D consensus sequence.

In some embodiments, a nucleic acid of the disclosure (e.g., a nucleic acid of any one of Subgroups I-IV) comprises at least one binding domain sequence with full complementarity to the target sequence. In some embodiments, the nucleic acid comprises at least one binding domain sequence with partial complementarity to the target sequence (e.g., comprising at least 95% complementarity to the target sequence). In some embodiments, the nucleic acid comprises at least one binding domain sequence with full complementarity to the target sequence and at least one binding domain sequence with partial complementarity to the target sequence (e.g., comprising at least 95% complementarity to the target sequence).

In some embodiments, the nucleic acid of the disclosure (e.g., a nucleic acid of any one of Subgroups I-IV) has a sequence of up to about 20,000 nucleotides in length. In some embodiments, the sequence is up to about 10,000 nucleotides in length. In some embodiments, the sequence is up to about 9,000 nucleotides in length. In some embodiments, the sequence is up to about 8,000 nucleotides in length. In some embodiments, the sequence is up to about 7,000 nucleotides in length. In some embodiments, the sequence is up to about 6,000 nucleotides in length. In some embodiments, the sequence is up to about 5,000 nucleotides in length. In some embodiments, the sequence is about 50 to about 500 nucleotides in length. In some embodiments, the sequence about 50 to about 1000 nucleotides in length. In some embodiments, the sequence about 100 to about 500 nucleotides in length. In some embodiments, the sequence about 100 to about 1000 nucleotides in length. In some embodiments, the sequence about 500 to about 1000 nucleotides in length. In some embodiments, the sequence about 500 to about 2000 nucleotides in length. In some embodiments, the sequence about 500 to about 3,000 nucleotides in length. In some embodiments, the sequence about 500 to about 4,000 nucleotides in length. In some embodiments, the sequence about 500 to about 5,000 nucleotides in length. In some embodiments, the sequence about 1,000 to about 5,000 nucleotides in length. In some embodiments, the sequence about 1,000 to about 10,000 nucleotides in length. In some embodiments, the sequence about 5,000 to about 15,000 nucleotides in length. In some embodiments, the sequence about 5,000 to about 20,000 nucleotides in length.

In some embodiments, a splice editor nucleic acid molecule of the disclosure comprises a sequence selected from Table 3 or a portion thereof. Table 3 provides exemplary nucleotide sequences of splice editor nucleic acids of the disclosure. As presented in the table, the regions of the sequence are demarcated by hyphens (-) and identification of the regions from 5′ to 3′ are provided as Region 1, Region 2, Region 3, and optionally Region 4. In some embodiments, a splice editor nucleic acid molecule of the disclosure comprises a sequence having the formula 5′-[A]-[B]-3′, wherein [A] is a nucleotide sequence selected from Table 3 and [B] is a sequence comprising from 5′ to 3′ a splice acceptor and one or more exonic sequences. In some embodiments, [A]comprises a nucleotide sequence selected from Table 3, wherein the RNA-binding domain is exchanged with an RNA-binding domain described herein.

In some embodiments, a splice editor nucleic acid molecule of the disclosure comprises from 5′ to 3′ one or more RNA binding domains described herein, a ncRNA sequence, an intronic sequence, a splice acceptor, and one or more exonic sequences, wherein the ncRNA sequence has about 90%, 95%, 98%, 99%, or 100% to a ncRNA sequence identified in Table 3. In some embodiments, a splice editor nucleic acid molecule of the disclosure comprises from 5′ to 3′ one or more exonic sequences, a splice donor, an intronic sequence, a ncRNA sequence, and one or more RNA binding domains described herein, wherein the ncRNA sequence has about 90%, 95%, 98%, 99%, or 100% to a ncRNA sequence identified in Table 3. In some embodiments, the intronic sequence has about 90%, 95%, 98%, 99%, or 100% to an intronic sequence identified in Table 3.

### Vectors

In some embodiments, the disclosure provides a vector comprising one or more splice editor nucleic acid described herein. As used herein, the term “vector” refers to a nucleic acid molecule capable of transporting another nucleic acid to which it has been linked. In some embodiments, the vector is a DNA vector. In some embodiments, the vector is circular. In some embodiments, the vector is linear. Non-limiting exemplary vectors include plasmids, phagemids, cosmids, artificial chromosomes, minichromosomes, transposons, viral vectors, and expression vectors.

In some embodiments, the vector is an expression vector, wherein the expression vector is capable of directing the expression of nucleic acids to which it is operably linked. As used herein, an “expression vector” or “recombinant expression vector” refers to a replicon, such as plasmid, phage, virus, or cosmid, to which another DNA segment, i.e. an “insert”, is attached so as to bring about the replication of the attached segment in a cell.

In some embodiments, the vector or expression vector is a plasmid. As used herein, a “plasmid” refers to a circular double-stranded DNA loop into which additional nucleic acid segments are ligated.

In some embodiments, the vector or expression vector is a viral vector, wherein additional nucleic acid segments are ligated into the viral genome. Non-limiting exemplary viral vectors include viral vectors based on vaccinia virus; poliovirus; adenovirus; adeno-associated virus; SV40; herpes simplex virus; human immunodeficiency virus; picornaviruses. Non-limiting exemplary viral vectors also include viral vectors based on a retrovirus such as a Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus. In some embodiments, the vectors is for use in eukaryotic target cells and includes, but is not limited to, pXT1, pSG5, pSVK3, pBPV, pMSG, and pSVLSV40 (Pharmacia).

In some embodiments, the vector comprises one or more transcription and/or translation control elements. In some embodiments, the more transcription and/or translation control elements used depends on the target cell population and the vector system. In some embodiments, any number of suitable transcription and translation control elements, including constitutive and inducible promoters, transcription enhancer elements, transcription terminators, etc. are used in the expression vector, such as those further described below.

In some embodiments, a vector comprising a splice editor nucleic acid of the disclosure is operably linked to a control element, e.g., a transcriptional control element, such as a promoter. In some embodiments, the transcriptional control element is functional in a eukaryotic cell, e.g., a mammalian cell, e.g., a human cell. In some embodiments, the splice editor nucleic acid sequence is operably linked to one or more control elements that enable expression in eukaryotic cells, e.g., mammalian cells, e.g., human cells.

In some embodiments, the expression vector comprises a promoter that is an inducible promoter (e.g., a heat shock promoter, tetracycline-regulated promoter, steroid-regulated promoter, metal-regulated promoter, estrogen receptor-regulated promoter, etc.). Examples of inducible promoters include, but are not limited to, T7 RNA polymerase promoter, T3 RNA polymerase promoter, Isopropyl-beta-D-thiogalactopyranoside (IPTG)-regulated promoter, lactose induced promoter, heat shock promoter, Tetracycline-regulated promoter (e.g., Tet-ON, Tet-OFF, etc.), steroid-regulated promoter, metal-regulated promoter, estrogen receptor-regulated promoter, etc. In some embodiments, an inducible promoters is regulated by molecules including, but not limited to, doxycycline; RNA polymerase, e.g., T7 RNA polymerase; an estrogen receptor; an estrogen receptor fusion; etc.

In some embodiments, the promoter is a constitutive promoter (e.g., CMV promoter, UBC promoter).

In some embodiments, the promoter is a spatially restricted and/or temporally restricted promoter (e.g., a tissue specific promoter, a cell type specific promoter, etc.). Spatially restricted promoters can also be referred to as enhancers, transcriptional control elements, control sequences, etc. Any convenient spatially restricted promoter is suitable for use in the present disclosure, and the choice of a suitable promoter (e.g., a liver-specific promoter, a brain specific promoter, a promoter that drives expression in a subset of neurons, a promoter that drives expression in the germline, a promoter that drives expression in the lungs, a promoter that drives expression in muscles, a promoter that drives expression in islet cells of the pancreas, etc.) will depend on the organism. For example, various spatially restricted promoters are known for plants, flies, worms, mammals, mice, etc. Thus, a spatially restricted promoter can be used to regulate the expression of a splice editor nucleic acid in a wide variety of different tissues and cell types, depending on the organism. Some spatially restricted promoters are also temporally restricted such that the promoter is in the “ON” state or “OFF’ state during specific stages of embryonic development or during specific stages of a biological process. For illustration purposes, examples of spatially restricted promoters include, but are not limited to, liver-specific promoters, neuron-specific promoters, adipocyte-specific promoters, cardiomyocyte-specific promoters, smooth muscle-specific promoters, photoreceptor-specific promoters, etc.

Suitable promoters for use in the present disclosure include those derived from viruses and are referred to herein as viral promoters, or they include those derived from an organism, including prokaryotic or eukaryotic organisms. In some embodiments, a suitable promoter for use in the present disclosure include any promoter that drives expression by an RNA polymerase (e.g., pol I, pol II, pol III).

Exemplary promoters include, but are not limited to, the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res.2003 Sep. 1; 31(17)), a human H1 promoter (H1), and the like.

Exemplary eukaryotic promoters (i.e., promoters functional in a eukaryotic cell) include, but are not limited to, those from cytomegalovirus (CMV) immediate early, herpes simplex virus (HSV) thymidine kinase, early and late SV40, long terminal repeats (LTRs) from retrovirus, human elongation factor-1 promoter (EF1), a hybrid construct comprising the cytomegalovirus (CMV) enhancer fused to the chicken beta-actin promoter (CAG), murine stem cell virus promoter (MSCV), phosphoglycerate kinase-1 locus promoter (PGK), and mouse metallothionein-I.

In some embodiments, the disclosure provides a vector comprising a splice editor nucleic acid described herein and an RNA polymerase III promoter (e.g., U6 and H1). Descriptions of and parameters for enhancing the use of such promoters are known in art, and additional information and approaches are regularly being described; see, e.g., Ma, H. et al., Molecular Therapy—Nucleic Acids 3, e161 (2014).

In some embodiments, the expression vector comprises a ribosome binding site for translation initiation and a transcription terminator. In some embodiments, the expression vector comprises appropriate sequences for amplifying expression. In some embodiments, the expression vector comprises nucleotide sequences encoding non-native tags (e.g., histidine tag, hemagglutinin tag, green fluorescent protein, etc.), for example, that are operably-linked to the splice editor nucleic acid.

Methods of introducing a nucleic acid to a host cell or a population of host cells are known in the art, and any known method can be used to introduce a nucleic acid (e.g., an expression construct) into a cell. In some embodiments, a splice editor nucleic acid molecule or vector comprising the splice editor nucleic acid molecule are provided to a population of cells using well-developed transfection techniques; see, e.g. Angel and Yanik (2010) PLoS ONE 5(7): e 11756, and the commercially available TransMessenger® reagents from Qiagen, StemfectTM RNA Transfection Kit from Stemgent, and TranslT®-mRNA Transfection Kit from Mims Bio LLC (See, also Beumer et al. (2008). PNAS 105(50):19821-19826).

In some embodiments, the splice editor nucleic acid molecule is introduced to the cell or a population of cells as an RNA. In some embodiments, the RNA has chemistries suitable for delivery, tolerability, and stability within cells, e.g., following in vivo or in vitro administration. In some embodiments, the RNA is modified, e.g., comprises a modified sugar moiety, a modified internucleoside linkage, a modified nucleoside, a modified nucleotide and/or combinations thereof. In some embodiments, the modified RNA exhibits one or more of the following properties: is not immune stimulatory; is nuclease resistant; has improved cell uptake; has increased half-life; has increased translation efficiency; and/or is not toxic to cells or mammals, e.g., following contact with cells in vivo or ex vivo or in vitro.

### Delivery Agents

In some embodiments, delivery of a splice editor nucleic acid described herein is performed by one or more methods described herein. In some embodiments, the splice editor nucleic acid is delivered by viral vectors, lipid nonaparticles (LNPs), synthetic polymers, or a combination thereof. In some embodiments, the methods of delivery described herein are suitable for administering a splice editor nucleic acid of the disclosure to a target cell population or target tissue for the purpose of cellular, ex vivo, or in vivo targeting of a pre-mRNA in the target cell or target tissue for trans-splicing.

In some embodiments, the delivery comprises administering the splice editor nucleic acid as RNA or DNA. In some embodiments, the delivery comprises administering the splice editor nucleic acid as a DNA formulated as an LNP or a polymeric nanoparticle. In some embodiments, the delivery comprises administering the splice editor nucleic acid as an RNA formulated as an LNP or a polymeric nanoparticle.

In some embodiments, the delivery comprises administering a recombinant expression vector comprising the splice editor nucleic acid (e.g., plasmid, viral vector). In some embodiments, the recombinant expression vector is a non-viral vector (e.g., a plasmid). In some embodiments, the recombinant expression vector is a viral vector (e.g., an AAV). In some embodiments, the delivery comprises formulation of the one or more recombinant expression vectors using LNPs or polymeric nanoparticles. In some embodiments, a combination of a viral vector and a non-viral delivery vehicle are used.

In some embodiments, the splice editor nucleic acid molecules are delivered by non-viral delivery vehicles including, but not limited to, nanoparticles, liposomes, ribonucleoproteins, positively charged peptides, small molecule-RNA conjugates, aptamer-RNA chimeras, and RNA-fusion protein complexes. Non-limiting exemplary non-viral delivery vehicles include those described in Peer and Lieberman, Gene Therapy, 18: 1127-1133 (2011) (which focuses on non-viral delivery vehicles for siRNA that are also useful for delivery of other polynucleotides).

### Viral Delivery

In some embodiments, the splice editor nucleic acid molecules are delivered by viral delivery vehicles, such as AAV. In some embodiments, the viral vector (e.g., AAV vector) comprises one or more splice editor nucleic acid described herein. In some embodiments, the cloning capacity of the viral vector is sufficient to deliver the splice editor nucleic acid.

In some embodiments, a recombinant adeno-associated virus (rAAV) vector is used for delivery. Techniques to produce rAAV particles, in which an AAV genome to be packaged that includes the polynucleotide to be delivered (e.g., nucleic acid encoding one or more gRNAs and/or a site-directed endonuclease), rep and cap genes, and helper virus functions are provided to a cell are standard in the art. Production of rAAV typically requires that the following components are present within a single cell (denoted herein as a packaging cell): a rAAV genome, AAV rep and cap genes separate from (i.e., not in) the rAAV genome, and helper virus functions. The AAV rep and cap genes can be from any AAV serotype for which recombinant virus can be derived, and can be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13 AAV rh.74 and tropism modified AAV vectors. Production of pseudotyped rAAV is disclosed in, for example, U.S. Pat. No. 7,056,602.

In some embodiments, a method of generating a packaging cell involves creating a cell line that stably expresses all of the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line can then be infected with a helper virus, such as adenovirus. The advantages of this method are that the cells are selectable and are suitable for large-scale production of rAAV. Other examples of suitable methods employ adenovirus or baculovirus, rather than plasmids, to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol.4:2072 (1984); Hermonat et al., Proc. Natl. Acad. Sci. USA, 81:6466 (1984); Tratschin et al., Mol. Cell. Biol.5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595.

In addition to adeno-associated viral vectors, other viral vectors can be used. Such viral vectors include, but are not limited to, adenovirus, lentivirus, alphavirus, enterovirus, pestivirus, baculovirus, herpesvirus, Epstein Barr virus, papovavirus, poxvirus, vaccinia virus, and herpes simplex virus.

### Nanoparticle Compositions

In some embodiments, the splice editor nucleic acids of the disclosure or a recombinant expression vector comprising the splice editor nucleic acid is delivered to a host cell (e.g., ex vivo) or a subject by a nanoparticle (e.g., a lipid nanoparticle). In some embodiments, the nucleic acid or expression vector is formulated in nanoparticles or other delivery vehicles, (e.g., polymeric nanoparticles) to facilitate cellular uptake and/or to protect them from degradation when delivered to a subject.

In some embodiments, a nanoparticle composition comprises a lipid. Lipid nanoparticles include, but are not limited to, liposomes and micelles. Any number of lipids may be present, including cationic and/or ionizable lipids, anionic lipids, neutral lipids, amphipathic lipids, conjugated lipids (e.g., PEGylated lipids), and/or structural lipids. Such lipids can be used alone or in combination.

Nanoparticles are ultrafine particles typically ranging between 1 and 100 to 500 nanometers (nm) in size with a surrounding interfacial layer and often exhibiting a size-related or size-dependent property. Nanoparticle compositions are myriad and encompass lipid nanoparticles (LNPs), liposomes (e.g., lipid vesicles), and lipoplexes. For example, a nanoparticle composition can be a liposome having a lipid bilayer with a diameter of 500 nm or less. In some embodiments, nanoparticle compositions are vesicles including one or more lipid bilayers. In certain embodiments, a nanoparticle composition includes two or more concentric bilayers separated by aqueous compartments. Lipid bilayers can be functionalized and/or crosslinked to one another. Lipid bilayers can include one or more ligands, proteins, or channels.

In some embodiments, the nanoparticle composition comprises a splice editor nucleic acid and/or a recombinant expression vector comprising the splice editor nucleic acid.

In some embodiments, the disclosure provides LNP compositions comprising: (a) a splice editor nucleic acid molecules described herein or an expression vector comprising the splice editor nucleic molecule; and (b) one or more lipid moieties selected from the group consisting of amino lipids, helper lipids, structural lipids, phospholipids, ionizable lipids, PEG lipids, lipoid, and cholesterol or cholesterol derivatives. In some embodiments, the disclosure provides LNP compositions comprising: (a) a splice editor nucleic acid molecules described herein or an expression vector comprising the splice editor nucleic molecule; and (b) one or more lipid moieties selected from the group consisting of ionizable lipids, amino lipids, anionic lipids, neutral lipids, amphipathic lipids, helper lipids, structural lipids, PEG lipids, and lipoids, and optionally (c) targeting moieties.

In some embodiments, the LNP composition comprise one or more lipid moieties promote or enhances cellular uptake by the apolipoprotein E (apoE)-low density lipoprotein receptor (LDLR) pathway. For example, certain ionizable lipids are known in the art for increasing cellular uptake of LNPs by the apoE-LDLR pathway (see, e.g., Semple, et al (2010) NAT BIOTECH 28:172). In some embodiments, the LNP composition comprises one or more lipid moieties that promote or enhances cellular uptake by an apoE-LDLR independent pathway.

In some embodiments, the LNPs of the present disclosure are formed by any method known in the art including, but not limited to, a continuous mixing method, a direct dilution process, and an in-line dilution process. Additional techniques and methods suitable for the preparation of the LNPs described herein include coacervation, microemulsions, supercritical fluid technologies, phase-inversion temperature (PIT) techniques.

### Pharmaceutical Compositions

In some embodiments, the disclosure provides pharmaceutical compositions comprising a splice editor nucleic acid, recombinant expression vector, or delivery system described herein combined with an appropriate pharmaceutically acceptable carrier or diluent.

In some embodiments, the pharmaceutical composition comprises (1) one or more splice editor nucleic acids described herein, and (2) a pharmaceutically acceptable carrier or diluent. In some embodiments, the pharmaceutical composition comprises (1) an expression vector comprising a splice editor nucleic acid described herein, and (2) a pharmaceutically acceptable carrier or diluent. In some embodiments, the pharmaceutical composition comprises one or more splice editor nucleic acids or recombinant expression vector (e.g., AAV) comprising the one or more splice editor nucleic acids formulated as a lipid composition (e.g., LNP), and (2) a pharmaceutically acceptable carrier or diluent. In some embodiments, the pharmaceutical composition comprises a therapeutically effective amount of the one or more splice editor nucleic acids or recombinant expression vectors.

Exemplary pharmaceutically acceptable excipients such as carriers, solvents, stabilizers, adjuvants, diluents, etc., depending upon the particular mode of administration and dosage form. Contemplated pharmaceutical compositions can be generally formulated to achieve a physiologically compatible pH, depending on the formulation and route of administration. In some embodiments, the compositions comprise a therapeutically effective amount of the one or more splice editor nucleic acids or recombinant expression vectors, together with one or more pharmaceutically acceptable excipients.

Suitable excipients can include, for example, carrier molecules that include large, slowly metabolized macromolecules. Other exemplary excipients can include antioxidants, chelating agents, carbohydrates, stearic acid, liquids such as oils, water, saline, glycerol and ethanol, wetting or emulsifying agents, pH buffering substances, and the like.

Pharmaceutical compositions can be formulated into preparations in solutions, suppositories, injections. In some embodiments, the pharmaceutical composition is formulated to result in systemic administration of the one or more splice editor nucleic acids or recombinant expression vectors, for example, following enteral or parenteral administration. In some embodiments, the pharmaceutical composition is formulated to result in localized administration of the one or more splice editor nucleic acids or recombinant expression vectors, for example, following regional administration or implantation. In some embodiments, the pharmaceutical composition is formulated for immediate activity or for sustained release of the one or more splice editor nucleic acids or recombinant expression vectors.

Typically, an effective amount of a splice editor nucleic acid, recombinant expression vectors, or delivery system described herein, can be provided, for example, for use in a method of treating a subject having a disease or disorder. Methods of calculating the effective amount or effective dose are within the skill of one of ordinary skill in the art. The final amount to be administered is dependent upon the route of administration and upon the nature of the disorder that is to be treated. A competent clinician will be able to determine an effective amount of the splice editor nucleic acid, recombinant expression vectors, or delivery system described herein to administer to the patient to halt or reverse the progression of the disorder.

In some embodiments, based on animal data, and other information available for the trans-splicing system, a clinician can determine the maximum safe dose for an individual, depending on the route of administration. For instance, an intravenously administered dose can be more than an intrathecally administered dose, given the greater body of fluid into which the therapeutic composition is being administered. Similarly, compositions which are rapidly cleared from the body can be administered at higher doses, or in repeated doses, in order to maintain a therapeutic concentration. Utilizing ordinary skill, the competent clinician will be able to optimize the dosage of a particular therapeutic in the course of routine clinical trials.

For inclusion in a medicament, a splice editor nucleic acid, recombinant expression vectors, or delivery system described herein can be obtained from a suitable commercial source. In some embodiments, therapies based on a splice editor nucleic acid, recombinant expression vectors, or delivery system described herein to be used for therapeutic administration, must be sterile. Therapeutic compositions can be generally placed into a container having a sterile access port, for example, an intravenous solution bag or vial having a stopper pierceable by a hypodermic injection needle. In some embodiments, the therapeutic components are stored in unit or multi-dose containers, for example, sealed ampules or vials, as an aqueous solution or as a lyophilized formulation for reconstitution.

### Methods and Use

In some embodiments, the disclosure provides cellular, ex vivo, and in vivo methods comprising use of the splice editor nucleic acid, recombinant expression vectors, or delivery system described herein to target trans-splicing of a target RNA (e.g., pre-mRNA) in a cell. In some embodiments, the methods comprise use of the splice editor nucleic acid, recombinant expression vectors, or delivery system described herein described herein to correct a mutation in a target RNA (e.g., pre-mRNA). In some embodiments, the disclosure provides methods of treating a patient with a disease or disorder, comprising administering a splice editor nucleic acid, recombinant expression vector, delivery system, or pharmaceutical composition described herein to target trans-splicing of a target RNA (e.g., pre-mRNA) in a target cell population and/or target tissue, thereby treating the disease or disorder.

### Cellular RNA Editing

In some embodiments, the method comprises introducing a splice editor nucleic acid, recombinant expression vector, delivery system, or pharmaceutical composition described herein to a cell or cell population. In some embodiments, the method comprises contacting the cell with a splice editor nucleic acid, expression vector, delivery system, or pharmaceutical composition described herein. In some embodiments, the cell is a eukaryotic cell. In some embodiments, the eukaryotic cell is a mammalian cell. In some embodiments, the eukaryotic cell is a rodent cell. In some embodiments, the eukaryotic cell is a human cell. In some embodiments, the cell is a patient-derived cell.

The splice editor nucleic acid, recombinant expression vector, delivery system, or pharmaceutical composition described herein may be introduced into the cell via any methods known in the art, such as, e.g., viral or bacteriophage infection, transfection, conjugation, protoplast fusion, lipofection, electroporation, calcium phosphate precipitation, polyethyleneimine (PEI)-mediated transfection, DEAE-dextran-mediated transfection, liposome-mediated transfection, particle gun technology, calcium phosphate precipitation, shear-driven cell permeation, fusion to a cell-penetrating peptide followed by cell contact, microinjection, and nanoparticle-mediated delivery. In some embodiments, the vector system may be introduced into the cell via viral infection.

In some embodiments, the disclosure provides a method for targeting trans-splicing of a target RNA (e.g., pre-mRNA) in a cell, the method comprising contacting the cell with a splice editor nucleic acid, recombinant expression vector, delivery system, or pharmaceutical composition described herein, wherein when the cell is contacted with the splice editor nucleic acid, recombinant expression vector, delivery system, or pharmaceutical composition, the one or more binding domains of the splice editor nucleic acid binds to the target RNA (e.g., pre-mRNA) and trans-splicing results in ligation of one or more exons of the target RNA (e.g., pre-mRNA) to one or more exons of the splice editor nucleic acid.

In some embodiments, the disclosure provides a method for targeting trans-splicing of a pre-mRNA in a cell or a population of cells comprising a disease-causing mutation, the method comprising contacting the cell or population of cells with a splice editor nucleic acid, recombinant expression vector, delivery system, or pharmaceutical composition described herein, wherein when the cell is contacted with the splice editor nucleic acid, recombinant expression vector, delivery system, or pharmaceutical composition, the one or more binding domains of the splice editor nucleic acid binds to the pre-mRNA and trans-splicing results in ligation of one or more exons of the pre-mRNA to one or more exons of the splice editor nucleic acid, thereby resulting in a mRNA lacking the disease-causing mutation.

In some embodiments, the disclosure provides a method for targeting trans-splicing of a pre-mRNA in a cell or a population of cells derived from a patient having a disease or disorder, the method comprising contacting the cell or population of cells with a splice editor nucleic acid, recombinant expression vector, delivery system, or pharmaceutical composition described herein, wherein when the cell or population of cells is contacted with the splice editor nucleic acid, recombinant expression vector, delivery system, or pharmaceutical composition, the one or more binding domains of the splice editor nucleic acid binds to the target RNA (e.g., pre-mRNA) and trans-splicing results in ligation of one or more exons of the target RNA (e.g., pre-mRNA) to one or more exons of the splice editor nucleic acid, wherein the cell or population of cells is reintroduced to the patient, thereby treating or ameliorating the disease or disorder.

### In Vivo RNA Editing

The present disclosure provides methods for treating a patient having a disease or disorder using the splice editor nucleic acid, recombinant expression vector, delivery system, or pharmaceutical composition described herein. In some embodiments, the disease or disorder is associated with one or more mutations in a target RNA, wherein the method targets trans-splicing of the target RNA to remove the one or more mutations.

In some embodiments, the disclosure provides a method of treating a patient having a disease or disorder, comprising administering to the patient a splice editor nucleic acid, recombinant expression vector, delivery system, or pharmaceutical composition described herein.

In some embodiments, the disclosure provides a method of treating a patient having a disease or disorder by targeting trans-splicing of a target RNA (e.g. pre-mRNA) in a target tissue or cell population, the method comprising administering to the patient a splice editor nucleic acid, recombinant expression vector, delivery system, or pharmaceutical composition described herein, wherein when the splice editor nucleic acid, recombinant expression vector, delivery system, or pharmaceutical composition is administered, the splice editor nucleic acid binds to a target RNA (e.g., pre-mRNA) and trans-splicing results in ligation of one or more exons of the target RNA (e.g., pre-mRNA) to one or more exons of the splice editor nucleic acid, thereby treating or ameliorating the disease or disorder.

In some embodiments, the disclosure provides a method of treating a patient having a disease or disorder associated with one or more mutations in a pre-mRNA in a target tissue or cell population, the method comprising administering to the patient a splice editor nucleic acid, recombinant expression vector, delivery system, or pharmaceutical composition described herein, wherein when the splice editor nucleic acid, recombinant expression vector, delivery system, or pharmaceutical composition is administered, the splice editor nucleic acid binds to a pre-mRNA and trans-splicing results in ligation of one or more exons of the pre-mRNA to one or more exons of the splice editor nucleic acid, wherein the trans-splicing results in an mRNA lacking the disease-causing mutation, thereby treating or ameliorating the disease or disorder.

In some embodiments, the route of administration is any sufficient for delivery of the splice editor nucleic acid, recombinant expression vector, delivery system, or pharmaceutical composition described herein to the target tissue or cell population as ascertained by one of skill in the art.

In some embodiments, administration of the splice editor nucleic acid, recombinant expression vector, delivery system, or pharmaceutical composition described herein results in correction of a mutation in a pre-mRNA in a target tissue or cell population in the patient.

The term “treatment” refers to the application of one or more methods described herein for the amelioration of a disease. In some embodiments, the specific procedure is the administration of a splice editor nucleic acid, recombinant expression vector, delivery system, or pharmaceutical composition described herein. “Treatment” of an individual (e.g. a mammal, such as a human) or a cell is any type of intervention used in an attempt to alter the natural course of the individual or cell. Treatment includes, but is not limited to, administration of a splice editor nucleic acid, recombinant expression vector, delivery system, or pharmaceutical composition described herein, and may be performed either prophylactically or subsequent to the initiation of a pathologic event or contact with an etiologic agent. Treatment includes any desirable effect on the symptoms or pathology of a disease or condition, and may include, for example, minimal changes or improvements in one or more measurable markers of the disease or condition, and may include, for example, minimal changes or improvements in one or more measurable markers of the disease or condition being treated.

The terms “patient,” “subject,” “individual,” and the like are used interchangeably herein, and refer to any animal amenable to the methods described herein. In some embodiments, the patient, subject, or individual is a human.

### Kits

The present disclosure provides kits for carrying out the methods described herein. In some embodiments, the kit comprises a splice editor nucleic acid, recombinant expression vector, delivery system, or pharmaceutical composition described herein.

In some embodiments, the kit comprises a splice editor nucleic acid, recombinant expression vector, delivery system or pharmaceutical composition described herein and a reagent for reconstitution and/or dilution of the splice editor nucleic acid, recombinant expression vector, delivery system or pharmaceutical composition.

In some embodiments, the kit comprise one or more additional reagents, where such additional reagents are selected from a buffer, a buffer for introducing the splice editor nucleic acid, recombinant expression vector, delivery system into a cell, a wash buffer, a control reagent, a control vector, a control polynucleotide, a reagent for in vitro production of the recombinant expression vector or delivery system, adaptors for sequencing and the like. A buffer can be a stabilization buffer, a reconstituting buffer, a diluting buffer, or the like. A kit can also comprise one or more components that can be used to facilitate or enhance the on-target binding or the trans-splicing of the splice editor nucleic acid.

In addition to the above-mentioned components, a kit can further comprise instructions for using the components of the kit to practice the methods. The instructions for practicing the methods can be recorded on a suitable recording medium. For example, the instructions can be printed on a substrate, such as paper or plastic, etc. The instructions can be present in the kits as a package insert, in the labeling of the container of the kit or components thereof (i.e., associated with the packaging or subpackaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g. via the Internet), can be provided. An example of this case is a kit that comprises a web address where the instructions can be viewed and/or from which the instructions can be downloaded. As with the instructions, this means for obtaining the instructions can be recorded on a suitable substrate.

In some embodiments, the kit comprises a container comprising the splice editor nucleic acid, the recombinant expression vector, the delivery system, or the pharmaceutical composition described herein, and instructions for use targeting trans-splicing of a target RNA (e.g., pre-mRNA) in a cell or a population of cells.

In some embodiments, the kit comprises a container comprising the splice editor nucleic acid, the recombinant expression vector, the delivery system, or the pharmaceutical composition described herein, and instructions for administering the splice editor nucleic acid, the recombinant expression vector, the delivery system, or the pharmaceutical composition to a patient in need thereof to target trans-splicing of a target RNA (e.g., pre-mRNA) in a cell or a population of cells of the patient.

### Definitions

As used herein, the term “pre-mRNA” refers to a precursor mRNA and is an RNA which contains both exons and intron(s). Pre-mRNA is a type of primary transcript that becomes a messenger RNA after processing. It is synthesized from a DNA template in the cell nucleus by transcription. In some embodiments, RNA is from a mammalian cell. In other embodiments, the RNA is from the mitochondria of a mammalian cell.

As used herein, the term “RNA-binding” is used to describe a molecule, protein, nucleic acid, or complex that specifically binds to RNA.

As used herein, a “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

### EXAMPLES

### Example 1: Selection of ncRNAs

This Example describes the methods used to identify ncRNAs for inclusion in splice editors capable of targeting a pre-mRNA and producing a trans-splicing event. As shown in FIG. 1E, it was determined that nearly all ncRNA sequences mined from public databases contain a sequence motif identified in Table 1. RNAlib-2.5.1 software was used to predict the secondary structure of the ncRNA sequences identified in the public domain, including metazoan U1 snRNA sequences, U11 snRNA sequences, U7 snRNA sequences, Sm sequences, and H/ACA snoRNA sequences. The predicted secondary structures were compared to known secondary structures using an RNA covariance model (see, e.g., Eddy, et al (1994) Nucleic Acids Research 22:2079-2088). ncRNA sequences having a predicted secondary structure with similarity to known secondary structures were selected for further evaluation. This approach provided more than 120,000 candidate ncRNA sequences. As shown in FIG. 1F, the candidate ncRNA sequences ranged from approximately 7 nucleotides to more than 300 nucleotides in length. Exemplary candidate ncRNA sequences that were identified by this computational analysis are set forth in SEQ ID NOs: 9-657.

### Example 2: Design and Testing of Splice Editors for Trans-Splicing

Splice editor nucleic acid molecules were designed for targeted trans-splicing. The nucleic acid molecules comprise a nucleotide sequence with (a) an intronic sequence having (i) at least one binding domain sequence complementary to a target sequence in a pre-mRNA, and (ii) a ncRNA sequence; (b) a splice site; and (c) at least one exonic sequence. The ncRNA sequences were selected from the candidate ncRNAs identified as described in Example 1. The splice editor nucleic acid molecules were engineered to incorporate the entire candidate ncRNA sequence or a portion thereof comprising a secondary structure and/or sequence motif identified in Table 1.

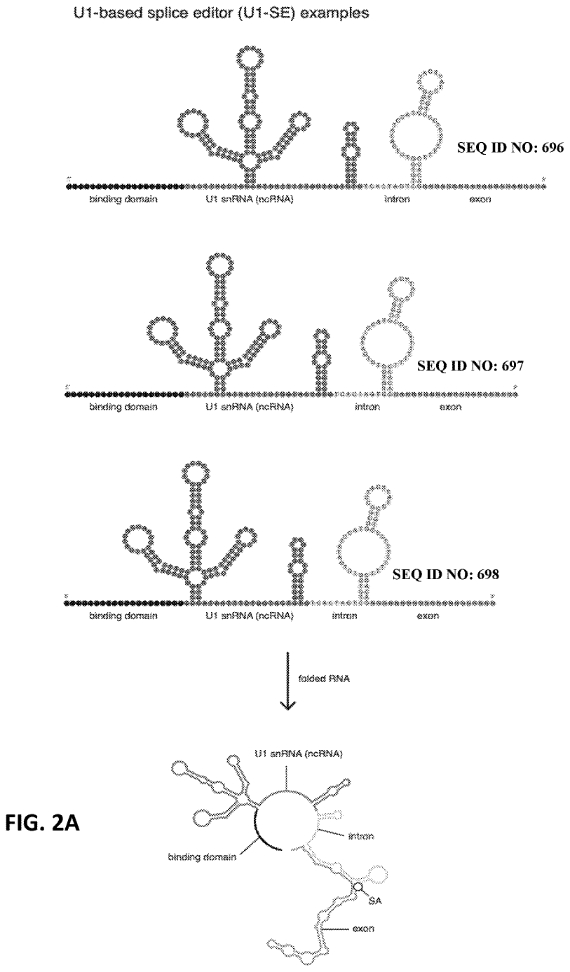

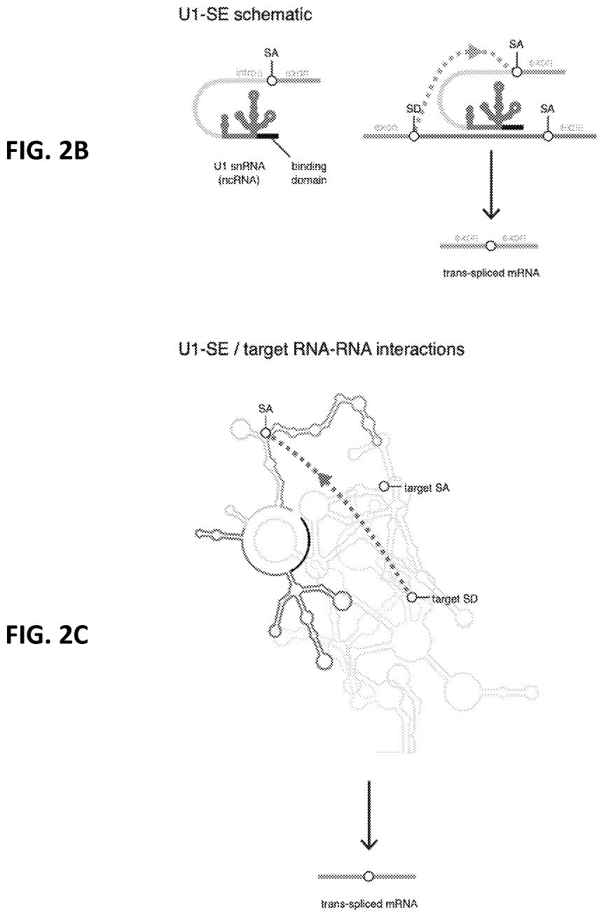

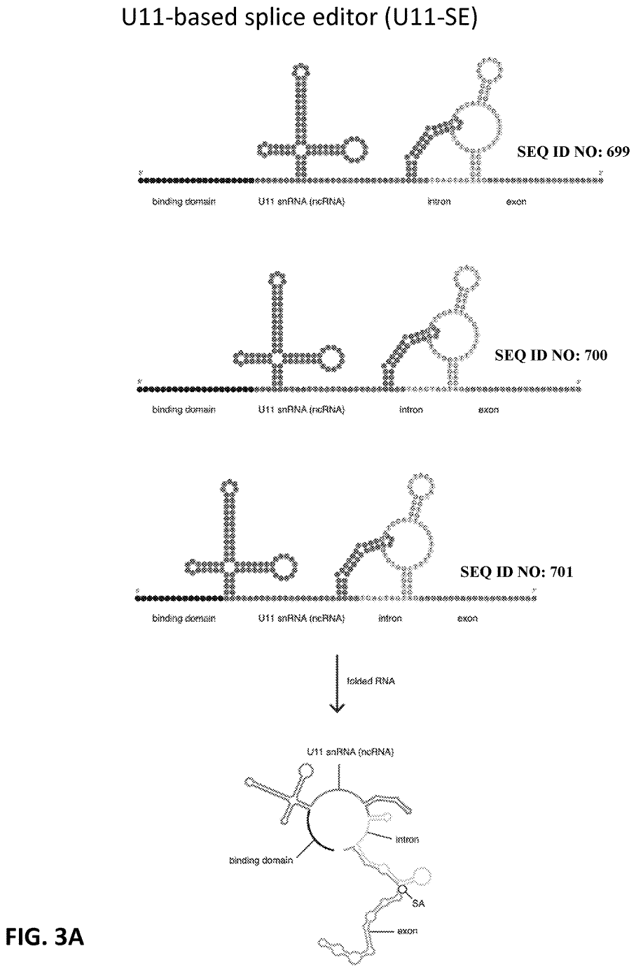

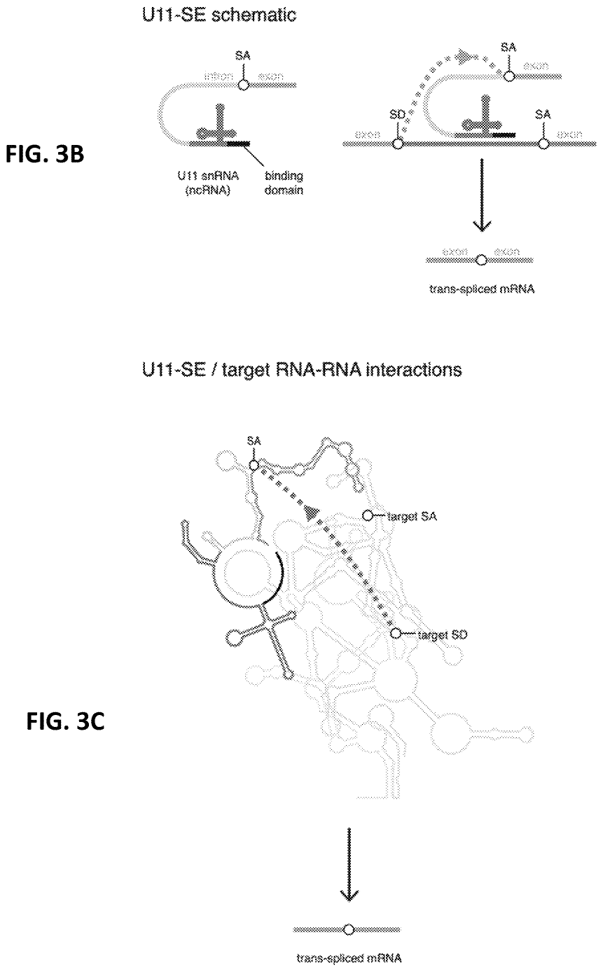

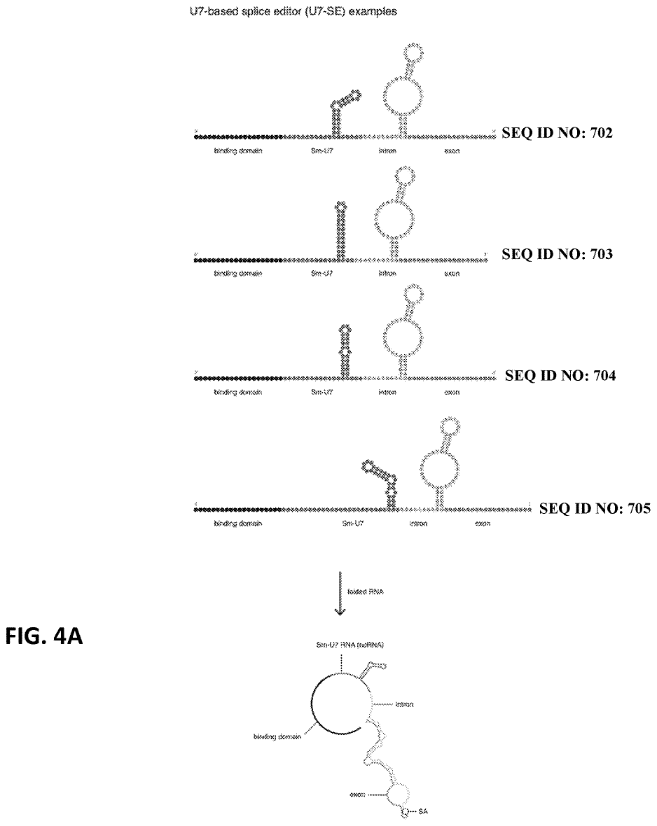

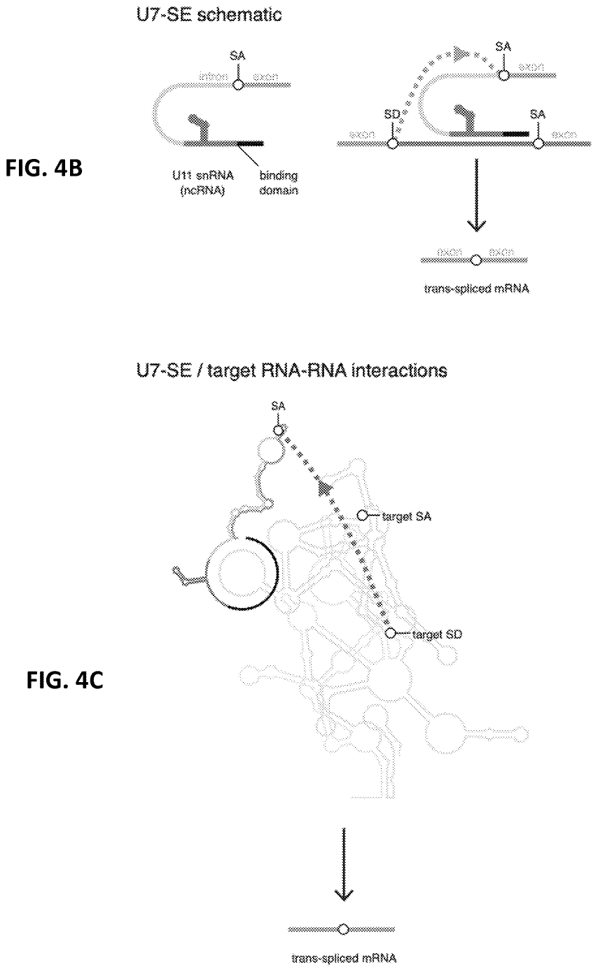

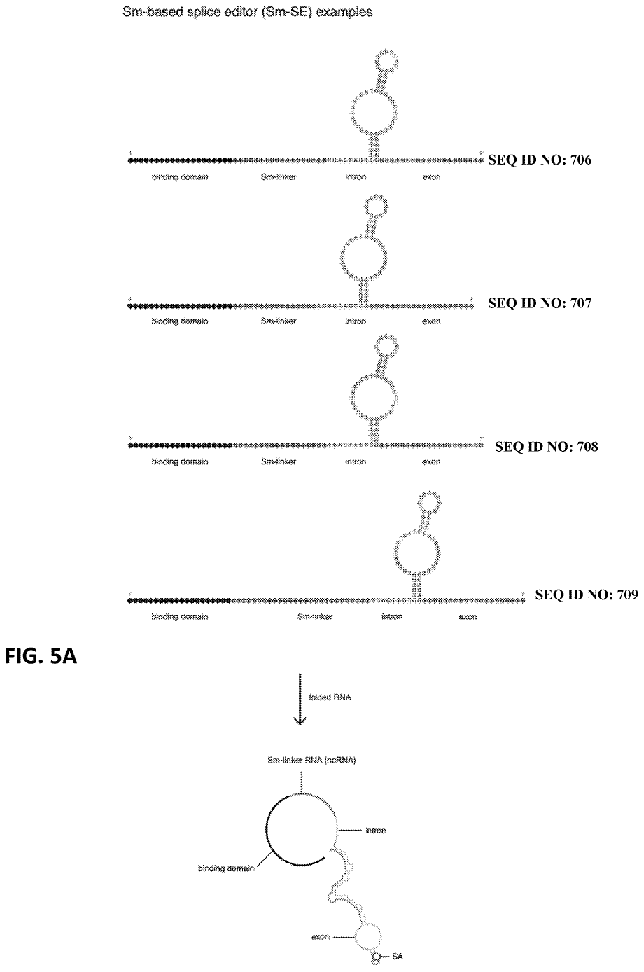

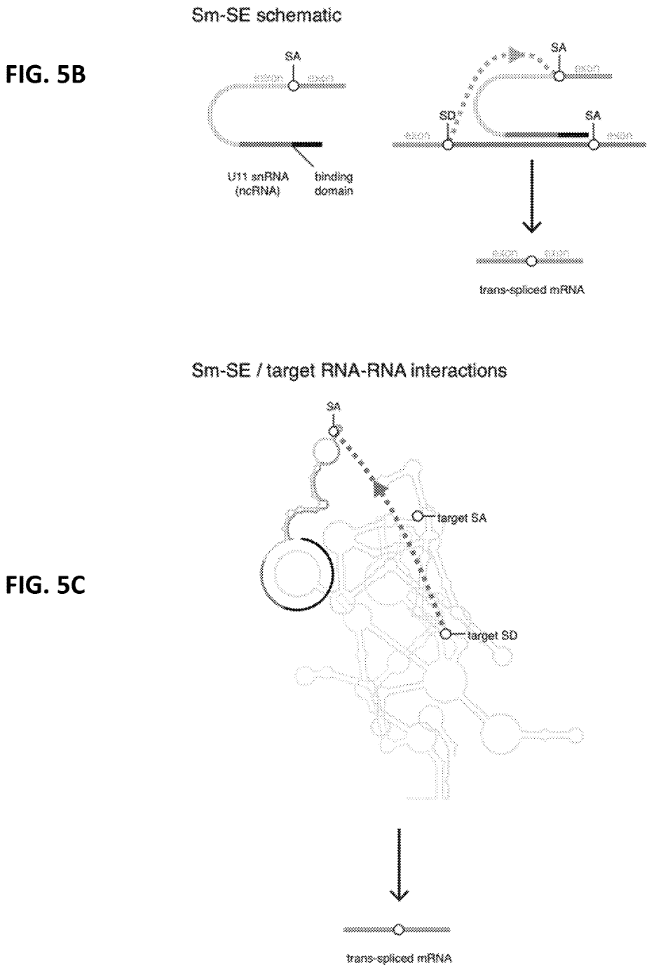

A first set of nucleic acid molecules were designed to have a ncRNA sequence derived from a snRNA and to undergo trans-splicing at a splice donor in a pre-mRNA (for correction of a mutation at the 5′end of an exon). The nucleic acid molecules had a nucleotide sequence arranged 5′ to 3′ (a) an intron having (i) a binding domain sequence, (ii) a snRNA sequence (a U1 snRNA; U11 snRNA; a Sm sequence motif and U7 snRNA; or a Sm sequence motif), (iii) a branch point, and (iv) a polypyrimidine tract; (b) a splice acceptor; and (c) an exon. Schematics of exemplary U1-based splice editor nucleic acid molecules are shown in FIG. 2A, FIG. 2B, and FIG. 2C and sequences are provided in the table of Table 3 (in rows of Table 3 in which the column titled “Region 2” is assigned a label of “U1_X_Y” in which X and Y are integers). Schematics of exemplary U11-based splice editor nucleic acid molecules are shown in FIG. 3A, FIG. 3B, and FIG. 3C and sequences are provided in the table of Table 3 (in rows of Table 3 in which the column titled “Region 2” is assigned a label of “U11_X_Y” in which X and Y are integers). Schematics of exemplary U7-based splice editor nucleic acid molecules are shown in FIG. 4A, FIG. 4B, and FIG. 4C and sequences are provided in Table 3 (in rows of Table 3 in which the column titled “Region 2” is assigned a label beginning with “Sm_Z” in which Z is an integer and the column titled “Region 3” is assigned a label beginning with “U7_X” in which X is an integer). Schematics of exemplary Sm-based splice editor nucleic acid molecules are shown in FIG. 5A, FIG. 5B, and FIG. 5C and sequences are provided in Table 3 (in rows of Table 3 in which the column titled “Region 2” is assigned a label beginning with “Sm_Z” in which Z is an integer and the column titled “Region 3” is assigned a label “adeno_intron”).

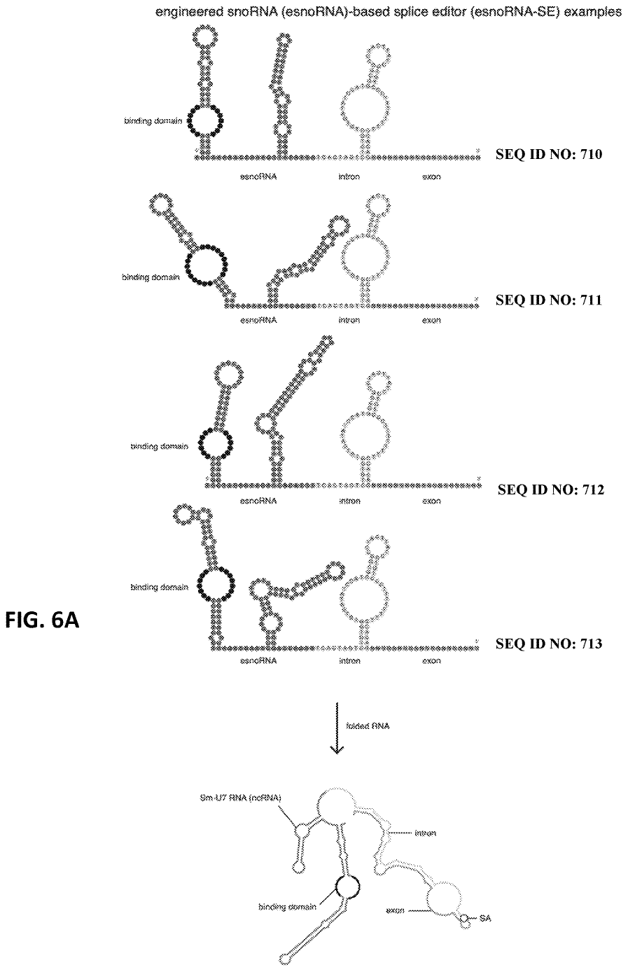

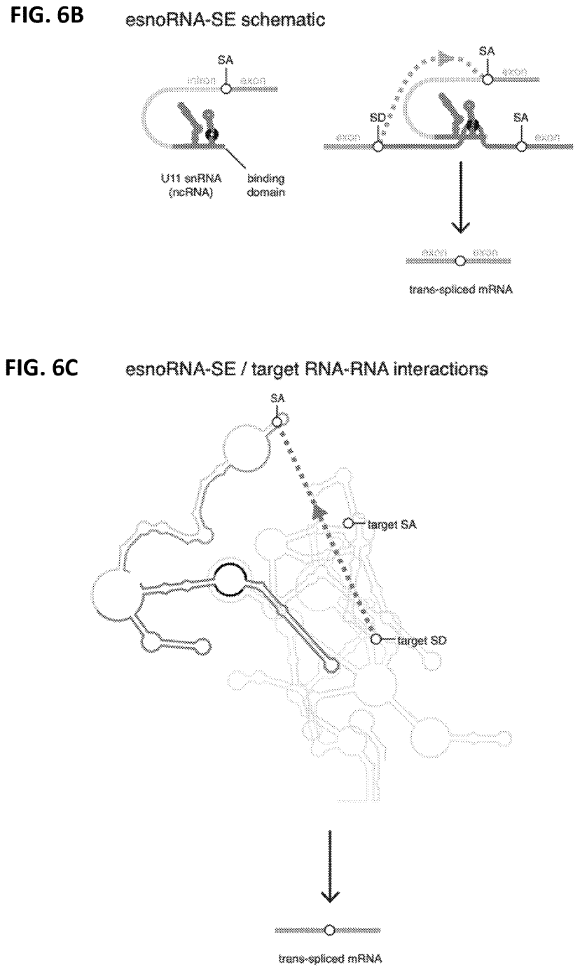

A second set of nucleic acid molecule were designed to have a ncRNA sequence derived from a H/ACA snoRNA and to undergo trans-splicing at a splice donor in a pre-mRNA (for correction of a mutation at the 5′end of an exon). The nucleic acid molecules had a nucleotide sequence arranged 5′ to 3′ (a) an intron having (i) a first and second binding domain inserted into an H/ACA box snoRNA sequence, (ii) a branch point, and (iii) a polypyrimidine tract; (b) a splice acceptor; and (c) an exon. Schematics of exemplary snoRNA-based splice editor nucleic acid molecules are shown in FIG. 6A, FIG. 6B, and FIG. 6C and sequences are provide in Table 3 (in rows of Table 3 in which the column titled “Region 1” is assigned a label beginning with “sno” “SNO” or “SCARNA”).

The nucleic acid molecules are evaluated for trans-splicing by using reporter cells, where correct RNA edits generate a mRNA that produces a fluorescent protein. Splice editors are be introduced to the reporter cells via viral or non-viral methods. Viral methods include but are not limited to lentivirus, AAV, and adenovirus. Non-viral methods include but are not limited to transfection or electroporation. The cells are first transfected with a splice donor reporter construct encoding a pre-mRNA under control of a CMV promoter, the pre-mRNA comprising a blue fluorescent protein (BFP), a self-cleaving p2A linker, a truncated GFP (5′GFP), and a splice donor. The BFP is used to confirm stable expression of the reporter construct. The reporter construct has a matrix metallopeptidase 9 (MMP9) intron 1 and exon 2 downstream the splice donor to ensure splicing an event can occur, followed by a bovine growth hormone polyadenylation signal (bGHpA) to allow for stable expression of the construct. The splice editor nucleic acids have an exon that is the second half of the truncated GFP (3′GFP) and trans-splicing results in expression of full-length GFP. Reporter cells with correct edits generate signal via a fluorescent reporter and are sorted via FACS. Sorted cells are sequenced to identify active splice editors.

### Example 3: Design and Testing of snoRNAs for Exon Skipping

In the experiments of this example, snoRNA guide constructs were developed that have hybridizing regions replaced to allow the snoRNA to serve as a guide for the RNP complex. The snoRNA guide constructs were tested for exon skipping in this example.

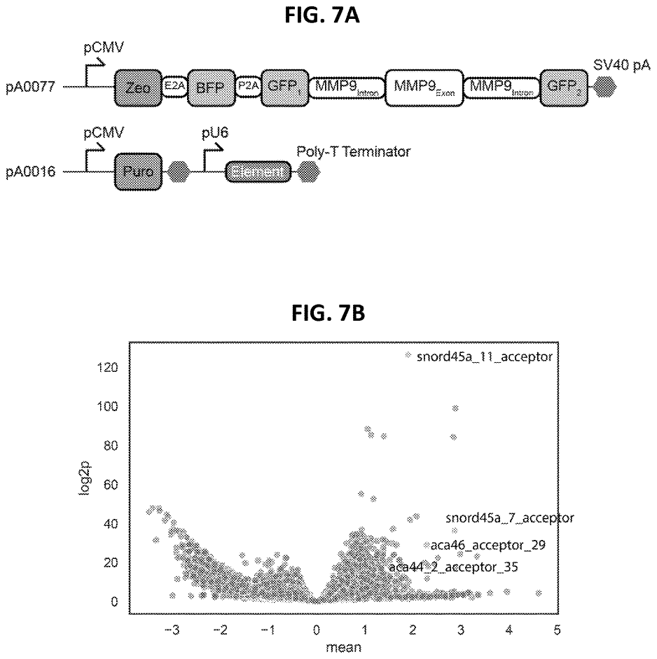

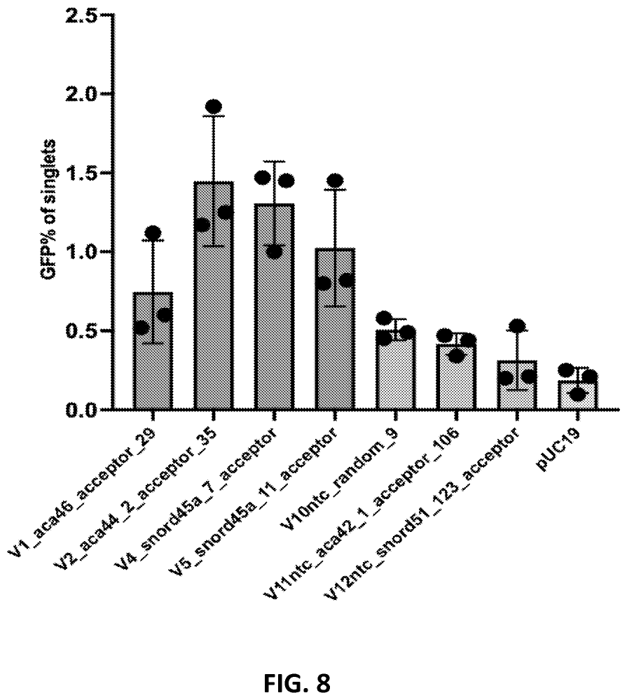

In these experiments, the snoRNA guide constructs were designed on a plasmid, along with a sequence that is complementary to a target sequence, to produce a snoRNP that occludes a splice site, and thereby allows for exon skipping. To test for exon skipping, splice acceptor targeting snoRNA guide molecules were designed on a plasmid (i.e., pA0077, FIG. 7A, Table 2), which also included intron and exon sites of the MMP9 gene and two GFP sites (FIG. 7A). An element was then introduced on a separate plasmid target (i.e., pA0016), which was present after the U6 promoter and before the poly-T terminator (FIG. 7A). The design of these constructs and plasmids allows the snoRNA to bind to the MMP9 gene splice site, thereby occluding the splice site and leading to GFP production, as shown in the volcano plot of FIG. 7B and the graph in FIG. 8.

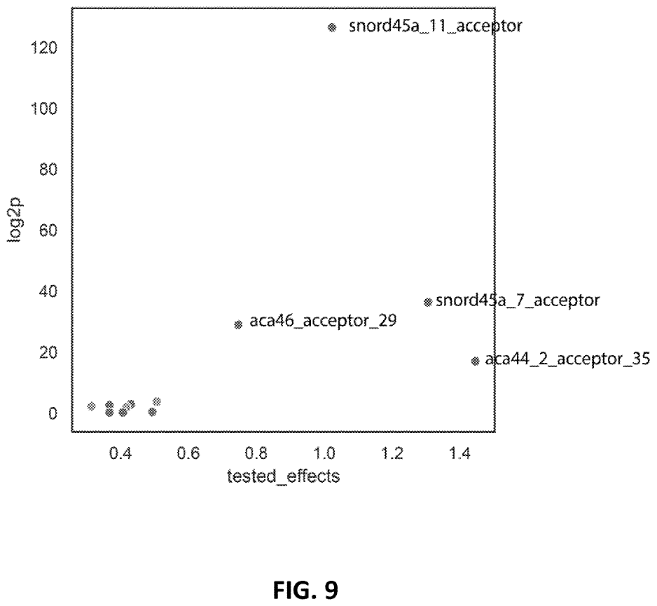

The four splice acceptor targeting snoRNA candidates of FIG. 7B (i.e., labeled “snord45a_11_acceptor”, “snord45a_7_acceptor”, “aca46_acceptor 29”, and “aca44_2_acceptor_35”, which were obtained from a high-throughput screen for exon skipping, and sequences shown in Table 4) were synthesized as IDT eBlocks and cloned into the pA0016 vector. The vector was transfected into a HEK293FT cell line that stably expresses a PiggyBAC-integrated MMP9 exon-skipping reporter (pA0077). This cell line was established by FACS sorting on reporter BFP expression for a highly-expressing single-cell clone. 100 ng element vector was transfected using Lipofectamine 2000 into HEK293FT cells seeded into a 96-well plate, such that cells were 9000 confluent at the time of transfection. 48 hours post-transfection, cells were analyzed for GFP % by flow cytometry compared to Non-targeting Controls (ntc) or cells treated with pUC19. FIG. 8 is a graph showing GFP production as a result of exon skipping from four different splice acceptors targeting snoRNAs (i.e., snord is C/D snoRNA, and snora or aca is T6/ACA, labeled “V_snord45a_11_acceptor”, “V4snord45a_7acceptor”, “V1_aca46_acceptor_29”, and “V2_aca44_2_acceptor_35”, and sequences shown in Table 4) compared to four randomized guides (i.e., “V10ntc_random_9”, “V11ntc_aca42_1_acceptor_106”, “V12ntc_snord51_123acceptor,” pUC19 (plasmid alone)). The results in FIG. 7A, FIG. 7B, FIG. 8, and FIG. 9 demonstrate the snoRNAs disclosed herein guide exon skipping.

TABLE 4
Splice Acceptor sequences targeting snoRNAs

| SEQ | | |
|---|---|---|
| ID | | |
| NO: | Reference | Sequence |
| 674 | snord45a_11_acceptor | GGTCAATGATGTGTTGGCATGTATCAGGTATTCCT |
| | | GTGGCTGATGTGTAATAACACTGGATGAAGGGAC |
| | | ACACACTGAGACCT |
| 675 | snord45a_11_acceptor | TATGATAGGGACTTAGGGTG-ggtcaatgatgtgttggcatgtat |
| | | caggtattcctgtggctgatgtgtaataacactggatgaagggac |
| | | acacactgagacct |
| 676 | snord45a_11_acceptor | GGTCAATGATGTGTTGGCATGTATCAGGTATTCCT |
| | | GTGGCTGATGTGTAATAACACTGGATGAAGGGAC |
| | | ACACACTGAGACCT-tatgatagggacttagggtg |
| 677 | snord45a_7_acceptor | GGTCAATGATGTGTTGGCATGTATGGTACAGGTA |
| | | TTCCTCTGATGTGTAATAACACTCTGTGGATGAAG |
| | | GGACACTGAGACCT |
| 678 | snord45a_7_acceptor | TATGATAGGGACTTAGGGTG- |
| | | ggtcaatgatgtgttggcatgtatggt |
| | | acaggtattcctctgatgtgtaataacactctgtggatgaaggga |
| | | cactgagacct |
| 679 | snord45a_7_acceptor | GGTCAATGATGTGTTGGCATGTATGGTACAGGTA |
| | | TTCCTCTGATGTGTAATAACACTCTGTGGATGAAG |
| | | GGACACTGAGACCT-tatgatagggacttagggtg |
| 680 | aca46_acceptor_29 | AGCACTATAAAGGGACCTGTGGATGGGAATATTC |
| | | CCCATTCTTGGTACACACATAGTGCAAAAGAATT |
| | | CCTGGCTCTCTGTTGCACAGCTGACTTGTGCCATT |
| | | CTGCTGTTGCTGTATAGAGTTAAGGAACATGG |
| 681 | aca46_acceptor_29 | TATGATAGGGACTTAGGGTG- |
| | | agcactataaagggacctgtggatgggaatattccccattcttgg |
| | | tacacacatagtgcaaaagaattcctggctctctgttgcacagct |
| | | gacttgtgccattctgctgttgctgtatagagttaaggaacatgg |
| 682 | aca46_acceptor_29 | AGCACTATAAAGGGACCTGTGGATGGGAATATTC |
| | | CCCATTCTTGGTACACACATAGTGCAAAAGAATT |
| | | CCTGGCTCTCTGTTGCACAGCTGACTTGTGCCATT |
| | | CTGCTGTTGCTGTATAGAGTTAAGGAACATGG- |
| | | tatgatagggacttagggtg |
| 683 | aca44_2_acceptor_35 | CAGCATGTTTCCAAGGGCTGTGGCTGGTCATAGC |
| | | CATGGGATCTCCAACTGCATGCAAGAGCAACCTG |
| | | GAAAGACACACACAGCGCAGGTCAGTACAATACC |
| | | TGCAAGCTGCATGCCAGCTTTCCTATAATG |
| 684 | aca44_2_acceptor_35 | TATGATAGGGACTTAGGGTG- |
| | | cagcatgtttccaagggctgtggctggtc |
| | | atagccatgggatctccaactgcatgcaagagcaacctggaaaga |
| | | cacacacagcgcaggtcagtacaatacctgcaagctgcatgccag |
| | | ctttcctataatg |
| 685 | aca44_2_acceptor_35 | CAGCATGTTTCCAAGGGCTGTGGCTGGTCATAGC |
| | | CATGGGATCTCCAACTGCATGCAAGAGCAACCTG |
| | | GAAAGACACACACAGCGCAGGTCAGTACAATACC |
| | | TGCAAGCTGCATGCCAGCTTTCCTATAATG- |
| | | tatgatagggacttagggtg |

Format for the above sequences:
{Binding Motif}-{Sm consensus/Sm rand}-{U7/U7 rand}_{filler}

### Example 4: Design and Testing of U7 snRNA for Trans-Splicing

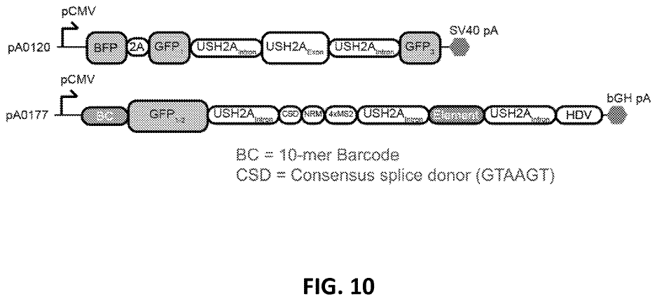

In the experiments of this Example, U7 snRNA guide constructs were developed and tested for trans-splicing. The U7 snRNA guide constructs were designed on a plasmid, along with a sequence that is complementary to a target sequence, to produce a U7 snRNA construct that occludes a splice site, and thereby allows for trans-splicing. To test for trans-splicing, U7 snRNA guides were designed on a plasmid (i.e., pA0120, FIG. 10, Table 2), which also included intron and exon sites of the USH2A gene and two GFP sites (FIG. 10).

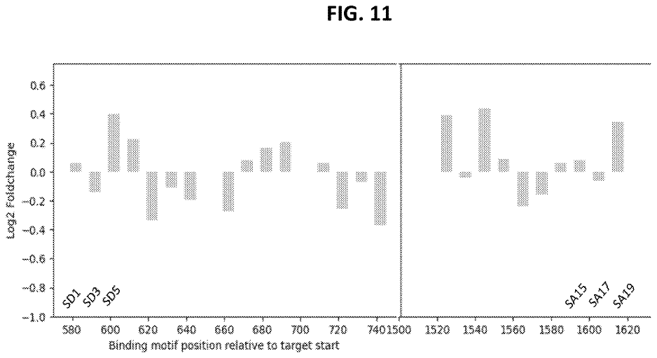

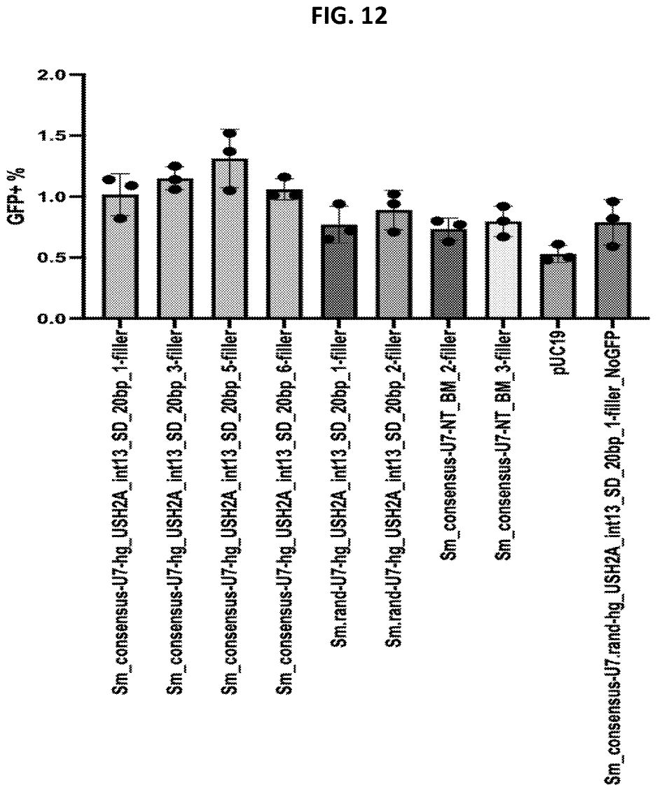

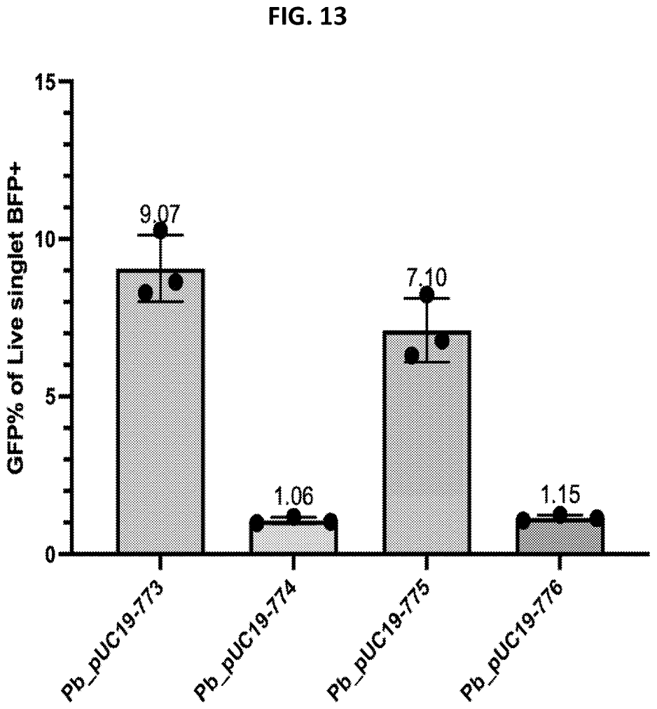

An element was then introduced on a separate plasmid target (i.e., pA0177), which was present after the pCMV promoter and before the bGHpA (FIG. 10), and included additional GFP sites. In these experiments, trans-splicing is observed by the expression of GFP, since the GFP site of the plasmid target (i.e., pA0177) will replace and restore the GFP site of the pA0120 plasmid, thereby demonstrating trans-splicing by GFP expression. FIG. 11 is a graph showing the binding of the U7 snRNA guide constructs to different target elements and the effects of trans-splicing. FIG. 12 is a graph showing independent validation of the results from FIG. 11. The first four bars from the left side show U7 snRNA constructs with specific targeting elements for trans-splicing, and the remaining bars on the graph show U7 snRNA constructs with non-targeting elements for trans-splicing. These experiments show, inter alia, that targeting near the splice donor enhances trans-splicing. FIG. 13 is a graph showing U7 snRNA targeting piggybac-integrated USH2A (773; hybridizing region and hairpin intact) compared to non-targeting guides (774 and 776), and mutated U7 snRNA hairpins (775; intact hybdrizing region, but hairpin region mutated). These experiments further show, inter alia, that targeting a complementary region with a U7 snRNA construct that has both the hairpin and hybridizing region leads to significantly higher trans-splicing compared to a non-targeting control.

TABLE 2

| Category | description | element_seq |
|---|---|---|
| SD | Sm_consensus-U7- | (SEQ ID NO: 658) ACACAGGCACTGGCCACTGA- |
| targeting | hg_USH2A_int13_SD_20b | AATTTTTGGAG- |
| | p_1-filler | (SEQ ID NO: 659) |
| | | taggctttctggcttttcaccggaaagcccct_AAATGATTAAATTAA |
| SD | Sm_consensus-U7- | (SEQ ID NO: 660) GATTAGGCACACACAGGCAC- |
| targeting | hg_USH2A_int13_SD_20b | AATTTTTGGAG- |
| | p_3-filler | (SEQ ID NO: 661) |
| | | taggctttctggcttttcaccggaaagcccct_AAATGATTAAATTAA |
| SD | Sm_consensus-U7- | (SEQ ID NO: 662) CTTCCTTGACGATTAGGCAC- |
| targeting | hg_USH2A_int13_SD_20b | AATTTTTGGAG- |
| | p_5-filler | (SEQ ID NO: 663) |
| | | taggctttctggcttttcaccggaaagcccct_AAATGATTAAATTAA |
| SD | Sm_consensus-U7- | (SEQ ID NO: 664) ACCTTCTTCCTTGACGATTA- |
| targeting | hg_USH2A_int13_SD_20b | AATTTTTGGAG- |
| | p_6-filler | (SEQ ID NO: 665) |
| | | taggctttctggcttttcaccggaaagcccct_AAATGATTAAATTAA |
| Random U7 | Sm.rand-U7- | (SEQ ID NO: 666) ACACAGGCACTGGCCACTGA- |
| control | hg_USH2A_int13_SD_20b | TGCGTGTCATT- |
| | p_1-filler | (SEQ ID NO: 667) |
| | | taggctttctggcttttcaccggaaagcccct_AAATGATTAAATTAA |
| Random U7 | Sm.rand-U7- | (SEQ ID NO: 668) GGCACACACAGGCACTGGCC- |
| control | hg_USH2A_int13_SD_20b | TGCGTGTCATT- |
| | p_2-filler | (SEQ ID NO: 669) |
| | | taggctttctggcttttcaccggaaagcccct_AAATGATTAAATTAA |
| Random Sm | Sm_consensus-U7- | (SEQ ID NO: 670) ACGAGCTCAGCCTATGCGAG- |
| control | NT_BM_2-filler | AATTTTTGGAG- |
| | | (SEQ ID NO: 671) |
| | | taggctttctggcttttcaccggaaagcccct_AAATGATTAAATTAA |
| Random Sm | Sm_consensus-U7- | (SEQ ID NO: 672) CCACCTACCCTATCGTGCGG- |
| control | NT_BM_3-filler | AATTTTTGGAG- |
| | | (SEQ ID NO: 673) |
| | | taggctttctggcttttcaccggaaagcccct_AAATGATTAAATTAA |

Format for the above sequences:
(Binding Motif}-{Sm consensus/Sm rand}-{U7/U7 rand}_{filler}

TABLE 3
SEQUENCE_LISTING

| SEQ | | |
|---|---|---|
| ID | Name/ | |
| NO | ID | Sequence |
| 1 | H box | ANANNA N = A, C, G, OR U |
| 2 | ACA | ACA |
| | box | |
| 3 | Sm | AAUUUUUGG |
| | consensus | |
| | motf | |
| 4 | Sm U7 | AAUUUGUCU |
| | motif | |
| 5 | C box | RUGAUGA R = A OR G |
| 6 | D box | CUGA |
| 7 | C′ | RUGAUGA |
| | box | |
| 8 | D′ | CUGA |
| | box | |
| 9 | U1_1_0 | AUACUUACGUAACAGGAGAAAAUACGGCCAUGAAGUUGGUGUUUCUCGGGG |
| | | GCGAUUUCUCCAUUGUACUCAGUAUGUGCUGACUGACUCCUGUUACUUCCA |
| | | CAUGUGGGGAAACUGGACUGUAAUUUGUGGUGGUGGGGAAUUGCGUUCGCG |
| | | C |
| 10 | U1_1_1 | UACUUACGUAACAGGAGAAAAUACGGCCAUGAAGUUGGUGUUUCUCGGGGG |
| | | CGAUUUCUCCAUUGUACUCAGUAUGUGCUGACUGACUCCUGUUACUUCCAC |
| | | AUGUGGGGAAACUGGACUGUAAUUUGUGGUGGUGGGGAAUUGCGUUCGCGC |
| | | U |
| 11 | U1_1_2 | ACUUACGUAACAGGAGAAAAUACGGCCAUGAAGUUGGUGUUUCUCGGGGGC |
| | | GAUUUCUCCAUUGUACUCAGUAUGUGCUGACUGACUCCUGUUACUUCCACA |
| | | UGUGGGGAAACUGGACUGUAAUUUGUGGUGGUGGGGAAUUGCGUUCGCGCU |
| | | U |
| 12 | U1_1_3 | CUUACGUAACAGGAGAAAAUACGGCCAUGAAGUUGGUGUUUCUCGGGGGCG |
| | | AUUUCUCCAUUGUACUCAGUAUGUGCUGACUGACUCCUGUUACUUCCACAU |
| | | GUGGGGAAACUGGACUGUAAUUUGUGGUGGUGGGGAAUUGCGUUCGCGCUU |
| | | U |
| 13 | U1_1_4 | UUACGUAACAGGAGAAAAUACGGCCAUGAAGUUGGUGUUUCUCGGGGGCGA |
| | | UUUCUCCAUUGUACUCAGUAUGUGCUGACUGACUCCUGUUACUUCCACAUG |
| | | UGGGGAAACUGGACUGUAAUUUGUGGUGGUGGGGAAUUGCGUUCGCGCUUU |
| | | C |
| 14 | U1_1_5 | UACGUAACAGGAGAAAAUACGGCCAUGAAGUUGGUGUUUCUCGGGGGCGAU |
| | | UUCUCCAUUGUACUCAGUAUGUGCUGACUGACUCCUGUUACUUCCACAUGU |
| | | GGGGAAACUGGACUGUAAUUUGUGGUGGUGGGGAAUUGCGUUCGCGCUUUC |
| | | U |
| 15 | U1_1_6 | ACGUAACAGGAGAAAAUACGGCCAUGAAGUUGGUGUUUCUCGGGGGCGAUU |
| | | UCUCCAUUGUACUCAGUAUGUGCUGACUGACUCCUGUUACUUCCACAUGUG |
| | | GGGAAACUGGACUGUAAUUUGUGGUGGUGGGGAAUUGCGUUCGCGCUUUCU |
| | | U |
| 16 | U1_1_7 | CGUAACAGGAGAAAAUACGGCCAUGAAGUUGGUGUUUCUCGGGGGCGAUUU |
| | | CUCCAUUGUACUCAGUAUGUGCUGACUGACUCCUGUUACUUCCACAUGUGG |
| | | GGAAACUGGACUGUAAUUUGUGGUGGUGGGGAAUUGCGUUCGCGCUUUCUU |
| | | C |
| 17 | U1_1_8 | GUAACAGGAGAAAAUACGGCCAUGAAGUUGGUGUUUCUCGGGGGCGAUUUC |
| | | UCCAUUGUACUCAGUAUGUGCUGACUGACUCCUGUUACUUCCACAUGUGGG |
| | | GAAACUGGACUGUAAUUUGUGGUGGUGGGGAAUUGCGUUCGCGCUUUCUUC |
| | | U |
| 18 | U1_1_9 | UAACAGGAGAAAAUACGGCCAUGAAGUUGGUGUUUCUCGGGGGCGAUUUCU |
| | | CCAUUGUACUCAGUAUGUGCUGACUGACUCCUGUUACUUCCACAUGUGGGG |
| | | AAACUGGACUGUAAUUUGUGGUGGUGGGGAAUUGCGUUCGCGCUUUCUUCU |
| 19 | U1_2_0 | AUAUUUACUUGGCAGGGGAGAUAACGUGACCACGAAGGUGGUUUUCCCAGG |
| | | GCUGAGGCUUAUUCAUUGUACUCCGGAUGUGCUGACCCCUGCGAUUUCCCC |
| | | AAAUGUGGGAAACUCGACUGCAUAAUUUGUGGUAGUGGGGGGCUGUGUCCG |
| | | U |
| 20 | U1_2_1 | UAUUUACUUGGCAGGGGAGAUAACGUGACCACGAAGGUGGUUUUCCCAGGG |
| | | CUGAGGCUUAUUCAUUGUACUCCGGAUGUGCUGACCCCUGCGAUUUCCCCA |
| | | AAUGUGGGAAACUCGACUGCAUAAUUUGUGGUAGUGGGGGGCUGUGUCCGU |
| | | G |
| 21 | U1_2_2 | AUUUACUUGGCAGGGGAGAUAACGUGACCACGAAGGUGGUUUUCCCAGGGC |
| | | UGAGGCUUAUUCAUUGUACUCCGGAUGUGCUGACCCCUGCGAUUUCCCCAA |
| | | AUGUGGGAAACUCGACUGCAUAAUUUGUGGUAGUGGGGGGCUGUGUCCGUG |
| | | C |
| 22 | U1_2_3 | UUUACUUGGCAGGGGAGAUAACGUGACCACGAAGGUGGUUUUCCCAGGGCU |
| | | GAGGCUUAUUCAUUGUACUCCGGAUGUGCUGACCCCUGCGAUUUCCCCAAA |
| | | UGUGGGAAACUCGACUGCAUAAUUUGUGGUAGUGGGGGGCUGUGUCCGUGC |
| | | U |
| 23 | U1_2_4 | UUACUUGGCAGGGGAGAUAACGUGACCACGAAGGUGGUUUUCCCAGGGCUG |
| | | AGGCUUAUUCAUUGUACUCCGGAUGUGCUGACCCCUGCGAUUUCCCCAAAU |
| | | GUGGGAAACUCGACUGCAUAAUUUGUGGUAGUGGGGGGCUGUGUCCGUGCU |
| | | U |
| 24 | U1_2_5 | UACUUGGCAGGGGAGAUAACGUGACCACGAAGGUGGUUUUCCCAGGGCUGA |
| | | GGCUUAUUCAUUGUACUCCGGAUGUGCUGACCCCUGCGAUUUCCCCAAAUG |
| | | UGGGAAACUCGACUGCAUAAUUUGUGGUAGUGGGGGGCUGUGUCCGUGCUU |
| | | U |
| 25 | U1_2_6 | ACUUGGCAGGGGAGAUAACGUGACCACGAAGGUGGUUUUCCCAGGGCUGAG |
| | | GCUUAUUCAUUGUACUCCGGAUGUGCUGACCCCUGCGAUUUCCCCAAAUGU |
| | | GGGAAACUCGACUGCAUAAUUUGUGGUAGUGGGGGGCUGUGUCCGUGCUUU |
| | | U |
| 26 | U1_2_7 | CUUGGCAGGGGAGAUAACGUGACCACGAAGGUGGUUUUCCCAGGGCUGAGG |
| | | CUUAUUCAUUGUACUCCGGAUGUGCUGACCCCUGCGAUUUCCCCAAAUGUG |
| | | GGAAACUCGACUGCAUAAUUUGUGGUAGUGGGGGGCUGUGUCCGUGCUUUU |
| | | C |
| 27 | U1_2_8 | UUGGCAGGGGAGAUAACGUGACCACGAAGGUGGUUUUCCCAGGGCUGAGGC |
| | | UUAUUCAUUGUACUCCGGAUGUGCUGACCCCUGCGAUUUCCCCAAAUGUGG |
| | | GAAACUCGACUGCAUAAUUUGUGGUAGUGGGGGGCUGUGUCCGUGCUUUUC |
| | | C |
| 28 | U1_2_9 | UGGCAGGGGAGAUAACGUGACCACGAAGGUGGUUUUCCCAGGGCUGAGGCU |
| | | UAUUCAUUGUACUCCGGAUGUGCUGACCCCUGCGAUUUCCCCAAAUGUGGG |
| | | AAACUCGACUGCAUAAUUUGUGGUAGUGGGGGGCUGUGUCCGUGCUUUUCC |
| | | C |
| 29 | U1_3_0 | AUACUUACCUGGCAGGGCAGAUACCAUGAUCUUAAAGGCAGUUUUCCCAGG |
| | | GCAAGGCUUAUCCAUUCCACUCUGGAUCCAUUAUAGGGGCAUGCUGAUCCC |
| | | UGGAAUUGCCCCAAAUGUGGGAAGCUCUACUGCAAAAUUUUUGGUAGUGAG |
| | | C |
| 30 | U1_3_1 | UACUUACCUGGCAGGGCAGAUACCAUGAUCUUAAAGGCAGUUUUCCCAGGG |
| | | CAAGGCUUAUCCAUUCCACUCUGGAUCCAUUAUAGGGGCAUGCUGAUCCCU |
| | | GGAAUUGCCCCAAAUGUGGGAAGCUCUACUGCAAAAUUUUUGGUAGUGAGC |
| | | G |
| 31 | U1_3_2 | ACUUACCUGGCAGGGCAGAUACCAUGAUCUUAAAGGCAGUUUUCCCAGGGC |
| | | AAGGCUUAUCCAUUCCACUCUGGAUCCAUUAUAGGGGCAUGCUGAUCCCUG |
| | | GAAUUGCCCCAAAUGUGGGAAGCUCUACUGCAAAAUUUUUGGUAGUGAGCG |
| | | A |
| 32 | U1_3_3 | CUUACCUGGCAGGGCAGAUACCAUGAUCUUAAAGGCAGUUUUCCCAGGGCA |
| | | AGGCUUAUCCAUUCCACUCUGGAUCCAUUAUAGGGGCAUGCUGAUCCCUGG |
| | | AAUUGCCCCAAAUGUGGGAAGCUCUACUGCAAAAUUUUUGGUAGUGAGCGA |
| | | U |
| 33 | U1_3_4 | UUACCUGGCAGGGCAGAUACCAUGAUCUUAAAGGCAGUUUUCCCAGGGCAA |
| | | GGCUUAUCCAUUCCACUCUGGAUCCAUUAUAGGGGCAUGCUGAUCCCUGGA |
| | | AUUGCCCCAAAUGUGGGAAGCUCUACUGCAAAAUUUUUGGUAGUGAGCGAU |
| | | G |
| 34 | U1_3_5 | UACCUGGCAGGGCAGAUACCAUGAUCUUAAAGGCAGUUUUCCCAGGGCAAG |
| | | GCUUAUCCAUUCCACUCUGGAUCCAUUAUAGGGGCAUGCUGAUCCCUGGAA |
| | | UUGCCCCAAAUGUGGGAAGCUCUACUGCAAAAUUUUUGGUAGUGAGCGAUG |
| | | G |
| 35 | U1_3_6 | ACCUGGCAGGGCAGAUACCAUGAUCUUAAAGGCAGUUUUCCCAGGGCAAGG |
| | | CUUAUCCAUUCCACUCUGGAUCCAUUAUAGGGGCAUGCUGAUCCCUGGAAU |
| | | UGCCCCAAAUGUGGGAAGCUCUACUGCAAAAUUUUUGGUAGUGAGCGAUGG |
| | | C |
| 36 | U1_3_7 | CCUGGCAGGGCAGAUACCAUGAUCUUAAAGGCAGUUUUCCCAGGGCAAGGC |
| | | UUAUCCAUUCCACUCUGGAUCCAUUAUAGGGGCAUGCUGAUCCCUGGAAUU |
| | | GCCCCAAAUGUGGGAAGCUCUACUGCAAAAUUUUUGGUAGUGAGCGAUGGC |
| | | A |
| 37 | U1_3_8 | CUGGCAGGGCAGAUACCAUGAUCUUAAAGGCAGUUUUCCCAGGGCAAGGCU |
| | | UAUCCAUUCCACUCUGGAUCCAUUAUAGGGGCAUGCUGAUCCCUGGAAUUG |
| | | CCCCAAAUGUGGGAAGCUCUACUGCAAAAUUUUUGGUAGUGAGCGAUGGCA |
| | | U |
| 38 | U1_3_9 | UGGCAGGGCAGAUACCAUGAUCUUAAAGGCAGUUUUCCCAGGGCAAGGCUU |
| | | AUCCAUUCCACUCUGGAUCCAUUAUAGGGGCAUGCUGAUCCCUGGAAUUGC |
| | | CCCAAAUGUGGGAAGCUCUACUGCAAAAUUUUUGGUAGUGAGCGAUGGCAU |
| | | U |
| 39 | U1_4_0 | AUACUUACCUGGCAGGGGAGAUACCAUGAUCACGAAGGUGGUUUUCCCAGG |
| | | GCGAGGCUUAUCCAUUGCACUCCGGAUGUGCUGACCCCUGCGAUUUCCCCA |
| | | AAUGUGGGAAACUCGACUGCAUAAUUUGUGGUAGUGGGGGACUGCGUUCGC |
| | | G |
| 40 | U1_4_1 | UACUUACCUGGCAGGGGAGAUACCAUGAUCACGAAGGUGGUUUUCCCAGGG |
| | | CGAGGCUUAUCCAUUGCACUCCGGAUGUGCUGACCCCUGCGAUUUCCCCAA |
| | | AUGUGGGAAACUCGACUGCAUAAUUUGUGGUAGUGGGGGACUGCGUUCGCG |
| | | C |
| 41 | U1_4_2 | ACUUACCUGGCAGGGGAGAUACCAUGAUCACGAAGGUGGUUUUCCCAGGGC |
| | | GAGGCUUAUCCAUUGCACUCCGGAUGUGCUGACCCCUGCGAUUUCCCCAAA |
| | | UGUGGGAAACUCGACUGCAUAAUUUGUGGUAGUGGGGGACUGCGUUCGCGC |
| | | U |
| 42 | U1_4_3 | CUUACCUGGCAGGGGAGAUACCAUGAUCACGAAGGUGGUUUUCCCAGGGCG |
| | | AGGCUUAUCCAUUGCACUCCGGAUGUGCUGACCCCUGCGAUUUCCCCAAAU |
| | | GUGGGAAACUCGACUGCAUAAUUUGUGGUAGUGGGGGACUGCGUUCGCGCU |
| | | U |
| 43 | U1_4_4 | UUACCUGGCAGGGGAGAUACCAUGAUCACGAAGGUGGUUUUCCCAGGGCGA |
| | | GGCUUAUCCAUUGCACUCCGGAUGUGCUGACCCCUGCGAUUUCCCCAAAUG |
| | | UGGGAAACUCGACUGCAUAAUUUGUGGUAGUGGGGGACUGCGUUCGCGCUU |
| | | U |
| 44 | U1_4_5 | UACCUGGCAGGGGAGAUACCAUGAUCACGAAGGUGGUUUUCCCAGGGCGAG |
| | | GCUUAUCCAUUGCACUCCGGAUGUGCUGACCCCUGCGAUUUCCCCAAAUGU |
| | | GGGAAACUCGACUGCAUAAUUUGUGGUAGUGGGGGACUGCGUUCGCGCUUU |
| | | C |
| 45 | U1_4_6 | ACCUGGCAGGGGAGAUACCAUGAUCACGAAGGUGGUUUUCCCAGGGCGAGG |
| | | CUUAUCCAUUGCACUCCGGAUGUGCUGACCCCUGCGAUUUCCCCAAAUGUG |
| | | GGAAACUCGACUGCAUAAUUUGUGGUAGUGGGGGACUGCGUUCGCGCUUUC |
| | | C |
| 46 | U1_4_7 | CCUGGCAGGGGAGAUACCAUGAUCACGAAGGUGGUUUUCCCAGGGCGAGGC |
| | | UUAUCCAUUGCACUCCGGAUGUGCUGACCCCUGCGAUUUCCCCAAAUGUGG |
| | | GAAACUCGACUGCAUAAUUUGUGGUAGUGGGGGACUGCGUUCGCGCUUUCC |
| | | C |
| 47 | U1_4_8 | CUGGCAGGGGAGAUACCAUGAUCACGAAGGUGGUUUUCCCAGGGCGAGGCU |
| | | UAUCCAUUGCACUCCGGAUGUGCUGACCCCUGCGAUUUCCCCAAAUGUGGG |
| | | AAACUCGACUGCAUAAUUUGUGGUAGUGGGGGACUGCGUUCGCGCUUUCCC |
| | | C |
| 48 | U1_4_9 | UGGCAGGGGAGAUACCAUGAUCACGAAGGUGGUUUUCCCAGGGCGAGGCUU |
| | | AUCCAUUGCACUCCGGAUGUGCUGACCCCUGCGAUUUCCCCAAAUGUGGGA |
| | | AACUCGACUGCAUAAUUUGUGGUAGUGGGGGACUGCGUUCGCGCUUUCCCC |
| | | U |
| 49 | U1_5_0 | AUACUUCCCUGACAGGGGAGAUACCUAGGAAUCCAACUUCCCAGGGCAAGA |
| | | CUGAUCUAUUGCACUAUGGAUGUGCCGACCCCUGAGAUUUACAAAAUUGUG |
| | | GGAAACUCAACUGCAUAAUUUAUGGAAAUGAAGGACUGUGUUUGCGCUUUC |
| | | A |
| 50 | U1_5_1 | UACUUCCCUGACAGGGGAGAUACCUAGGAAUCCAACUUCCCAGGGCAAGAC |
| | | UGAUCUAUUGCACUAUGGAUGUGCCGACCCCUGAGAUUUACAAAAUUGUGG |
| | | GAAACUCAACUGCAUAAUUUAUGGAAAUGAAGGACUGUGUUUGCGCUUUCA |
| 51 | U1_5_2 | ACUUCCCUGACAGGGGAGAUACCUAGGAAUCCAACUUCCCAGGGCAAGACU |
| | | GAUCUAUUGCACUAUGGAUGUGCCGACCCCUGAGAUUUACAAAAUUGUGGG |
| | | AAACUCAACUGCAUAAUUUAUGGAAAUGAAGGACUGUGUUUGCGCUUUCA |
| 52 | U1_5_3 | CUUCCCUGACAGGGGAGAUACCUAGGAAUCCAACUUCCCAGGGCAAGACUG |
| | | AUCUAUUGCACUAUGGAUGUGCCGACCCCUGAGAUUUACAAAAUUGUGGGA |
| | | AACUCAACUGCAUAAUUUAUGGAAAUGAAGGACUGUGUUUGCGCUUUCA |
| 53 | U1_5_4 | UUCCCUGACAGGGGAGAUACCUAGGAAUCCAACUUCCCAGGGCAAGACUGA |
| | | UCUAUUGCACUAUGGAUGUGCCGACCCCUGAGAUUUACAAAAUUGUGGGAA |
| | | ACUCAACUGCAUAAUUUAUGGAAAUGAAGGACUGUGUUUGCGCUUUCA |
| 54 | U1_5_5 | UCCCUGACAGGGGAGAUACCUAGGAAUCCAACUUCCCAGGGCAAGACUGAU |
| | | CUAUUGCACUAUGGAUGUGCCGACCCCUGAGAUUUACAAAAUUGUGGGAAA |
| | | CUCAACUGCAUAAUUUAUGGAAAUGAAGGACUGUGUUUGCGCUUUCA |
| 55 | U1_5_6 | CCCUGACAGGGGAGAUACCUAGGAAUCCAACUUCCCAGGGCAAGACUGAUC |
| | | UAUUGCACUAUGGAUGUGCCGACCCCUGAGAUUUACAAAAUUGUGGGAAAC |
| | | UCAACUGCAUAAUUUAUGGAAAUGAAGGACUGUGUUUGCGCUUUCA |
| 56 | U1_5_7 | CCUGACAGGGGAGAUACCUAGGAAUCCAACUUCCCAGGGCAAGACUGAUCU |
| | | AUUGCACUAUGGAUGUGCCGACCCCUGAGAUUUACAAAAUUGUGGGAAACU |
| | | CAACUGCAUAAUUUAUGGAAAUGAAGGACUGUGUUUGCGCUUUCA |
| 57 | U1_5_8 | CUGACAGGGGAGAUACCUAGGAAUCCAACUUCCCAGGGCAAGACUGAUCUA |
| | | UUGCACUAUGGAUGUGCCGACCCCUGAGAUUUACAAAAUUGUGGGAAACUC |
| | | AACUGCAUAAUUUAUGGAAAUGAAGGACUGUGUUUGCGCUUUCA |
| 58 | U1_5_9 | UGACAGGGGAGAUACCUAGGAAUCCAACUUCCCAGGGCAAGACUGAUCUAU |
| | | UGCACUAUGGAUGUGCCGACCCCUGAGAUUUACAAAAUUGUGGGAAACUCA |
| | | ACUGCAUAAUUUAUGGAAAUGAAGGACUGUGUUUGCGCUUUCA |
| 59 | U1_6_0 | AAACUUCUCUGCCAGGGGAGAUACCAUGAUCACAAAGGUGGCUUUCUCAGG |
| | | GAAAGGCUGAUUUAUUGCACUCCAGAUGUGCUGACCCCUGAGAUUUCUCCA |
| | | AAUGUGGGAAACUCAGCUGCAUAAUUUGUGGAAGCGAAGGACUGUGUUUGU |
| | | G |
| 60 | U1_6_1 | AACUUCUCUGCCAGGGGAGAUACCAUGAUCACAAAGGUGGCUUUCUCAGGG |
| | | AAAGGCUGAUUUAUUGCACUCCAGAUGUGCUGACCCCUGAGAUUUCUCCAA |
| | | AUGUGGGAAACUCAGCUGCAUAAUUUGUGGAAGCGAAGGACUGUGUUUGUG |
| | | C |
| 61 | U1_6_2 | ACUUCUCUGCCAGGGGAGAUACCAUGAUCACAAAGGUGGCUUUCUCAGGGA |
| | | AAGGCUGAUUUAUUGCACUCCAGAUGUGCUGACCCCUGAGAUUUCUCCAAA |
| | | UGUGGGAAACUCAGCUGCAUAAUUUGUGGAAGCGAAGGACUGUGUUUGUGC |
| | | U |
| 62 | U1_6_3 | CUUCUCUGCCAGGGGAGAUACCAUGAUCACAAAGGUGGCUUUCUCAGGGAA |
| | | AGGCUGAUUUAUUGCACUCCAGAUGUGCUGACCCCUGAGAUUUCUCCAAAU |
| | | GUGGGAAACUCAGCUGCAUAAUUUGUGGAAGCGAAGGACUGUGUUUGUGCU |
| | | U |
| 63 | U1_6_4 | UUCUCUGCCAGGGGAGAUACCAUGAUCACAAAGGUGGCUUUCUCAGGGAAA |
| | | GGCUGAUUUAUUGCACUCCAGAUGUGCUGACCCCUGAGAUUUCUCCAAAUG |
| | | UGGGAAACUCAGCUGCAUAAUUUGUGGAAGCGAAGGACUGUGUUUGUGCUU |
| | | U |
| 64 | U1_6_5 | UCUCUGCCAGGGGAGAUACCAUGAUCACAAAGGUGGCUUUCUCAGGGAAAG |
| | | GCUGAUUUAUUGCACUCCAGAUGUGCUGACCCCUGAGAUUUCUCCAAAUGU |
| | | GGGAAACUCAGCUGCAUAAUUUGUGGAAGCGAAGGACUGUGUUUGUGCUUU |
| | | C |
| 65 | U1_6_6 | CUCUGCCAGGGGAGAUACCAUGAUCACAAAGGUGGCUUUCUCAGGGAAAGG |
| | | CUGAUUUAUUGCACUCCAGAUGUGCUGACCCCUGAGAUUUCUCCAAAUGUG |
| | | GGAAACUCAGCUGCAUAAUUUGUGGAAGCGAAGGACUGUGUUUGUGCUUUC |
| | | A |
| 66 | U1_6_7 | UCUGCCAGGGGAGAUACCAUGAUCACAAAGGUGGCUUUCUCAGGGAAAGGC |
| | | UGAUUUAUUGCACUCCAGAUGUGCUGACCCCUGAGAUUUCUCCAAAUGUGG |
| | | GAAACUCAGCUGCAUAAUUUGUGGAAGCGAAGGACUGUGUUUGUGCUUUCA |
| | | U |
| 67 | U1_6_8 | CUGCCAGGGGAGAUACCAUGAUCACAAAGGUGGCUUUCUCAGGGAAAGGCU |
| | | GAUUUAUUGCACUCCAGAUGUGCUGACCCCUGAGAUUUCUCCAAAUGUGGG |
| | | AAACUCAGCUGCAUAAUUUGUGGAAGCGAAGGACUGUGUUUGUGCUUUCAU |
| | | G |
| 68 | U1_6_9 | UGCCAGGGGAGAUACCAUGAUCACAAAGGUGGCUUUCUCAGGGAAAGGCUG |
| | | AUUUAUUGCACUCCAGAUGUGCUGACCCCUGAGAUUUCUCCAAAUGUGGGA |
| | | AACUCAGCUGCAUAAUUUGUGGAAGCGAAGGACUGUGUUUGUGCUUUCAUG |
| | | U |
| 69 | U1_7_0 | AUACUUCUCUGCCAGGGGAGAUACUGUGAUCAUGAAGGUGGCUUUCCCAGG |
| | | GCAAGGCUGAUCUAUUGCAUUCUGGAUGUGCUGACUCCUACGAUUUCCCCA |
| | | AGUGUGGGAAACUCAACUGCAUAAUUUGUGGAAGUAAUGACUGUGUUUGCA |
| | | C |
| 70 | U1_7_1 | UACUUCUCUGCCAGGGGAGAUACUGUGAUCAUGAAGGUGGCUUUCCCAGGG |
| | | CAAGGCUGAUCUAUUGCAUUCUGGAUGUGCUGACUCCUACGAUUUCCCCAA |
| | | GUGUGGGAAACUCAACUGCAUAAUUUGUGGAAGUAAUGACUGUGUUUGCAC |
| | | U |
| 71 | U1_7_2 | ACUUCUCUGCCAGGGGAGAUACUGUGAUCAUGAAGGUGGCUUUCCCAGGGC |
| | | AAGGCUGAUCUAUUGCAUUCUGGAUGUGCUGACUCCUACGAUUUCCCCAAG |
| | | UGUGGGAAACUCAACUGCAUAAUUUGUGGAAGUAAUGACUGUGUUUGCACU |
| | | U |
| 72 | U1_7_3 | CUUCUCUGCCAGGGGAGAUACUGUGAUCAUGAAGGUGGCUUUCCCAGGGCA |
| | | AGGCUGAUCUAUUGCAUUCUGGAUGUGCUGACUCCUACGAUUUCCCCAAGU |
| | | GUGGGAAACUCAACUGCAUAAUUUGUGGAAGUAAUGACUGUGUUUGCACUU |
| | | U |
| 73 | U1_7_4 | UUCUCUGCCAGGGGAGAUACUGUGAUCAUGAAGGUGGCUUUCCCAGGGCAA |
| | | GGCUGAUCUAUUGCAUUCUGGAUGUGCUGACUCCUACGAUUUCCCCAAGUG |
| | | UGGGAAACUCAACUGCAUAAUUUGUGGAAGUAAUGACUGUGUUUGCACUUU |
| | | C |
| 74 | U1_7_5 | UCUCUGCCAGGGGAGAUACUGUGAUCAUGAAGGUGGCUUUCCCAGGGCAAG |
| | | GCUGAUCUAUUGCAUUCUGGAUGUGCUGACUCCUACGAUUUCCCCAAGUGU |
| | | GGGAAACUCAACUGCAUAAUUUGUGGAAGUAAUGACUGUGUUUGCACUUUC |
| | | A |
| 75 | U1_7_6 | CUCUGCCAGGGGAGAUACUGUGAUCAUGAAGGUGGCUUUCCCAGGGCAAGG |
| | | CUGAUCUAUUGCAUUCUGGAUGUGCUGACUCCUACGAUUUCCCCAAGUGUG |
| | | GGAAACUCAACUGCAUAAUUUGUGGAAGUAAUGACUGUGUUUGCACUUUCA |
| | | C |
| 76 | U1_7_7 | UCUGCCAGGGGAGAUACUGUGAUCAUGAAGGUGGCUUUCCCAGGGCAAGGC |
| | | UGAUCUAUUGCAUUCUGGAUGUGCUGACUCCUACGAUUUCCCCAAGUGUGG |
| | | GAAACUCAACUGCAUAAUUUGUGGAAGUAAUGACUGUGUUUGCACUUUCAC |
| | | G |
| 77 | U1_7_8 | CUGCCAGGGGAGAUACUGUGAUCAUGAAGGUGGCUUUCCCAGGGCAAGGCU |
| | | GAUCUAUUGCAUUCUGGAUGUGCUGACUCCUACGAUUUCCCCAAGUGUGGG |
| | | AAACUCAACUGCAUAAUUUGUGGAAGUAAUGACUGUGUUUGCACUUUCACG |
| | | U |
| 78 | U1_7_9 | UGCCAGGGGAGAUACUGUGAUCAUGAAGGUGGCUUUCCCAGGGCAAGGCUG |
| | | AUCUAUUGCAUUCUGGAUGUGCUGACUCCUACGAUUUCCCCAAGUGUGGGA |
| | | AACUCAACUGCAUAAUUUGUGGAAGUAAUGACUGUGUUUGCACUUUCACGU |
| 79 | U1_8_0 | AUACUUCCCUGCCAGGGGAGAUACCGUGAUCAUGAAGGUGGCUUUCCCAGG |
| | | GAAAGGCCGAUAUGUUGCACUCUAGAUGUGCUGACCCGUGAGAUUUCCCCA |
| | | AAUGUGGGACACUCAAAUGCAUAAUUGGUGGAAGUGAAGGACUGUGUUUGU |
| | | G |
| 80 | U1_8_1 | UACUUCCCUGCCAGGGGAGAUACCGUGAUCAUGAAGGUGGCUUUCCCAGGG |
| | | AAAGGCCGAUAUGUUGCACUCUAGAUGUGCUGACCCGUGAGAUUUCCCCAA |
| | | AUGUGGGACACUCAAAUGCAUAAUUGGUGGAAGUGAAGGACUGUGUUUGUG |
| | | C |
| 81 | U1_8_2 | ACUUCCCUGCCAGGGGAGAUACCGUGAUCAUGAAGGUGGCUUUCCCAGGGA |
| | | AAGGCCGAUAUGUUGCACUCUAGAUGUGCUGACCCGUGAGAUUUCCCCAAA |
| | | UGUGGGACACUCAAAUGCAUAAUUGGUGGAAGUGAAGGACUGUGUUUGUGC |
| | | U |
| 82 | U1_8_3 | CUUCCCUGCCAGGGGAGAUACCGUGAUCAUGAAGGUGGCUUUCCCAGGGAA |
| | | AGGCCGAUAUGUUGCACUCUAGAUGUGCUGACCCGUGAGAUUUCCCCAAAU |
| | | GUGGGACACUCAAAUGCAUAAUUGGUGGAAGUGAAGGACUGUGUUUGUGCU |
| | | U |
| 83 | U1_8_4 | UUCCCUGCCAGGGGAGAUACCGUGAUCAUGAAGGUGGCUUUCCCAGGGAAA |
| | | GGCCGAUAUGUUGCACUCUAGAUGUGCUGACCCGUGAGAUUUCCCCAAAUG |
| | | UGGGACACUCAAAUGCAUAAUUGGUGGAAGUGAAGGACUGUGUUUGUGCUU |
| | | U |
| 84 | U1_8_5 | UCCCUGCCAGGGGAGAUACCGUGAUCAUGAAGGUGGCUUUCCCAGGGAAAG |
| | | GCCGAUAUGUUGCACUCUAGAUGUGCUGACCCGUGAGAUUUCCCCAAAUGU |
| | | GGGACACUCAAAUGCAUAAUUGGUGGAAGUGAAGGACUGUGUUUGUGCUUU |
| | | C |
| 85 | U1_8_6 | CCCUGCCAGGGGAGAUACCGUGAUCAUGAAGGUGGCUUUCCCAGGGAAAGG |
| | | CCGAUAUGUUGCACUCUAGAUGUGCUGACCCGUGAGAUUUCCCCAAAUGUG |
| | | GGACACUCAAAUGCAUAAUUGGUGGAAGUGAAGGACUGUGUUUGUGCUUUC |
| | | A |
| 86 | U1_8_7 | CCUGCCAGGGGAGAUACCGUGAUCAUGAAGGUGGCUUUCCCAGGGAAAGGC |
| | | CGAUAUGUUGCACUCUAGAUGUGCUGACCCGUGAGAUUUCCCCAAAUGUGG |
| | | GACACUCAAAUGCAUAAUUGGUGGAAGUGAAGGACUGUGUUUGUGCUUUCA |
| 87 | U1_8_8 | CUGCCAGGGGAGAUACCGUGAUCAUGAAGGUGGCUUUCCCAGGGAAAGGCC |
| | | GAUAUGUUGCACUCUAGAUGUGCUGACCCGUGAGAUUUCCCCAAAUGUGGG |
| | | ACACUCAAAUGCAUAAUUGGUGGAAGUGAAGGACUGUGUUUGUGCUUUCA |
| 88 | U1_8_9 | UGCCAGGGGAGAUACCGUGAUCAUGAAGGUGGCUUUCCCAGGGAAAGGCCG |
| | | AUAUGUUGCACUCUAGAUGUGCUGACCCGUGAGAUUUCCCCAAAUGUGGGA |
| | | CACUCAAAUGCAUAAUUGGUGGAAGUGAAGGACUGUGUUUGUGCUUUCA |
| 89 | U1_9_0 | UUAACUACCUGACAGAGGAGAUACUGUGAUCAUGAAAGUGGUUUUCCUAGG |
| | | GCAAGACUUAUCCGUUGCACUCCAGAUGUGCUGACUCAUGCAAUUUCCCCA |
| | | AAUGUGGGAAACUCGACUACAUAAUUUCUGGUGGUAGGGGACUGCGUUCAU |
| | | G |
| 90 | U1_9_1 | UAACUACCUGACAGAGGAGAUACUGUGAUCAUGAAAGUGGUUUUCCUAGGG |
| | | CAAGACUUAUCCGUUGCACUCCAGAUGUGCUGACUCAUGCAAUUUCCCCAA |
| | | AUGUGGGAAACUCGACUACAUAAUUUCUGGUGGUAGGGGACUGCGUUCAUG |
| | | U |
| 91 | U1_9_2 | AACUACCUGACAGAGGAGAUACUGUGAUCAUGAAAGUGGUUUUCCUAGGGC |
| | | AAGACUUAUCCGUUGCACUCCAGAUGUGCUGACUCAUGCAAUUUCCCCAAA |
| | | UGUGGGAAACUCGACUACAUAAUUUCUGGUGGUAGGGGACUGCGUUCAUGU |
| | | U |
| 92 | U1_9_3 | ACUACCUGACAGAGGAGAUACUGUGAUCAUGAAAGUGGUUUUCCUAGGGCA |
| | | AGACUUAUCCGUUGCACUCCAGAUGUGCUGACUCAUGCAAUUUCCCCAAAU |
| | | GUGGGAAACUCGACUACAUAAUUUCUGGUGGUAGGGGACUGCGUUCAUGUU |
| | | C |
| 93 | U1_9_4 | CUACCUGACAGAGGAGAUACUGUGAUCAUGAAAGUGGUUUUCCUAGGGCAA |
| | | GACUUAUCCGUUGCACUCCAGAUGUGCUGACUCAUGCAAUUUCCCCAAAUG |
| | | UGGGAAACUCGACUACAUAAUUUCUGGUGGUAGGGGACUGCGUUCAUGUUC |
| | | U |
| 94 | U1_9_5 | UACCUGACAGAGGAGAUACUGUGAUCAUGAAAGUGGUUUUCCUAGGGCAAG |
| | | ACUUAUCCGUUGCACUCCAGAUGUGCUGACUCAUGCAAUUUCCCCAAAUGU |
| | | GGGAAACUCGACUACAUAAUUUCUGGUGGUAGGGGACUGCGUUCAUGUUCU |
| | | C |
| 95 | U1_9_6 | ACCUGACAGAGGAGAUACUGUGAUCAUGAAAGUGGUUUUCCUAGGGCAAGA |
| | | CUUAUCCGUUGCACUCCAGAUGUGCUGACUCAUGCAAUUUCCCCAAAUGUG |
| | | GGAAACUCGACUACAUAAUUUCUGGUGGUAGGGGACUGCGUUCAUGUUCUC |
| | | C |
| 96 | U1_9_7 | CCUGACAGAGGAGAUACUGUGAUCAUGAAAGUGGUUUUCCUAGGGCAAGAC |
| | | UUAUCCGUUGCACUCCAGAUGUGCUGACUCAUGCAAUUUCCCCAAAUGUGG |
| | | GAAACUCGACUACAUAAUUUCUGGUGGUAGGGGACUGCGUUCAUGUUCUCC |
| | | C |
| 97 | U1_9_8 | CUGACAGAGGAGAUACUGUGAUCAUGAAAGUGGUUUUCCUAGGGCAAGACU |
| | | UAUCCGUUGCACUCCAGAUGUGCUGACUCAUGCAAUUUCCCCAAAUGUGGG |
| | | AAACUCGACUACAUAAUUUCUGGUGGUAGGGGACUGCGUUCAUGUUCUCCC |
| | | C |
| 98 | U1_9_9 | UGACAGAGGAGAUACUGUGAUCAUGAAAGUGGUUUUCCUAGGGCAAGACUU |
| | | AUCCGUUGCACUCCAGAUGUGCUGACUCAUGCAAUUUCCCCAAAUGUGGGA |
| | | AACUCGACUACAUAAUUUCUGGUGGUAGGGGACUGCGUUCAUGUUCUCCCC |
| | | U |
| 99 | U1_10_0 | AGACUUAUCUGGCAGGGGAGACACCAUGAACAUGAGGAUAGUUUUCCCAAG |
| | | GCAAGUUUCAACCCUUGCACUCUAGAUGAUGAGAUUACUUAAUGGGUACAA |
| | | UGCAUGUGAUUUGGGUAAUGGAUACUGUAGAAACACUGACGUCAC |
| 100 | U1_10_1 | GACUUAUCUGGCAGGGGAGACACCAUGAACAUGAGGAUAGUUUUCCCAAGG |
| | | CAAGUUUCAACCCUUGCACUCUAGAUGAUGAGAUUACUUAAUGGGUACAAU |
| | | GCAUGUGAUUUGGGUAAUGGAUACUGUAGAAACACUGACGUCAC |
| 101 | U1_10_2 | ACUUAUCUGGCAGGGGAGACACCAUGAACAUGAGGAUAGUUUUCCCAAGGC |
| | | AAGUUUCAACCCUUGCACUCUAGAUGAUGAGAUUACUUAAUGGGUACAAUG |
| | | CAUGUGAUUUGGGUAAUGGAUACUGUAGAAACACUGACGUCAC |
| 102 | U1_10_3 | CUUAUCUGGCAGGGGAGACACCAUGAACAUGAGGAUAGUUUUCCCAAGGCA |
| | | AGUUUCAACCCUUGCACUCUAGAUGAUGAGAUUACUUAAUGGGUACAAUGC |
| | | AUGUGAUUUGGGUAAUGGAUACUGUAGAAACACUGACGUCAC |
| 103 | U1_10_4 | UUAUCUGGCAGGGGAGACACCAUGAACAUGAGGAUAGUUUUCCCAAGGCAA |
| | | GUUUCAACCCUUGCACUCUAGAUGAUGAGAUUACUUAAUGGGUACAAUGCA |
| | | UGUGAUUUGGGUAAUGGAUACUGUAGAAACACUGACGUCAC |
| 104 | U1_10_5 | UAUCUGGCAGGGGAGACACCAUGAACAUGAGGAUAGUUUUCCCAAGGCAAG |
| | | UUUCAACCCUUGCACUCUAGAUGAUGAGAUUACUUAAUGGGUACAAUGCAU |
| | | GUGAUUUGGGUAAUGGAUACUGUAGAAACACUGACGUCAC |
| 105 | U1_10_6 | AUCUGGCAGGGGAGACACCAUGAACAUGAGGAUAGUUUUCCCAAGGCAAGU |
| | | UUCAACCCUUGCACUCUAGAUGAUGAGAUUACUUAAUGGGUACAAUGCAUG |
| | | UGAUUUGGGUAAUGGAUACUGUAGAAACACUGACGUCAC |
| 106 | U1_10_7 | UCUGGCAGGGGAGACACCAUGAACAUGAGGAUAGUUUUCCCAAGGCAAGUU |
| | | UCAACCCUUGCACUCUAGAUGAUGAGAUUACUUAAUGGGUACAAUGCAUGU |
| | | GAUUUGGGUAAUGGAUACUGUAGAAACACUGACGUCAC |
| 107 | U1_10_8 | CUGGCAGGGGAGACACCAUGAACAUGAGGAUAGUUUUCCCAAGGCAAGUUU |
| | | CAACCCUUGCACUCUAGAUGAUGAGAUUACUUAAUGGGUACAAUGCAUGUG |
| | | AUUUGGGUAAUGGAUACUGUAGAAACACUGACGUCAC |
| 108 | U1_10_9 | UGGCAGGGGAGACACCAUGAACAUGAGGAUAGUUUUCCCAAGGCAAGUUUC |
| | | AACCCUUGCACUCUAGAUGAUGAGAUUACUUAAUGGGUACAAUGCAUGUGA |
| | | UUUGGGUAAUGGAUACUGUAGAAACACUGACGUCAC |
| 109 | U1_11_0 | AGACUCAUCUGGCAGCGGAGAUACCAUGAACAUCACGGUAGUUUUCCCAAA |
| | | GCAAGUUUCCACCCUUGCACUCCGGAUGAUGAGACAUUACUUAAUGGAUAC |
| | | AAUGCAUGUGAUUUGAGUGAUGGAUACUGUGGAAACACCGACUUCAC |
| 110 | U1_11_1 | GACUCAUCUGGCAGCGGAGAUACCAUGAACAUCACGGUAGUUUUCCCAAAG |
| | | CAAGUUUCCACCCUUGCACUCCGGAUGAUGAGACAUUACUUAAUGGAUACA |
| | | AUGCAUGUGAUUUGAGUGAUGGAUACUGUGGAAACACCGACUUCAC |
| 111 | U1_11_2 | ACUCAUCUGGCAGCGGAGAUACCAUGAACAUCACGGUAGUUUUCCCAAAGC |
| | | AAGUUUCCACCCUUGCACUCCGGAUGAUGAGACAUUACUUAAUGGAUACAA |
| | | UGCAUGUGAUUUGAGUGAUGGAUACUGUGGAAACACCGACUUCAC |
| 112 | U1_11_3 | CUCAUCUGGCAGCGGAGAUACCAUGAACAUCACGGUAGUUUUCCCAAAGCA |
| | | AGUUUCCACCCUUGCACUCCGGAUGAUGAGACAUUACUUAAUGGAUACAAU |
| | | GCAUGUGAUUUGAGUGAUGGAUACUGUGGAAACACCGACUUCAC |
| 113 | U1_11_4 | UCAUCUGGCAGCGGAGAUACCAUGAACAUCACGGUAGUUUUCCCAAAGCAA |
| | | GUUUCCACCCUUGCACUCCGGAUGAUGAGACAUUACUUAAUGGAUACAAUG |
| | | CAUGUGAUUUGAGUGAUGGAUACUGUGGAAACACCGACUUCAC |
| 114 | U1_11_5 | CAUCUGGCAGCGGAGAUACCAUGAACAUCACGGUAGUUUUCCCAAAGCAAG |
| | | UUUCCACCCUUGCACUCCGGAUGAUGAGACAUUACUUAAUGGAUACAAUGC |
| | | AUGUGAUUUGAGUGAUGGAUACUGUGGAAACACCGACUUCAC |
| 115 | U1_11_6 | AUCUGGCAGCGGAGAUACCAUGAACAUCACGGUAGUUUUCCCAAAGCAAGU |
| | | UUCCACCCUUGCACUCCGGAUGAUGAGACAUUACUUAAUGGAUACAAUGCA |
| | | UGUGAUUUGAGUGAUGGAUACUGUGGAAACACCGACUUCAC |
| 116 | U1_11_7 | UCUGGCAGCGGAGAUACCAUGAACAUCACGGUAGUUUUCCCAAAGCAAGUU |
| | | UCCACCCUUGCACUCCGGAUGAUGAGACAUUACUUAAUGGAUACAAUGCAU |
| | | GUGAUUUGAGUGAUGGAUACUGUGGAAACACCGACUUCAC |
| 117 | U1_11_8 | CUGGCAGCGGAGAUACCAUGAACAUCACGGUAGUUUUCCCAAAGCAAGUUU |
| | | CCACCCUUGCACUCCGGAUGAUGAGACAUUACUUAAUGGAUACAAUGCAUG |
| | | UGAUUUGAGUGAUGGAUACUGUGGAAACACCGACUUCAC |
| 118 | U1_11_9 | UGGCAGCGGAGAUACCAUGAACAUCACGGUAGUUUUCCCAAAGCAAGUUUC |
| | | CACCCUUGCACUCCGGAUGAUGAGACAUUACUUAAUGGAUACAAUGCAUGU |
| | | GAUUUGAGUGAUGGAUACUGUGGAAACACCGACUUCAC |
| 119 | U1_12_0 | AGACUUGGCAGGGGAGAUAGCAUGAUCACGAAGGUGGUUUUCCCAAGGCAA |
| | | GAUUUAUUCACUGCACUCUGGAUGUGCUGACCCCUACGAUUUCCGCCCAAU |
| | | GGGGAAGCUUGACUGCUUAGUUUGUUGUGGCAGGGGACUGUGUUCACACUU |
| | | U |
| 120 | U1_12_1 | GACUUGGCAGGGGAGAUAGCAUGAUCACGAAGGUGGUUUUCCCAAGGCAAG |
| | | AUUUAUUCACUGCACUCUGGAUGUGCUGACCCCUACGAUUUCCGCCCAAUG |
| | | GGGAAGCUUGACUGCUUAGUUUGUUGUGGCAGGGGACUGUGUUCACACUUU |
| | | U |
| 121 | U1_12_2 | ACUUGGCAGGGGAGAUAGCAUGAUCACGAAGGUGGUUUUCCCAAGGCAAGA |
| | | UUUAUUCACUGCACUCUGGAUGUGCUGACCCCUACGAUUUCCGCCCAAUGG |
| | | GGAAGCUUGACUGCUUAGUUUGUUGUGGCAGGGGACUGUGUUCACACUUUU |
| | | C |
| 122 | U1_12_3 | CUUGGCAGGGGAGAUAGCAUGAUCACGAAGGUGGUUUUCCCAAGGCAAGAU |
| | | UUAUUCACUGCACUCUGGAUGUGCUGACCCCUACGAUUUCCGCCCAAUGGG |
| | | GAAGCUUGACUGCUUAGUUUGUUGUGGCAGGGGACUGUGUUCACACUUUUC |
| | | C |
| 123 | U1_12_4 | UUGGCAGGGGAGAUAGCAUGAUCACGAAGGUGGUUUUCCCAAGGCAAGAUU |
| | | UAUUCACUGCACUCUGGAUGUGCUGACCCCUACGAUUUCCGCCCAAUGGGG |
| | | AAGCUUGACUGCUUAGUUUGUUGUGGCAGGGGACUGUGUUCACACUUUUCC |
| | | C |
| 124 | U1_12_5 | UGGCAGGGGAGAUAGCAUGAUCACGAAGGUGGUUUUCCCAAGGCAAGAUUU |
| | | AUUCACUGCACUCUGGAUGUGCUGACCCCUACGAUUUCCGCCCAAUGGGGA |
| | | AGCUUGACUGCUUAGUUUGUUGUGGCAGGGGACUGUGUUCACACUUUUCCC |
| | | G |
| 125 | U1_12_6 | GGCAGGGGAGAUAGCAUGAUCACGAAGGUGGUUUUCCCAAGGCAAGAUUUA |
| | | UUCACUGCACUCUGGAUGUGCUGACCCCUACGAUUUCCGCCCAAUGGGGAA |
| | | GCUUGACUGCUUAGUUUGUUGUGGCAGGGGACUGUGUUCACACUUUUCCCG |
| | | G |
| 126 | U1_12_7 | GCAGGGGAGAUAGCAUGAUCACGAAGGUGGUUUUCCCAAGGCAAGAUUUAU |
| | | UCACUGCACUCUGGAUGUGCUGACCCCUACGAUUUCCGCCCAAUGGGGAAG |
| | | CUUGACUGCUUAGUUUGUUGUGGCAGGGGACUGUGUUCACACUUUUCCCGG |
| 127 | U1_12_8 | CAGGGGAGAUAGCAUGAUCACGAAGGUGGUUUUCCCAAGGCAAGAUUUAUU |
| | | CACUGCACUCUGGAUGUGCUGACCCCUACGAUUUCCGCCCAAUGGGGAAGC |
| | | UUGACUGCUUAGUUUGUUGUGGCAGGGGACUGUGUUCACACUUUUCCCGG |
| 128 | U1_12_9 | AGGGGAGAUAGCAUGAUCACGAAGGUGGUUUUCCCAAGGCAAGAUUUAUUC |
| | | ACUGCACUCUGGAUGUGCUGACCCCUACGAUUUCCGCCCAAUGGGGAAGCU |
| | | UGACUGCUUAGUUUGUUGUGGCAGGGGACUGUGUUCACACUUUUCCCGG |
| 129 | U1_13_0 | AUACUUAACAUGGCAGAGGAGAUAUCAUAAUCACAAAGGUAGUUUUCCCAG |
| | | GGCAAGCCUUAUCCACUGCAUUCCAGAUGUGCUCACCUCUGUGGUUUCCCC |
| | | AAAUGUGGAAAACUGGACUGCAUAAUUUGUGGUAGCGGGGGACUGCAUUAA |
| | | U |
| 130 | U1_13_1 | UACUUAACAUGGCAGAGGAGAUAUCAUAAUCACAAAGGUAGUUUUCCCAGG |
| | | GCAAGCCUUAUCCACUGCAUUCCAGAUGUGCUCACCUCUGUGGUUUCCCCA |
| | | AAUGUGGAAAACUGGACUGCAUAAUUUGUGGUAGCGGGGGACUGCAUUAAU |
| | | A |
| 131 | U1_13_2 | ACUUAACAUGGCAGAGGAGAUAUCAUAAUCACAAAGGUAGUUUUCCCAGGG |
| | | CAAGCCUUAUCCACUGCAUUCCAGAUGUGCUCACCUCUGUGGUUUCCCCAA |
| | | AUGUGGAAAACUGGACUGCAUAAUUUGUGGUAGCGGGGGACUGCAUUAAUA |
| | | C |
| 132 | U1_13_3 | CUUAACAUGGCAGAGGAGAUAUCAUAAUCACAAAGGUAGUUUUCCCAGGGC |
| | | AAGCCUUAUCCACUGCAUUCCAGAUGUGCUCACCUCUGUGGUUUCCCCAAA |
| | | UGUGGAAAACUGGACUGCAUAAUUUGUGGUAGCGGGGGACUGCAUUAAUAC |
| | | C |
| 133 | U1_13_4 | UUAACAUGGCAGAGGAGAUAUCAUAAUCACAAAGGUAGUUUUCCCAGGGCA |
| | | AGCCUUAUCCACUGCAUUCCAGAUGUGCUCACCUCUGUGGUUUCCCCAAAU |
| | | GUGGAAAACUGGACUGCAUAAUUUGUGGUAGCGGGGGACUGCAUUAAUACC |
| | | U |
| 134 | U1_13_5 | UAACAUGGCAGAGGAGAUAUCAUAAUCACAAAGGUAGUUUUCCCAGGGCAA |
| | | GCCUUAUCCACUGCAUUCCAGAUGUGCUCACCUCUGUGGUUUCCCCAAAUG |
| | | UGGAAAACUGGACUGCAUAAUUUGUGGUAGCGGGGGACUGCAUUAAUACCU |
| | | U |
| 135 | U1_13_6 | AACAUGGCAGAGGAGAUAUCAUAAUCACAAAGGUAGUUUUCCCAGGGCAAG |
| | | CCUUAUCCACUGCAUUCCAGAUGUGCUCACCUCUGUGGUUUCCCCAAAUGU |
| | | GGAAAACUGGACUGCAUAAUUUGUGGUAGCGGGGGACUGCAUUAAUACCUU |
| | | C |
| 136 | U1_13_7 | ACAUGGCAGAGGAGAUAUCAUAAUCACAAAGGUAGUUUUCCCAGGGCAAGC |
| | | CUUAUCCACUGCAUUCCAGAUGUGCUCACCUCUGUGGUUUCCCCAAAUGUG |
| | | GAAAACUGGACUGCAUAAUUUGUGGUAGCGGGGGACUGCAUUAAUACCUUC |
| | | C |
| 137 | U1_13_8 | CAUGGCAGAGGAGAUAUCAUAAUCACAAAGGUAGUUUUCCCAGGGCAAGCC |
| | | UUAUCCACUGCAUUCCAGAUGUGCUCACCUCUGUGGUUUCCCCAAAUGUGG |
| | | AAAACUGGACUGCAUAAUUUGUGGUAGCGGGGGACUGCAUUAAUACCUUCC |
| | | U |
| 138 | U1_13_9 | AUGGCAGAGGAGAUAUCAUAAUCACAAAGGUAGUUUUCCCAGGGCAAGCCU |
| | | UAUCCACUGCAUUCCAGAUGUGCUCACCUCUGUGGUUUCCCCAAAUGUGGA |
| | | AAACUGGACUGCAUAAUUUGUGGUAGCGGGGGACUGCAUUAAUACCUUCCU |
| | | C |
| 139 | U11_1_0 | AAAAAGGGCUUCUGCUGUGAGUGGCACACAUAGGGCAUCGUUUGCUCUUGG |
| | | UGCCAGAAUCAACAUCAAGAGAUUUCAGAAGCAUAAUUUUUUGGUACUUGG |
| | | GCAGCUGGUGAUCAUUGGUCCUGUAGCCCUU |
| 140 | U11_1_1 | AAAAGGGCUUCUGCUGUGAGUGGCACACAUAGGGCAUCGUUUGCUCUUGGU |
| | | GCCAGAAUCAACAUCAAGAGAUUUCAGAAGCAUAAUUUUUUGGUACUUGGG |
| | | CAGCUGGUGAUCAUUGGUCCUGUAGCCCUU |
| 141 | U11_1_2 | AAAGGGCUUCUGCUGUGAGUGGCACACAUAGGGCAUCGUUUGCUCUUGGUG |
| | | CCAGAAUCAACAUCAAGAGAUUUCAGAAGCAUAAUUUUUUGGUACUUGGGC |
| | | AGCUGGUGAUCAUUGGUCCUGUAGCCCUU |
| 142 | U11_1_3 | AAGGGCUUCUGCUGUGAGUGGCACACAUAGGGCAUCGUUUGCUCUUGGUGC |
| | | CAGAAUCAACAUCAAGAGAUUUCAGAAGCAUAAUUUUUUGGUACUUGGGCA |
| | | GCUGGUGAUCAUUGGUCCUGUAGCCCUU |
| 143 | U11_1_4 | AGGGCUUCUGCUGUGAGUGGCACACAUAGGGCAUCGUUUGCUCUUGGUGCC |
| | | AGAAUCAACAUCAAGAGAUUUCAGAAGCAUAAUUUUUUGGUACUUGGGCAG |
| | | CUGGUGAUCAUUGGUCCUGUAGCCCUU |
| 144 | U11_1_5 | GGGCUUCUGCUGUGAGUGGCACACAUAGGGCAUCGUUUGCUCUUGGUGCCA |
| | | GAAUCAACAUCAAGAGAUUUCAGAAGCAUAAUUUUUUGGUACUUGGGCAGC |
| | | UGGUGAUCAUUGGUCCUGUAGCCCUU |
| 145 | U11_1_6 | GGCUUCUGCUGUGAGUGGCACACAUAGGGCAUCGUUUGCUCUUGGUGCCAG |
| | | AAUCAACAUCAAGAGAUUUCAGAAGCAUAAUUUUUUGGUACUUGGGCAGCU |
| | | GGUGAUCAUUGGUCCUGUAGCCCUU |
| 146 | U11_1_7 | GCUUCUGCUGUGAGUGGCACACAUAGGGCAUCGUUUGCUCUUGGUGCCAGA |
| | | AUCAACAUCAAGAGAUUUCAGAAGCAUAAUUUUUUGGUACUUGGGCAGCUG |
| | | GUGAUCAUUGGUCCUGUAGCCCUU |
| 147 | U11_1_8 | CUUCUGCUGUGAGUGGCACACAUAGGGCAUCGUUUGCUCUUGGUGCCAGAA |
| | | UCAACAUCAAGAGAUUUCAGAAGCAUAAUUUUUUGGUACUUGGGCAGCUGG |
| | | UGAUCAUUGGUCCUGUAGCCCUU |
| 148 | U11_1_9 | UUCUGCUGUGAGUGGCACACAUAGGGCAUCGUUUGCUCUUGGUGCCAGAAU |
| | | CAACAUCAAGAGAUUUCAGAAGCAUAAUUUUUUGGUACUUGGGCAGCUGGU |
| | | GAUCAUUGGUCCUGUAGCCCUU |
| 149 | U11_2_0 | AAAAAAAGCUGCUGUUGUGAGUGAUACAUGCAGGGCAACUUGAUUGCUCUU |
| | | AGUGCAGAAUUGACAUCAAGGAAUUUUGGAAGUAUAAUUUUUUGGCAGGUG |
| | | GAUAGCUGGUUGUAUUAGUCCAUUCUC |
| 150 | U11_2_1 | AAAAAAGCUGCUGUUGUGAGUGAUACAUGCAGGGCAACUUGAUUGCUCUUA |
| | | GUGCAGAAUUGACAUCAAGGAAUUUUGGAAGUAUAAUUUUUUGGCAGGUGG |
| | | AUAGCUGGUUGUAUUAGUCCAUUCUC |
| 151 | U11_2_2 | AAAAAGCUGCUGUUGUGAGUGAUACAUGCAGGGCAACUUGAUUGCUCUUAG |
| | | UGCAGAAUUGACAUCAAGGAAUUUUGGAAGUAUAAUUUUUUGGCAGGUGGA |
| | | UAGCUGGUUGUAUUAGUCCAUUCUC |
| 152 | U11_2_3 | AAAAGCUGCUGUUGUGAGUGAUACAUGCAGGGCAACUUGAUUGCUCUUAGU |
| | | GCAGAAUUGACAUCAAGGAAUUUUGGAAGUAUAAUUUUUUGGCAGGUGGAU |
| | | AGCUGGUUGUAUUAGUCCAUUCUC |
| 153 | U11_2_4 | AAAGCUGCUGUUGUGAGUGAUACAUGCAGGGCAACUUGAUUGCUCUUAGUG |
| | | CAGAAUUGACAUCAAGGAAUUUUGGAAGUAUAAUUUUUUGGCAGGUGGAUA |
| | | GCUGGUUGUAUUAGUCCAUUCUC |
| 154 | U11_2_5 | AAGCUGCUGUUGUGAGUGAUACAUGCAGGGCAACUUGAUUGCUCUUAGUGC |
| | | AGAAUUGACAUCAAGGAAUUUUGGAAGUAUAAUUUUUUGGCAGGUGGAUAG |
| | | CUGGUUGUAUUAGUCCAUUCUC |
| 155 | U11_2_6 | AGCUGCUGUUGUGAGUGAUACAUGCAGGGCAACUUGAUUGCUCUUAGUGCA |
| | | GAAUUGACAUCAAGGAAUUUUGGAAGUAUAAUUUUUUGGCAGGUGGAUAGC |
| | | UGGUUGUAUUAGUCCAUUCUC |
| 156 | U11_2_7 | GCUGCUGUUGUGAGUGAUACAUGCAGGGCAACUUGAUUGCUCUUAGUGCAG |
| | | AAUUGACAUCAAGGAAUUUUGGAAGUAUAAUUUUUUGGCAGGUGGAUAGCU |
| | | GGUUGUAUUAGUCCAUUCUC |
| 157 | U11_2_8 | CUGCUGUUGUGAGUGAUACAUGCAGGGCAACUUGAUUGCUCUUAGUGCAGA |
| | | AUUGACAUCAAGGAAUUUUGGAAGUAUAAUUUUUUGGCAGGUGGAUAGCUG |
| | | GUUGUAUUAGUCCAUUCUC |
| 158 | U11_2_9 | UGCUGUUGUGAGUGAUACAUGCAGGGCAACUUGAUUGCUCUUAGUGCAGAA |
| | | UUGACAUCAAGGAAUUUUGGAAGUAUAAUUUUUUGGCAGGUGGAUAGCUGG |
| | | UUGUAUUAGUCCAUUCUC |
| 159 | U11_3_0 | AAAAAGGGCUUCUGUCAUGAGUGGCACACAUAGGACAACUCAAUUUCUCUU |
| | | CAUGCAGAAUAAACAUCAAGAGAUUUUGGAAGCGUAAUUUUUGGUAGUUGG |
| | | GCAGCUGGUGAUCACUGGUGCCAGCACCCUU |
| 160 | U11_3_1 | AAAAGGGCUUCUGUCAUGAGUGGCACACAUAGGACAACUCAAUUUCUCUUC |
| | | AUGCAGAAUAAACAUCAAGAGAUUUUGGAAGCGUAAUUUUUGGUAGUUGGG |
| | | CAGCUGGUGAUCACUGGUGCCAGCACCCUU |
| 161 | U11_3_2 | AAAGGGCUUCUGUCAUGAGUGGCACACAUAGGACAACUCAAUUUCUCUUCA |
| | | UGCAGAAUAAACAUCAAGAGAUUUUGGAAGCGUAAUUUUUGGUAGUUGGGC |
| | | AGCUGGUGAUCACUGGUGCCAGCACCCUU |
| 162 | U11_3_3 | AAGGGCUUCUGUCAUGAGUGGCACACAUAGGACAACUCAAUUUCUCUUCAU |
| | | GCAGAAUAAACAUCAAGAGAUUUUGGAAGCGUAAUUUUUGGUAGUUGGGCA |
| | | GCUGGUGAUCACUGGUGCCAGCACCCUU |
| 163 | U11_3_4 | AGGGCUUCUGUCAUGAGUGGCACACAUAGGACAACUCAAUUUCUCUUCAUG |
| | | CAGAAUAAACAUCAAGAGAUUUUGGAAGCGUAAUUUUUGGUAGUUGGGCAG |
| | | CUGGUGAUCACUGGUGCCAGCACCCUU |
| 164 | U11_3_5 | GGGCUUCUGUCAUGAGUGGCACACAUAGGACAACUCAAUUUCUCUUCAUGC |
| | | AGAAUAAACAUCAAGAGAUUUUGGAAGCGUAAUUUUUGGUAGUUGGGCAGC |
| | | UGGUGAUCACUGGUGCCAGCACCCUU |
| 165 | U11_3_6 | GGCUUCUGUCAUGAGUGGCACACAUAGGACAACUCAAUUUCUCUUCAUGCA |
| | | GAAUAAACAUCAAGAGAUUUUGGAAGCGUAAUUUUUGGUAGUUGGGCAGCU |
| | | GGUGAUCACUGGUGCCAGCACCCUU |
| 166 | U11_3_7 | GCUUCUGUCAUGAGUGGCACACAUAGGACAACUCAAUUUCUCUUCAUGCAG |
| | | AAUAAACAUCAAGAGAUUUUGGAAGCGUAAUUUUUGGUAGUUGGGCAGCUG |
| | | GUGAUCACUGGUGCCAGCACCCUU |
| 167 | U11_3_8 | CUUCUGUCAUGAGUGGCACACAUAGGACAACUCAAUUUCUCUUCAUGCAGA |
| | | AUAAACAUCAAGAGAUUUUGGAAGCGUAAUUUUUGGUAGUUGGGCAGCUGG |
| | | UGAUCACUGGUGCCAGCACCCUU |
| 168 | U11_3_9 | UUCUGUCAUGAGUGGCACACAUAGGACAACUCAAUUUCUCUUCAUGCAGAA |
| | | UAAACAUCAAGAGAUUUUGGAAGCGUAAUUUUUGGUAGUUGGGCAGCUGGU |
| | | GAUCACUGGUGCCAGCACCCUU |
| 169 | U11_4_0 | AAAAAGGGCUUCUGUCAUGAGUGGCCUAUGUAGGGCAACCAGAUUGCUCUU |
| | | CAUGUGGAAUUGACAUCAAGAGCUUUCAGAAGUGUAUUUUUUGGAAGUUGG |
| | | GCAGCUGGUAAUCAUUGGUCUUGGCAUCCUU |
| 170 | U11_4_1 | AAAAGGGCUUCUGUCAUGAGUGGCCUAUGUAGGGCAACCAGAUUGCUCUUC |
| | | AUGUGGAAUUGACAUCAAGAGCUUUCAGAAGUGUAUUUUUUGGAAGUUGGG |
| | | CAGCUGGUAAUCAUUGGUCUUGGCAUCCUU |
| 171 | U11_4_2 | AAAGGGCUUCUGUCAUGAGUGGCCUAUGUAGGGCAACCAGAUUGCUCUUCA |
| | | UGUGGAAUUGACAUCAAGAGCUUUCAGAAGUGUAUUUUUUGGAAGUUGGGC |
| | | AGCUGGUAAUCAUUGGUCUUGGCAUCCUU |
| 172 | U11_4_3 | AAGGGCUUCUGUCAUGAGUGGCCUAUGUAGGGCAACCAGAUUGCUCUUCAU |
| | | GUGGAAUUGACAUCAAGAGCUUUCAGAAGUGUAUUUUUUGGAAGUUGGGCA |
| | | GCUGGUAAUCAUUGGUCUUGGCAUCCUU |
| 173 | U11_4_4 | AGGGCUUCUGUCAUGAGUGGCCUAUGUAGGGCAACCAGAUUGCUCUUCAUG |
| | | UGGAAUUGACAUCAAGAGCUUUCAGAAGUGUAUUUUUUGGAAGUUGGGCAG |
| | | CUGGUAAUCAUUGGUCUUGGCAUCCUU |
| 174 | U11_4_5 | GGGCUUCUGUCAUGAGUGGCCUAUGUAGGGCAACCAGAUUGCUCUUCAUGU |
| | | GGAAUUGACAUCAAGAGCUUUCAGAAGUGUAUUUUUUGGAAGUUGGGCAGC |
| | | UGGUAAUCAUUGGUCUUGGCAUCCUU |
| 175 | U11_4_6 | GGCUUCUGUCAUGAGUGGCCUAUGUAGGGCAACCAGAUUGCUCUUCAUGUG |
| | | GAAUUGACAUCAAGAGCUUUCAGAAGUGUAUUUUUUGGAAGUUGGGCAGCU |
| | | GGUAAUCAUUGGUCUUGGCAUCCUU |
| 176 | U11_4_7 | GCUUCUGUCAUGAGUGGCCUAUGUAGGGCAACCAGAUUGCUCUUCAUGUGG |
| | | AAUUGACAUCAAGAGCUUUCAGAAGUGUAUUUUUUGGAAGUUGGGCAGCUG |
| | | GUAAUCAUUGGUCUUGGCAUCCUU |
| 177 | U11_4_8 | CUUCUGUCAUGAGUGGCCUAUGUAGGGCAACCAGAUUGCUCUUCAUGUGGA |
| | | AUUGACAUCAAGAGCUUUCAGAAGUGUAUUUUUUGGAAGUUGGGCAGCUGG |
| | | UAAUCAUUGGUCUUGGCAUCCUU |
| 178 | U11_4_9 | UUCUGUCAUGAGUGGCCUAUGUAGGGCAACCAGAUUGCUCUUCAUGUGGAA |
| | | UUGACAUCAAGAGCUUUCAGAAGUGUAUUUUUUGGAAGUUGGGCAGCUGGU |
| | | AAUCAUUGGUCUUGGCAUCCUU |
| 179 | U11_5_0 | AAAGGGGGCUUCUGUCAUGAGUGGCACACAUUGGGCAACUCAACUGCUCUU |
| | | CAUGAGGAAUCAACAUCAGGAGGUUUUGGAAGAAUGAUUUUUUUGGUAGUU |
| | | GGGCAGCUUGUGAGAAAAAAAUGUUUUCAGGCAAAUCCUC |
| 180 | U11_5_1 | AAGGGGGCUUCUGUCAUGAGUGGCACACAUUGGGCAACUCAACUGCUCUUC |
| | | AUGAGGAAUCAACAUCAGGAGGUUUUGGAAGAAUGAUUUUUUUGGUAGUUG |
| | | GGCAGCUUGUGAGAAAAAAAUGUUUUCAGGCAAAUCCUC |
| 181 | U11_5_2 | AGGGGGCUUCUGUCAUGAGUGGCACACAUUGGGCAACUCAACUGCUCUUCA |
| | | UGAGGAAUCAACAUCAGGAGGUUUUGGAAGAAUGAUUUUUUUGGUAGUUGG |
| | | GCAGCUUGUGAGAAAAAAAUGUUUUCAGGCAAAUCCUC |
| 182 | U11_5_3 | GGGGGCUUCUGUCAUGAGUGGCACACAUUGGGCAACUCAACUGCUCUUCAU |
| | | GAGGAAUCAACAUCAGGAGGUUUUGGAAGAAUGAUUUUUUUGGUAGUUGGG |
| | | CAGCUUGUGAGAAAAAAAUGUUUUCAGGCAAAUCCUC |
| 183 | U11_5_4 | GGGGCUUCUGUCAUGAGUGGCACACAUUGGGCAACUCAACUGCUCUUCAUG |
| | | AGGAAUCAACAUCAGGAGGUUUUGGAAGAAUGAUUUUUUUGGUAGUUGGGC |
| | | AGCUUGUGAGAAAAAAAUGUUUUCAGGCAAAUCCUC |
| 184 | U11_5_5 | GGGCUUCUGUCAUGAGUGGCACACAUUGGGCAACUCAACUGCUCUUCAUGA |
| | | GGAAUCAACAUCAGGAGGUUUUGGAAGAAUGAUUUUUUUGGUAGUUGGGCA |
| | | GCUUGUGAGAAAAAAAUGUUUUCAGGCAAAUCCUC |
| 185 | U11_5_6 | GGCUUCUGUCAUGAGUGGCACACAUUGGGCAACUCAACUGCUCUUCAUGAG |
| | | GAAUCAACAUCAGGAGGUUUUGGAAGAAUGAUUUUUUUGGUAGUUGGGCAG |
| | | CUUGUGAGAAAAAAAUGUUUUCAGGCAAAUCCUC |
| 186 | U11_5_7 | GCUUCUGUCAUGAGUGGCACACAUUGGGCAACUCAACUGCUCUUCAUGAGG |
| | | AAUCAACAUCAGGAGGUUUUGGAAGAAUGAUUUUUUUGGUAGUUGGGCAGC |
| | | UUGUGAGAAAAAAAUGUUUUCAGGCAAAUCCUC |
| 187 | U11_5_8 | CUUCUGUCAUGAGUGGCACACAUUGGGCAACUCAACUGCUCUUCAUGAGGA |
| | | AUCAACAUCAGGAGGUUUUGGAAGAAUGAUUUUUUUGGUAGUUGGGCAGCU |
| | | UGUGAGAAAAAAAUGUUUUCAGGCAAAUCCUC |
| 188 | U11_5_9 | UUCUGUCAUGAGUGGCACACAUUGGGCAACUCAACUGCUCUUCAUGAGGAA |
| | | UCAACAUCAGGAGGUUUUGGAAGAAUGAUUUUUUUGGUAGUUGGGCAGCUU |
| | | GUGAGAAAAAAAUGUUUUCAGGCAAAUCCUC |
| 189 | U11_6_0 | AAAAAGGGCUUCUGUCGUGAGUGGCACACGUAGGGCAACUCGAUUGCUCUG |
| | | CGUGCGGAAUCGACAUCAAGAGAUUUCGGAAGCAUAAUUUUUUGGUAUUUG |
| | | GGCAGCUGGUGAUCGUUGGUCCCGGCGCCCUU |
| 190 | U11_6_1 | AAAAGGGCUUCUGUCGUGAGUGGCACACGUAGGGCAACUCGAUUGCUCUGC |
| | | GUGCGGAAUCGACAUCAAGAGAUUUCGGAAGCAUAAUUUUUUGGUAUUUGG |
| | | GCAGCUGGUGAUCGUUGGUCCCGGCGCCCUU |
| 191 | U11_6_2 | AAAGGGCUUCUGUCGUGAGUGGCACACGUAGGGCAACUCGAUUGCUCUGCG |
| | | UGCGGAAUCGACAUCAAGAGAUUUCGGAAGCAUAAUUUUUUGGUAUUUGGG |
| | | CAGCUGGUGAUCGUUGGUCCCGGCGCCCUU |
| 192 | U11_6_3 | AAGGGCUUCUGUCGUGAGUGGCACACGUAGGGCAACUCGAUUGCUCUGCGU |
| | | GCGGAAUCGACAUCAAGAGAUUUCGGAAGCAUAAUUUUUUGGUAUUUGGGC |
| | | AGCUGGUGAUCGUUGGUCCCGGCGCCCUU |
| 193 | U11_6_4 | AGGGCUUCUGUCGUGAGUGGCACACGUAGGGCAACUCGAUUGCUCUGCGUG |
| | | CGGAAUCGACAUCAAGAGAUUUCGGAAGCAUAAUUUUUUGGUAUUUGGGCA |
| | | GCUGGUGAUCGUUGGUCCCGGCGCCCUU |
| 194 | U11_6_5 | GGGCUUCUGUCGUGAGUGGCACACGUAGGGCAACUCGAUUGCUCUGCGUGC |
| | | GGAAUCGACAUCAAGAGAUUUCGGAAGCAUAAUUUUUUGGUAUUUGGGCAG |
| | | CUGGUGAUCGUUGGUCCCGGCGCCCUU |
| 195 | U11_6_6 | GGCUUCUGUCGUGAGUGGCACACGUAGGGCAACUCGAUUGCUCUGCGUGCG |
| | | GAAUCGACAUCAAGAGAUUUCGGAAGCAUAAUUUUUUGGUAUUUGGGCAGC |
| | | UGGUGAUCGUUGGUCCCGGCGCCCUU |
| 196 | U11_6_7 | GCUUCUGUCGUGAGUGGCACACGUAGGGCAACUCGAUUGCUCUGCGUGCGG |
| | | AAUCGACAUCAAGAGAUUUCGGAAGCAUAAUUUUUUGGUAUUUGGGCAGCU |
| | | GGUGAUCGUUGGUCCCGGCGCCCUU |
| 197 | U11_6_8 | CUUCUGUCGUGAGUGGCACACGUAGGGCAACUCGAUUGCUCUGCGUGCGGA |
| | | AUCGACAUCAAGAGAUUUCGGAAGCAUAAUUUUUUGGUAUUUGGGCAGCUG |
| | | GUGAUCGUUGGUCCCGGCGCCCUU |
| 198 | U11_6_9 | UUCUGUCGUGAGUGGCACACGUAGGGCAACUCGAUUGCUCUGCGUGCGGAA |
| | | UCGACAUCAAGAGAUUUCGGAAGCAUAAUUUUUUGGUAUUUGGGCAGCUGG |
| | | UGAUCGUUGGUCCCGGCGCCCUU |
| 199 | U11_7_0 | AAAAAGGGCUUCUGUCGUGAGUGGCACACGUAGGGCAACUCGAUUGCUCUG |
| | | CGUGCGGAAUCGACAUCAAGAGAUUUCGGAAGCAUAAUUUUUUGGUAUUUG |
| | | GGCAGCUGGUGAUCGUUGGUCCCGGCGCC |
| 200 | U11_7_1 | AAAAGGGCUUCUGUCGUGAGUGGCACACGUAGGGCAACUCGAUUGCUCUGC |
| | | GUGCGGAAUCGACAUCAAGAGAUUUCGGAAGCAUAAUUUUUUGGUAUUUGG |
| | | GCAGCUGGUGAUCGUUGGUCCCGGCGCC |
| 201 | U11_7_2 | AAAGGGCUUCUGUCGUGAGUGGCACACGUAGGGCAACUCGAUUGCUCUGCG |
| | | UGCGGAAUCGACAUCAAGAGAUUUCGGAAGCAUAAUUUUUUGGUAUUUGGG |
| | | CAGCUGGUGAUCGUUGGUCCCGGCGCC |
| 202 | U11_7_3 | AAGGGCUUCUGUCGUGAGUGGCACACGUAGGGCAACUCGAUUGCUCUGCGU |
| | | GCGGAAUCGACAUCAAGAGAUUUCGGAAGCAUAAUUUUUUGGUAUUUGGGC |
| | | AGCUGGUGAUCGUUGGUCCCGGCGCC |
| 203 | U11_7_4 | AGGGCUUCUGUCGUGAGUGGCACACGUAGGGCAACUCGAUUGCUCUGCGUG |
| | | CGGAAUCGACAUCAAGAGAUUUCGGAAGCAUAAUUUUUUGGUAUUUGGGCA |
| | | GCUGGUGAUCGUUGGUCCCGGCGCC |
| 204 | U11_7_5 | GGGCUUCUGUCGUGAGUGGCACACGUAGGGCAACUCGAUUGCUCUGCGUGC |
| | | GGAAUCGACAUCAAGAGAUUUCGGAAGCAUAAUUUUUUGGUAUUUGGGCAG |
| | | CUGGUGAUCGUUGGUCCCGGCGCC |
| 205 | U11_7_6 | GGCUUCUGUCGUGAGUGGCACACGUAGGGCAACUCGAUUGCUCUGCGUGCG |
| | | GAAUCGACAUCAAGAGAUUUCGGAAGCAUAAUUUUUUGGUAUUUGGGCAGC |
| | | UGGUGAUCGUUGGUCCCGGCGCC |
| 206 | U11_7_7 | GCUUCUGUCGUGAGUGGCACACGUAGGGCAACUCGAUUGCUCUGCGUGCGG |
| | | AAUCGACAUCAAGAGAUUUCGGAAGCAUAAUUUUUUGGUAUUUGGGCAGCU |
| | | GGUGAUCGUUGGUCCCGGCGCC |
| 207 | U11_7_8 | CUUCUGUCGUGAGUGGCACACGUAGGGCAACUCGAUUGCUCUGCGUGCGGA |
| | | AUCGACAUCAAGAGAUUUCGGAAGCAUAAUUUUUUGGUAUUUGGGCAGCUG |
| | | GUGAUCGUUGGUCCCGGCGCC |
| 208 | U11_7_9 | UUCUGUCGUGAGUGGCACACGUAGGGCAACUCGAUUGCUCUGCGUGCGGAA |
| | | UCGACAUCAAGAGAUUUCGGAAGCAUAAUUUUUUGGUAUUUGGGCAGCUGG |
| | | UGAUCGUUGGUCCCGGCGCC |
| 209 | Sm_ | AAUUUUUGGAG |
| | consensus | |
| 210 | Sm_1 | AAUUUUUGGAG |
| 211 | Sm_2 | UAUUUUUGGAG |
| 212 | Sm_3 | GAUUUUUGGAG |
| 213 | Sm_4 | CAUUUUUGGAG |
| 214 | Sm_5 | AUUUUUUGGAG |
| 215 | Sm_6 | AGUUUUUGGAG |
| 216 | Sm_7 | ACUUUUUGGAG |
| 217 | Sm_8 | AAAUUUUGGAG |
| 218 | Sm_9 | AAGUUUUGGAG |
| 219 | Sm_10 | AACUUUUGGAG |
| 220 | Sm_11 | AAUAUUUGGAG |
| 221 | Sm_12 | AAUGUUUGGAG |
| 222 | Sm_13 | AAUCUUUGGAG |
| 223 | Sm_14 | AAUUAUUGGAG |
| 224 | Sm_15 | AAUUGUUGGAG |
| 225 | Sm_16 | AAUUCUUGGAG |
| 226 | Sm_17 | AAUUUAUGGAG |
| 227 | Sm_18 | AAUUUGUGGAG |
| 228 | Sm_19 | AAUUUCUGGAG |
| 229 | Sm_20 | AAUUUUAGGAG |
| 230 | Sm_21 | AAUUUUGGGAG |
| 231 | Sm_22 | AAUUUUCGGAG |
| 232 | Sm_23 | AAUUUUUAGAG |
| 233 | Sm_24 | AAUUUUUUGAG |
| 234 | Sm_25 | AAUUUUUCGAG |
| 235 | Sm_26 | AAUUUUUGAAG |
| 236 | Sm_27 | AAUUUUUGUAG |
| 237 | Sm_28 | AAUUUUUGCAG |
| 238 | Sm_29 | AAUUUUUGGUG |
| 239 | Sm_30 | AAUUUUUGGGG |
| 240 | Sm_31 | AAUUUUUGGCG |
| 241 | Sm_32 | AAUUUUUGGAA |
| 242 | Sm_33 | AAUUUUUGGAU |
| 243 | Sm_34 | AAUUUUUGGAC |
| 244 | Sm_35 | AAUGUUUUGAG |
| 245 | Sm_36 | AAUUUGUGUAG |
| 246 | Sm_37 | AGUUUUUGGAA |
| 247 | Sm_38 | AUUUUGUGGAG |
| 248 | Sm_39 | AAUGUUUGCAG |
| 249 | Sm_40 | AAUGUUGGGAG |
| 250 | Sm_41 | AAUUGCUGGAG |
| 251 | Sm_42 | AAUUUUAGCAG |
| 252 | Sm_43 | AAGGUUUGGAG |
| 253 | Sm_44 | GAUUUGUGGAG |
| 254 | Sm_45 | ACUUUUUGGAA |
| 255 | Sm_46 | AAAUUAUGGAG |
| 256 | Sm_47 | AAUUUUGCGAG |
| 257 | Sm_48 | AAUUUCUGGAA |
| 258 | Sm_49 | UAUUUUUGGUG |
| 259 | Sm_50 | GAUUUUAGGAG |
| 260 | Sm_51 | AAUCUGUGGAG |
| 261 | Sm_52 | AAAUUUUAGAG |
| 262 | Sm_53 | AAUUUUUCGAC |
| 263 | Sm_54 | AAUUUUUGGGU |
| 264 | Sm_55 | AAUCUUUGGAA |
| 265 | Sm_56 | AAUUAUUGGUG |
| 266 | Sm_57 | AAUUUUAAGAG |
| 267 | Sm_58 | AAUGUUAGGAG |
| 268 | Sm_59 | AAUUUUAGAAG |
| 269 | Sm_60 | AUUUUUUGGCG |
| 270 | Sm_61 | AACUUUUGGUG |
| 271 | Sm_62 | AAUGUUUGGAU |
| 272 | Sm_63 | AAUUUUAGGAU |
| 273 | Sm_64 | AAUUUUUGAUG |
| 274 | Sm_65 | AAUUCUUGGUG |
| 275 | Sm_66 | AAUCCUUGGAG |
| 276 | Sm_67 | AAUUUUUCAAG |
| 277 | Sm_68 | CAUUUUUGCAG |
| 278 | Sm_69 | CAUUUUUCGAG |
| 279 | Sm_70 | AAUUUUGGGCG |
| 280 | Sm_71 | AGUUUUUGCAG |
| 281 | Sm_72 | AAAAUUUGGAG |
| 282 | Sm_73 | AACUCUUGGAG |
| 283 | Sm_74 | AAUUUGUGGAC |
| 284 | Sm_75 | ACUUUUUGGUG |
| 285 | Sm_76 | AUUUUUUGAAG |
| 286 | Sm_77 | AUUUUUUGGAA |
| 287 | Sm_78 | AAGUUAUGGAG |
| 288 | Sm_79 | CAUUUCUGGAG |
| 289 | Sm_80 | AGUUCUUGGAG |
| 290 | Sm_81 | AAUUUUGGAAG |
| 291 | Sm_82 | AGUAUUUGGAG |
| 292 | Sm_83 | AAUUUUUGGGC |
| 293 | Sm_84 | AAUUCUUGUAG |
| 294 | Sm_85 | AAUUUUGGGAA |
| 295 | Sm_86 | UAUUUUUUGAG |
| 296 | Sm_87 | AACUUUUGUAG |
| 297 | Sm_88 | AAUUAUUGCAG |
| 298 | Sm_89 | AAUUUUUGGUA |
| 299 | Sm_90 | AAUUUUCGAAG |
| 300 | Sm_91 | UAUCUUUGGAG |
| 301 | Sm_92 | AAUUUUUCGUG |
| 302 | Sm_93 | AAUCUUUGGCG |
| 303 | Sm_94 | GAUUUAUGGAG |
| 304 | Sm_95 | AAUUUUUCGAA |
| 305 | Sm_96 | CCUUUUUGGAG |
| 306 | Sm_97 | UAUUUUUGAAG |
| 307 | Sm_98 | AAUUUUUGUAA |
| 308 | Sm_99 | AAUUUUGAGAG |
| 309 | Sm_100 | AAGUAUUGGAG |
| 310 | Sm_101 | AAUUUUAGGAC |
| 311 | Sm_102 | AAUUUUUAAAG |
| 312 | Sm_103 | AAUUCUUGGAU |
| 313 | Sm_104 | AAUUUGUGGUG |
| 314 | Sm_105 | AAUUUUCGGGG |
| 315 | Sm_106 | ACUUUUUUGAG |
| 316 | Sm_107 | AAGUUUCGGAG |
| 317 | Sm_108 | ACUUUUGGGAG |
| 318 | Sm_109 | GACUUUUGGAG |
| 319 | Sm_110 | CUUUUUUGGAG |
| 320 | Sm_111 | CAUUGUUGGAG |
| 321 | Sm_112 | AAUAUUUUGAG |
| 322 | Sm_113 | AAGUUUUGGCG |
| 323 | Sm_114 | AAUUUUAGGAA |
| 324 | Sm_115 | AACUUGUGGAG |
| 325 | Sm_116 | AUUUUUUGGGG |
| 326 | Sm_117 | AAUUUUUAGAU |
| 327 | Sm_118 | UAUUUUGGGAG |
| 328 | Sm_119 | AAUUUGUGGGG |
| 329 | Sm_120 | AAUUUUUAGGG |
| 330 | Sm_121 | AAUUUUCGUAG |
| 331 | Sm_122 | AAUUUUCUGAG |
| 332 | Sm_123 | GAUUCUUGGAG |
| 333 | Sm_124 | AAUCUUUGGAU |
| 334 | Sm_125 | AAAUUUUGGCG |
| 335 | Sm_126 | AAUCAUUGGAG |
| 336 | Sm_127 | AACUUUUGGCG |
| 337 | Sm_128 | AAUUUUUGGGA |
| 338 | Sm_129 | AAUGUUUGGUG |
| 339 | Sm_130 | AAUUUAUGCAG |
| 340 | Sm_131 | AAUCUCUGGAG |
| 341 | Sm_132 | AGUUUGUGGAG |
| 342 | Sm_133 | AAGUUUUGGAU |
| 343 | Sm_134 | UAUUUUUGGCG |
| 344 | Sm_135 | AAUUUCUGAAG |
| 345 | Sm_136 | UAUUUCUGGAG |
| 346 | Sm_137 | AAUUUUUGCUG |
| 347 | Sm_138 | AAAUCUUGGAG |
| 348 | Sm_139 | AAAUUUUUGAG |
| 349 | Sm_140 | UAUUUUUGUAG |
| 350 | Sm_141 | GAUUAUUGGAG |
| 351 | Sm_142 | AAGUUCUGGAG |
| 352 | Sm_143 | AAUCUUUGAAG |
| 353 | Sm_144 | AAAUAUUGGAG |
| 354 | Sm_145 | AACUAUUGGAG |
| 355 | Sm_146 | AAUAUAUGGAG |
| 356 | Sm_147 | AUUCUUUGGAG |
| 357 | Sm_148 | GAUUUUUGAAG |
| 358 | Sm_149 | AAGUUUUGGUG |
| 359 | Sm_150 | AAUCUUUGCAG |
| 360 | Sm_151 | ACUUUUUGGCG |
| 361 | Sm_152 | GAUUUUGGGAG |
| 362 | Sm_153 | CAUUUUUGGAA |
| 363 | Sm_154 | AAUUUUUUGAC |
| 364 | Sm_155 | AAAUUUUGCAG |
| 365 | Sm_156 | ACUUUUUGAAG |
| 366 | Sm_157 | AGUCUUUGGAG |
| 367 | Sm_158 | AGUUUAUGGAG |
| 368 | Sm_159 | ACUUCUUGGAG |
| 369 | Sm_160 | AAUUUUUUCAG |
| 370 | Sm_161 | AAUUUUCGGAU |
| 371 | Sm_162 | AAUUUUUGUAU |
| 372 | Sm_163 | AAUGUCUGGAG |
| 373 | Sm_164 | AAUUGUUGGGG |
| 374 | Sm_165 | AAUUUAUGGAC |
| 375 | Sm_166 | AAUUUAUGAAG |
| 376 | Sm_167 | AAUUUAUGGGG |
| 377 | Sm_168 | AAUUUGUCGAG |
| 378 | Sm_169 | AAUUGUUGGAA |
| 379 | Sm_170 | UGUUUUUGGAG |
| 380 | Sm_171 | AAAUUUAGGAG |
| 381 | Sm_172 | AAUUUUUUGUG |
| 382 | Sm_173 | CAUUCUUGGAG |
| 383 | Sm_174 | AAUUCUCGGAG |
| 384 | Sm_175 | AAUUUUUAUAG |
| 385 | Sm_176 | ACUUUUUAGAG |
| 386 | Sm_177 | AAUUGUUAGAG |
| 387 | Sm_178 | CGUUUUUGGAG |
| 388 | Sm_179 | AAUUUUUGCGG |
| 389 | Sm_181 | AAGUUUUGAAG |
| 390 | Sm_181 | AAUUUUCGGAA |
| 391 | Sm_182 | AAUUUUUCCAG |
| 392 | Sm_183 | AUUUUUUGUAG |
| 393 | Sm_184 | AAUCUUUAGAG |
| 394 | Sm_185 | AAUUUUACGAG |
| 395 | Sm_186 | CAUUUUUGGCG |
| 396 | Sm_187 | AAUUUGCGGAG |
| 397 | Sm_188 | CAUCUUUGGAG |
| 398 | Sm_189 | AACUUUUGAAG |
| 399 | Sm_190 | AAUUUUUCUAG |
| 400 | U7_1 | CAGGUUUUCCAGUGUUCACUGAAAUUUGUCUCU |
| 401 | U7_2 | AAGGUUUUCUGGUCUUUAUCAGAAAGCCUCC |
| 402 | U7_3 | CAGGUUUUCUGGUUUUCACUGCAAAACCCCA |
| 403 | U7_4 | CAGACUUUACGGUGCUUACCAGAAAGCUCCC |
| 404 | U7_5 | CACGUUUUCCGGUUGUCACUCCCAGGUAGGCUGGGGAAGAGGCAU |
| 405 | U7_6 | CAGUCUUUCCAGUUUUUGCCGGAAAGCCCCU |
| 406 | U7_7 | CAGGCUUUCUGGUUUUUGCCAGAAAGCCCCC |
| 407 | U7_8 | CAGGUUUUUCAGUUUUCACCAGAAUGCCCAC |
| 408 | U7_9 | CAGGUUUUCUGGUCUUUACUGGAAAGCCCAA |
| 409 | U7_10 | CAGGCUUUCCGGUUUUUACCAGAAAGCCCCC |
| 410 | U7_11 | CAGGUUUUUCGGUUUUUACUGGAAAGCCCCA |
| 411 | U7_12 | CAGGCUUUCCAGUAAAUACAGGAAAGCCCUC |
| 412 | U7_13 | CAGGCUUUCCGGUUUUUGCUGGAAAGACUCC |
| 413 | U7_14 | AAGGUUUUCUGGUCUUUACUGAAAAGCCCCA |
| 414 | U7_15 | UGGGCUUUCUGUUUUUUACGAGAAAGUCCUC |
| 415 | U7_16 | CAGAUUUCUCAGUUUUCACUGGAAACCCUU |
| 416 | U7_17 | CAGGUUUUCCUGUCUUCACCGGAACUCCCCA |
| 417 | U7_18 | CAGGUUUUCUGGUUUGCACUAGAAAAACCAUA |
| 418 | U7_19 | CAGAUUUUCUGGUUUUUACCAGAAAAUUUAA |
| 419 | U7_20 | CAGGUCUUCUUGUUUUUACUGGAAAAUCCUC |
| 420 | U7_21 | CAGGUUUUCCGGUCUUCACCAGAAAACCCCU |
| 421 | U7_22 | CAGGUUUUCCGAUUUUUACUGGAAAGCCCUU |
| 422 | U7_23 | CAGGUUUUCUGGUUUUCCAGAAAACCUCC |
| 423 | U7_24 | CAGGUUUUCCAGUUUUCACUGGAACCUCU |
| 424 | U7_25 | CCGGCUUUCCAGUUUUGCCGGAAAGCCCCC |
| 425 | U7_26 | CAGGUUUUCUGGUUUUUACCAGAACUUAA |
| 426 | U7_27 | CACGUUUUCCGGUUGUCACUCCCAGGUGGGCUCGGGAAGAGGCAU |
| 427 | U7_28 | CAGGCUUUCCGGUCUUUACCGGAAAGCCUAU |
| 428 | U7_29 | CAGGCUUUCUGGUUUUUGCCGGAAAGCCCUC |
| 429 | U7_30 | CAGGCUUUCAGGUCUUUGCCAGAAAGCCCCA |
| 430 | U7_31 | CAGGCUUUCUUGUUUUCACUGGAAGCCUCC |
| 431 | U7_32 | CAGGUUUUUCAGUCUUCACCGGAAAGCUCCC |
| 432 | U7_33 | CAGAUUUUCUGGUUUUCACCACGAAAGAAAUGUCAUU |
| 433 | U7_34 | CAGUUCUCCUGGAUUUUACAGGAAAACCCC |
| 434 | U7_35 | CAGGCUUUCUGGCAUUUGCCAGAAAGCCCUG |
| 435 | U7_36 | CAGACUUUCCUGUUUUUAACAGAAAGCCCCC |
| 436 | U7_37 | CAGGCUUUCCGGUUUUUGCUGGAAAGCUCCC |
| 437 | U7_38 | CAGCUUUUCCAGUUUUCACUGAGGUAAA |
| 438 | U7_39 | UAGGUUUUCUGGUUUUUAUUGGGAAAUCACA |
| 439 | U7_40 | CAGGGUUUCUGGUGGUACCAGCAGACUCCACA |
| 440 | U7_41 | CAAGUUUCCCAGUUUUCACUGGAACCUCCG |
| 441 | U7_42 | CAGAUUUUCCAGUUUUUACUAGAAACCCCCC |
| 442 | U7_43 | CAGGGUUUCCAGUUUUCACUGUAUACCAUCC |
| 443 | U7_44 | CAAGUUCUCUGGUCUUCACUGGAAAACCCAU |
| 444 | U7_45 | UAGAUUUUCUGGUCUUUACCAGAAAGACUCC |
| 445 | U7_46 | GCAGGUUUUCUGGUUUUUAUUGGAAAACCUUC |
| 446 | U7_47 | CAGGUUUUCCACUUUUCACCAGAAACUGCCUCU |
| 447 | U7_48 | UAGGCUUUCCGGUUUUUGCAGGAAAGCCCCC |
| 448 | U7_49 | CAGGUUUUCCAGUCUUUACCAGAAAGCCACU |
| 449 | U7_50 | CAGGUUUUUUGGUCUUCACAGGAAACUUCCCCU |
| 450 | U7_51 | CAGGUUUUCUGCUUUUCACUGGAAGACUCCC |
| 451 | U7_52 | CAGACUUUCCAGUUUUUGCCAGAAAGCCCAC |
| 452 | U7_53 | CAGGCUUUCUGGUUUUUGCCCAAAAGCCUCU |
| 453 | U7_54 | CAGGUUUUCCAAUCUUCACUGAAAAGCUUUA |
| 454 | U7_55 | CUGGUUCCUAGUCUUCACUGGAAGCACCCUC |
| 455 | U7_56 | GGCUUUCCGCUCUUCAUUGGAAAGCCCAU |
| 456 | U7_57 | CAGGUUUUUCAGUUUUUAGCAGAAAACCUCC |
| 457 | U7_58 | CAGGUUCUCUGGUUUUUACUGAAACCAAA |
| 458 | U7_59 | CAGGUUUUCCAGUCUUCACUGGAAAGCCCUU |
| 459 | U7_60 | CAGGCUUUCUGGUUUUUGCCGGAAGGCCUCC |
| 460 | U7_61 | CAGGUUUUCUGUUUUUUAACUAGAAAACUCCC |
| 461 | U7_62 | UAGGCUUUCUGGUUUUUGCUGGAAAGCCCCC |
| 462 | U7_63 | CAGGUUUCCCGGUUUUUACCAGAAAAAUCUAA |
| 463 | U7_64 | CAGGUUUUCCAGUCUUUACCAGAAAUGCCCCC |
| 464 | U7_65 | GGUUUUCUGGUCAUCGCUGGAAACACCCUC |
| 465 | U7_66 | CAACCUUUCUGGUUUUUGCUAGAAAGUUCUC |
| 466 | U7_67 | CAGGCCUUUGGGUCUUUACUGGAAAACCCCU |
| 467 | U7_68 | UAUGUUUUCCGGUUUUCACUCCCAGGUAGGCUCGG |
| 468 | U7_69 | CAAAUUUUCUGGCUUUUCCAGGAAAAUCCCC |
| 469 | U7_70 | UAGGUUUUCCAGUGACCAGAAAAUCCUC |
| 470 | U7_71 | CAGGUCUUCGGGUUUUUAACUGGAAACCUUC |
| 471 | U7_72 | GCAGGCUUUCCAGUUUUUUCUGGAAAGCCUCA |
| 472 | U7_73 | GGCUUUCUGGUUUUUACUGGAAAGCCCCC |
| 473 | U7_74 | CAGGUUUCCCAGUAUUCACCGGAAAGCUCCA |
| 474 | U7_75 | CAGGCUUUCUAGUUUUCCCACACUAAGAAACAAAAAAACCUGU |
| 475 | U7_76 | GCAAGCUUUCUGGUUUUGGCCAAAAAGCCACC |
| 476 | U7_77 | UUCUGGUUUUCACCAGAAACCACU |
| 477 | U7_78 | GCAAGUUUUCUAGCCUGUACCUGAAAGCCUCA |
| 478 | U7_79 | AAGUUUUCUUGUUUUUACCAGAAAACUCCU |
| 479 | U7_80 | GGCUUUCCAAUUUUUGCCAGAAAGCCCCC |
| 480 | U7_81 | AGCUGGUUUUCUGGUUUUCACCGGAAGACCCAU |
| 481 | U7_82 | CAGGCCUUCUGGUUUUUGCUGGGAAGUCCCCA |
| 482 | U7_83 | CAGGUUUUCCAGUUUGUACCAGAAAACCCCU |
| 483 | U7_84 | CAGGUUUCACCCAAAAACCCAC |
| 484 | U7_85 | UAGGCUUUCUGGCUUUUUACCGGAAAGCCCCU |
| 485 | U7_86 | CAGGGUUUCUGGUUUUCACCAGGAAACAAAA |
| 486 | U7_87 | CAGGUUUUCCUGUCUUUACUAGAAACCCCUC |
| 487 | U7_88 | CAGGUUUUAUAGGUUUUACCAGAAAACUUCC |
| 488 | U7_89 | CAGGCUUCCCAAUUUUUGCUGGAAAGCCCCU |
| 489 | U7_90 | CAGGCUUUUUGGGUUAGGUCAGAAAGUCCCC |
| 490 | U7_91 | CAGGUUUUUGGUUUUUUAGCGAAACCCUC |
| 491 | U7_92 | CAGGCUUUCUGGUUUUCACCAUAAAAUGCCCCA |
| 492 | U7_93 | CAGGUCUUCGGGGUUUUACAAAAAGAAGAAGAAAUCCACCCCC |
| 493 | U7_94 | CAAGUUCUCUGGUCUCCCAACAAGAAACCCCC |
| 494 | U7_95 | CAGGAUUUCUGGUUUCUGGUGGAAAGCUCCCAU |
| 495 | U7_96 | AGGCUUUCCAGUUUUUGCUGGAAAGCCCCU |
| 496 | U7_97 | CAGGUUUUCCAGUCUUUGUCAAAUGCCUUC |
| 497 | U7_98 | CAGGCUUUCCAGUUUUUGCCAGUGUGAACCCAAA |
| 498 | U7_99 | CAGGCUUUCUGGUUUUUAUCAGAAAGCCUCC |
| 499 | U7_100 | GAGGUUUUCUGGUUUUCACCAGAACCAACCCUU |
| 500 | U7_101 | CAGAUUUUCCAGCUUUUACUGGAAGCCCCCU |
| 501 | U7_102 | CAGGUUUUCUGGUUUUCACUGGAAAAUACCUCA |
| 502 | U7_103 | CAUGUUUUUUGGUGUUAAUAAAAACCCGCACAC |
| 503 | U7_104 | AAGGCUUUCCGGUUUUUGCAGGAAAGCAACC |
| 504 | U7_105 | AGUCUUUCCGGGUUUUGUCAGAAAGGCCCU |
| 505 | U7_106 | GCAGGUUUUCUGGUUUCUACUGGAAGCUUCU |
| 506 | U7_107 | CAGGCUUUCUGGUUUUUACUGGAAAGCCCCU |
| 507 | U7_108 | CAGGCUUUCCAAUUUUUGCCAGAAAGCUCCC |
| 508 | U7_109 | CAGGUUUUCUGGUUCUCACCAGAAAACGCCA |
| 509 | U7_110 | CAGGUUUUCUGGUCUUCACUGGAAGACCACU |
| 510 | U7_111 | CAGGUGGUUUUCCGGUCUUUACCAGUAAGCCCCC |
| 511 | U7_112 | CAGGUUUUCCAGUUUCUACCAGAAACCCCUA |
| 512 | U7_113 | CAGGUUUUCUGGUCUUUACCAGAAAAGUCUCC |
| 513 | U7_114 | CCAGGCUUUCUGGUUUUUACCGGAAAGCCCCG |
| 514 | U7_115 | CAGGUUUUUCACUUUUUACUAGAAAAACCAGU |
| 515 | U7_116 | UAGGCUUUCUGGGAGGUCUUUACCAGAAAGCCUCC |
| 516 | U7_117 | UACAUUUUCCAGUUUUCACCAGGAAUCUGCCU |
| 517 | U7_118 | UAGGUUUUCCAGUUUUCACCAGAAAAUCCCG |
| 518 | U7_119 | CAGGUUUUCUGGUUUUUACGAGACUCCCCCA |
| 519 | U7_120 | CAGGCUUUCUGUUUUUUGCUAAAAUCCUCC |
| 520 | U7_121 | CUGGGCGUACUGGCUCACGCCUAUAAUCCCAA |
| 521 | U7_122 | GGCUCUCCGGUUUUUGCCAGAAUGCCCAC |
| 522 | U7_123 | UGGGUUUUCUGGUGAAAACUAGACUGCCCCC |
| 523 | U7_124 | CAGGCUUUCUGGUUUUUACAGGAAGGCCCCU |
| 524 | U7_125 | GGUUUUCUGGUUCUCACUGGAAAACCCCC |
| 525 | U7_126 | CAGGCUUUCCGGUAAAGACUGGAAAGCCCCU |
| 526 | U7_127 | CAGGUUUUCCAGUUUUUACCAGAAAACUCUC |
| 527 | U7_110_ | AAGGUUUUCUGGUCUUCACUGGAAGACCACU |
| | C1A | |
| 528 | U7_110_ | GAGGUUUUCUGGUCUUCACUGGAAGACCACU |
| | C1G | |
| 529 | U7_110_ | UAGGUUUUCUGGUCUUCACUGGAAGACCACU |
| | C1T | |
| 530 | U7_110_ | CCGGUUUUCUGGUCUUCACUGGAAGACCACU |
| | A2C | |
| 531 | U7_110_ | CGGGUUUUCUGGUCUUCACUGGAAGACCACU |
| | A2G | |
| 532 | U7_110_ | CUGGUUUUCUGGUCUUCACUGGAAGACCACU |
| | A2T | |
| 533 | U7_110_ | CAUGUUUUCUGGUCUUCACUGGAAGACAACU |
| | G3T_C28A | |
| 534 | U7_110_ | CACGUUUUCUGGUCUUCACUGGAAGACGACU |
| | G3C_C28G | |
| 535 | U7_110_ | CAAGUUUUCUGGUCUUCACUGGAAGACUACU |
| | G3A_C28T | |
| 536 | U7_110_ | CAGCUUUUCUGGUCUUCACUGGAAGAGCACU |
| | G4C_C27G | |
| 537 | U7_110_ | CAGAUUUUCUGGUCUUCACUGGAAGAUCACU |
| | G4A_C27T | |
| 538 | U7_110_ | CAGUUUUUCUGGUCUUCACUGGAAGAACACU |
| | G4T_C27A | |
| 539 | U7_110_ | CAGGGUUUCUGGUCUUCACUGGAAGCCCACU |
| | T5G_A26C | |
| 540 | U7_110_ | CAGGAUUUCUGGUCUUCACUGGAAGUCCACU |
| | T5A_A26T | |
| 541 | U7_110_ | CAGGCUUUCUGGUCUUCACUGGAAGGCCACU |
| | T5C_ | |
| | A26G | |
| 542 | U7_110_ | CAGGUGUUCUGGUCUUCACUGGAACACCACU |
| | T6G_G25C | |
| 543 | U7_110_ | CAGGUAUUCUGGUCUUCACUGGAAUACCACU |
| | T6A_G25T | |
| 544 | U7_110_ | CAGGUCUUCUGGUCUUCACUGGAAGACCACU |
| | T6C_G25G | |
| 545 | U7_110_ | CAGGUUUUCUGGUCUUCACUGGAAAACCACU |
| | T6T_G25A | |
| 546 | U7_110_ | CAGGUUCUCUGGUCUUCACUGGAGGACCACU |
| | T7C_A24G | |
| 547 | U7_110_ | CAGGUUGUCUGGUCUUCACUGGACGACCACU |
| | T7G_A24C | |
| 548 | U7_110_ | CAGGUUAUCUGGUCUUCACUGGAUGACCACU |
| | T7A_A24T | |
| 549 | U7_110_ | CAGGUUUACUGGUCUUCACUGGUAGACCACU |
| | T8A_A23T | |
| 550 | U7_110_ | CAGGUUUGCUGGUCUUCACUGGCAGACCACU |
| | T8G_A23C | |
| 551 | U7_110_ | CAGGUUUCCUGGUCUUCACUGGGAGACCACU |
| | T8C_A23G | |
| 552 | U7_11_ | CAGGUUUUUUGGUCUUCACUGAAAGACCACU |
| | 0C9T_G22A | |
| 553 | U7_110_ | CAGGUUUUGUGGUCUUCACUGCAAGACCACU |
| | C9G_G22C | |
| 554 | U7_110_ | CAGGUUUUAUGGUCUUCACUGUAAGACCACU |
| | C9A_G22T | |
| 555 | U7_110_ | CAGGUUUUCCGGUCUUCACUGGAAGACCACU |
| | T10C_G21G | |
| 556 | U7_110_ | CAGGUUUUCAGGUCUUCACUUGAAGACCACU |
| | T10A_G21T | |
| 557 | U7_110_ | CAGGUUUUCUGGUCUUCACUAGAAGACCACU |
| | T10T_G21A | |
| 558 | U7_110_ | CAGGUUUUCGGGUCUUCACUCGAAGACCACU |
| | T10G_G21C | |
| 559 | U7_110_ | CAGGUUUUCUUGUCUUCACAGGAAGACCACU |
| | G11T_T20A | |
| 560 | U7_110_ | CAGGUUUUCUAGUCUUCACUGGAAGACCACU |
| | G11A_T20T | |
| 561 | U7_110_ | CAGGUUUUCUGGUCUUCACCGGAAGACCACU |
| | G11G_T20C | |
| 562 | U7_110_ | CAGGUUUUCUCGUCUUCACGGGAAGACCACU |
| | G11C_T20G | |
| 563 | U7_110_ | CAGGUUUUCUGUUCUUCAAUGGAAGACCACU |
| | G12T_C19A | |
| 564 | U7_110_ | CAGGUUUUCUGAUCUUCAUUGGAAGACCACU |
| | G12A_C19T | |
| 565 | U7_110_ | CAGGUUUUCUGCUCUUCAGUGGAAGACCACU |
| | G12C_C19G | |
| 566 | U7_110_ | CAGGUUUUCUGGCCUUCGCUGGAAGACCACU |
| | T13C_G | |
| | A18 | |
| 567 | U7_110_ | CAGGUUUUCUGGGCUUCCCUGGAAGACCACU |
| | T13G_A18C | |
| 568 | U7_110_ | CAGGUUUUCUGGACUUCUCUGGAAGACCACU |
| | T13A_A18T | |
| 569 | U7_110_ | CAGGUUUUCUGGUUUUCACUGGAAGACCACU |
| | C14T | |
| 570 | U7_110_ | CAGGUUUUCUGGUAUUCACUGGAAGACCACU |
| | C14A | |
| 571 | U7_110_ | CAGGUUUUCUGGUGUUCACUGGAAGACCACU |
| | C14G | |
| 572 | U7_110_ | CAGGUUUUCUGGUCAUCACUGGAAGACCACU |
| | T15A | |
| 573 | U7_110_ | CAGGUUUUCUGGUCGUCACUGGAAGACCACU |
| | T15G | |
| 574 | U7_110_ | CAGGUUUUCUGGUCCUCACUGGAAGACCACU |
| | T15C | |
| 575 | U7_110_ | CAGGUUUUCUGGUCUACACUGGAAGACCACU |
| | T16A | |
| 576 | U7_110_ | CAGGUUUUCUGGUCUGCACUGGAAGACCACU |
| | T16G | |
| 577 | U7_110_ | CAGGUUUUCUGGUCUCCACUGGAAGACCACU |
| | T16C | |
| 578 | U7_110_ | CAGGUUUUCUGGUCUUAACUGGAAGACCACU |
| | C17A | |
| 579 | U7_110_ | CAGGUUUUCUGGUCUUGACUGGAAGACCACU |
| | C17G | |
| 580 | U7_110_ | CAGGUUUUCUGGUCUUUACUGGAAGACCACU |
| | C17T | |
| 581 | U7_110_ | CAGGUUUUCUGGUCUUCACUGGAAGACCGCU |
| | A29G | |
| 582 | U7_110_ | CAGGUUUUCUGGUCUUCACUGGAAGACCCCU |
| | A29C | |
| 583 | U7_110_ | CAGGUUUUCUGGUCUUCACUGGAAGACCUCU |
| | A29T | |
| 584 | U7_110_ | CAGGUUUUCUGGUCUUCACUGGAAGACCAGU |
| | C30G | |
| 585 | U7_110_ | CAGGUUUUCUGGUCUUCACUGGAAGACCAUU |
| | C30T | |
| 586 | U7_110_ | CAGGUUUUCUGGUCUUCACUGGAAGACCAAU |
| | C30A | |
| 587 | U7_110_ | CAGGUUUUCUGGUCUUCACUGGAAGACCACA |
| | T31A | |
| 588 | U7_110_ | CAGGUUUUCUGGUCUUCACUGGAAGACCACG |
| | T31G | |
| 589 | U7_110_ | CAGGUUUUCUGGUCUUCACUGGAAGACCACC |
| | T31C | |
| 590 | SNORA38 | UCCUCCUACAAAGGCGUGUCUGUGGUUCCCUGUCUUUGGACACGUAAGAAU |
| | | UGGAGGAAAAUAAAUGUGGAUUUGGGAAACUUUGAGGCCAGCUUGCUUCUU |
| | | GCAGGCUCAUGAUCAACCAAUCUCACAUAA |
| 591 | SNORA24 | CUCCAUGUAUCUUUGGGACCUGUCAGCCGUGGCAGUCUCCCUUCCUAGCCA |
| | | UGGAAGAGCAUAUCCUUGUUUAUUGGCAAAGCUGUCACCAUUUAAUUGGUA |
| | | UCAGAUUCUGACUUGCACAAGUAACAUUC |
| 592 | SNORA72 | CUGCGAAUAUUCUCGCUGUUCUGAUUUUGUAAUAGUCAGGACAGGCUAAAC |
| | | AUUCGCUAUAUUAAGACCAUGCAUGUGUCCCCAAACCUAGUUCUUUCCCUA |
| | | GGUCUGGUUUCAUAAAUGCUGGUGAUAAAC |
| 593 | SNORA15 | GCAUGGCCGAAUACUGUGUUUUUAUCAGUAGUUUACACAGCCAGACACCAU |
| | | GCAAAAGCAGUCUUCCCUUUAGAAUGACUGAUGGUAUGCUAAGGUUUUUCA |
| | | UAGCAUAUCAUUAUUAAAGGUGAAUACAAAU |
| 594 | SNORA8 | UGCACUGCAUGGUAUCUGCACUCAGCAGUUUACACCUGCUAGGGUGUUCAA |
| | | AGGUCAGUGCUAUAGAAAUUCAGUAUCUGGCAUCGUUGGUUUUCUUGGCUU |
| | | UGUGCUUGUUAAACCUGGUAUUUCUACUGAUACAGUA |
| 595 | SNORA52 | UGGUCCAUCCUAAUCCCUGCCGGUCCAUCUGUGGCCUGCCAGGUUUCGCUU |
| | | GUGGACCAGAGCACCCUAGAAGCCUCACCCGAGGAGUGAGCAGGGCUCCAG |
| | | UGGGCUCACGUCAUGGGCACUUCUAGACACUC |
| 596 | SNORA60 | CACCUGCAUUCAAAAAUGAUCACGGGCUGCCUGUGCUCUGGUCAUCAAUAA |
| | | CGCAGGGAGAGGAAUUGCUGAAAGCCGUUUCCCGUGUUUGGAGGGUUCACA |
| | | CCUGUCCCUUUCAAAUGCUGGCGCUUUCACACAC |
| 597 | SNORA14A | UGCAUUCUUAAACCCUCUUGGUGGCUUCCCUGUAAAUGCUUCCAAGAUAUG |
| | | AGCGAAUGCUAUAGAAAUUGCAGGAAAGUCCAAAGGGCUGCGCGUCUCCUG |
| | | UGGCUCAGUCUUAUUUCAUACCUGCAACAUCU |
| 598 | SNORA68 | AUUGCACCUAAACCCAAGAAUCACUGUUUCUUAUAGCGGUGGUUUAAACAG |
| | | AGGUGCAAACAGCAAGCGGAUCUUGUCGCCUUUGGGGGGCUGUGGCCGUGC |
| | | CCCUCAAAGUGAAUUUGGAGGUUCCACAACU |
| 599 | SNORA20 | CUUCCCAUUUAUUUGCUGCUUGUAGUCUCACAGUGAUACGAGCAGUUAUAC |
| | | GCAUGGGAUAAAAUAACAUUGGGCCACUGUAAAUUGAGAUGAAGUAACCAU |
| | | UUUCAUCUCUUCUGCAGGGACUAGACAUUG |
| 600 | SNORA14B | CUGCAUUCUUAAACCCUCUUGGUAGCUUCGUUCUAAGUGCUUCCAAGAUAU |
| | | GAGUGAAUGCUAUAGAAAUUGCAGGGGAGUCCAAAGGGCUGCGCUUCUCCC |
| | | GUGGCUCAGUCUUAUUUCAUACCUGCGACAUCU |
| 601 | SNORA63 | AGGCAGGAUCUAGUUACAUUGUAGCUGUGAAGUGCUGCAUUGUCUUUGCCC |
| | | CCUGCUCAAAAUAAAACUGUUACCUUUCAAGCCCUGUCUGCCAUGGUGCUG |
| | | UAGCAGCAGGGAUGUUUGGUCUCAUACAU |
| 602 | SCARNA4 | ACUGGAGGACUAAGGAGGCUGGGUCUGAUGAGGCAAGAUUUUGCUGAUACA |
| | | UUGCUCCUAGAAAAAAGGGUUGGCAAGAGCAGCCCUGGAGACUCACACGGC |
| | | UGACUGUUCUACCCAACACUC |
| 603 | SNORA63C | AAGCAGGAUUCAGACUACAAUAUAGCUGUUAAGUGCUGUAUUGUCAUUCCC |
| | | CCUGCUCAAAUUAAAGUUGUUUCUUAACUAUACCCAUCUGCUAUUCUGUAG |
| | | CAGCCAGGGAUGCUUGGUCACAUACAU |
| 604 | SNORA51 | GGCUUCCUAGUACUUACCAUGGUCUGUGUUCUUACGCUGACUGUAUAGAAA |
| | | CAGGAGGCAGAGUAAACCGACCCCACAUAUACCUCAGCCCAGGCCCUGUGC |
| | | UGCGUCUGUAUUGUGAAUCAGGAGACAUGG |
| 605 | SCARNA21 | UGGCUCGAUUUCCUGGGGGGUGGUCUCAGCCCACUCCACCUCCCCUCAGCC |
| | | GAGCCUAGAGUAGAGGGGCCAGGCAUCCUCCCCAGGGGAGGGGCGUUGAAG |
| | | CAAGGAGCCUCUCCUGGGCUGUCCUAGCCUCACAUUU |
| 606 | SNORA18 | GUUGAGGUCUAUCCCGAUAGGUCUUUUCCUGUAGCCUGCACGUUGUUGGAA |
| | | AUGCCUCAUAGAGUAACUCUGUGAUUUUACUUUACUUACAGGACUAUUGUU |
| | | ACAUCUGUGGGAAGGAACCACAAGACAGUU |
| 607 | SNORA68B | UUCUCACCUAAACCCAAGAAUCACUGUUUCUUAUAGCGGUGGUUUAAACAG |
| | | AGGUGCAAACAGCAAGUGAAUCUCGUCGCCUUUGCGGGGCUGUGGCCAUGC |
| | | CCCUCAAAGGAAAUUUGGAGGCUCUACAGCC |
| 608 | SNORA25 | AGGUCAUUUCAAAGAGGUCUUGUGAGGCUGUGAAACCAAGAGCUCUUAACA |
| | | CUGCGACCAAAGAUGGAAGUUCUCUAUAGGAUGCCAUGGCAUUUGAUGGUG |
| | | CUAUGUUUUCUUGAGGAGAUAUAAGA |
| 609 | SNORA38B | CCCUCCUACAAAGGCAUGUCUAUAGUUCCUUGUCUUUGGACAUGUAAGAAU |
| | | UGGAGGCAAAGAAAUGUGGACUUGGAGAAAUCUGGGGCCAGCUUGCUCUCC |
| | | GCAGGCUCAAGAUCAACCAUCCCACAUAG |
| 610 | SNORA77 | GCAGACUCACUAUGCACCUGACUGUACUUCCAGGCAGGUGCUUUUUCUGUC |
| | | UGCCAGAGAAACAUUCCAGGGUGCUGUGGCUGCCUCACCUAUCCAGGGCGA |
| | | UGCAGCUCCCUGGGGACACAGGU |
| 611 | SNORA77B | GCAGACUCACUCUGCAUCUGACUAUACUUCCAGGCGGGUGCUUUUUCUGUC |
| | | UGCCAGAUAAACAUUCCAGGGUGCUGUGGCCGCCUCACGUAUCCAGAGUGA |
| | | UGCAGCUCCCUGGGGACACAGGU |
| 612 | SNORA79B | UGAUGGCUGUUCCUCUCACUGCUUGAAGCCUUAGGCAGUGGGAUUUUGAUC |
| | | CAUCAUAUAUCAAAAAUGGCUUAUCUUCACUCAGGGCACCAUGAGGAUGGG |
| | | CUGGCUGUCCGUUAGUGCCUUCUGAUUUUUGCGGAGUCAAACAAUU |
| 613 | SNORA70EL5 | CUGGGGAAUUCAAACUUGUGUUAAGAAAAUGUGUCCCAGUGUGCAAUGGCU |
| | | GCAAACAGCAGCUUCCUUGGUAGUGUAUGCAGCCUGUUUGUUGUACGGGUU |
| | | GCUCUAAAGGGCCUUGGAGACAGUC |
| 614 | SNORA25L6 | AGGUCACUUCAAAGAGGGCUUGUGGGGCUGUGAAACCAAGAGGUCUUAACA |
| | | GUAUGACCAAAAACUGAAGUUCUCUAUAGGAUGCUGUAGCACUCAAUGGUG |
| | | CUAUGUUUUCCUCAGGAGAUAUGA |
| 615 | SNORA104 | CUGUCCGUUGCUGGCUUCACAAGUACUAGUAUAAUUUUUAAAAUGUUUUAU |
| | | UAUUUUGAAAAUAAUGUUGUAAUUCAUGCCAGGGACUGACAAAAGACUUGA |
| | | GACAGGAUGGUUAUUCUUGUCAGCUAAGGUCACAUUG |
| 616 | SNORA98 | UCCAAACAGACACUGAUGGCACCUUCUGCCAUUUAGGAAUUUGUUUUAAAA |
| | | CAGACAUUUGUCUAGAUAUUUCCUUUGUGGCCUCCUCCCCAUCAAAAGUCA |
| | | AUCAAACAUCG |
| 617 | ACA59 | GCCCUAUGUUAAAAUUUUAAUUCUGCACUUACUAACUAUCUUGGGAACCUU |
| | | GGGCAAGUAACCAACCUCUUGUGCUUUGGUUUCCUCAUUGGUAAAAUGGGG |
| | | AUAACAGUACUUACCUCACAGAGUUGUUGAGAGGAACAAAU |
| 618 | SNORA63B | AUGCAGGUACUGUUACAAUACAACUGAUGUGUUUUGUUGUCGUUCCCCCUG |
| | | CUUAAAGCACUUGAUGCAUAACUCUGUCUACCUUCAUUCCGUAGUAAGACA |
| | | GAGACGCUUGGCUUCAGACAUUU |
| 619 | SNORA120 | UCACUGCCCUGCUCACCCUUCCUGAGUCCGGCGGCAAGGGUAACUCUGGGA |
| | | GCAUCGUAGAGGGCAGAGAAGAAGAAACCCUGAGGUCCCAUUAUGUCAGCC |
| | | CCUUCUAUCACACGGGAGGAGACUGAGGACAGAAAGGGAACAGAG |
| 620 | SNORA116 | CUUUUCUCAGUGGUGCAAGAAGAUUAAGCCACAUUCUGGCUUUAGAGAGGC |
| | | AUUUCUGAGAGAGAUGAAGGACACUUCGUUCCCCAGCCCCAACCUAAGCAU |
| | | GUGACUGUACUCACCUUGUCAGAUGCUGUUGGAACCUGGCUGACA |
| 621 | SCARNA15 | CUGGAGGACUAAGGAGGCUGGGUCUGAUGAGGCAAGAUUUUGCUGAUACAU |
| | | UGCUCCUAGAAAAAAGGGUUGGCAAGAGCAGCCCUGGAGACUCACACGGCU |
| | | GACUGUUCUACCCAACACUC |
| 622 | snoU109 | UAGUGUGGAACUGUCUACUCCUCAUUCCUGUGGAAGCAGGAAUACAUUCAU |
| | | AACAUGCUCCAUUAAAAAAGGAGUUCUAGGCCAGGCAGCGUGCCUCAUGCC |
| | | UGGAAUCCCAGCACUU |
| 623 | SNORA68L2 | AUACCUAAACCCAAGAAUCACUUUCUUAUAGUGAUGAUUUAAACAGAUGCA |
| | | AACAGCGAGCACAUCUUGUCACCUUUGCGGGACUGUGGCUGUGCCCCUCGC |
| | | AGUAAAUUUGGAGGUUCUACAUCC |
| 624 | SNORA63E | AGGCAGGAUCUAGUUACAUUGUAGCUGUGAAGUGCUGCAUUGUCUUUGCCC |
| | | CCUGCUCAAAAUAAAACUGUUACCUUUCAAGCCCUGUCUGCCAUGGUGCUG |
| | | UAGCAGCAGGGAUGUUUGGUCUCAUACAUGU |
| 625 | SNORA75 | CACUAGAAGACAGAAUUCACAGAAGUAGCAUUUCACCUUUUGCCUUUACAG |
| | | AAGUAUAUUUGGCUGUUUUGUGAGACAUUC |
| 626 | SNORA95 | ACUUUUACAGGUAGAAUAGUAAAGCACAGUGUUGAUUGCCCAAGAUUUAUU |
| | | UUACUUUGAAAAAAUUAGAAAUUUAUUACUAUAGCAAAUGUCUAGAACUUU |
| | | GGAAACAAGU |
| 627 | SNORA31B | AUGCAUCUAUUUGACAGACCUGGAGCAGUUGCUAUCUGCUGCUAUGGUUUC |
| | | CACCACAGAUGCAAGAAGAACAUGUCCUUGCGCUUUCCGUCUGUCUAAUUG |
| | | UGGCAGCUGAGAUUGAAUAGAGGAAUACAGGA |
| 628 | SNORA50_1 | AAGCACUGCCUUUGAACCUGAUGUGUCUUGUUUGUAGCUUCACGGGCCAAG |
| | | CAACAGUGCUAGAGCAUAACGACUUGUUAUAACUGGGGCUCUUCAGCUCUC |
| | | AACUGAACUGCUCUUUUAAAAACAAGGUACAUUU |
| 629 | SNORA50_2 | AAGCACUGCCUUUGAACCUGAUGUGUCUUGUUUGUAGCUUCACGGGCCAAG |
| | | CAACAGUGCUAGAGCAUAACGACUUGUUAUAACUGGGGCUCUUCAGCUCUC |
| | | AACUGAACUGCUCUUUUAAAAACAAGGUACAUUU |
| 630 | ACA13 | AGCCUUUGUGUUGCCCAUUCACUUUGGAAACUAGUGAAUGUGGUGUCAAAA |
| | | AAGGCGUAAAUUAAACGCUUUGCAGCCUUUUCCUGCCCUUAAAUUUGAUAC |
| | | CUUUGGUGUAGGAGCUGCAUAAGUAACAGUU |
| 631 | ACA36_2 | UUCCAAAGUGUUGAGUUCAGUCCAGGGCAGCUUCCCUGUUCUGUUAAUUAA |
| | | ACUUUGGGACAUUAAAAUGGGCUAAGGGAGAUGAUUGGGUAGAAAGUAUUA |
| | | UUCUAUUCAUUUGCCUCCCAGCCUACAAAA |
| 632 | ACA28_1 | AAGCAACACUCUGUGGCAGAUGAUCAAAACUGUCUGACACAAUUUGAGCUU |
| | | GCUAUAGCAAGAAAGUCUAACCUAUUCCGGUGUUCUCUCUCCCAUGAGACA |
| | | AGCCGUUAUAUAGACUUAAACAGU |
| 633 | ACA28_2 | AAGCAACACUCUGUGGCAGAUGAUCAAAACUGUCUGACACAAUUUGAGCUU |
| | | GCUAUAGCAAGAAAGUCUAACCUAUUCCGGUGUUCUCUCUCCCAUGAGACA |
| | | AGCCGUUAUAUAGACUUAAACAGU |
| 634 | ACA41 | UUCCACAGCUACUGGUCUGCAGCUGUUCUUAUGGUAGCAGUUGUGGCAUUC |
| | | CUCUGUGGGAAAGAAACUGUUAACACAAACACCUCUUUCUUAGCAAAACAG |
| | | AAAGUGGGUAUAUAUGUGUGACAGACACAA |
| 635 | ACA5_2 | UGCAGCCGUGUCAAAUUCAGUACCUGUCCUAUGCAUGGUAGGCACUGGCCC |
| | | AGAAGGCUGCCACAGAAACACUGUGACUCAUGGGCCCUGUUCCUGUGUCCC |
| | | AGGCUCAGGGAUAAAUUUGGUUACAGACAUCA |
| 636 | ACA8_1 | UGCACUGCAUGGUAUCUGCACUCAGCAGUUUACACCUGCUAGGGUGUUCAA |
| | | AGGUCAGUGCUAUAGAAAUUCAGUAUCUGGCAUCGUUGGUUUUCUUGGCUU |
| | | UGUGCUUGUUAAACCUGGUAUUUCUACUGAUACAGUA |
| 637 | ACA8_2 | UGCACUGCAUGGUAUCUGCACUCAGCAGUUUACACCUGCUAGGGUGUUCAA |
| | | AGGUCAGUGCUAUAGAAAUUCAGUAUCUGGCAUCGUUGGUUUUCUUGGCUU |
| | | UGUGCUUGUUAAACCUGGUAUUUCUACUGAUACAGUA |
| 638 | ACA5_1 | UGCAGCCGUGUCAAUUCAGUACCUGUCCUAUGCAUGGUAGGCACUGGCCCA |
| | | GAAGGCUGCCACAGAAACACUGUGACUCAUGGGCCCUGUUCCUGUGUCCCA |
| | | GGCUCAGGGAUAAAUUUGGUUACAGACAUCA |
| 639 | ACA36_1 | UUCCAAAGUGUUGAGUUCAGUCCAGGGCAGCUUCCCUGUUCUGUUAAUUAA |
| | | ACUUUGGGACAUUAAAAUGGGCUAAGGGAGAUGAUUGGGUAGAAAGUAUUA |
| | | UUCUAUUCAUUUGCCUCCCAGCCUACAAAA |
| 640 | ACA42_1 | UGGUAAUGGAUUUAUGGUGGGUCCUUCUCUGUGGGCCUCUCAUAGUGUACC |
| | | CAUGCCAUAGCAAAUGGCAGCCUCGAACCAUUGCCCAGUCCCCUUACCUGU |
| | | GGGCUGUGAGCACUGAAGGGGGUUGCACAGUG |
| 641 | ACA42_2 | UGGUAAUGGAUUUAUGGUGGGUCCUUCUCUGUGGGCCUCUCAUAGUGUACC |
| | | CAUGCCAUAGCAAAUGGCAGCCUCGAACCAUUGCCCAGUCCCCUUACCUGU |
| | | GGGCUGUGAGCACUGAAGGGGGUUGCACAGUG |
| 642 | ACA44_1 | CAGCAUGUUUCCAAGGGCUGUGGCUGGUCAUAGCCAUGGGAUCUCCAACUG |
| | | CAUGCAAGAGCAACCUGGAAAGACUUUGACAGCGCAGGUCAGUACAAUACC |
| | | UGCAAGCUGCCACUCAGCUUUCCUAUAAUG |
| 643 | ACA44_2 | CAGCAUGUUUCCAAGGGCUGUGGCUGGUCAUAGCCAUGGGAUCUCCAACUG |
| | | CAUGCAAGAGCAACCUGGAAAGACUUUGACAGCGCAGGUCAGUACAAUACC |
| | | UGCAAGCUGCCACUCAGCUUUCCUAUAAUG |
| 644 | ACA10_2 | GGUCUCUCAGCUCCGCUUAACCACACGGGUCCAGUGUGUGCUUGGCGUGUU |
| | | UUCAGGGAGGCAGAGAAAGGCUCUCCUAAUGCACGACAGACCCGCCCAGAA |
| | | UGGCCUCUCUGUUCCUAGGAGUGCGACAAUU |
| 645 | ACA46 | AGCACUAUAUUUAAACCUGUGGAUGGGAAUAUUCCCCAUUCUUGGUUACGC |
| | | UGUAGUGCAAAAGAAUUCCUGGCUCUCUGUUGCACAGCUGACUUGUGCCAU |
| | | UCUGCUGUUGCUGUAUAGAGUUAAGGAACAUGG |
| 646 | ACA10_1 | GGUCUCUCAGCUCCGCUUAACCACACGGGUCCAGUGUGUGCUUGGCGUGUU |
| | | UUCAGGGAGGCAGAGAAAGGCUCUCCUAAUGCACGACAGACCCGCCCAGAA |
| | | UGGCCUCUCUGUUCCUAGGAGUGCGACAAUU |
| 647 | ACA1 | UGCCUCAUUCUAGAGAAUGGGCACUGUUGAUCAUGGUGUCCAAAAAUAGUU |
| | | AAUGUGGCUAAAUUGAGACAGGUUAUGCUUCCAUCACAGUAUGCAUAUUGC |
| | | AGUGGUGACAAUGAGACCUGUAACAUUU |
| 648 | ACA2A_1 | UAGGCCCUGAAUCAAGACCAAUGGUUUGCUGUAGCUGUUGGUUUCAAACAG |
| | | GAGCUAAGAGUGAUGUCUUCCUUGUGGUCUGUUGGCUAUUCAGUAUUCCAG |
| | | UGCGAAUUGCCAAUUCAGUUGGAAGAAACAUAG |
| 649 | ACA2A_2 | UAGGCCCUGAAUCAAGACCAAUGGUUUGCUGUAGCUGUUGGUUUCAAACAG |
| | | GAGCUAAGAGUGAUGUCUUCCUUGUGGUCUGUUGGCUAUUCAGUAUUCCAG |
| | | UGCGAAUUGCCAAUUCAGUUGGAAGAAACAUAG |
| 650 | ACA3_1 | AUCGAGGCUAGAGUCACGCUUGGGUAUCGGCUAUUGCCUGAGUGUGCUAGA |
| | | GUCCUCGAAGAGUAACUGCUGACCUUAUUCACUGGCUGUGGGCCUUAUGGC |
| | | ACAGUCAGUCACCAGGUUAGAGACAUGC |
| 651 | ACA3_2 | AUCGAGGCUAGAGUCACGCUUGGGUAUCGGCUAUUGCCUGAGUGUGCUAGA |
| | | GUCCUCGAAGAGUAACUGCUGACCUUAUUCACUGGCUGUGGGCCUUAUGGC |
| | | ACAGUCAGUCACCAGGUUAGAGACAUGC |
| 652 | ACA6 | UGCACACUAUUAAAGCUCAGGGUGGAGGCCAGUCUUGGCUCAUGAACUUCU |
| | | GAGUGUCGGAAGUGUGCUAUAUCAAUGGCAGGAUUUUCGCUAACACCAGUA |
| | | GAGCUUGCCUCUAUGACUGGAGUUUGGUAGUACUCGCUGCCACAUAG |
| 653 | ACA7_1 | GACCUCCUGGGAUCGCAUCUGGAGAGUGCCUAGUAUUCUGCCAGCUUCGGA |
| | | AAGGGAGGGAAAGCAAGCCUGGCAGAGGCACCCAUUCCAUUCCCAGCUUGC |
| | | UCCGUAGCUGGCGAUUGGAAGACACUCUGCGACAGUG |
| 654 | ACA7_2 | GACCUCCUGGGAUCGCAUCUGGAGAGUGCCUAGUAUUCUGCCAGCUUCGGA |
| | | AAGGGAGGGAAAGCAAGCCUGGCAGAGGCACCCAUUCCAUUCCCAGCUUGC |
| | | UCCGUAGCUGGCGAUUGGAAGACACUCUGCGACAGUG |
| 655 | ACA9_1 | UAGCAAGCCUCCAGCGUGCUUGGGUCUGCGGUGACCCUAUGCAUUCCUUCA |
| | | GUGCUUGCUAGAACAGUUUUGAAACGGUUUGAGGCCUUGCCCUGCUCCAUC |
| | | CAGAGCAAGGUUAUAGAAAUUUCAGACAAUG |
| 656 | ACA9_2 | UAGCAAGCCUCCAGCGUGCUUGGGUCUGCGGUGACCCUAUGCAUUCCUUCA |
| | | GUGCUUGCUAGAACAGUUUUGAAACGGUUUGAGGCCUUGCCCUGCUCCAUC |
| | | CAGAGCAAGGUUAUAGAAAUUUCAGACAAUG |
| 657 | ACA16 | UUGGCCCUUAUCGAAGCUGCAGCUGCUUCCGCAUAGCUGCUGUGGUCAAAA |
| | | AGGAGCCCAGAGUGACAGUUUUCCUUGACGGUCGCCGUUCUGUUUGUUGUA |
| | | ACUGAUCUGCAACAUUUUGGGAAAAUACAGUU |

1. A nucleic acid for targeting trans-splicing of a pre-mRNA in a cell, the nucleic acid comprising a nucleotide sequence comprising
(a) at least one intronic sequence comprising (i) one or more binding domain sequences of about 4 to about 300 nucleotides each with complementarity to a pre-mRNA target sequence; and (ii) a non-coding RNA (ncRNA) sequence of about 7 to about 300 nucleotides in length which forms a secondary structure and/or comprises a sequence motif to direct the one or more binding domains to the pre-mRNA target sequence;
(b) a splice acceptor and/or splice donor sequence; and
(c) at least one exonic sequence.

2. The nucleic acid of claim 1, wherein the one or more binding domain sequences is at least about 5 to about 10, about 5 to about 15, about 5 to about 20, about 10 to about 15, about 10 to about 20, about 15 to about 20, or about 20, 19, 18, 17, 16, 15, 14, 13, 12, 11, 10, 9, 8, 7, 6, or nucleotides in length.

3. The nucleic acid of claim 1 or 2, wherein the one or more binding domain sequences is less than about 250 to about 300, about 200 to about 300, about 150 to about 300, about 100 to about 300, about 50 to about 300, about 100 to about 250, about 100 to about 200, about 100 to about 150, about 50 to about 250, about 50 to about 200, about 50 to about 150, about 50 to about 100, or about 300, 250, 200, 150, 100, or 50 nucleotides in length.

4. The nucleic acid of any one of claims 1-3, wherein the one or more binding domain sequences is about 5 to about 20, about 5 to about 30, about 5 to about 40, about 5 to about 50, about 10 to about 50, about 10 to about 100, about 20 to about 100, about 30 to about 100, about to about 100, about 50 to about 100, about 50 to about 150, about 50 to about 200, about 50 to about 250, about 100 to about 150, about 100 to about 200, about 100 to about 250, or about 100 to about 300 nucleotides in length.

5. The nucleic acid of any one of claims 1-4, comprising one binding domain sequence.

6. The nucleic acid of any one of claims 1-4, comprising at least two binding domain sequences.

7. The nucleic acid of claim 6, comprising 3, 4, 5, 6, 7, 8, 9, or 10 binding domain sequences.

8. The nucleic acid of any one of claims 1-7, wherein when the nucleic acid is introduced to the cell an exon in the pre-mRNA is targeted for trans-splicing.

9. The nucleic acid of claim 8, wherein the target sequence is positioned in a region of the pre-mRNA comprising the exon targeted for trans-splicing.

10. The nucleic acid of claim 9, wherein the target sequence is positioned proximal to a splice site.

11. The nucleic acid of claim 9 or 10, wherein the target sequence is positioned proximal to a splice donor or a splice acceptor.

12. The nucleic acid of any one of claims 1-11, wherein the ncRNA sequence is selected from an snRNA, a snoRNA, a lncRNA, an rRNA, a ribozyme, an sRNA, a scaRNA, and a vault RNA.

13. The nucleic acid of claim 12, wherein the ncRNA sequence is an snRNA.

14. The nucleic acid of claim 13, wherein the snRNA is selected from a U7 snRNA, a U1 snRNA, a U2 snRNA, a U4 snRNA, a U4atac snRNA, a U5 snRNA, a U6 snRNA, a U6atac snRNA, a U11 snRNA, and a U12 snRNA.

15. The nucleic acid of claim 12, wherein the ncRNA sequence is a snoRNA.

16. The nucleic acid of claim 15, wherein the snoRNA comprises an H/ACA box or C/D box.

17. The nucleic acid of any one of claims 1-16, wherein the ncRNA sequence assembles into an RNP.

18. The nucleic acid of any one of claims 1-17, wherein the ncRNA sequence comprises a sequence motif that assembles into an RNP.

19. The nucleic acid of any one of claims 1-17, wherein the ncRNA sequence comprises a secondary structure that assembles into an RNP.

20. The nucleic acid of any one of claims 1-17, wherein the ncRNA sequence comprises a sequence motif and a secondary structure that assembles into an RNP.

21. The nucleic acid of claim 19 or 20, wherein the secondary structure comprises one or more stem loops.

22. The nucleic acid of any one of claims 17-21, wherein the RNP is selected from a small nuclear RNP (snRNP), a small nucleolar RNP (snoRNP), a small cajal body RNP (scaRNP), and a combination thereof.

23. The nucleic acid of claim 22, wherein the RNP is selected from U1, U2, U4, U4atac, U5, U6, U6atac, U7, U11, and U12.

24. The nucleic acid of claim 22, wherein the RNP is selected from a C/D box snoRNP and a H/ACA box snoRNP.

25. The nucleic acid of any one of claims 1-24, wherein the ncRNA comprises an Sm sequence motif.

26. The nucleic acid of claim 25, wherein the Sm sequence motif assembles with an Sm or Lsm protein into an RNP.

27. The nucleic acid of claim 25, wherein the Sm or Lsm proteins are selected from a B/B′, D3, D2, D1, E, F, G, LSm5, LSm7, LSm4, LSm8, LSm2, LSm3, LSm6 and LSm10 proteins.

28. The nucleic acid of any one of claims 1-27, comprising a splice acceptor.

29. The nucleic acid of any one of claims 1-27, comprising a splice donor.

30. The nucleic acid of any one of claims 1-29, wherein the at least one intronic sequences comprises one or more splicing signals.

31. The nucleic acid of claim 30, wherein the one or more splicing signals are selected from an exonic splicing enhancer (ESE), an intronic splicing enhancer (ISE), an exonic splicing silencer (ESS), intronic splicing silencer (ISS), a polypyrimidine tract, a branch point, and a combination thereof.

32. The nucleic acid of any one of claims 1-31, wherein the at least one intronic sequences comprises a branch point and a polypyrimidine tract.

33. The nucleic acid of any one of claims 1-32, comprising 1, 2, 3, 4, 5, 6, 7, 8, 9, or 10 exons.

34. A nucleic acid for targeting trans-splicing of a pre-mRNA in a cell, the nucleic acid comprising a nucleotide sequence comprising from 5′ to 3′:
(a) at least one intronic sequences comprising (i) one or more binding domain sequences of about 4 to about 300 nucleotides each with complementarity to a pre-mRNA target sequence; (ii) a non-coding RNA (ncRNA) sequence of about 7 to about 300 nucleotides in length which forms a secondary structure and/or comprises a sequence motif to direct the one or more binding domains to the pre-mRNA target sequence; and (iii) one or more splicing signals;
(b) a splice acceptor; and
(c) at least one exonic sequences.

35. The nucleic acid of claim 34, wherein when the nucleic acid is introduced to the cell an exon in the pre-mRNA is targeted for trans-splicing.

36. The nucleic acid of claim 34 or 35, wherein the target sequence is positioned upstream the exon in the pre-mRNA targeted for trans-splicing.

37. The nucleic acid of claim 36, wherein the target sequence is positioned proximal to a splice site.

38. The nucleic acid of claim 36 or 37, wherein the target sequence is positioned proximal to a splice acceptor or a splice donor.

39. The nucleic acid of any one of claims 35-38, wherein trans-splicing occurs between a splice donor upstream the exon in the pre-mRNA and the splice acceptor of the nucleic acid.

40. The nucleic acid of any one of claims 35-39, wherein trans-splicing results in ligation of the 3′end of an exon upstream the splice donor in the pre-mRNA with the 5′end of the at least one exonic sequence of the nucleic acid.

41. The nucleic acid of any one of claims 34-40, wherein the one or more splicing signals comprises a branch point and a polypyrimidine tract.

42. A nucleic acid for targeting trans-splicing of a pre-mRNA in a cell, the nucleic acid comprising a nucleotide sequence comprising from 5′ to 3′:
(a) at least one exonic sequence;
(b) a splice donor;
(c) at least one intronic sequence comprising (i) a non-coding RNA (ncRNA) sequence of about 7 to about 300 nucleotides in length, and (ii) one or more binding domain sequences of about 4 to about 300 nucleotides each with complementarity to a pre-mRNA target sequence, wherein the ncRNA forms a secondary structure and/or comprises a sequence motif to direct the one or more binding domains to the pre-mRNA target sequence.

43. The nucleic acid of claim 42, wherein when the nucleic acid is introduced to the cell an exon in the pre-mRNA is targeted for trans-splicing.

44. The nucleic acid of claim 43, wherein the target sequence is positioned downstream the exon in the pre-mRNA.

45. The nucleic acid of claim 44, wherein the target sequence is positioned proximal to a splice site.

46. The nucleic acid of claim 44 or 45, wherein the target sequence is positioned proximal to a splice donor or a splice acceptor.

47. The nucleic acid of any one of claims 43-46, wherein trans-splicing occurs between the splice donor of the nucleic acid and a splice acceptor downstream the exon in the pre-mRNA.

48. The nucleic acid of any one of claims 43-47, wherein trans-splicing results in ligation of the 3′end of the at least one exonic sequence of the nucleic acid with the 5′end of an exon downstream the splice acceptor in the pre-mRNA.

49. The nucleic acid of any one of claims 34-48, wherein the ncRNA sequence is an snRNA.

50. The nucleic acid of claim 49, wherein the snRNA is selected from a U1 snRNA, a U2 snRNA, a U4 snRNA, a U4atac snRNA, a U5 snRNA, a U6 snRNA, a U6atac snRNA, a U11 snRNA, a U12 snRNA, and a U7 snRNA.

51. The nucleic acid of claim 49 or 50, wherein the snRNA assembles into an snRNP.

52. The nucleic acid of claim 49 or 50, wherein the snRNA is a U1 snRNA.

53. The nucleic acid of claim 52, wherein the U1 snRNA assembles into a U1 RNP.

54. The nucleic acid of claim 49 or 50, wherein the snRNA is a U11 snRNA.

55. The nucleic acid of claim 54, wherein the U11 snRNA assembles into a U11 RNP.

56. The nucleic acid of claim 49 or 50, wherein the snRNA is a U7 snRNA.

57. The nucleic acid of claim 56, wherein the U7 snRNA assembles into a U7 RNP.

58. The nucleic acid of claim 49 or 50, wherein the ncRNA sequence comprises an Sm sequence motif.

59. The nucleic acid of claim 58, wherein the ncRNA sequence comprises an Sm sequence motif and a U7 snRNA.

60. The nucleic acid of claim 58 or 59, wherein the Sm sequence motif comprises a sequence set forth in SEQ ID NOs: 3 and 4.

61. The nucleic acid of any one of claims 58-60, wherein the Sm sequence motif assembles with an Sm protein into an RNP.

62. The nucleic acid of claim 61, wherein the Sm protein is selected from a B/B′, D3, D2, D1, E, F, and G Sm protein.

63. The nucleic acid of any one of claims 34-48, wherein the ncRNA sequence comprises a sequence having at least 80% sequence identity to a sequence selected from SEQ ID NOs: 9-589 or a portion thereof.

64. The nucleic acid of claim 63, wherein the ncRNA sequence comprises a region of about 7 to about 40 nucleotides in length, wherein the region comprises an Sm sequence motif.

65. The nucleic acid of claim 63, wherein the ncRNA sequence comprises a region of about to about 300 nucleotides in length, wherein the region comprises a secondary structure and/or an Sm sequence motif.

66. The nucleic acid of any one of claims 34-65, comprising one binding domain sequence.

67. The nucleic acid of any one of claims 34-65, comprising more than one binding domain sequence

68. The nucleic acid of any one of claims 34-65, wherein the one or more binding domain sequences is about 5 to about 20, about 5 to about 30, about 5 to about 40, about 5 to about 50, about 10 to about 50, about 10 to about 100, about 20 to about 100, about 30 to about 100, about to about 100, about 50 to about 100, about 50 to about 150, about 50 to about 200, about 50 to about 250, about 100 to about 150, about 100 to about 200, about 100 to about 250, or about 100 to about 300 nucleotides in length.

69. A nucleic acid for targeting trans-splicing of a pre-mRNA in a cell, the nucleic acid comprising a nucleotide sequence comprising from 5′ to 3′
(a) at least one intronic sequence comprising (i) a ncRNA sequence comprising an H/ACA box or a C/D box and one or more binding domain sequences of about 4 to about 30 nucleotides each with complementarity to a pre-mRNA target sequence; and (ii) one or more splicing signals;
(b) a splice acceptor; and
(c) at least one exonic sequence.

70. The nucleic acid of claim 69, wherein when the nucleic acid is introduced to the cell an exon in the pre-mRNA is targeted for trans-splicing.

71. The nucleic acid of claim 69 or 70, wherein the target sequence is positioned upstream the exon in the pre-mRNA.

72. The nucleic acid of claim 71, wherein the target sequence is positioned proximal to a splice site.

73. The nucleic acid of claim 71 or 72, wherein the target sequence is positioned proximal to a splice donor or splice acceptor.

74. The nucleic acid of any one of claims 70-73, wherein trans-splicing occurs between a splice donor upstream the exon in the pre-mRNA and the splice acceptor of the nucleic acid.

75. The nucleic acid of any one of claims 70-73, wherein trans-splicing results in ligation of the 3′ end of an exon upstream the splice donor in the pre-mRNA with the 5′end of the at least one exonic sequence of the nucleic acid.

76. The nucleic acid of any one of claims 69-75, wherein the one or more splicing signals comprises a branch point and a polypyrimidine tract.

77. A nucleic acid for targeting trans-splicing of a pre-mRNA in a cell, the nucleic acid comprising a nucleotide sequence comprising from 5′ to 3′
(a) at least one exonic sequence;
(b) a splice donor; and
(c) at least one intronic sequence comprising a ncRNA sequence comprising an H/ACA box or a C/D box and one or more binding domain sequences of about 4 to about 30 nucleotides each with complementarity to a pre-mRNA target sequence.

78. The nucleic acid of claim 77, wherein when the nucleic acid is introduced to the cell an exon in the pre-mRNA is targeted for trans-splicing.

79. The nucleic acid of claim 78, wherein the target sequence is positioned downstream the exon in the pre-mRNA.

80. The nucleic acid of claim 79, wherein the target sequence is positioned proximal to a splice site.

81. The nucleic acid of claim 79 or 80, wherein the target sequence is positioned proximal to a splice donor or a splice acceptor.

82. The nucleic acid of any one of claims 78-81, wherein trans-splicing occurs between the splice donor of the nucleic acid and a splice acceptor downstream the exon in the pre-mRNA.

83. The nucleic acid of any one of claims 78-81, wherein trans-splicing results in ligation of the 3′end of the at least one exonic sequence of the nucleic acid with the 5′end of an exon downstream the splice acceptor in the pre-mRNA.

84. The nucleic acid of any one of claims 69-83, wherein the ncRNA sequence comprises an H/ACA box comprising 5′ to 3′ an H consensus sequence and an ACA consensus sequence.

85. The nucleic acid of claim 84, comprising at least one binding domain sequence positioned
(i) upstream the H consensus sequence;
(ii) downstream the ACA consensus sequence;
(iii) between the H consensus sequence and the ACA consensus sequence; or
(iv) a combination of (i)-(iii).

86. The nucleic acid of any one of claims 69-83, wherein the ncRNA sequence comprises C/D box comprising 5′ to 3′ a C consensus sequence, a D′ consensus sequence, a C′ consensus sequence, and a D consensus sequence.

87. The nucleic acid of claim 86, comprising at least one binding domain positioned
(i) upstream the C consensus sequence;
(ii) between the C consensus sequence and the D′ consensus sequence;
(iii) between the C′ consensus sequence and the D consensus sequence;
(iv) downstream the D consensus sequence; or
(iv) a combination of (i)-(iii).

88. The nucleic acid of any one of claims 78-83, wherein the ncRNA sequence comprises a sequence having at least 80% sequence identity to a sequence selected from SEQ ID NOs: 590-657 or a portion thereof.

89. The nucleic acid of claim 88, wherein the ncRNA sequence comprises a region of about to about 300 nucleotides in length and comprising an H consensus sequence and an ACA consensus sequence.

90. The nucleic acid of any one of claims 69-89, wherein the ncRNA sequence comprises one binding domain sequence.

91. The nucleic acid of any one of claims 69-89, wherein the ncRNA sequence comprises more than one binding domain sequence.

92. The nucleic acid of any one of claims 1-91, comprising at least one binding domain sequence with full complementarity to the pre-mRNA target sequence.

93. The nucleic acid of any one of claims 1-92, comprising at least one binding domain sequence with partial complementarity to the pre-mRNA target sequence.

94. The nucleic acid of claim 93, wherein the at least one binding domain sequence comprises one or more mismatches relative to the pre-mRNA target sequence.

95. The nucleic acid of claim 93 or 94, wherein the at least one binding domain sequence has at least 95% complementarity to the pre-mRNA target sequence.

96. The nucleic acid of any one of claims 1-95, wherein the nucleic acid comprises a sequence up to about 20,000 nucleotides in length.

97. The nucleic acid of any one of claims 1-96, wherein the nucleic acid comprises a sequence of about 50 to about 500, about 50 to about 1000, about 100 to about 500, about 100 to about 1000, about 500 to about 1000, about 500 to about 2000, about 500 to about 3,000, about 500 to about 4,000, about 500 to about 5,000, about 1,000 to about 5,000, about 1,000 to about 10,000, about 5,000 to about 15,000, or about 5,000 to about 20,000 nucleotides in length.

98. The nucleic acid of any one of claims 1-97, wherein the nucleic acid is introduced to the cell as an RNA.

99. The nucleic acid of any one of claims 1-97, wherein the nucleic acid is introduced to the cell as a DNA.

100. The nucleic acid of any one of claims 1-99, wherein the nucleic acid is introduced to the cell by a viral vector.

101. The nucleic acid of claim 100, wherein the viral vector is an AAV.

102. The nucleic acid of any one of claims 1-100, wherein the nucleic acid is introduced to the cell by a non-viral vector.

103. The nucleic acid of any one of claims 1-102, wherein introduction of the nucleic acid to the cell results in an efficiency of trans-splicing that is greater than a nucleic acid lacking the ncRNA sequence.

104. The nucleic acid of claim 103, wherein the efficiency of trans-splicing is greater than about 10%, about 20%, about 30%, about 40%, about 50%, about 60%, about 70%, about 80%, about 90%, or about 99%.

105. The nucleic acid of any one of claims 1-104, wherein the nucleic acid is formulated as a lipid nanoparticle.

106. A viral vector comprising the nucleic acid of any one of claims 1-104.

107. A lipid nanoparticle comprising the nucleic acid of any one of claims 1-105.

108. A cell comprising the nucleic acid of any one of claims 1-105, the viral vector of claim 106, or the lipid nanoparticle of claim 107.

109. A pharmaceutical composition comprising the nucleic acid of any one of claims 1-105, the viral vector of claim 106, the lipid nanoparticle of claim 107, and a pharmaceutically acceptable carrier.

110. A pharmaceutical composition comprising the cell of claim 108, and a pharmaceutically acceptable carrier.

111. A method of targeting trans-splicing of a pre-mRNA in a cell, the method comprising contacting the cell with the nucleic acid of any one of claims 1-105, the viral vector of claim 106, the lipid nanoparticle of claim 107, or the pharmaceutical composition of claim 109-110, wherein when the nucleic acid, the viral vector, the lipid nanoparticle, or the pharmaceutical composition contacts the cell, the one or more binding domain sequences bind to the pre-mRNA, thereby targeting the pre-mRNA for trans-splicing.

112. A method of correcting a mutation in a pre-mRNA in a cell, the method comprising contacting the cell with the nucleic acid of any one of claims 1-105, the viral vector of claim 106, the lipid nanoparticle of claim 107, or the pharmaceutical composition of claim 109-110, wherein when the nucleic acid, the viral vector, the lipid nanoparticle, or the pharmaceutical composition contacts the cell, the one or more binding domain sequences bind to the pre-mRNA at a location proximal to the mutation, and wherein trans-splicing replaces one or more exons in the pre-mRNA comprising the mutation, thereby correcting the mutation.

113. A method of treating a patient with a disease or disorder associated with a mutation in a pre-mRNA, the method comprising administering to the patient an effective amount of the nucleic acid of any one of claims 1-105, the viral vector of claim 106, the lipid nanoparticle of claim 107, or the pharmaceutical composition of claim 109-110, wherein when the nucleic acid, the viral vector, the lipid nanoparticle, or the pharmaceutical composition is administered, the one or more binding domain sequences bind to the pre-mRNA at a location proximal to the mutation, and wherein trans-splicing replaces one or more exons in the pre-mRNA comprising the mutation, thereby correcting the mutation.

114. The method of claim 113, wherein trans-splicing results in an mRNA that alleviates the disease or does not cause or contribute to the disease.

115. The nucleic acid of any one of claims 1-105, the viral vector of claim 106, the lipid nanoparticle of claim 107, or the pharmaceutical composition of claim 109-110 for use in treating a patient with a disease or disorder associated with a mutation in a pre-mRNA, the treatment comprising administering to the patient the nucleic acid, the viral vector, the lipid nanoparticle, or the pharmaceutical composition, wherein when the nucleic acid, the viral vector, the lipid nanoparticle, or the pharmaceutical composition is administered, the one or more binding domain sequences bind to the pre-mRNA at a location proximal to the mutation, and wherein trans-splicing replaces one or more exons in the pre-mRNA comprising the mutation, thereby correcting the mutation.

116. The nucleic acid of any one of claims 1-105, the viral vector of claim 106, the lipid nanoparticle of claim 107, or the pharmaceutical composition of claim 109-110 for the manufacture of a medicament for use in treating a patient with a disease or disorder associated with a mutation in a pre-mRNA, the treatment comprising administering to the patient the medicament, wherein when the medicament is administered, the one or more binding domain sequences of the nucleic acid binds to the pre-mRNA at a location proximal to the mutation, and wherein trans-splicing replaces one or more exons in the pre-mRNA comprising the mutation, thereby correcting the mutation.

117. A kit comprising a container comprising the nucleic acid of any one of claims 1-105, the viral vector of claim 106, the lipid nanoparticle of claim 107, or the pharmaceutical composition of claim 109-110, with instructions for use in correcting a mutation in a pre-mRNA.


SEQUENCE LISTING (713 sequences)

SEQ ID NO: 1-8 - skipped (8 entries)

SEQ ID NO: 9 - RNA, 154 nt, synthetic construct
  source 1..154: mol_type = other RNA
atacttacgtaacaggagaaaatacggccatgaagttggtgtttctcgggggcgatttctccattgtactcagtatgtgctgactgactcctgttacttccacatgtggggaaactggactgtaatttgtggtggtggggaattgcgttcgcgc

SEQ ID NO: 10 - RNA, 154 nt, synthetic construct
  source 1..154: mol_type = other RNA
tacttacgtaacaggagaaaatacggccatgaagttggtgtttctcgggggcgatttctccattgtactcagtatgtgctgactgactcctgttacttccacatgtggggaaactggactgtaatttgtggtggtggggaattgcgttcgcgct

SEQ ID NO: 11 - RNA, 154 nt, synthetic construct
  source 1..154: mol_type = other RNA
acttacgtaacaggagaaaatacggccatgaagttggtgtttctcgggggcgatttctccattgtactcagtatgtgctgactgactcctgttacttccacatgtggggaaactggactgtaatttgtggtggtggggaattgcgttcgcgctt

SEQ ID NO: 12 - RNA, 154 nt, synthetic construct
  source 1..154: mol_type = other RNA
cttacgtaacaggagaaaatacggccatgaagttggtgtttctcgggggcgatttctccattgtactcagtatgtgctgactgactcctgttacttccacatgtggggaaactggactgtaatttgtggtggtggggaattgcgttcgcgcttt

SEQ ID NO: 13 - RNA, 154 nt, synthetic construct
  source 1..154: mol_type = other RNA
ttacgtaacaggagaaaatacggccatgaagttggtgtttctcgggggcgatttctccattgtactcagtatgtgctgactgactcctgttacttccacatgtggggaaactggactgtaatttgtggtggtggggaattgcgttcgcgctttc

SEQ ID NO: 14 - RNA, 154 nt, synthetic construct
  source 1..154: mol_type = other RNA
tacgtaacaggagaaaatacggccatgaagttggtgtttctcgggggcgatttctccattgtactcagtatgtgctgactgactcctgttacttccacatgtggggaaactggactgtaatttgtggtggtggggaattgcgttcgcgctttct

SEQ ID NO: 15 - RNA, 154 nt, synthetic construct
  source 1..154: mol_type = other RNA
acgtaacaggagaaaatacggccatgaagttggtgtttctcgggggcgatttctccattgtactcagtatgtgctgactgactcctgttacttccacatgtggggaaactggactgtaatttgtggtggtggggaattgcgttcgcgctttctt

SEQ ID NO: 16 - RNA, 154 nt, synthetic construct
  source 1..154: mol_type = other RNA
cgtaacaggagaaaatacggccatgaagttggtgtttctcgggggcgatttctccattgtactcagtatgtgctgactgactcctgttacttccacatgtggggaaactggactgtaatttgtggtggtggggaattgcgttcgcgctttcttc

SEQ ID NO: 17 - RNA, 154 nt, synthetic construct
  source 1..154: mol_type = other RNA
gtaacaggagaaaatacggccatgaagttggtgtttctcgggggcgatttctccattgtactcagtatgtgctgactgactcctgttacttccacatgtggggaaactggactgtaatttgtggtggtggggaattgcgttcgcgctttcttct

SEQ ID NO: 18 - RNA, 153 nt, synthetic construct
  source 1..153: mol_type = other RNA
taacaggagaaaatacggccatgaagttggtgtttctcgggggcgatttctccattgtactcagtatgtgctgactgactcctgttacttccacatgtggggaaactggactgtaatttgtggtggtggggaattgcgttcgcgctttcttct

SEQ ID NO: 19 - RNA, 154 nt, synthetic construct
  source 1..154: mol_type = other RNA
atatttacttggcaggggagataacgtgaccacgaaggtggttttcccagggctgaggcttattcattgtactccggatgtgctgacccctgcgatttccccaaatgtgggaaactcgactgcataatttgtggtagtggggggctgtgtccgt

SEQ ID NO: 20 - RNA, 154 nt, synthetic construct
  source 1..154: mol_type = other RNA
tatttacttggcaggggagataacgtgaccacgaaggtggttttcccagggctgaggcttattcattgtactccggatgtgctgacccctgcgatttccccaaatgtgggaaactcgactgcataatttgtggtagtggggggctgtgtccgtg

SEQ ID NO: 21 - RNA, 154 nt, synthetic construct
  source 1..154: mol_type = other RNA
atttacttggcaggggagataacgtgaccacgaaggtggttttcccagggctgaggcttattcattgtactccggatgtgctgacccctgcgatttccccaaatgtgggaaactcgactgcataatttgtggtagtggggggctgtgtccgtgc

SEQ ID NO: 22 - RNA, 154 nt, synthetic construct
  source 1..154: mol_type = other RNA
tttacttggcaggggagataacgtgaccacgaaggtggttttcccagggctgaggcttattcattgtactccggatgtgctgacccctgcgatttccccaaatgtgggaaactcgactgcataatttgtggtagtggggggctgtgtccgtgct

SEQ ID NO: 23 - RNA, 154 nt, synthetic construct
  source 1..154: mol_type = other RNA
ttacttggcaggggagataacgtgaccacgaaggtggttttcccagggctgaggcttattcattgtactccggatgtgctgacccctgcgatttccccaaatgtgggaaactcgactgcataatttgtggtagtggggggctgtgtccgtgctt

SEQ ID NO: 24 - RNA, 154 nt, synthetic construct
  source 1..154: mol_type = other RNA
tacttggcaggggagataacgtgaccacgaaggtggttttcccagggctgaggcttattcattgtactccggatgtgctgacccctgcgatttccccaaatgtgggaaactcgactgcataatttgtggtagtggggggctgtgtccgtgcttt

SEQ ID NO: 25 - RNA, 154 nt, synthetic construct
  source 1..154: mol_type = other RNA
acttggcaggggagataacgtgaccacgaaggtggttttcccagggctgaggcttattcattgtactccggatgtgctgacccctgcgatttccccaaatgtgggaaactcgactgcataatttgtggtagtggggggctgtgtccgtgctttt

SEQ ID NO: 26 - RNA, 154 nt, synthetic construct
  source 1..154: mol_type = other RNA
cttggcaggggagataacgtgaccacgaaggtggttttcccagggctgaggcttattcattgtactccggatgtgctgacccctgcgatttccccaaatgtgggaaactcgactgcataatttgtggtagtggggggctgtgtccgtgcttttc

SEQ ID NO: 27 - RNA, 154 nt, synthetic construct
  source 1..154: mol_type = other RNA
ttggcaggggagataacgtgaccacgaaggtggttttcccagggctgaggcttattcattgtactccggatgtgctgacccctgcgatttccccaaatgtgggaaactcgactgcataatttgtggtagtggggggctgtgtccgtgcttttcc

SEQ ID NO: 28 - RNA, 154 nt, synthetic construct
  source 1..154: mol_type = other RNA
tggcaggggagataacgtgaccacgaaggtggttttcccagggctgaggcttattcattgtactccggatgtgctgacccctgcgatttccccaaatgtgggaaactcgactgcataatttgtggtagtggggggctgtgtccgtgcttttccc

SEQ ID NO: 29 - RNA, 154 nt, synthetic construct
  source 1..154: mol_type = other RNA
atacttacctggcagggcagataccatgatcttaaaggcagttttcccagggcaaggcttatccattccactctggatccattataggggcatgctgatccctggaattgccccaaatgtgggaagctctactgcaaaatttttggtagtgagc

SEQ ID NO: 30 - RNA, 154 nt, synthetic construct
  source 1..154: mol_type = other RNA
tacttacctggcagggcagataccatgatcttaaaggcagttttcccagggcaaggcttatccattccactctggatccattataggggcatgctgatccctggaattgccccaaatgtgggaagctctactgcaaaatttttggtagtgagcg

SEQ ID NO: 31 - RNA, 154 nt, synthetic construct
  source 1..154: mol_type = other RNA
acttacctggcagggcagataccatgatcttaaaggcagttttcccagggcaaggcttatccattccactctggatccattataggggcatgctgatccctggaattgccccaaatgtgggaagctctactgcaaaatttttggtagtgagcga

SEQ ID NO: 32 - RNA, 154 nt, synthetic construct
  source 1..154: mol_type = other RNA
cttacctggcagggcagataccatgatcttaaaggcagttttcccagggcaaggcttatccattccactctggatccattataggggcatgctgatccctggaattgccccaaatgtgggaagctctactgcaaaatttttggtagtgagcgat

SEQ ID NO: 33 - RNA, 154 nt, synthetic construct
  source 1..154: mol_type = other RNA
ttacctggcagggcagataccatgatcttaaaggcagttttcccagggcaaggcttatccattccactctggatccattataggggcatgctgatccctggaattgccccaaatgtgggaagctctactgcaaaatttttggtagtgagcgatg

SEQ ID NO: 34 - RNA, 154 nt, synthetic construct
  source 1..154: mol_type = other RNA
tacctggcagggcagataccatgatcttaaaggcagttttcccagggcaaggcttatccattccactctggatccattataggggcatgctgatccctggaattgccccaaatgtgggaagctctactgcaaaatttttggtagtgagcgatgg

SEQ ID NO: 35 - RNA, 154 nt, synthetic construct
  source 1..154: mol_type = other RNA
acctggcagggcagataccatgatcttaaaggcagttttcccagggcaaggcttatccattccactctggatccattataggggcatgctgatccctggaattgccccaaatgtgggaagctctactgcaaaatttttggtagtgagcgatggc

SEQ ID NO: 36 - RNA, 154 nt, synthetic construct
  source 1..154: mol_type = other RNA
cctggcagggcagataccatgatcttaaaggcagttttcccagggcaaggcttatccattccactctggatccattataggggcatgctgatccctggaattgccccaaatgtgggaagctctactgcaaaatttttggtagtgagcgatggca

SEQ ID NO: 37 - RNA, 154 nt, synthetic construct
  source 1..154: mol_type = other RNA
ctggcagggcagataccatgatcttaaaggcagttttcccagggcaaggcttatccattccactctggatccattataggggcatgctgatccctggaattgccccaaatgtgggaagctctactgcaaaatttttggtagtgagcgatggcat

SEQ ID NO: 38 - RNA, 154 nt, synthetic construct
  source 1..154: mol_type = other RNA
tggcagggcagataccatgatcttaaaggcagttttcccagggcaaggcttatccattccactctggatccattataggggcatgctgatccctggaattgccccaaatgtgggaagctctactgcaaaatttttggtagtgagcgatggcatt

SEQ ID NO: 39 - RNA, 154 nt, synthetic construct
  source 1..154: mol_type = other RNA
atacttacctggcaggggagataccatgatcacgaaggtggttttcccagggcgaggcttatccattgcactccggatgtgctgacccctgcgatttccccaaatgtgggaaactcgactgcataatttgtggtagtgggggactgcgttcgcg

SEQ ID NO: 40 - RNA, 154 nt, synthetic construct
  source 1..154: mol_type = other RNA
tacttacctggcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgc

SEQ ID NO: 41 - RNA, 154 nt, synthetic construct
  source 1..154: mol_type = other RNA
acttacctggcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgct

SEQ ID NO: 42 - RNA, 154 nt, synthetic construct
  source 1..154: mol_type = other RNA
cttacctggcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgctt

SEQ ID NO: 43 - RNA, 154 nt, synthetic construct
  source 1..154: mol_type = other RNA
ttacctggcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgcttt

SEQ ID NO: 44 - RNA, 154 nt, synthetic construct
  source 1..154: mol_type = other RNA
tacctggcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgctttc

SEQ ID NO: 45 - RNA, 154 nt, synthetic construct
  source 1..154: mol_type = other RNA
acctggcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgctttcc

SEQ ID NO: 46 - RNA, 154 nt, synthetic construct
  source 1..154: mol_type = other RNA
cctggcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgctttccc

SEQ ID NO: 47 - RNA, 154 nt, synthetic construct
  source 1..154: mol_type = other RNA
ctggcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgctttcccc

SEQ ID NO: 48 - RNA, 154 nt, synthetic construct
  source 1..154: mol_type = other RNA
tggcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgctttcccct

SEQ ID NO: 49 - RNA, 154 nt, synthetic construct
  source 1..154: mol_type = other RNA
atacttccctgacaggggagatacctaggaatccaacttcccagggcaagactgatctattgcactatggatgtgccgacccctgagatttacaaaattgtgggaaactcaactgcataatttatggaaatgaaggactgtgtttgcgctttca

SEQ ID NO: 50 - RNA, 153 nt, synthetic construct
  source 1..153: mol_type = other RNA
tacttccctgacaggggagatacctaggaatccaacttcccagggcaagactgatctattgcactatggatgtgccgacccctgagatttacaaaattgtgggaaactcaactgcataatttatggaaatgaaggactgtgtttgcgctttca

SEQ ID NO: 51 - RNA, 152 nt, synthetic construct
  source 1..152: mol_type = other RNA
acttccctgacaggggagatacctaggaatccaacttcccagggcaagactgatctattgcactatggatgtgccgacccctgagatttacaaaattgtgggaaactcaactgcataatttatggaaatgaaggactgtgtttgcgctttca

SEQ ID NO: 52 - RNA, 151 nt, synthetic construct
  source 1..151: mol_type = other RNA
cttccctgacaggggagatacctaggaatccaacttcccagggcaagactgatctattgcactatggatgtgccgacccctgagatttacaaaattgtgggaaactcaactgcataatttatggaaatgaaggactgtgtttgcgctttca

SEQ ID NO: 53 - RNA, 150 nt, synthetic construct
  source 1..150: mol_type = other RNA
ttccctgacaggggagatacctaggaatccaacttcccagggcaagactgatctattgcactatggatgtgccgacccctgagatttacaaaattgtgggaaactcaactgcataatttatggaaatgaaggactgtgtttgcgctttca

SEQ ID NO: 54 - RNA, 149 nt, synthetic construct
  source 1..149: mol_type = other RNA
tccctgacaggggagatacctaggaatccaacttcccagggcaagactgatctattgcactatggatgtgccgacccctgagatttacaaaattgtgggaaactcaactgcataatttatggaaatgaaggactgtgtttgcgctttca

SEQ ID NO: 55 - RNA, 148 nt, synthetic construct
  source 1..148: mol_type = other RNA
ccctgacaggggagatacctaggaatccaacttcccagggcaagactgatctattgcactatggatgtgccgacccctgagatttacaaaattgtgggaaactcaactgcataatttatggaaatgaaggactgtgtttgcgctttca

SEQ ID NO: 56 - RNA, 147 nt, synthetic construct
  source 1..147: mol_type = other RNA
cctgacaggggagatacctaggaatccaacttcccagggcaagactgatctattgcactatggatgtgccgacccctgagatttacaaaattgtgggaaactcaactgcataatttatggaaatgaaggactgtgtttgcgctttca

SEQ ID NO: 57 - RNA, 146 nt, synthetic construct
  source 1..146: mol_type = other RNA
ctgacaggggagatacctaggaatccaacttcccagggcaagactgatctattgcactatggatgtgccgacccctgagatttacaaaattgtgggaaactcaactgcataatttatggaaatgaaggactgtgtttgcgctttca

SEQ ID NO: 58 - RNA, 145 nt, synthetic construct
  source 1..145: mol_type = other RNA
tgacaggggagatacctaggaatccaacttcccagggcaagactgatctattgcactatggatgtgccgacccctgagatttacaaaattgtgggaaactcaactgcataatttatggaaatgaaggactgtgtttgcgctttca

SEQ ID NO: 59 - RNA, 154 nt, synthetic construct
  source 1..154: mol_type = other RNA
aaacttctctgccaggggagataccatgatcacaaaggtggctttctcagggaaaggctgatttattgcactccagatgtgctgacccctgagatttctccaaatgtgggaaactcagctgcataatttgtggaagcgaaggactgtgtttgtg

SEQ ID NO: 60 - RNA, 154 nt, synthetic construct
  source 1..154: mol_type = other RNA
aacttctctgccaggggagataccatgatcacaaaggtggctttctcagggaaaggctgatttattgcactccagatgtgctgacccctgagatttctccaaatgtgggaaactcagctgcataatttgtggaagcgaaggactgtgtttgtgc

SEQ ID NO: 61 - RNA, 154 nt, synthetic construct
  source 1..154: mol_type = other RNA
acttctctgccaggggagataccatgatcacaaaggtggctttctcagggaaaggctgatttattgcactccagatgtgctgacccctgagatttctccaaatgtgggaaactcagctgcataatttgtggaagcgaaggactgtgtttgtgct

SEQ ID NO: 62 - RNA, 154 nt, synthetic construct
  source 1..154: mol_type = other RNA
cttctctgccaggggagataccatgatcacaaaggtggctttctcagggaaaggctgatttattgcactccagatgtgctgacccctgagatttctccaaatgtgggaaactcagctgcataatttgtggaagcgaaggactgtgtttgtgctt

SEQ ID NO: 63 - RNA, 154 nt, synthetic construct
  source 1..154: mol_type = other RNA
ttctctgccaggggagataccatgatcacaaaggtggctttctcagggaaaggctgatttattgcactccagatgtgctgacccctgagatttctccaaatgtgggaaactcagctgcataatttgtggaagcgaaggactgtgtttgtgcttt

SEQ ID NO: 64 - RNA, 154 nt, synthetic construct
  source 1..154: mol_type = other RNA
tctctgccaggggagataccatgatcacaaaggtggctttctcagggaaaggctgatttattgcactccagatgtgctgacccctgagatttctccaaatgtgggaaactcagctgcataatttgtggaagcgaaggactgtgtttgtgctttc

SEQ ID NO: 65 - RNA, 154 nt, synthetic construct
  source 1..154: mol_type = other RNA
ctctgccaggggagataccatgatcacaaaggtggctttctcagggaaaggctgatttattgcactccagatgtgctgacccctgagatttctccaaatgtgggaaactcagctgcataatttgtggaagcgaaggactgtgtttgtgctttca

SEQ ID NO: 66 - RNA, 154 nt, synthetic construct
  source 1..154: mol_type = other RNA
tctgccaggggagataccatgatcacaaaggtggctttctcagggaaaggctgatttattgcactccagatgtgctgacccctgagatttctccaaatgtgggaaactcagctgcataatttgtggaagcgaaggactgtgtttgtgctttcat

SEQ ID NO: 67 - RNA, 154 nt, synthetic construct
  source 1..154: mol_type = other RNA
ctgccaggggagataccatgatcacaaaggtggctttctcagggaaaggctgatttattgcactccagatgtgctgacccctgagatttctccaaatgtgggaaactcagctgcataatttgtggaagcgaaggactgtgtttgtgctttcatg

SEQ ID NO: 68 - RNA, 154 nt, synthetic construct
  source 1..154: mol_type = other RNA
tgccaggggagataccatgatcacaaaggtggctttctcagggaaaggctgatttattgcactccagatgtgctgacccctgagatttctccaaatgtgggaaactcagctgcataatttgtggaagcgaaggactgtgtttgtgctttcatgt

SEQ ID NO: 69 - RNA, 154 nt, synthetic construct
  source 1..154: mol_type = other RNA
atacttctctgccaggggagatactgtgatcatgaaggtggctttcccagggcaaggctgatctattgcattctggatgtgctgactcctacgatttccccaagtgtgggaaactcaactgcataatttgtggaagtaatgactgtgtttgcac

SEQ ID NO: 70 - RNA, 154 nt, synthetic construct
  source 1..154: mol_type = other RNA
tacttctctgccaggggagatactgtgatcatgaaggtggctttcccagggcaaggctgatctattgcattctggatgtgctgactcctacgatttccccaagtgtgggaaactcaactgcataatttgtggaagtaatgactgtgtttgcact

SEQ ID NO: 71 - RNA, 154 nt, synthetic construct
  source 1..154: mol_type = other RNA
acttctctgccaggggagatactgtgatcatgaaggtggctttcccagggcaaggctgatctattgcattctggatgtgctgactcctacgatttccccaagtgtgggaaactcaactgcataatttgtggaagtaatgactgtgtttgcactt

SEQ ID NO: 72 - RNA, 154 nt, synthetic construct
  source 1..154: mol_type = other RNA
cttctctgccaggggagatactgtgatcatgaaggtggctttcccagggcaaggctgatctattgcattctggatgtgctgactcctacgatttccccaagtgtgggaaactcaactgcataatttgtggaagtaatgactgtgtttgcacttt

SEQ ID NO: 73 - RNA, 154 nt, synthetic construct
  source 1..154: mol_type = other RNA
ttctctgccaggggagatactgtgatcatgaaggtggctttcccagggcaaggctgatctattgcattctggatgtgctgactcctacgatttccccaagtgtgggaaactcaactgcataatttgtggaagtaatgactgtgtttgcactttc

SEQ ID NO: 74 - RNA, 154 nt, synthetic construct
  source 1..154: mol_type = other RNA
tctctgccaggggagatactgtgatcatgaaggtggctttcccagggcaaggctgatctattgcattctggatgtgctgactcctacgatttccccaagtgtgggaaactcaactgcataatttgtggaagtaatgactgtgtttgcactttca

SEQ ID NO: 75 - RNA, 154 nt, synthetic construct
  source 1..154: mol_type = other RNA
ctctgccaggggagatactgtgatcatgaaggtggctttcccagggcaaggctgatctattgcattctggatgtgctgactcctacgatttccccaagtgtgggaaactcaactgcataatttgtggaagtaatgactgtgtttgcactttcac

SEQ ID NO: 76 - RNA, 154 nt, synthetic construct
  source 1..154: mol_type = other RNA
tctgccaggggagatactgtgatcatgaaggtggctttcccagggcaaggctgatctattgcattctggatgtgctgactcctacgatttccccaagtgtgggaaactcaactgcataatttgtggaagtaatgactgtgtttgcactttcacg

SEQ ID NO: 77 - RNA, 154 nt, synthetic construct
  source 1..154: mol_type = other RNA
ctgccaggggagatactgtgatcatgaaggtggctttcccagggcaaggctgatctattgcattctggatgtgctgactcctacgatttccccaagtgtgggaaactcaactgcataatttgtggaagtaatgactgtgtttgcactttcacgt

SEQ ID NO: 78 - RNA, 153 nt, synthetic construct
  source 1..153: mol_type = other RNA
tgccaggggagatactgtgatcatgaaggtggctttcccagggcaaggctgatctattgcattctggatgtgctgactcctacgatttccccaagtgtgggaaactcaactgcataatttgtggaagtaatgactgtgtttgcactttcacgt

SEQ ID NO: 79 - RNA, 154 nt, synthetic construct
  source 1..154: mol_type = other RNA
atacttccctgccaggggagataccgtgatcatgaaggtggctttcccagggaaaggccgatatgttgcactctagatgtgctgacccgtgagatttccccaaatgtgggacactcaaatgcataattggtggaagtgaaggactgtgtttgtg

SEQ ID NO: 80 - RNA, 154 nt, synthetic construct
  source 1..154: mol_type = other RNA
tacttccctgccaggggagataccgtgatcatgaaggtggctttcccagggaaaggccgatatgttgcactctagatgtgctgacccgtgagatttccccaaatgtgggacactcaaatgcataattggtggaagtgaaggactgtgtttgtgc

SEQ ID NO: 81 - RNA, 154 nt, synthetic construct
  source 1..154: mol_type = other RNA
acttccctgccaggggagataccgtgatcatgaaggtggctttcccagggaaaggccgatatgttgcactctagatgtgctgacccgtgagatttccccaaatgtgggacactcaaatgcataattggtggaagtgaaggactgtgtttgtgct

SEQ ID NO: 82 - RNA, 154 nt, synthetic construct
  source 1..154: mol_type = other RNA
cttccctgccaggggagataccgtgatcatgaaggtggctttcccagggaaaggccgatatgttgcactctagatgtgctgacccgtgagatttccccaaatgtgggacactcaaatgcataattggtggaagtgaaggactgtgtttgtgctt

SEQ ID NO: 83 - RNA, 154 nt, synthetic construct
  source 1..154: mol_type = other RNA
ttccctgccaggggagataccgtgatcatgaaggtggctttcccagggaaaggccgatatgttgcactctagatgtgctgacccgtgagatttccccaaatgtgggacactcaaatgcataattggtggaagtgaaggactgtgtttgtgcttt

SEQ ID NO: 84 - RNA, 154 nt, synthetic construct
  source 1..154: mol_type = other RNA
tccctgccaggggagataccgtgatcatgaaggtggctttcccagggaaaggccgatatgttgcactctagatgtgctgacccgtgagatttccccaaatgtgggacactcaaatgcataattggtggaagtgaaggactgtgtttgtgctttc

SEQ ID NO: 85 - RNA, 154 nt, synthetic construct
  source 1..154: mol_type = other RNA
ccctgccaggggagataccgtgatcatgaaggtggctttcccagggaaaggccgatatgttgcactctagatgtgctgacccgtgagatttccccaaatgtgggacactcaaatgcataattggtggaagtgaaggactgtgtttgtgctttca

SEQ ID NO: 86 - RNA, 153 nt, synthetic construct
  source 1..153: mol_type = other RNA
cctgccaggggagataccgtgatcatgaaggtggctttcccagggaaaggccgatatgttgcactctagatgtgctgacccgtgagatttccccaaatgtgggacactcaaatgcataattggtggaagtgaaggactgtgtttgtgctttca

SEQ ID NO: 87 - RNA, 152 nt, synthetic construct
  source 1..152: mol_type = other RNA
ctgccaggggagataccgtgatcatgaaggtggctttcccagggaaaggccgatatgttgcactctagatgtgctgacccgtgagatttccccaaatgtgggacactcaaatgcataattggtggaagtgaaggactgtgtttgtgctttca

SEQ ID NO: 88 - RNA, 151 nt, synthetic construct
  source 1..151: mol_type = other RNA
tgccaggggagataccgtgatcatgaaggtggctttcccagggaaaggccgatatgttgcactctagatgtgctgacccgtgagatttccccaaatgtgggacactcaaatgcataattggtggaagtgaaggactgtgtttgtgctttca

SEQ ID NO: 89 - RNA, 154 nt, synthetic construct
  source 1..154: mol_type = other RNA
ttaactacctgacagaggagatactgtgatcatgaaagtggttttcctagggcaagacttatccgttgcactccagatgtgctgactcatgcaatttccccaaatgtgggaaactcgactacataatttctggtggtaggggactgcgttcatg

SEQ ID NO: 90 - RNA, 154 nt, synthetic construct
  source 1..154: mol_type = other RNA
taactacctgacagaggagatactgtgatcatgaaagtggttttcctagggcaagacttatccgttgcactccagatgtgctgactcatgcaatttccccaaatgtgggaaactcgactacataatttctggtggtaggggactgcgttcatgt

SEQ ID NO: 91 - RNA, 154 nt, synthetic construct
  source 1..154: mol_type = other RNA
aactacctgacagaggagatactgtgatcatgaaagtggttttcctagggcaagacttatccgttgcactccagatgtgctgactcatgcaatttccccaaatgtgggaaactcgactacataatttctggtggtaggggactgcgttcatgtt

SEQ ID NO: 92 - RNA, 154 nt, synthetic construct
  source 1..154: mol_type = other RNA
actacctgacagaggagatactgtgatcatgaaagtggttttcctagggcaagacttatccgttgcactccagatgtgctgactcatgcaatttccccaaatgtgggaaactcgactacataatttctggtggtaggggactgcgttcatgttc

SEQ ID NO: 93 - RNA, 154 nt, synthetic construct
  source 1..154: mol_type = other RNA
ctacctgacagaggagatactgtgatcatgaaagtggttttcctagggcaagacttatccgttgcactccagatgtgctgactcatgcaatttccccaaatgtgggaaactcgactacataatttctggtggtaggggactgcgttcatgttct

SEQ ID NO: 94 - RNA, 154 nt, synthetic construct
  source 1..154: mol_type = other RNA
tacctgacagaggagatactgtgatcatgaaagtggttttcctagggcaagacttatccgttgcactccagatgtgctgactcatgcaatttccccaaatgtgggaaactcgactacataatttctggtggtaggggactgcgttcatgttctc

SEQ ID NO: 95 - RNA, 154 nt, synthetic construct
  source 1..154: mol_type = other RNA
acctgacagaggagatactgtgatcatgaaagtggttttcctagggcaagacttatccgttgcactccagatgtgctgactcatgcaatttccccaaatgtgggaaactcgactacataatttctggtggtaggggactgcgttcatgttctcc

SEQ ID NO: 96 - RNA, 154 nt, synthetic construct
  source 1..154: mol_type = other RNA
cctgacagaggagatactgtgatcatgaaagtggttttcctagggcaagacttatccgttgcactccagatgtgctgactcatgcaatttccccaaatgtgggaaactcgactacataatttctggtggtaggggactgcgttcatgttctccc

SEQ ID NO: 97 - RNA, 154 nt, synthetic construct
  source 1..154: mol_type = other RNA
ctgacagaggagatactgtgatcatgaaagtggttttcctagggcaagacttatccgttgcactccagatgtgctgactcatgcaatttccccaaatgtgggaaactcgactacataatttctggtggtaggggactgcgttcatgttctcccc

SEQ ID NO: 98 - RNA, 154 nt, synthetic construct
  source 1..154: mol_type = other RNA
tgacagaggagatactgtgatcatgaaagtggttttcctagggcaagacttatccgttgcactccagatgtgctgactcatgcaatttccccaaatgtgggaaactcgactacataatttctggtggtaggggactgcgttcatgttctcccct

SEQ ID NO: 99 - RNA, 147 nt, synthetic construct
  source 1..147: mol_type = other RNA
agacttatctggcaggggagacaccatgaacatgaggatagttttcccaaggcaagtttcaacccttgcactctagatgatgagattacttaatgggtacaatgcatgtgatttgggtaatggatactgtagaaacactgacgtcac

SEQ ID NO: 100 - RNA, 146 nt, synthetic construct
  source 1..146: mol_type = other RNA
gacttatctggcaggggagacaccatgaacatgaggatagttttcccaaggcaagtttcaacccttgcactctagatgatgagattacttaatgggtacaatgcatgtgatttgggtaatggatactgtagaaacactgacgtcac

SEQ ID NO: 101 - RNA, 145 nt, synthetic construct
  source 1..145: mol_type = other RNA
acttatctggcaggggagacaccatgaacatgaggatagttttcccaaggcaagtttcaacccttgcactctagatgatgagattacttaatgggtacaatgcatgtgatttgggtaatggatactgtagaaacactgacgtcac

SEQ ID NO: 102 - RNA, 144 nt, synthetic construct
  source 1..144: mol_type = other RNA
cttatctggcaggggagacaccatgaacatgaggatagttttcccaaggcaagtttcaacccttgcactctagatgatgagattacttaatgggtacaatgcatgtgatttgggtaatggatactgtagaaacactgacgtcac

SEQ ID NO: 103 - RNA, 143 nt, synthetic construct
  source 1..143: mol_type = other RNA
ttatctggcaggggagacaccatgaacatgaggatagttttcccaaggcaagtttcaacccttgcactctagatgatgagattacttaatgggtacaatgcatgtgatttgggtaatggatactgtagaaacactgacgtcac

SEQ ID NO: 104 - RNA, 142 nt, synthetic construct
  source 1..142: mol_type = other RNA
tatctggcaggggagacaccatgaacatgaggatagttttcccaaggcaagtttcaacccttgcactctagatgatgagattacttaatgggtacaatgcatgtgatttgggtaatggatactgtagaaacactgacgtcac

SEQ ID NO: 105 - RNA, 141 nt, synthetic construct
  source 1..141: mol_type = other RNA
atctggcaggggagacaccatgaacatgaggatagttttcccaaggcaagtttcaacccttgcactctagatgatgagattacttaatgggtacaatgcatgtgatttgggtaatggatactgtagaaacactgacgtcac

SEQ ID NO: 106 - RNA, 140 nt, synthetic construct
  source 1..140: mol_type = other RNA
tctggcaggggagacaccatgaacatgaggatagttttcccaaggcaagtttcaacccttgcactctagatgatgagattacttaatgggtacaatgcatgtgatttgggtaatggatactgtagaaacactgacgtcac

SEQ ID NO: 107 - RNA, 139 nt, synthetic construct
  source 1..139: mol_type = other RNA
ctggcaggggagacaccatgaacatgaggatagttttcccaaggcaagtttcaacccttgcactctagatgatgagattacttaatgggtacaatgcatgtgatttgggtaatggatactgtagaaacactgacgtcac

SEQ ID NO: 108 - RNA, 138 nt, synthetic construct
  source 1..138: mol_type = other RNA
tggcaggggagacaccatgaacatgaggatagttttcccaaggcaagtttcaacccttgcactctagatgatgagattacttaatgggtacaatgcatgtgatttgggtaatggatactgtagaaacactgacgtcac

SEQ ID NO: 109 - RNA, 149 nt, synthetic construct
  source 1..149: mol_type = other RNA
agactcatctggcagcggagataccatgaacatcacggtagttttcccaaagcaagtttccacccttgcactccggatgatgagacattacttaatggatacaatgcatgtgatttgagtgatggatactgtggaaacaccgacttcac

SEQ ID NO: 110 - RNA, 148 nt, synthetic construct
  source 1..148: mol_type = other RNA
gactcatctggcagcggagataccatgaacatcacggtagttttcccaaagcaagtttccacccttgcactccggatgatgagacattacttaatggatacaatgcatgtgatttgagtgatggatactgtggaaacaccgacttcac

SEQ ID NO: 111 - RNA, 147 nt, synthetic construct
  source 1..147: mol_type = other RNA
actcatctggcagcggagataccatgaacatcacggtagttttcccaaagcaagtttccacccttgcactccggatgatgagacattacttaatggatacaatgcatgtgatttgagtgatggatactgtggaaacaccgacttcac

SEQ ID NO: 112 - RNA, 146 nt, synthetic construct
  source 1..146: mol_type = other RNA
ctcatctggcagcggagataccatgaacatcacggtagttttcccaaagcaagtttccacccttgcactccggatgatgagacattacttaatggatacaatgcatgtgatttgagtgatggatactgtggaaacaccgacttcac

SEQ ID NO: 113 - RNA, 145 nt, synthetic construct
  source 1..145: mol_type = other RNA
tcatctggcagcggagataccatgaacatcacggtagttttcccaaagcaagtttccacccttgcactccggatgatgagacattacttaatggatacaatgcatgtgatttgagtgatggatactgtggaaacaccgacttcac

SEQ ID NO: 114 - RNA, 144 nt, synthetic construct
  source 1..144: mol_type = other RNA
catctggcagcggagataccatgaacatcacggtagttttcccaaagcaagtttccacccttgcactccggatgatgagacattacttaatggatacaatgcatgtgatttgagtgatggatactgtggaaacaccgacttcac

SEQ ID NO: 115 - RNA, 143 nt, synthetic construct
  source 1..143: mol_type = other RNA
atctggcagcggagataccatgaacatcacggtagttttcccaaagcaagtttccacccttgcactccggatgatgagacattacttaatggatacaatgcatgtgatttgagtgatggatactgtggaaacaccgacttcac

SEQ ID NO: 116 - RNA, 142 nt, synthetic construct
  source 1..142: mol_type = other RNA
tctggcagcggagataccatgaacatcacggtagttttcccaaagcaagtttccacccttgcactccggatgatgagacattacttaatggatacaatgcatgtgatttgagtgatggatactgtggaaacaccgacttcac

SEQ ID NO: 117 - RNA, 141 nt, synthetic construct
  source 1..141: mol_type = other RNA
ctggcagcggagataccatgaacatcacggtagttttcccaaagcaagtttccacccttgcactccggatgatgagacattacttaatggatacaatgcatgtgatttgagtgatggatactgtggaaacaccgacttcac

SEQ ID NO: 118 - RNA, 140 nt, synthetic construct
  source 1..140: mol_type = other RNA
tggcagcggagataccatgaacatcacggtagttttcccaaagcaagtttccacccttgcactccggatgatgagacattacttaatggatacaatgcatgtgatttgagtgatggatactgtggaaacaccgacttcac

SEQ ID NO: 119 - RNA, 154 nt, synthetic construct
  source 1..154: mol_type = other RNA
agacttggcaggggagatagcatgatcacgaaggtggttttcccaaggcaagatttattcactgcactctggatgtgctgacccctacgatttccgcccaatggggaagcttgactgcttagtttgttgtggcaggggactgtgttcacacttt

SEQ ID NO: 120 - RNA, 154 nt, synthetic construct
  source 1..154: mol_type = other RNA
gacttggcaggggagatagcatgatcacgaaggtggttttcccaaggcaagatttattcactgcactctggatgtgctgacccctacgatttccgcccaatggggaagcttgactgcttagtttgttgtggcaggggactgtgttcacactttt

SEQ ID NO: 121 - RNA, 154 nt, synthetic construct
  source 1..154: mol_type = other RNA
acttggcaggggagatagcatgatcacgaaggtggttttcccaaggcaagatttattcactgcactctggatgtgctgacccctacgatttccgcccaatggggaagcttgactgcttagtttgttgtggcaggggactgtgttcacacttttc

SEQ ID NO: 122 - RNA, 154 nt, synthetic construct
  source 1..154: mol_type = other RNA
cttggcaggggagatagcatgatcacgaaggtggttttcccaaggcaagatttattcactgcactctggatgtgctgacccctacgatttccgcccaatggggaagcttgactgcttagtttgttgtggcaggggactgtgttcacacttttcc

SEQ ID NO: 123 - RNA, 154 nt, synthetic construct
  source 1..154: mol_type = other RNA
ttggcaggggagatagcatgatcacgaaggtggttttcccaaggcaagatttattcactgcactctggatgtgctgacccctacgatttccgcccaatggggaagcttgactgcttagtttgttgtggcaggggactgtgttcacacttttccc

SEQ ID NO: 124 - RNA, 154 nt, synthetic construct
  source 1..154: mol_type = other RNA
tggcaggggagatagcatgatcacgaaggtggttttcccaaggcaagatttattcactgcactctggatgtgctgacccctacgatttccgcccaatggggaagcttgactgcttagtttgttgtggcaggggactgtgttcacacttttcccg

SEQ ID NO: 125 - RNA, 154 nt, synthetic construct
  source 1..154: mol_type = other RNA
ggcaggggagatagcatgatcacgaaggtggttttcccaaggcaagatttattcactgcactctggatgtgctgacccctacgatttccgcccaatggggaagcttgactgcttagtttgttgtggcaggggactgtgttcacacttttcccgg

SEQ ID NO: 126 - RNA, 153 nt, synthetic construct
  source 1..153: mol_type = other RNA
gcaggggagatagcatgatcacgaaggtggttttcccaaggcaagatttattcactgcactctggatgtgctgacccctacgatttccgcccaatggggaagcttgactgcttagtttgttgtggcaggggactgtgttcacacttttcccgg

SEQ ID NO: 127 - RNA, 152 nt, synthetic construct
  source 1..152: mol_type = other RNA
caggggagatagcatgatcacgaaggtggttttcccaaggcaagatttattcactgcactctggatgtgctgacccctacgatttccgcccaatggggaagcttgactgcttagtttgttgtggcaggggactgtgttcacacttttcccgg

SEQ ID NO: 128 - RNA, 151 nt, synthetic construct
  source 1..151: mol_type = other RNA
aggggagatagcatgatcacgaaggtggttttcccaaggcaagatttattcactgcactctggatgtgctgacccctacgatttccgcccaatggggaagcttgactgcttagtttgttgtggcaggggactgtgttcacacttttcccgg

SEQ ID NO: 129 - RNA, 154 nt, synthetic construct
  source 1..154: mol_type = other RNA
atacttaacatggcagaggagatatcataatcacaaaggtagttttcccagggcaagccttatccactgcattccagatgtgctcacctctgtggtttccccaaatgtggaaaactggactgcataatttgtggtagcgggggactgcattaat

SEQ ID NO: 130 - RNA, 154 nt, synthetic construct
  source 1..154: mol_type = other RNA
tacttaacatggcagaggagatatcataatcacaaaggtagttttcccagggcaagccttatccactgcattccagatgtgctcacctctgtggtttccccaaatgtggaaaactggactgcataatttgtggtagcgggggactgcattaata

SEQ ID NO: 131 - RNA, 154 nt, synthetic construct
  source 1..154: mol_type = other RNA
acttaacatggcagaggagatatcataatcacaaaggtagttttcccagggcaagccttatccactgcattccagatgtgctcacctctgtggtttccccaaatgtggaaaactggactgcataatttgtggtagcgggggactgcattaatac

SEQ ID NO: 132 - RNA, 154 nt, synthetic construct
  source 1..154: mol_type = other RNA
cttaacatggcagaggagatatcataatcacaaaggtagttttcccagggcaagccttatccactgcattccagatgtgctcacctctgtggtttccccaaatgtggaaaactggactgcataatttgtggtagcgggggactgcattaatacc

SEQ ID NO: 133 - RNA, 154 nt, synthetic construct
  source 1..154: mol_type = other RNA
ttaacatggcagaggagatatcataatcacaaaggtagttttcccagggcaagccttatccactgcattccagatgtgctcacctctgtggtttccccaaatgtggaaaactggactgcataatttgtggtagcgggggactgcattaatacct

SEQ ID NO: 134 - RNA, 154 nt, synthetic construct
  source 1..154: mol_type = other RNA
taacatggcagaggagatatcataatcacaaaggtagttttcccagggcaagccttatccactgcattccagatgtgctcacctctgtggtttccccaaatgtggaaaactggactgcataatttgtggtagcgggggactgcattaatacctt

SEQ ID NO: 135 - RNA, 154 nt, synthetic construct
  source 1..154: mol_type = other RNA
aacatggcagaggagatatcataatcacaaaggtagttttcccagggcaagccttatccactgcattccagatgtgctcacctctgtggtttccccaaatgtggaaaactggactgcataatttgtggtagcgggggactgcattaataccttc

SEQ ID NO: 136 - RNA, 154 nt, synthetic construct
  source 1..154: mol_type = other RNA
acatggcagaggagatatcataatcacaaaggtagttttcccagggcaagccttatccactgcattccagatgtgctcacctctgtggtttccccaaatgtggaaaactggactgcataatttgtggtagcgggggactgcattaataccttcc

SEQ ID NO: 137 - RNA, 154 nt, synthetic construct
  source 1..154: mol_type = other RNA
catggcagaggagatatcataatcacaaaggtagttttcccagggcaagccttatccactgcattccagatgtgctcacctctgtggtttccccaaatgtggaaaactggactgcataatttgtggtagcgggggactgcattaataccttcct

SEQ ID NO: 138 - RNA, 154 nt, synthetic construct
  source 1..154: mol_type = other RNA
atggcagaggagatatcataatcacaaaggtagttttcccagggcaagccttatccactgcattccagatgtgctcacctctgtggtttccccaaatgtggaaaactggactgcataatttgtggtagcgggggactgcattaataccttcctc

SEQ ID NO: 139 - RNA, 133 nt, synthetic construct
  source 1..133: mol_type = other RNA
aaaaagggcttctgctgtgagtggcacacatagggcatcgtttgctcttggtgccagaatcaacatcaagagatttcagaagcataattttttggtacttgggcagctggtgatcattggtcctgtagccctt

SEQ ID NO: 140 - RNA, 132 nt, synthetic construct
  source 1..132: mol_type = other RNA
aaaagggcttctgctgtgagtggcacacatagggcatcgtttgctcttggtgccagaatcaacatcaagagatttcagaagcataattttttggtacttgggcagctggtgatcattggtcctgtagccctt

SEQ ID NO: 141 - RNA, 131 nt, synthetic construct
  source 1..131: mol_type = other RNA
aaagggcttctgctgtgagtggcacacatagggcatcgtttgctcttggtgccagaatcaacatcaagagatttcagaagcataattttttggtacttgggcagctggtgatcattggtcctgtagccctt

SEQ ID NO: 142 - RNA, 130 nt, synthetic construct
  source 1..130: mol_type = other RNA
aagggcttctgctgtgagtggcacacatagggcatcgtttgctcttggtgccagaatcaacatcaagagatttcagaagcataattttttggtacttgggcagctggtgatcattggtcctgtagccctt

SEQ ID NO: 143 - RNA, 129 nt, synthetic construct
  source 1..129: mol_type = other RNA
agggcttctgctgtgagtggcacacatagggcatcgtttgctcttggtgccagaatcaacatcaagagatttcagaagcataattttttggtacttgggcagctggtgatcattggtcctgtagccctt

SEQ ID NO: 144 - RNA, 128 nt, synthetic construct
  source 1..128: mol_type = other RNA
gggcttctgctgtgagtggcacacatagggcatcgtttgctcttggtgccagaatcaacatcaagagatttcagaagcataattttttggtacttgggcagctggtgatcattggtcctgtagccctt

SEQ ID NO: 145 - RNA, 127 nt, synthetic construct
  source 1..127: mol_type = other RNA
ggcttctgctgtgagtggcacacatagggcatcgtttgctcttggtgccagaatcaacatcaagagatttcagaagcataattttttggtacttgggcagctggtgatcattggtcctgtagccctt

SEQ ID NO: 146 - RNA, 126 nt, synthetic construct
  source 1..126: mol_type = other RNA
gcttctgctgtgagtggcacacatagggcatcgtttgctcttggtgccagaatcaacatcaagagatttcagaagcataattttttggtacttgggcagctggtgatcattggtcctgtagccctt

SEQ ID NO: 147 - RNA, 125 nt, synthetic construct
  source 1..125: mol_type = other RNA
cttctgctgtgagtggcacacatagggcatcgtttgctcttggtgccagaatcaacatcaagagatttcagaagcataattttttggtacttgggcagctggtgatcattggtcctgtagccctt

SEQ ID NO: 148 - RNA, 124 nt, synthetic construct
  source 1..124: mol_type = other RNA
ttctgctgtgagtggcacacatagggcatcgtttgctcttggtgccagaatcaacatcaagagatttcagaagcataattttttggtacttgggcagctggtgatcattggtcctgtagccctt

SEQ ID NO: 149 - RNA, 129 nt, synthetic construct
  source 1..129: mol_type = other RNA
aaaaaaagctgctgttgtgagtgatacatgcagggcaacttgattgctcttagtgcagaattgacatcaaggaattttggaagtataattttttggcaggtggatagctggttgtattagtccattctc

SEQ ID NO: 150 - RNA, 128 nt, synthetic construct
  source 1..128: mol_type = other RNA
aaaaaagctgctgttgtgagtgatacatgcagggcaacttgattgctcttagtgcagaattgacatcaaggaattttggaagtataattttttggcaggtggatagctggttgtattagtccattctc

SEQ ID NO: 151 - RNA, 127 nt, synthetic construct
  source 1..127: mol_type = other RNA
aaaaagctgctgttgtgagtgatacatgcagggcaacttgattgctcttagtgcagaattgacatcaaggaattttggaagtataattttttggcaggtggatagctggttgtattagtccattctc

SEQ ID NO: 152 - RNA, 126 nt, synthetic construct
  source 1..126: mol_type = other RNA
aaaagctgctgttgtgagtgatacatgcagggcaacttgattgctcttagtgcagaattgacatcaaggaattttggaagtataattttttggcaggtggatagctggttgtattagtccattctc

SEQ ID NO: 153 - RNA, 125 nt, synthetic construct
  source 1..125: mol_type = other RNA
aaagctgctgttgtgagtgatacatgcagggcaacttgattgctcttagtgcagaattgacatcaaggaattttggaagtataattttttggcaggtggatagctggttgtattagtccattctc

SEQ ID NO: 154 - RNA, 124 nt, synthetic construct
  source 1..124: mol_type = other RNA
aagctgctgttgtgagtgatacatgcagggcaacttgattgctcttagtgcagaattgacatcaaggaattttggaagtataattttttggcaggtggatagctggttgtattagtccattctc

SEQ ID NO: 155 - RNA, 123 nt, synthetic construct
  source 1..123: mol_type = other RNA
agctgctgttgtgagtgatacatgcagggcaacttgattgctcttagtgcagaattgacatcaaggaattttggaagtataattttttggcaggtggatagctggttgtattagtccattctc

SEQ ID NO: 156 - RNA, 122 nt, synthetic construct
  source 1..122: mol_type = other RNA
gctgctgttgtgagtgatacatgcagggcaacttgattgctcttagtgcagaattgacatcaaggaattttggaagtataattttttggcaggtggatagctggttgtattagtccattctc

SEQ ID NO: 157 - RNA, 121 nt, synthetic construct
  source 1..121: mol_type = other RNA
ctgctgttgtgagtgatacatgcagggcaacttgattgctcttagtgcagaattgacatcaaggaattttggaagtataattttttggcaggtggatagctggttgtattagtccattctc

SEQ ID NO: 158 - RNA, 120 nt, synthetic construct
  source 1..120: mol_type = other RNA
tgctgttgtgagtgatacatgcagggcaacttgattgctcttagtgcagaattgacatcaaggaattttggaagtataattttttggcaggtggatagctggttgtattagtccattctc

SEQ ID NO: 159 - RNA, 133 nt, synthetic construct
  source 1..133: mol_type = other RNA
aaaaagggcttctgtcatgagtggcacacataggacaactcaatttctcttcatgcagaataaacatcaagagattttggaagcgtaatttttggtagttgggcagctggtgatcactggtgccagcaccctt

SEQ ID NO: 160 - RNA, 132 nt, synthetic construct
  source 1..132: mol_type = other RNA
aaaagggcttctgtcatgagtggcacacataggacaactcaatttctcttcatgcagaataaacatcaagagattttggaagcgtaatttttggtagttgggcagctggtgatcactggtgccagcaccctt

SEQ ID NO: 161 - RNA, 131 nt, synthetic construct
  source 1..131: mol_type = other RNA
aaagggcttctgtcatgagtggcacacataggacaactcaatttctcttcatgcagaataaacatcaagagattttggaagcgtaatttttggtagttgggcagctggtgatcactggtgccagcaccctt

SEQ ID NO: 162 - RNA, 130 nt, synthetic construct
  source 1..130: mol_type = other RNA
aagggcttctgtcatgagtggcacacataggacaactcaatttctcttcatgcagaataaacatcaagagattttggaagcgtaatttttggtagttgggcagctggtgatcactggtgccagcaccctt

SEQ ID NO: 163 - RNA, 129 nt, synthetic construct
  source 1..129: mol_type = other RNA
agggcttctgtcatgagtggcacacataggacaactcaatttctcttcatgcagaataaacatcaagagattttggaagcgtaatttttggtagttgggcagctggtgatcactggtgccagcaccctt

SEQ ID NO: 164 - RNA, 128 nt, synthetic construct
  source 1..128: mol_type = other RNA
gggcttctgtcatgagtggcacacataggacaactcaatttctcttcatgcagaataaacatcaagagattttggaagcgtaatttttggtagttgggcagctggtgatcactggtgccagcaccctt

SEQ ID NO: 165 - RNA, 127 nt, synthetic construct
  source 1..127: mol_type = other RNA
ggcttctgtcatgagtggcacacataggacaactcaatttctcttcatgcagaataaacatcaagagattttggaagcgtaatttttggtagttgggcagctggtgatcactggtgccagcaccctt

SEQ ID NO: 166 - RNA, 126 nt, synthetic construct
  source 1..126: mol_type = other RNA
gcttctgtcatgagtggcacacataggacaactcaatttctcttcatgcagaataaacatcaagagattttggaagcgtaatttttggtagttgggcagctggtgatcactggtgccagcaccctt

SEQ ID NO: 167 - RNA, 125 nt, synthetic construct
  source 1..125: mol_type = other RNA
cttctgtcatgagtggcacacataggacaactcaatttctcttcatgcagaataaacatcaagagattttggaagcgtaatttttggtagttgggcagctggtgatcactggtgccagcaccctt

SEQ ID NO: 168 - RNA, 124 nt, synthetic construct
  source 1..124: mol_type = other RNA
ttctgtcatgagtggcacacataggacaactcaatttctcttcatgcagaataaacatcaagagattttggaagcgtaatttttggtagttgggcagctggtgatcactggtgccagcaccctt

SEQ ID NO: 169 - RNA, 133 nt, synthetic construct
  source 1..133: mol_type = other RNA
aaaaagggcttctgtcatgagtggcctatgtagggcaaccagattgctcttcatgtggaattgacatcaagagctttcagaagtgtattttttggaagttgggcagctggtaatcattggtcttggcatcctt

SEQ ID NO: 170 - RNA, 132 nt, synthetic construct
  source 1..132: mol_type = other RNA
aaaagggcttctgtcatgagtggcctatgtagggcaaccagattgctcttcatgtggaattgacatcaagagctttcagaagtgtattttttggaagttgggcagctggtaatcattggtcttggcatcctt

SEQ ID NO: 171 - RNA, 131 nt, synthetic construct
  source 1..131: mol_type = other RNA
aaagggcttctgtcatgagtggcctatgtagggcaaccagattgctcttcatgtggaattgacatcaagagctttcagaagtgtattttttggaagttgggcagctggtaatcattggtcttggcatcctt

SEQ ID NO: 172 - RNA, 130 nt, synthetic construct
  source 1..130: mol_type = other RNA
aagggcttctgtcatgagtggcctatgtagggcaaccagattgctcttcatgtggaattgacatcaagagctttcagaagtgtattttttggaagttgggcagctggtaatcattggtcttggcatcctt

SEQ ID NO: 173 - RNA, 129 nt, synthetic construct
  source 1..129: mol_type = other RNA
agggcttctgtcatgagtggcctatgtagggcaaccagattgctcttcatgtggaattgacatcaagagctttcagaagtgtattttttggaagttgggcagctggtaatcattggtcttggcatcctt

SEQ ID NO: 174 - RNA, 128 nt, synthetic construct
  source 1..128: mol_type = other RNA
gggcttctgtcatgagtggcctatgtagggcaaccagattgctcttcatgtggaattgacatcaagagctttcagaagtgtattttttggaagttgggcagctggtaatcattggtcttggcatcctt

SEQ ID NO: 175 - RNA, 127 nt, synthetic construct
  source 1..127: mol_type = other RNA
ggcttctgtcatgagtggcctatgtagggcaaccagattgctcttcatgtggaattgacatcaagagctttcagaagtgtattttttggaagttgggcagctggtaatcattggtcttggcatcctt

SEQ ID NO: 176 - RNA, 126 nt, synthetic construct
  source 1..126: mol_type = other RNA
gcttctgtcatgagtggcctatgtagggcaaccagattgctcttcatgtggaattgacatcaagagctttcagaagtgtattttttggaagttgggcagctggtaatcattggtcttggcatcctt

SEQ ID NO: 177 - RNA, 125 nt, synthetic construct
  source 1..125: mol_type = other RNA
cttctgtcatgagtggcctatgtagggcaaccagattgctcttcatgtggaattgacatcaagagctttcagaagtgtattttttggaagttgggcagctggtaatcattggtcttggcatcctt

SEQ ID NO: 178 - RNA, 124 nt, synthetic construct
  source 1..124: mol_type = other RNA
ttctgtcatgagtggcctatgtagggcaaccagattgctcttcatgtggaattgacatcaagagctttcagaagtgtattttttggaagttgggcagctggtaatcattggtcttggcatcctt

SEQ ID NO: 179 - RNA, 142 nt, synthetic construct
  source 1..142: mol_type = other RNA
aaagggggcttctgtcatgagtggcacacattgggcaactcaactgctcttcatgaggaatcaacatcaggaggttttggaagaatgatttttttggtagttgggcagcttgtgagaaaaaaatgttttcaggcaaatcctc

SEQ ID NO: 180 - RNA, 141 nt, synthetic construct
  source 1..141: mol_type = other RNA
aagggggcttctgtcatgagtggcacacattgggcaactcaactgctcttcatgaggaatcaacatcaggaggttttggaagaatgatttttttggtagttgggcagcttgtgagaaaaaaatgttttcaggcaaatcctc

SEQ ID NO: 181 - RNA, 140 nt, synthetic construct
  source 1..140: mol_type = other RNA
agggggcttctgtcatgagtggcacacattgggcaactcaactgctcttcatgaggaatcaacatcaggaggttttggaagaatgatttttttggtagttgggcagcttgtgagaaaaaaatgttttcaggcaaatcctc

SEQ ID NO: 182 - RNA, 139 nt, synthetic construct
  source 1..139: mol_type = other RNA
gggggcttctgtcatgagtggcacacattgggcaactcaactgctcttcatgaggaatcaacatcaggaggttttggaagaatgatttttttggtagttgggcagcttgtgagaaaaaaatgttttcaggcaaatcctc

SEQ ID NO: 183 - RNA, 138 nt, synthetic construct
  source 1..138: mol_type = other RNA
ggggcttctgtcatgagtggcacacattgggcaactcaactgctcttcatgaggaatcaacatcaggaggttttggaagaatgatttttttggtagttgggcagcttgtgagaaaaaaatgttttcaggcaaatcctc

SEQ ID NO: 184 - RNA, 137 nt, synthetic construct
  source 1..137: mol_type = other RNA
gggcttctgtcatgagtggcacacattgggcaactcaactgctcttcatgaggaatcaacatcaggaggttttggaagaatgatttttttggtagttgggcagcttgtgagaaaaaaatgttttcaggcaaatcctc

SEQ ID NO: 185 - RNA, 136 nt, synthetic construct
  source 1..136: mol_type = other RNA
ggcttctgtcatgagtggcacacattgggcaactcaactgctcttcatgaggaatcaacatcaggaggttttggaagaatgatttttttggtagttgggcagcttgtgagaaaaaaatgttttcaggcaaatcctc

SEQ ID NO: 186 - RNA, 135 nt, synthetic construct
  source 1..135: mol_type = other RNA
gcttctgtcatgagtggcacacattgggcaactcaactgctcttcatgaggaatcaacatcaggaggttttggaagaatgatttttttggtagttgggcagcttgtgagaaaaaaatgttttcaggcaaatcctc

SEQ ID NO: 187 - RNA, 134 nt, synthetic construct
  source 1..134: mol_type = other RNA
cttctgtcatgagtggcacacattgggcaactcaactgctcttcatgaggaatcaacatcaggaggttttggaagaatgatttttttggtagttgggcagcttgtgagaaaaaaatgttttcaggcaaatcctc

SEQ ID NO: 188 - RNA, 133 nt, synthetic construct
  source 1..133: mol_type = other RNA
ttctgtcatgagtggcacacattgggcaactcaactgctcttcatgaggaatcaacatcaggaggttttggaagaatgatttttttggtagttgggcagcttgtgagaaaaaaatgttttcaggcaaatcctc

SEQ ID NO: 189 - RNA, 134 nt, synthetic construct
  source 1..134: mol_type = other RNA
aaaaagggcttctgtcgtgagtggcacacgtagggcaactcgattgctctgcgtgcggaatcgacatcaagagatttcggaagcataattttttggtatttgggcagctggtgatcgttggtcccggcgccctt

SEQ ID NO: 190 - RNA, 133 nt, synthetic construct
  source 1..133: mol_type = other RNA
aaaagggcttctgtcgtgagtggcacacgtagggcaactcgattgctctgcgtgcggaatcgacatcaagagatttcggaagcataattttttggtatttgggcagctggtgatcgttggtcccggcgccctt

SEQ ID NO: 191 - RNA, 132 nt, synthetic construct
  source 1..132: mol_type = other RNA
aaagggcttctgtcgtgagtggcacacgtagggcaactcgattgctctgcgtgcggaatcgacatcaagagatttcggaagcataattttttggtatttgggcagctggtgatcgttggtcccggcgccctt

SEQ ID NO: 192 - RNA, 131 nt, synthetic construct
  source 1..131: mol_type = other RNA
aagggcttctgtcgtgagtggcacacgtagggcaactcgattgctctgcgtgcggaatcgacatcaagagatttcggaagcataattttttggtatttgggcagctggtgatcgttggtcccggcgccctt

SEQ ID NO: 193 - RNA, 130 nt, synthetic construct
  source 1..130: mol_type = other RNA
agggcttctgtcgtgagtggcacacgtagggcaactcgattgctctgcgtgcggaatcgacatcaagagatttcggaagcataattttttggtatttgggcagctggtgatcgttggtcccggcgccctt

SEQ ID NO: 194 - RNA, 129 nt, synthetic construct
  source 1..129: mol_type = other RNA
gggcttctgtcgtgagtggcacacgtagggcaactcgattgctctgcgtgcggaatcgacatcaagagatttcggaagcataattttttggtatttgggcagctggtgatcgttggtcccggcgccctt

SEQ ID NO: 195 - RNA, 128 nt, synthetic construct
  source 1..128: mol_type = other RNA
ggcttctgtcgtgagtggcacacgtagggcaactcgattgctctgcgtgcggaatcgacatcaagagatttcggaagcataattttttggtatttgggcagctggtgatcgttggtcccggcgccctt

SEQ ID NO: 196 - RNA, 127 nt, synthetic construct
  source 1..127: mol_type = other RNA
gcttctgtcgtgagtggcacacgtagggcaactcgattgctctgcgtgcggaatcgacatcaagagatttcggaagcataattttttggtatttgggcagctggtgatcgttggtcccggcgccctt

SEQ ID NO: 197 - RNA, 126 nt, synthetic construct
  source 1..126: mol_type = other RNA
cttctgtcgtgagtggcacacgtagggcaactcgattgctctgcgtgcggaatcgacatcaagagatttcggaagcataattttttggtatttgggcagctggtgatcgttggtcccggcgccctt

SEQ ID NO: 198 - RNA, 125 nt, synthetic construct
  source 1..125: mol_type = other RNA
ttctgtcgtgagtggcacacgtagggcaactcgattgctctgcgtgcggaatcgacatcaagagatttcggaagcataattttttggtatttgggcagctggtgatcgttggtcccggcgccctt

SEQ ID NO: 199 - RNA, 131 nt, synthetic construct
  source 1..131: mol_type = other RNA
aaaaagggcttctgtcgtgagtggcacacgtagggcaactcgattgctctgcgtgcggaatcgacatcaagagatttcggaagcataattttttggtatttgggcagctggtgatcgttggtcccggcgcc

SEQ ID NO: 200 - RNA, 130 nt, synthetic construct
  source 1..130: mol_type = other RNA
aaaagggcttctgtcgtgagtggcacacgtagggcaactcgattgctctgcgtgcggaatcgacatcaagagatttcggaagcataattttttggtatttgggcagctggtgatcgttggtcccggcgcc

SEQ ID NO: 201 - RNA, 129 nt, synthetic construct
  source 1..129: mol_type = other RNA
aaagggcttctgtcgtgagtggcacacgtagggcaactcgattgctctgcgtgcggaatcgacatcaagagatttcggaagcataattttttggtatttgggcagctggtgatcgttggtcccggcgcc

SEQ ID NO: 202 - RNA, 128 nt, synthetic construct
  source 1..128: mol_type = other RNA
aagggcttctgtcgtgagtggcacacgtagggcaactcgattgctctgcgtgcggaatcgacatcaagagatttcggaagcataattttttggtatttgggcagctggtgatcgttggtcccggcgcc

SEQ ID NO: 203 - RNA, 127 nt, synthetic construct
  source 1..127: mol_type = other RNA
agggcttctgtcgtgagtggcacacgtagggcaactcgattgctctgcgtgcggaatcgacatcaagagatttcggaagcataattttttggtatttgggcagctggtgatcgttggtcccggcgcc

SEQ ID NO: 204 - RNA, 126 nt, synthetic construct
  source 1..126: mol_type = other RNA
gggcttctgtcgtgagtggcacacgtagggcaactcgattgctctgcgtgcggaatcgacatcaagagatttcggaagcataattttttggtatttgggcagctggtgatcgttggtcccggcgcc

SEQ ID NO: 205 - RNA, 125 nt, synthetic construct
  source 1..125: mol_type = other RNA
ggcttctgtcgtgagtggcacacgtagggcaactcgattgctctgcgtgcggaatcgacatcaagagatttcggaagcataattttttggtatttgggcagctggtgatcgttggtcccggcgcc

SEQ ID NO: 206 - RNA, 124 nt, synthetic construct
  source 1..124: mol_type = other RNA
gcttctgtcgtgagtggcacacgtagggcaactcgattgctctgcgtgcggaatcgacatcaagagatttcggaagcataattttttggtatttgggcagctggtgatcgttggtcccggcgcc

SEQ ID NO: 207 - RNA, 123 nt, synthetic construct
  source 1..123: mol_type = other RNA
cttctgtcgtgagtggcacacgtagggcaactcgattgctctgcgtgcggaatcgacatcaagagatttcggaagcataattttttggtatttgggcagctggtgatcgttggtcccggcgcc

SEQ ID NO: 208 - RNA, 122 nt, synthetic construct
  source 1..122: mol_type = other RNA
ttctgtcgtgagtggcacacgtagggcaactcgattgctctgcgtgcggaatcgacatcaagagatttcggaagcataattttttggtatttgggcagctggtgatcgttggtcccggcgcc

SEQ ID NO: 209 - RNA, 11 nt, synthetic construct
  source 1..11: mol_type = other RNA
aatttttggag

SEQ ID NO: 210 - RNA, 11 nt, synthetic construct
  source 1..11: mol_type = other RNA
aatttttggag

SEQ ID NO: 211 - RNA, 11 nt, synthetic construct
  source 1..11: mol_type = other RNA
tatttttggag

SEQ ID NO: 212 - RNA, 11 nt, synthetic construct
  source 1..11: mol_type = other RNA
gatttttggag

SEQ ID NO: 213 - RNA, 11 nt, synthetic construct
  source 1..11: mol_type = other RNA
catttttggag

SEQ ID NO: 214 - RNA, 11 nt, synthetic construct
  source 1..11: mol_type = other RNA
attttttggag

SEQ ID NO: 215 - RNA, 11 nt, synthetic construct
  source 1..11: mol_type = other RNA
agtttttggag

SEQ ID NO: 216 - RNA, 11 nt, synthetic construct
  source 1..11: mol_type = other RNA
actttttggag

SEQ ID NO: 217 - RNA, 11 nt, synthetic construct
  source 1..11: mol_type = other RNA
aaattttggag

SEQ ID NO: 218 - RNA, 11 nt, synthetic construct
  source 1..11: mol_type = other RNA
aagttttggag

SEQ ID NO: 219 - RNA, 11 nt, synthetic construct
  source 1..11: mol_type = other RNA
aacttttggag

SEQ ID NO: 220 - RNA, 11 nt, synthetic construct
  source 1..11: mol_type = other RNA
aatatttggag

SEQ ID NO: 221 - RNA, 11 nt, synthetic construct
  source 1..11: mol_type = other RNA
aatgtttggag

SEQ ID NO: 222 - RNA, 11 nt, synthetic construct
  source 1..11: mol_type = other RNA
aatctttggag

SEQ ID NO: 223 - RNA, 11 nt, synthetic construct
  source 1..11: mol_type = other RNA
aattattggag

SEQ ID NO: 224 - RNA, 11 nt, synthetic construct
  source 1..11: mol_type = other RNA
aattgttggag

SEQ ID NO: 225 - RNA, 11 nt, synthetic construct
  source 1..11: mol_type = other RNA
aattcttggag

SEQ ID NO: 226 - RNA, 11 nt, synthetic construct
  source 1..11: mol_type = other RNA
aatttatggag

SEQ ID NO: 227 - RNA, 11 nt, synthetic construct
  source 1..11: mol_type = other RNA
aatttgtggag

SEQ ID NO: 228 - RNA, 11 nt, synthetic construct
  source 1..11: mol_type = other RNA
aatttctggag

SEQ ID NO: 229 - RNA, 11 nt, synthetic construct
  source 1..11: mol_type = other RNA
aattttaggag

SEQ ID NO: 230 - RNA, 11 nt, synthetic construct
  source 1..11: mol_type = other RNA
aattttgggag

SEQ ID NO: 231 - RNA, 11 nt, synthetic construct
  source 1..11: mol_type = other RNA
aattttcggag

SEQ ID NO: 232 - RNA, 11 nt, synthetic construct
  source 1..11: mol_type = other RNA
aatttttagag

SEQ ID NO: 233 - RNA, 11 nt, synthetic construct
  source 1..11: mol_type = other RNA
aattttttgag

SEQ ID NO: 234 - RNA, 11 nt, synthetic construct
  source 1..11: mol_type = other RNA
aatttttcgag

SEQ ID NO: 235 - RNA, 11 nt, synthetic construct
  source 1..11: mol_type = other RNA
aatttttgaag

SEQ ID NO: 236 - RNA, 11 nt, synthetic construct
  source 1..11: mol_type = other RNA
aatttttgtag

SEQ ID NO: 237 - RNA, 11 nt, synthetic construct
  source 1..11: mol_type = other RNA
aatttttgcag

SEQ ID NO: 238 - RNA, 11 nt, synthetic construct
  source 1..11: mol_type = other RNA
aatttttggtg

SEQ ID NO: 239 - RNA, 11 nt, synthetic construct
  source 1..11: mol_type = other RNA
aatttttgggg

SEQ ID NO: 240 - RNA, 11 nt, synthetic construct
  source 1..11: mol_type = other RNA
aatttttggcg

SEQ ID NO: 241 - RNA, 11 nt, synthetic construct
  source 1..11: mol_type = other RNA
aatttttggaa

SEQ ID NO: 242 - RNA, 11 nt, synthetic construct
  source 1..11: mol_type = other RNA
aatttttggat

SEQ ID NO: 243 - RNA, 11 nt, synthetic construct
  source 1..11: mol_type = other RNA
aatttttggac

SEQ ID NO: 244 - RNA, 11 nt, synthetic construct
  source 1..11: mol_type = other RNA
aatgttttgag

SEQ ID NO: 245 - RNA, 11 nt, synthetic construct
  source 1..11: mol_type = other RNA
aatttgtgtag

SEQ ID NO: 246 - RNA, 11 nt, synthetic construct
  source 1..11: mol_type = other RNA
agtttttggaa

SEQ ID NO: 247 - RNA, 11 nt, synthetic construct
  source 1..11: mol_type = other RNA
attttgtggag

SEQ ID NO: 248 - RNA, 11 nt, synthetic construct
  source 1..11: mol_type = other RNA
aatgtttgcag

SEQ ID NO: 249 - RNA, 11 nt, synthetic construct
  source 1..11: mol_type = other RNA
aatgttgggag

SEQ ID NO: 250 - RNA, 11 nt, synthetic construct
  source 1..11: mol_type = other RNA
aattgctggag

SEQ ID NO: 251 - RNA, 11 nt, synthetic construct
  source 1..11: mol_type = other RNA
aattttagcag

SEQ ID NO: 252 - RNA, 11 nt, synthetic construct
  source 1..11: mol_type = other RNA
aaggtttggag

SEQ ID NO: 253 - RNA, 11 nt, synthetic construct
  source 1..11: mol_type = other RNA
gatttgtggag

SEQ ID NO: 254 - RNA, 11 nt, synthetic construct
  source 1..11: mol_type = other RNA
actttttggaa

SEQ ID NO: 255 - RNA, 11 nt, synthetic construct
  source 1..11: mol_type = other RNA
aaattatggag

SEQ ID NO: 256 - RNA, 11 nt, synthetic construct
  source 1..11: mol_type = other RNA
aattttgcgag

SEQ ID NO: 257 - RNA, 11 nt, synthetic construct
  source 1..11: mol_type = other RNA
aatttctggaa

SEQ ID NO: 258 - RNA, 11 nt, synthetic construct
  source 1..11: mol_type = other RNA
tatttttggtg

SEQ ID NO: 259 - RNA, 11 nt, synthetic construct
  source 1..11: mol_type = other RNA
gattttaggag

SEQ ID NO: 260 - RNA, 11 nt, synthetic construct
  source 1..11: mol_type = other RNA
aatctgtggag

SEQ ID NO: 261 - RNA, 11 nt, synthetic construct
  source 1..11: mol_type = other RNA
aaattttagag

SEQ ID NO: 262 - RNA, 11 nt, synthetic construct
  source 1..11: mol_type = other RNA
aatttttcgac

SEQ ID NO: 263 - RNA, 11 nt, synthetic construct
  source 1..11: mol_type = other RNA
aatttttgggt

SEQ ID NO: 264 - RNA, 11 nt, synthetic construct
  source 1..11: mol_type = other RNA
aatctttggaa

SEQ ID NO: 265 - RNA, 11 nt, synthetic construct
  source 1..11: mol_type = other RNA
aattattggtg

SEQ ID NO: 266 - RNA, 11 nt, synthetic construct
  source 1..11: mol_type = other RNA
aattttaagag

SEQ ID NO: 267 - RNA, 11 nt, synthetic construct
  source 1..11: mol_type = other RNA
aatgttaggag

SEQ ID NO: 268 - RNA, 11 nt, synthetic construct
  source 1..11: mol_type = other RNA
aattttagaag

SEQ ID NO: 269 - RNA, 11 nt, synthetic construct
  source 1..11: mol_type = other RNA
attttttggcg

SEQ ID NO: 270 - RNA, 11 nt, synthetic construct
  source 1..11: mol_type = other RNA
aacttttggtg

SEQ ID NO: 271 - RNA, 11 nt, synthetic construct
  source 1..11: mol_type = other RNA
aatgtttggat

SEQ ID NO: 272 - RNA, 11 nt, synthetic construct
  source 1..11: mol_type = other RNA
aattttaggat

SEQ ID NO: 273 - RNA, 11 nt, synthetic construct
  source 1..11: mol_type = other RNA
aatttttgatg

SEQ ID NO: 274 - RNA, 11 nt, synthetic construct
  source 1..11: mol_type = other RNA
aattcttggtg

SEQ ID NO: 275 - RNA, 11 nt, synthetic construct
  source 1..11: mol_type = other RNA
aatccttggag

SEQ ID NO: 276 - RNA, 11 nt, synthetic construct
  source 1..11: mol_type = other RNA
aatttttcaag

SEQ ID NO: 277 - RNA, 11 nt, synthetic construct
  source 1..11: mol_type = other RNA
catttttgcag

SEQ ID NO: 278 - RNA, 11 nt, synthetic construct
  source 1..11: mol_type = other RNA
catttttcgag

SEQ ID NO: 279 - RNA, 11 nt, synthetic construct
  source 1..11: mol_type = other RNA
aattttgggcg

SEQ ID NO: 280 - RNA, 11 nt, synthetic construct
  source 1..11: mol_type = other RNA
agtttttgcag

SEQ ID NO: 281 - RNA, 11 nt, synthetic construct
  source 1..11: mol_type = other RNA
aaaatttggag

SEQ ID NO: 282 - RNA, 11 nt, synthetic construct
  source 1..11: mol_type = other RNA
aactcttggag

SEQ ID NO: 283 - RNA, 11 nt, synthetic construct
  source 1..11: mol_type = other RNA
aatttgtggac

SEQ ID NO: 284 - RNA, 11 nt, synthetic construct
  source 1..11: mol_type = other RNA
actttttggtg

SEQ ID NO: 285 - RNA, 11 nt, synthetic construct
  source 1..11: mol_type = other RNA
attttttgaag

SEQ ID NO: 286 - RNA, 11 nt, synthetic construct
  source 1..11: mol_type = other RNA
attttttggaa

SEQ ID NO: 287 - RNA, 11 nt, synthetic construct
  source 1..11: mol_type = other RNA
aagttatggag

SEQ ID NO: 288 - RNA, 11 nt, synthetic construct
  source 1..11: mol_type = other RNA
catttctggag

SEQ ID NO: 289 - RNA, 11 nt, synthetic construct
  source 1..11: mol_type = other RNA
agttcttggag

SEQ ID NO: 290 - RNA, 11 nt, synthetic construct
  source 1..11: mol_type = other RNA
aattttggaag

SEQ ID NO: 291 - RNA, 11 nt, synthetic construct
  source 1..11: mol_type = other RNA
agtatttggag

SEQ ID NO: 292 - RNA, 11 nt, synthetic construct
  source 1..11: mol_type = other RNA
aatttttgggc

SEQ ID NO: 293 - RNA, 11 nt, synthetic construct
  source 1..11: mol_type = other RNA
aattcttgtag

SEQ ID NO: 294 - RNA, 11 nt, synthetic construct
  source 1..11: mol_type = other RNA
aattttgggaa

SEQ ID NO: 295 - RNA, 11 nt, synthetic construct
  source 1..11: mol_type = other RNA
tattttttgag

SEQ ID NO: 296 - RNA, 11 nt, synthetic construct
  source 1..11: mol_type = other RNA
aacttttgtag

SEQ ID NO: 297 - RNA, 11 nt, synthetic construct
  source 1..11: mol_type = other RNA
aattattgcag

SEQ ID NO: 298 - RNA, 11 nt, synthetic construct
  source 1..11: mol_type = other RNA
aatttttggta

SEQ ID NO: 299 - RNA, 11 nt, synthetic construct
  source 1..11: mol_type = other RNA
aattttcgaag

SEQ ID NO: 300 - RNA, 11 nt, synthetic construct
  source 1..11: mol_type = other RNA
tatctttggag

SEQ ID NO: 301 - RNA, 11 nt, synthetic construct
  source 1..11: mol_type = other RNA
aatttttcgtg

SEQ ID NO: 302 - RNA, 11 nt, synthetic construct
  source 1..11: mol_type = other RNA
aatctttggcg

SEQ ID NO: 303 - RNA, 11 nt, synthetic construct
  source 1..11: mol_type = other RNA
gatttatggag

SEQ ID NO: 304 - RNA, 11 nt, synthetic construct
  source 1..11: mol_type = other RNA
aatttttcgaa

SEQ ID NO: 305 - RNA, 11 nt, synthetic construct
  source 1..11: mol_type = other RNA
cctttttggag

SEQ ID NO: 306 - RNA, 11 nt, synthetic construct
  source 1..11: mol_type = other RNA
tatttttgaag

SEQ ID NO: 307 - RNA, 11 nt, synthetic construct
  source 1..11: mol_type = other RNA
aatttttgtaa

SEQ ID NO: 308 - RNA, 11 nt, synthetic construct
  source 1..11: mol_type = other RNA
aattttgagag

SEQ ID NO: 309 - RNA, 11 nt, synthetic construct
  source 1..11: mol_type = other RNA
aagtattggag

SEQ ID NO: 310 - RNA, 11 nt, synthetic construct
  source 1..11: mol_type = other RNA
aattttaggac

SEQ ID NO: 311 - RNA, 11 nt, synthetic construct
  source 1..11: mol_type = other RNA
aatttttaaag

SEQ ID NO: 312 - RNA, 11 nt, synthetic construct
  source 1..11: mol_type = other RNA
aattcttggat

SEQ ID NO: 313 - RNA, 11 nt, synthetic construct
  source 1..11: mol_type = other RNA
aatttgtggtg

SEQ ID NO: 314 - RNA, 11 nt, synthetic construct
  source 1..11: mol_type = other RNA
aattttcgggg

SEQ ID NO: 315 - RNA, 11 nt, synthetic construct
  source 1..11: mol_type = other RNA
acttttttgag

SEQ ID NO: 316 - RNA, 11 nt, synthetic construct
  source 1..11: mol_type = other RNA
aagtttcggag

SEQ ID NO: 317 - RNA, 11 nt, synthetic construct
  source 1..11: mol_type = other RNA
acttttgggag

SEQ ID NO: 318 - RNA, 11 nt, synthetic construct
  source 1..11: mol_type = other RNA
gacttttggag

SEQ ID NO: 319 - RNA, 11 nt, synthetic construct
  source 1..11: mol_type = other RNA
cttttttggag

SEQ ID NO: 320 - RNA, 11 nt, synthetic construct
  source 1..11: mol_type = other RNA
cattgttggag

SEQ ID NO: 321 - RNA, 11 nt, synthetic construct
  source 1..11: mol_type = other RNA
aatattttgag

SEQ ID NO: 322 - RNA, 11 nt, synthetic construct
  source 1..11: mol_type = other RNA
aagttttggcg

SEQ ID NO: 323 - RNA, 11 nt, synthetic construct
  source 1..11: mol_type = other RNA
aattttaggaa

SEQ ID NO: 324 - RNA, 11 nt, synthetic construct
  source 1..11: mol_type = other RNA
aacttgtggag

SEQ ID NO: 325 - RNA, 11 nt, synthetic construct
  source 1..11: mol_type = other RNA
attttttgggg

SEQ ID NO: 326 - RNA, 11 nt, synthetic construct
  source 1..11: mol_type = other RNA
aatttttagat

SEQ ID NO: 327 - RNA, 11 nt, synthetic construct
  source 1..11: mol_type = other RNA
tattttgggag

SEQ ID NO: 328 - RNA, 11 nt, synthetic construct
  source 1..11: mol_type = other RNA
aatttgtgggg

SEQ ID NO: 329 - RNA, 11 nt, synthetic construct
  source 1..11: mol_type = other RNA
aatttttaggg

SEQ ID NO: 330 - RNA, 11 nt, synthetic construct
  source 1..11: mol_type = other RNA
aattttcgtag

SEQ ID NO: 331 - RNA, 11 nt, synthetic construct
  source 1..11: mol_type = other RNA
aattttctgag

SEQ ID NO: 332 - RNA, 11 nt, synthetic construct
  source 1..11: mol_type = other RNA
gattcttggag

SEQ ID NO: 333 - RNA, 11 nt, synthetic construct
  source 1..11: mol_type = other RNA
aatctttggat

SEQ ID NO: 334 - RNA, 11 nt, synthetic construct
  source 1..11: mol_type = other RNA
aaattttggcg

SEQ ID NO: 335 - RNA, 11 nt, synthetic construct
  source 1..11: mol_type = other RNA
aatcattggag

SEQ ID NO: 336 - RNA, 11 nt, synthetic construct
  source 1..11: mol_type = other RNA
aacttttggcg

SEQ ID NO: 337 - RNA, 11 nt, synthetic construct
  source 1..11: mol_type = other RNA
aatttttggga

SEQ ID NO: 338 - RNA, 11 nt, synthetic construct
  source 1..11: mol_type = other RNA
aatgtttggtg

SEQ ID NO: 339 - RNA, 11 nt, synthetic construct
  source 1..11: mol_type = other RNA
aatttatgcag

SEQ ID NO: 340 - RNA, 11 nt, synthetic construct
  source 1..11: mol_type = other RNA
aatctctggag

SEQ ID NO: 341 - RNA, 11 nt, synthetic construct
  source 1..11: mol_type = other RNA
agtttgtggag

SEQ ID NO: 342 - RNA, 11 nt, synthetic construct
  source 1..11: mol_type = other RNA
aagttttggat

SEQ ID NO: 343 - RNA, 11 nt, synthetic construct
  source 1..11: mol_type = other RNA
tatttttggcg

SEQ ID NO: 344 - RNA, 11 nt, synthetic construct
  source 1..11: mol_type = other RNA
aatttctgaag

SEQ ID NO: 345 - RNA, 11 nt, synthetic construct
  source 1..11: mol_type = other RNA
tatttctggag

SEQ ID NO: 346 - RNA, 11 nt, synthetic construct
  source 1..11: mol_type = other RNA
aatttttgctg

SEQ ID NO: 347 - RNA, 11 nt, synthetic construct
  source 1..11: mol_type = other RNA
aaatcttggag

SEQ ID NO: 348 - RNA, 11 nt, synthetic construct
  source 1..11: mol_type = other RNA
aaatttttgag

SEQ ID NO: 349 - RNA, 11 nt, synthetic construct
  source 1..11: mol_type = other RNA
tatttttgtag

SEQ ID NO: 350 - RNA, 11 nt, synthetic construct
  source 1..11: mol_type = other RNA
gattattggag

SEQ ID NO: 351 - RNA, 11 nt, synthetic construct
  source 1..11: mol_type = other RNA
aagttctggag

SEQ ID NO: 352 - RNA, 11 nt, synthetic construct
  source 1..11: mol_type = other RNA
aatctttgaag

SEQ ID NO: 353 - RNA, 11 nt, synthetic construct
  source 1..11: mol_type = other RNA
aaatattggag

SEQ ID NO: 354 - RNA, 11 nt, synthetic construct
  source 1..11: mol_type = other RNA
aactattggag

SEQ ID NO: 355 - RNA, 11 nt, synthetic construct
  source 1..11: mol_type = other RNA
aatatatggag

SEQ ID NO: 356 - RNA, 11 nt, synthetic construct
  source 1..11: mol_type = other RNA
attctttggag

SEQ ID NO: 357 - RNA, 11 nt, synthetic construct
  source 1..11: mol_type = other RNA
gatttttgaag

SEQ ID NO: 358 - RNA, 11 nt, synthetic construct
  source 1..11: mol_type = other RNA
aagttttggtg

SEQ ID NO: 359 - RNA, 11 nt, synthetic construct
  source 1..11: mol_type = other RNA
aatctttgcag

SEQ ID NO: 360 - RNA, 11 nt, synthetic construct
  source 1..11: mol_type = other RNA
actttttggcg

SEQ ID NO: 361 - RNA, 11 nt, synthetic construct
  source 1..11: mol_type = other RNA
gattttgggag

SEQ ID NO: 362 - RNA, 11 nt, synthetic construct
  source 1..11: mol_type = other RNA
catttttggaa

SEQ ID NO: 363 - RNA, 11 nt, synthetic construct
  source 1..11: mol_type = other RNA
aattttttgac

SEQ ID NO: 364 - RNA, 11 nt, synthetic construct
  source 1..11: mol_type = other RNA
aaattttgcag

SEQ ID NO: 365 - RNA, 11 nt, synthetic construct
  source 1..11: mol_type = other RNA
actttttgaag

SEQ ID NO: 366 - RNA, 11 nt, synthetic construct
  source 1..11: mol_type = other RNA
agtctttggag

SEQ ID NO: 367 - RNA, 11 nt, synthetic construct
  source 1..11: mol_type = other RNA
agtttatggag

SEQ ID NO: 368 - RNA, 11 nt, synthetic construct
  source 1..11: mol_type = other RNA
acttcttggag

SEQ ID NO: 369 - RNA, 11 nt, synthetic construct
  source 1..11: mol_type = other RNA
aattttttcag

SEQ ID NO: 370 - RNA, 11 nt, synthetic construct
  source 1..11: mol_type = other RNA
aattttcggat

SEQ ID NO: 371 - RNA, 11 nt, synthetic construct
  source 1..11: mol_type = other RNA
aatttttgtat

SEQ ID NO: 372 - RNA, 11 nt, synthetic construct
  source 1..11: mol_type = other RNA
aatgtctggag

SEQ ID NO: 373 - RNA, 11 nt, synthetic construct
  source 1..11: mol_type = other RNA
aattgttgggg

SEQ ID NO: 374 - RNA, 11 nt, synthetic construct
  source 1..11: mol_type = other RNA
aatttatggac

SEQ ID NO: 375 - RNA, 11 nt, synthetic construct
  source 1..11: mol_type = other RNA
aatttatgaag

SEQ ID NO: 376 - RNA, 11 nt, synthetic construct
  source 1..11: mol_type = other RNA
aatttatgggg

SEQ ID NO: 377 - RNA, 11 nt, synthetic construct
  source 1..11: mol_type = other RNA
aatttgtcgag

SEQ ID NO: 378 - RNA, 11 nt, synthetic construct
  source 1..11: mol_type = other RNA
aattgttggaa

SEQ ID NO: 379 - RNA, 11 nt, synthetic construct
  source 1..11: mol_type = other RNA
tgtttttggag

SEQ ID NO: 380 - RNA, 11 nt, synthetic construct
  source 1..11: mol_type = other RNA
aaatttaggag

SEQ ID NO: 381 - RNA, 11 nt, synthetic construct
  source 1..11: mol_type = other RNA
aattttttgtg

SEQ ID NO: 382 - RNA, 11 nt, synthetic construct
  source 1..11: mol_type = other RNA
cattcttggag

SEQ ID NO: 383 - RNA, 11 nt, synthetic construct
  source 1..11: mol_type = other RNA
aattctcggag

SEQ ID NO: 384 - RNA, 11 nt, synthetic construct
  source 1..11: mol_type = other RNA
aatttttatag

SEQ ID NO: 385 - RNA, 11 nt, synthetic construct
  source 1..11: mol_type = other RNA
actttttagag

SEQ ID NO: 386 - RNA, 11 nt, synthetic construct
  source 1..11: mol_type = other RNA
aattgttagag

SEQ ID NO: 387 - RNA, 11 nt, synthetic construct
  source 1..11: mol_type = other RNA
cgtttttggag

SEQ ID NO: 388 - RNA, 11 nt, synthetic construct
  source 1..11: mol_type = other RNA
aatttttgcgg

SEQ ID NO: 389 - RNA, 11 nt, synthetic construct
  source 1..11: mol_type = other RNA
aagttttgaag

SEQ ID NO: 390 - RNA, 11 nt, synthetic construct
  source 1..11: mol_type = other RNA
aattttcggaa

SEQ ID NO: 391 - RNA, 11 nt, synthetic construct
  source 1..11: mol_type = other RNA
aatttttccag

SEQ ID NO: 392 - RNA, 11 nt, synthetic construct
  source 1..11: mol_type = other RNA
attttttgtag

SEQ ID NO: 393 - RNA, 11 nt, synthetic construct
  source 1..11: mol_type = other RNA
aatctttagag

SEQ ID NO: 394 - RNA, 11 nt, synthetic construct
  source 1..11: mol_type = other RNA
aattttacgag

SEQ ID NO: 395 - RNA, 11 nt, synthetic construct
  source 1..11: mol_type = other RNA
catttttggcg

SEQ ID NO: 396 - RNA, 11 nt, synthetic construct
  source 1..11: mol_type = other RNA
aatttgcggag

SEQ ID NO: 397 - RNA, 11 nt, synthetic construct
  source 1..11: mol_type = other RNA
catctttggag

SEQ ID NO: 398 - RNA, 11 nt, synthetic construct
  source 1..11: mol_type = other RNA
aacttttgaag

SEQ ID NO: 399 - RNA, 11 nt, synthetic construct
  source 1..11: mol_type = other RNA
aatttttctag

SEQ ID NO: 400 - RNA, 11 nt, synthetic construct
  source 1..11: mol_type = other RNA
gaagtagtgat

SEQ ID NO: 401 - RNA, 11 nt, synthetic construct
  source 1..11: mol_type = other RNA
tttacagcaag

SEQ ID NO: 402 - RNA, 11 nt, synthetic construct
  source 1..11: mol_type = other RNA
aattcactact

SEQ ID NO: 403 - RNA, 11 nt, synthetic construct
  source 1..11: mol_type = other RNA
tgaacagaacc

SEQ ID NO: 404 - RNA, 11 nt, synthetic construct
  source 1..11: mol_type = other RNA
gaataggtttg

SEQ ID NO: 405 - RNA, 11 nt, synthetic construct
  source 1..11: mol_type = other RNA
atgaaagttca

SEQ ID NO: 406 - RNA, 11 nt, synthetic construct
  source 1..11: mol_type = other RNA
gagtctgcgtg

SEQ ID NO: 407 - RNA, 11 nt, synthetic construct
  source 1..11: mol_type = other RNA
tccgagttcca

SEQ ID NO: 408 - RNA, 11 nt, synthetic construct
  source 1..11: mol_type = other RNA
tagccggtact

SEQ ID NO: 409 - RNA, 11 nt, synthetic construct
  source 1..11: mol_type = other RNA
tcatgaatgtg

SEQ ID NO: 410 - RNA, 33 nt, synthetic construct
  source 1..33: mol_type = other RNA
caggttttccagtgttcactgaaatttgtctct

SEQ ID NO: 411 - RNA, 31 nt, synthetic construct
  source 1..31: mol_type = other RNA
aaggttttctggtctttatcagaaagcctcc

SEQ ID NO: 412 - RNA, 31 nt, synthetic construct
  source 1..31: mol_type = other RNA
caggttttctggttttcactgcaaaacccca

SEQ ID NO: 413 - RNA, 31 nt, synthetic construct
  source 1..31: mol_type = other RNA
cagactttacggtgcttaccagaaagctccc

SEQ ID NO: 414 - RNA, 45 nt, synthetic construct
  source 1..45: mol_type = other RNA
cacgttttccggttgtcactcccaggtaggctggggaagaggcat

SEQ ID NO: 415 - RNA, 31 nt, synthetic construct
  source 1..31: mol_type = other RNA
cagtctttccagtttttgccggaaagcccct

SEQ ID NO: 416 - RNA, 31 nt, synthetic construct
  source 1..31: mol_type = other RNA
caggctttctggtttttgccagaaagccccc

SEQ ID NO: 417 - RNA, 31 nt, synthetic construct
  source 1..31: mol_type = other RNA
caggtttttcagttttcaccagaatgcccac

SEQ ID NO: 418 - RNA, 31 nt, synthetic construct
  source 1..31: mol_type = other RNA
caggttttctggtctttactggaaagcccaa

SEQ ID NO: 419 - RNA, 31 nt, synthetic construct
  source 1..31: mol_type = other RNA
caggctttccggtttttaccagaaagccccc

SEQ ID NO: 420 - RNA, 31 nt, synthetic construct
  source 1..31: mol_type = other RNA
caggtttttcggtttttactggaaagcccca

SEQ ID NO: 421 - RNA, 31 nt, synthetic construct
  source 1..31: mol_type = other RNA
caggctttccagtaaatacaggaaagccctc

SEQ ID NO: 422 - RNA, 31 nt, synthetic construct
  source 1..31: mol_type = other RNA
caggctttccggtttttgctggaaagactcc

SEQ ID NO: 423 - RNA, 31 nt, synthetic construct
  source 1..31: mol_type = other RNA
aaggttttctggtctttactgaaaagcccca

SEQ ID NO: 424 - RNA, 31 nt, synthetic construct
  source 1..31: mol_type = other RNA
tgggctttctgttttttacgagaaagtcctc

SEQ ID NO: 425 - RNA, 30 nt, synthetic construct
  source 1..30: mol_type = other RNA
cagatttctcagttttcactggaaaccctt

SEQ ID NO: 426 - RNA, 31 nt, synthetic construct
  source 1..31: mol_type = other RNA
caggttttcctgtcttcaccggaactcccca

SEQ ID NO: 427 - RNA, 32 nt, synthetic construct
  source 1..32: mol_type = other RNA
caggttttctggtttgcactagaaaaaccata

SEQ ID NO: 428 - RNA, 31 nt, synthetic construct
  source 1..31: mol_type = other RNA
cagattttctggtttttaccagaaaatttaa

SEQ ID NO: 429 - RNA, 31 nt, synthetic construct
  source 1..31: mol_type = other RNA
caggtcttcttgtttttactggaaaatcctc

SEQ ID NO: 430 - RNA, 31 nt, synthetic construct
  source 1..31: mol_type = other RNA
caggttttccggtcttcaccagaaaacccct

SEQ ID NO: 431 - RNA, 31 nt, synthetic construct
  source 1..31: mol_type = other RNA
caggttttccgatttttactggaaagccctt

SEQ ID NO: 432 - RNA, 29 nt, synthetic construct
  source 1..29: mol_type = other RNA
caggttttctggttttccagaaaacctcc

SEQ ID NO: 433 - RNA, 29 nt, synthetic construct
  source 1..29: mol_type = other RNA
caggttttccagttttcactggaacctct

SEQ ID NO: 434 - RNA, 30 nt, synthetic construct
  source 1..30: mol_type = other RNA
ccggctttccagttttgccggaaagccccc

SEQ ID NO: 435 - RNA, 29 nt, synthetic construct
  source 1..29: mol_type = other RNA
caggttttctggtttttaccagaacttaa

SEQ ID NO: 436 - RNA, 45 nt, synthetic construct
  source 1..45: mol_type = other RNA
cacgttttccggttgtcactcccaggtgggctcgggaagaggcat

SEQ ID NO: 437 - RNA, 31 nt, synthetic construct
  source 1..31: mol_type = other RNA
caggctttccggtctttaccggaaagcctat

SEQ ID NO: 438 - RNA, 31 nt, synthetic construct
  source 1..31: mol_type = other RNA
caggctttctggtttttgccggaaagccctc

SEQ ID NO: 439 - RNA, 31 nt, synthetic construct
  source 1..31: mol_type = other RNA
caggctttcaggtctttgccagaaagcccca

SEQ ID NO: 440 - RNA, 30 nt, synthetic construct
  source 1..30: mol_type = other RNA
caggctttcttgttttcactggaagcctcc

SEQ ID NO: 441 - RNA, 31 nt, synthetic construct
  source 1..31: mol_type = other RNA
caggtttttcagtcttcaccggaaagctccc

SEQ ID NO: 442 - RNA, 37 nt, synthetic construct
  source 1..37: mol_type = other RNA
cagattttctggttttcaccacgaaagaaatgtcatt

SEQ ID NO: 443 - RNA, 30 nt, synthetic construct
  source 1..30: mol_type = other RNA
cagttctcctggattttacaggaaaacccc

SEQ ID NO: 444 - RNA, 31 nt, synthetic construct
  source 1..31: mol_type = other RNA
caggctttctggcatttgccagaaagccctg

SEQ ID NO: 445 - RNA, 31 nt, synthetic construct
  source 1..31: mol_type = other RNA
cagactttcctgtttttaacagaaagccccc

SEQ ID NO: 446 - RNA, 31 nt, synthetic construct
  source 1..31: mol_type = other RNA
caggctttccggtttttgctggaaagctccc

SEQ ID NO: 447 - RNA, 28 nt, synthetic construct
  source 1..28: mol_type = other RNA
cagcttttccagttttcactgaggtaaa

SEQ ID NO: 448 - RNA, 31 nt, synthetic construct
  source 1..31: mol_type = other RNA
taggttttctggtttttattgggaaatcaca

SEQ ID NO: 449 - RNA, 32 nt, synthetic construct
  source 1..32: mol_type = other RNA
cagggtttctggtggtaccagcagactccaca

SEQ ID NO: 450 - RNA, 30 nt, synthetic construct
  source 1..30: mol_type = other RNA
caagtttcccagttttcactggaacctccg

SEQ ID NO: 451 - RNA, 31 nt, synthetic construct
  source 1..31: mol_type = other RNA
cagattttccagtttttactagaaacccccc

SEQ ID NO: 452 - RNA, 31 nt, synthetic construct
  source 1..31: mol_type = other RNA
cagggtttccagttttcactgtataccatcc

SEQ ID NO: 453 - RNA, 31 nt, synthetic construct
  source 1..31: mol_type = other RNA
caagttctctggtcttcactggaaaacccat

SEQ ID NO: 454 - RNA, 31 nt, synthetic construct
  source 1..31: mol_type = other RNA
tagattttctggtctttaccagaaagactcc

SEQ ID NO: 455 - RNA, 32 nt, synthetic construct
  source 1..32: mol_type = other RNA
gcaggttttctggtttttattggaaaaccttc

SEQ ID NO: 456 - RNA, 33 nt, synthetic construct
  source 1..33: mol_type = other RNA
caggttttccacttttcaccagaaactgcctct

SEQ ID NO: 457 - RNA, 31 nt, synthetic construct
  source 1..31: mol_type = other RNA
taggctttccggtttttgcaggaaagccccc

SEQ ID NO: 458 - RNA, 31 nt, synthetic construct
  source 1..31: mol_type = other RNA
caggttttccagtctttaccagaaagccact

SEQ ID NO: 459 - RNA, 33 nt, synthetic construct
  source 1..33: mol_type = other RNA
caggttttttggtcttcacaggaaacttcccct

SEQ ID NO: 460 - RNA, 31 nt, synthetic construct
  source 1..31: mol_type = other RNA
caggttttctgcttttcactggaagactccc

SEQ ID NO: 461 - RNA, 31 nt, synthetic construct
  source 1..31: mol_type = other RNA
cagactttccagtttttgccagaaagcccac

SEQ ID NO: 462 - RNA, 31 nt, synthetic construct
  source 1..31: mol_type = other RNA
caggctttctggtttttgcccaaaagcctct

SEQ ID NO: 463 - RNA, 31 nt, synthetic construct
  source 1..31: mol_type = other RNA
caggttttccaatcttcactgaaaagcttta

SEQ ID NO: 464 - RNA, 31 nt, synthetic construct
  source 1..31: mol_type = other RNA
ctggttcctagtcttcactggaagcaccctc

SEQ ID NO: 465 - RNA, 29 nt, synthetic construct
  source 1..29: mol_type = other RNA
ggctttccgctcttcattggaaagcccat

SEQ ID NO: 466 - RNA, 31 nt, synthetic construct
  source 1..31: mol_type = other RNA
caggtttttcagtttttagcagaaaacctcc

SEQ ID NO: 467 - RNA, 29 nt, synthetic construct
  source 1..29: mol_type = other RNA
caggttctctggtttttactgaaaccaaa

SEQ ID NO: 468 - RNA, 31 nt, synthetic construct
  source 1..31: mol_type = other RNA
caggttttccagtcttcactggaaagccctt

SEQ ID NO: 469 - RNA, 31 nt, synthetic construct
  source 1..31: mol_type = other RNA
caggctttctggtttttgccggaaggcctcc

SEQ ID NO: 470 - RNA, 32 nt, synthetic construct
  source 1..32: mol_type = other RNA
caggttttctgttttttaactagaaaactccc

SEQ ID NO: 471 - RNA, 31 nt, synthetic construct
  source 1..31: mol_type = other RNA
taggctttctggtttttgctggaaagccccc

SEQ ID NO: 472 - RNA, 32 nt, synthetic construct
  source 1..32: mol_type = other RNA
caggtttcccggtttttaccagaaaaatctaa

SEQ ID NO: 473 - RNA, 32 nt, synthetic construct
  source 1..32: mol_type = other RNA
caggttttccagtctttaccagaaatgccccc

SEQ ID NO: 474 - RNA, 30 nt, synthetic construct
  source 1..30: mol_type = other RNA
ggttttctggtcatcgctggaaacaccctc

SEQ ID NO: 475 - RNA, 31 nt, synthetic construct
  source 1..31: mol_type = other RNA
caacctttctggtttttgctagaaagttctc

SEQ ID NO: 476 - RNA, 31 nt, synthetic construct
  source 1..31: mol_type = other RNA
caggcctttgggtctttactggaaaacccct

SEQ ID NO: 477 - RNA, 35 nt, synthetic construct
  source 1..35: mol_type = other RNA
tatgttttccggttttcactcccaggtaggctcgg

SEQ ID NO: 478 - RNA, 31 nt, synthetic construct
  source 1..31: mol_type = other RNA
caaattttctggcttttccaggaaaatcccc

SEQ ID NO: 479 - RNA, 28 nt, synthetic construct
  source 1..28: mol_type = other RNA
taggttttccagtgaccagaaaatcctc

SEQ ID NO: 480 - RNA, 31 nt, synthetic construct
  source 1..31: mol_type = other RNA
caggtcttcgggtttttaactggaaaccttc

SEQ ID NO: 481 - RNA, 32 nt, synthetic construct
  source 1..32: mol_type = other RNA
gcaggctttccagttttttctggaaagcctca

SEQ ID NO: 482 - RNA, 29 nt, synthetic construct
  source 1..29: mol_type = other RNA
ggctttctggtttttactggaaagccccc

SEQ ID NO: 483 - RNA, 31 nt, synthetic construct
  source 1..31: mol_type = other RNA
caggtttcccagtattcaccggaaagctcca

SEQ ID NO: 484 - RNA, 43 nt, synthetic construct
  source 1..43: mol_type = other RNA
caggctttctagttttcccacactaagaaacaaaaaaacctgt

SEQ ID NO: 485 - RNA, 32 nt, synthetic construct
  source 1..32: mol_type = other RNA
gcaagctttctggttttggccaaaaagccacc

SEQ ID NO: 486 - RNA, 24 nt, synthetic construct
  source 1..24: mol_type = other RNA
ttctggttttcaccagaaaccact

SEQ ID NO: 487 - RNA, 32 nt, synthetic construct
  source 1..32: mol_type = other RNA
gcaagttttctagcctgtacctgaaagcctca

SEQ ID NO: 488 - RNA, 30 nt, synthetic construct
  source 1..30: mol_type = other RNA
aagttttcttgtttttaccagaaaactcct

SEQ ID NO: 489 - RNA, 29 nt, synthetic construct
  source 1..29: mol_type = other RNA
ggctttccaatttttgccagaaagccccc

SEQ ID NO: 490 - RNA, 33 nt, synthetic construct
  source 1..33: mol_type = other RNA
agctggttttctggttttcaccggaagacccat

SEQ ID NO: 491 - RNA, 32 nt, synthetic construct
  source 1..32: mol_type = other RNA
caggccttctggtttttgctgggaagtcccca

SEQ ID NO: 492 - RNA, 31 nt, synthetic construct
  source 1..31: mol_type = other RNA
caggttttccagtttgtaccagaaaacccct

SEQ ID NO: 493 - RNA, 22 nt, synthetic construct
  source 1..22: mol_type = other RNA
caggtttcacccaaaaacccac

SEQ ID NO: 494 - RNA, 32 nt, synthetic construct
  source 1..32: mol_type = other RNA
taggctttctggctttttaccggaaagcccct

SEQ ID NO: 495 - RNA, 31 nt, synthetic construct
  source 1..31: mol_type = other RNA
cagggtttctggttttcaccaggaaacaaaa

SEQ ID NO: 496 - RNA, 31 nt, synthetic construct
  source 1..31: mol_type = other RNA
caggttttcctgtctttactagaaacccctc

SEQ ID NO: 497 - RNA, 31 nt, synthetic construct
  source 1..31: mol_type = other RNA
caggttttataggttttaccagaaaacttcc

SEQ ID NO: 498 - RNA, 31 nt, synthetic construct
  source 1..31: mol_type = other RNA
caggcttcccaatttttgctggaaagcccct

SEQ ID NO: 499 - RNA, 31 nt, synthetic construct
  source 1..31: mol_type = other RNA
caggctttttgggttaggtcagaaagtcccc

SEQ ID NO: 500 - RNA, 29 nt, synthetic construct
  source 1..29: mol_type = other RNA
caggtttttggttttttagcgaaaccctc

SEQ ID NO: 501 - RNA, 33 nt, synthetic construct
  source 1..33: mol_type = other RNA
caggctttctggttttcaccataaaatgcccca

SEQ ID NO: 502 - RNA, 43 nt, synthetic construct
  source 1..43: mol_type = other RNA
caggtcttcggggttttacaaaaagaagaagaaatccaccccc

SEQ ID NO: 503 - RNA, 32 nt, synthetic construct
  source 1..32: mol_type = other RNA
caagttctctggtctcccaacaagaaaccccc

SEQ ID NO: 504 - RNA, 33 nt, synthetic construct
  source 1..33: mol_type = other RNA
caggatttctggtttctggtggaaagctcccat

SEQ ID NO: 505 - RNA, 30 nt, synthetic construct
  source 1..30: mol_type = other RNA
aggctttccagtttttgctggaaagcccct

SEQ ID NO: 506 - RNA, 30 nt, synthetic construct
  source 1..30: mol_type = other RNA
caggttttccagtctttgtcaaatgccttc

SEQ ID NO: 507 - RNA, 34 nt, synthetic construct
  source 1..34: mol_type = other RNA
caggctttccagtttttgccagtgtgaacccaaa

SEQ ID NO: 508 - RNA, 31 nt, synthetic construct
  source 1..31: mol_type = other RNA
caggctttctggtttttatcagaaagcctcc

SEQ ID NO: 509 - RNA, 33 nt, synthetic construct
  source 1..33: mol_type = other RNA
gaggttttctggttttcaccagaaccaaccctt

SEQ ID NO: 510 - RNA, 31 nt, synthetic construct
  source 1..31: mol_type = other RNA
cagattttccagcttttactggaagccccct

SEQ ID NO: 511 - RNA, 33 nt, synthetic construct
  source 1..33: mol_type = other RNA
caggttttctggttttcactggaaaatacctca

SEQ ID NO: 512 - RNA, 33 nt, synthetic construct
  source 1..33: mol_type = other RNA
catgttttttggtgttaataaaaacccgcacac

SEQ ID NO: 513 - RNA, 31 nt, synthetic construct
  source 1..31: mol_type = other RNA
aaggctttccggtttttgcaggaaagcaacc

SEQ ID NO: 514 - RNA, 30 nt, synthetic construct
  source 1..30: mol_type = other RNA
agtctttccgggttttgtcagaaaggccct

SEQ ID NO: 515 - RNA, 31 nt, synthetic construct
  source 1..31: mol_type = other RNA
gcaggttttctggtttctactggaagcttct

SEQ ID NO: 516 - RNA, 31 nt, synthetic construct
  source 1..31: mol_type = other RNA
caggctttctggtttttactggaaagcccct

SEQ ID NO: 517 - RNA, 31 nt, synthetic construct
  source 1..31: mol_type = other RNA
caggctttccaatttttgccagaaagctccc

SEQ ID NO: 518 - RNA, 31 nt, synthetic construct
  source 1..31: mol_type = other RNA
caggttttctggttctcaccagaaaacgcca

SEQ ID NO: 519 - RNA, 31 nt, synthetic construct
  source 1..31: mol_type = other RNA
caggttttctggtcttcactggaagaccact

SEQ ID NO: 520 - RNA, 34 nt, synthetic construct
  source 1..34: mol_type = other RNA
caggtggttttccggtctttaccagtaagccccc

SEQ ID NO: 521 - RNA, 31 nt, synthetic construct
  source 1..31: mol_type = other RNA
caggttttccagtttctaccagaaaccccta

SEQ ID NO: 522 - RNA, 32 nt, synthetic construct
  source 1..32: mol_type = other RNA
caggttttctggtctttaccagaaaagtctcc

SEQ ID NO: 523 - RNA, 32 nt, synthetic construct
  source 1..32: mol_type = other RNA
ccaggctttctggtttttaccggaaagccccg

SEQ ID NO: 524 - RNA, 32 nt, synthetic construct
  source 1..32: mol_type = other RNA
caggtttttcactttttactagaaaaaccagt

SEQ ID NO: 525 - RNA, 35 nt, synthetic construct
  source 1..35: mol_type = other RNA
taggctttctgggaggtctttaccagaaagcctcc

SEQ ID NO: 526 - RNA, 32 nt, synthetic construct
  source 1..32: mol_type = other RNA
tacattttccagttttcaccaggaatctgcct

SEQ ID NO: 527 - RNA, 31 nt, synthetic construct
  source 1..31: mol_type = other RNA
taggttttccagttttcaccagaaaatcccg

SEQ ID NO: 528 - RNA, 31 nt, synthetic construct
  source 1..31: mol_type = other RNA
caggttttctggtttttacgagactccccca

SEQ ID NO: 529 - RNA, 30 nt, synthetic construct
  source 1..30: mol_type = other RNA
caggctttctgttttttgctaaaatcctcc

SEQ ID NO: 530 - RNA, 32 nt, synthetic construct
  source 1..32: mol_type = other RNA
ctgggcgtactggctcacgcctataatcccaa

SEQ ID NO: 531 - RNA, 29 nt, synthetic construct
  source 1..29: mol_type = other RNA
ggctctccggtttttgccagaatgcccac

SEQ ID NO: 532 - RNA, 31 nt, synthetic construct
  source 1..31: mol_type = other RNA
tgggttttctggtgaaaactagactgccccc

SEQ ID NO: 533 - RNA, 31 nt, synthetic construct
  source 1..31: mol_type = other RNA
caggctttctggtttttacaggaaggcccct

SEQ ID NO: 534 - RNA, 29 nt, synthetic construct
  source 1..29: mol_type = other RNA
ggttttctggttctcactggaaaaccccc

SEQ ID NO: 535 - RNA, 31 nt, synthetic construct
  source 1..31: mol_type = other RNA
caggctttccggtaaagactggaaagcccct

SEQ ID NO: 536 - RNA, 31 nt, synthetic construct
  source 1..31: mol_type = other RNA
caggttttccagtttttaccagaaaactctc

SEQ ID NO: 537 - RNA, 31 nt, synthetic construct
  source 1..31: mol_type = other RNA
aaggttttctggtcttcactggaagaccact

SEQ ID NO: 538 - RNA, 31 nt, synthetic construct
  source 1..31: mol_type = other RNA
gaggttttctggtcttcactggaagaccact

SEQ ID NO: 539 - RNA, 31 nt, synthetic construct
  source 1..31: mol_type = other RNA
taggttttctggtcttcactggaagaccact

SEQ ID NO: 540 - RNA, 31 nt, synthetic construct
  source 1..31: mol_type = other RNA
ccggttttctggtcttcactggaagaccact

SEQ ID NO: 541 - RNA, 31 nt, synthetic construct
  source 1..31: mol_type = other RNA
cgggttttctggtcttcactggaagaccact

SEQ ID NO: 542 - RNA, 31 nt, synthetic construct
  source 1..31: mol_type = other RNA
ctggttttctggtcttcactggaagaccact

SEQ ID NO: 543 - RNA, 31 nt, synthetic construct
  source 1..31: mol_type = other RNA
catgttttctggtcttcactggaagacaact

SEQ ID NO: 544 - RNA, 31 nt, synthetic construct
  source 1..31: mol_type = other RNA
cacgttttctggtcttcactggaagacgact

SEQ ID NO: 545 - RNA, 31 nt, synthetic construct
  source 1..31: mol_type = other RNA
caagttttctggtcttcactggaagactact

SEQ ID NO: 546 - RNA, 31 nt, synthetic construct
  source 1..31: mol_type = other RNA
cagcttttctggtcttcactggaagagcact

SEQ ID NO: 547 - RNA, 31 nt, synthetic construct
  source 1..31: mol_type = other RNA
cagattttctggtcttcactggaagatcact

SEQ ID NO: 548 - RNA, 31 nt, synthetic construct
  source 1..31: mol_type = other RNA
cagtttttctggtcttcactggaagaacact

SEQ ID NO: 549 - RNA, 31 nt, synthetic construct
  source 1..31: mol_type = other RNA
cagggtttctggtcttcactggaagcccact

SEQ ID NO: 550 - RNA, 31 nt, synthetic construct
  source 1..31: mol_type = other RNA
caggatttctggtcttcactggaagtccact

SEQ ID NO: 551 - RNA, 31 nt, synthetic construct
  source 1..31: mol_type = other RNA
caggctttctggtcttcactggaaggccact

SEQ ID NO: 552 - RNA, 31 nt, synthetic construct
  source 1..31: mol_type = other RNA
caggtgttctggtcttcactggaacaccact

SEQ ID NO: 553 - RNA, 31 nt, synthetic construct
  source 1..31: mol_type = other RNA
caggtattctggtcttcactggaataccact

SEQ ID NO: 554 - RNA, 31 nt, synthetic construct
  source 1..31: mol_type = other RNA
caggtcttctggtcttcactggaagaccact

SEQ ID NO: 555 - RNA, 31 nt, synthetic construct
  source 1..31: mol_type = other RNA
caggttttctggtcttcactggaaaaccact

SEQ ID NO: 556 - RNA, 31 nt, synthetic construct
  source 1..31: mol_type = other RNA
caggttctctggtcttcactggaggaccact

SEQ ID NO: 557 - RNA, 31 nt, synthetic construct
  source 1..31: mol_type = other RNA
caggttgtctggtcttcactggacgaccact

SEQ ID NO: 558 - RNA, 31 nt, synthetic construct
  source 1..31: mol_type = other RNA
caggttatctggtcttcactggatgaccact

SEQ ID NO: 559 - RNA, 31 nt, synthetic construct
  source 1..31: mol_type = other RNA
caggtttactggtcttcactggtagaccact

SEQ ID NO: 560 - RNA, 31 nt, synthetic construct
  source 1..31: mol_type = other RNA
caggtttgctggtcttcactggcagaccact

SEQ ID NO: 561 - RNA, 31 nt, synthetic construct
  source 1..31: mol_type = other RNA
caggtttcctggtcttcactgggagaccact

SEQ ID NO: 562 - RNA, 31 nt, synthetic construct
  source 1..31: mol_type = other RNA
caggttttttggtcttcactgaaagaccact

SEQ ID NO: 563 - RNA, 31 nt, synthetic construct
  source 1..31: mol_type = other RNA
caggttttgtggtcttcactgcaagaccact

SEQ ID NO: 564 - RNA, 31 nt, synthetic construct
  source 1..31: mol_type = other RNA
caggttttatggtcttcactgtaagaccact

SEQ ID NO: 565 - RNA, 31 nt, synthetic construct
  source 1..31: mol_type = other RNA
caggttttccggtcttcactggaagaccact

SEQ ID NO: 566 - RNA, 31 nt, synthetic construct
  source 1..31: mol_type = other RNA
caggttttcaggtcttcacttgaagaccact

SEQ ID NO: 567 - RNA, 31 nt, synthetic construct
  source 1..31: mol_type = other RNA
caggttttctggtcttcactagaagaccact

SEQ ID NO: 568 - RNA, 31 nt, synthetic construct
  source 1..31: mol_type = other RNA
caggttttcgggtcttcactcgaagaccact

SEQ ID NO: 569 - RNA, 31 nt, synthetic construct
  source 1..31: mol_type = other RNA
caggttttcttgtcttcacaggaagaccact

SEQ ID NO: 570 - RNA, 31 nt, synthetic construct
  source 1..31: mol_type = other RNA
caggttttctagtcttcactggaagaccact

SEQ ID NO: 571 - RNA, 31 nt, synthetic construct
  source 1..31: mol_type = other RNA
caggttttctggtcttcaccggaagaccact

SEQ ID NO: 572 - RNA, 31 nt, synthetic construct
  source 1..31: mol_type = other RNA
caggttttctcgtcttcacgggaagaccact

SEQ ID NO: 573 - RNA, 31 nt, synthetic construct
  source 1..31: mol_type = other RNA
caggttttctgttcttcaatggaagaccact

SEQ ID NO: 574 - RNA, 31 nt, synthetic construct
  source 1..31: mol_type = other RNA
caggttttctgatcttcattggaagaccact

SEQ ID NO: 575 - RNA, 31 nt, synthetic construct
  source 1..31: mol_type = other RNA
caggttttctgctcttcagtggaagaccact

SEQ ID NO: 576 - RNA, 31 nt, synthetic construct
  source 1..31: mol_type = other RNA
caggttttctggccttcgctggaagaccact

SEQ ID NO: 577 - RNA, 31 nt, synthetic construct
  source 1..31: mol_type = other RNA
caggttttctgggcttccctggaagaccact

SEQ ID NO: 578 - RNA, 31 nt, synthetic construct
  source 1..31: mol_type = other RNA
caggttttctggacttctctggaagaccact

SEQ ID NO: 579 - RNA, 31 nt, synthetic construct
  source 1..31: mol_type = other RNA
caggttttctggttttcactggaagaccact

SEQ ID NO: 580 - RNA, 31 nt, synthetic construct
  source 1..31: mol_type = other RNA
caggttttctggtattcactggaagaccact

SEQ ID NO: 581 - RNA, 31 nt, synthetic construct
  source 1..31: mol_type = other RNA
caggttttctggtgttcactggaagaccact

SEQ ID NO: 582 - RNA, 31 nt, synthetic construct
  source 1..31: mol_type = other RNA
caggttttctggtcatcactggaagaccact

SEQ ID NO: 583 - RNA, 31 nt, synthetic construct
  source 1..31: mol_type = other RNA
caggttttctggtcgtcactggaagaccact

SEQ ID NO: 584 - RNA, 31 nt, synthetic construct
  source 1..31: mol_type = other RNA
caggttttctggtcctcactggaagaccact

SEQ ID NO: 585 - RNA, 31 nt, synthetic construct
  source 1..31: mol_type = other RNA
caggttttctggtctacactggaagaccact

SEQ ID NO: 586 - RNA, 31 nt, synthetic construct
  source 1..31: mol_type = other RNA
caggttttctggtctgcactggaagaccact

SEQ ID NO: 587 - RNA, 31 nt, synthetic construct
  source 1..31: mol_type = other RNA
caggttttctggtctccactggaagaccact

SEQ ID NO: 588 - RNA, 31 nt, synthetic construct
  source 1..31: mol_type = other RNA
caggttttctggtcttaactggaagaccact

SEQ ID NO: 589 - RNA, 31 nt, synthetic construct
  source 1..31: mol_type = other RNA
caggttttctggtcttgactggaagaccact

SEQ ID NO: 590 - RNA, 31 nt, synthetic construct
  source 1..31: mol_type = other RNA
caggttttctggtctttactggaagaccact

SEQ ID NO: 591 - RNA, 31 nt, synthetic construct
  source 1..31: mol_type = other RNA
caggttttctggtcttcactggaagaccgct

SEQ ID NO: 592 - RNA, 31 nt, synthetic construct
  source 1..31: mol_type = other RNA
caggttttctggtcttcactggaagacccct

SEQ ID NO: 593 - RNA, 31 nt, synthetic construct
  source 1..31: mol_type = other RNA
caggttttctggtcttcactggaagacctct

SEQ ID NO: 594 - RNA, 31 nt, synthetic construct
  source 1..31: mol_type = other RNA
caggttttctggtcttcactggaagaccagt

SEQ ID NO: 595 - RNA, 31 nt, synthetic construct
  source 1..31: mol_type = other RNA
caggttttctggtcttcactggaagaccatt

SEQ ID NO: 596 - RNA, 31 nt, synthetic construct
  source 1..31: mol_type = other RNA
caggttttctggtcttcactggaagaccaat

SEQ ID NO: 597 - RNA, 31 nt, synthetic construct
  source 1..31: mol_type = other RNA
caggttttctggtcttcactggaagaccaca

SEQ ID NO: 598 - RNA, 31 nt, synthetic construct
  source 1..31: mol_type = other RNA
caggttttctggtcttcactggaagaccacg

SEQ ID NO: 599 - RNA, 31 nt, synthetic construct
  source 1..31: mol_type = other RNA
caggttttctggtcttcactggaagaccacc

SEQ ID NO: 600 - RNA, 132 nt, synthetic construct
  source 1..132: mol_type = other RNA
tcctcctacaaaggcgtgtctgtggttccctgtctttggacacgtaagaattggaggaaaataaatgtggatttgggaaactttgaggccagcttgcttcttgcaggctcatgatcaaccaatctcacataa

SEQ ID NO: 601 - RNA, 131 nt, synthetic construct
  source 1..131: mol_type = other RNA
ctccatgtatctttgggacctgtcagccgtggcagtctcccttcctagccatggaagagcatatccttgtttattggcaaagctgtcaccatttaattggtatcagattctgacttgcacaagtaacattc

SEQ ID NO: 602 - RNA, 132 nt, synthetic construct
  source 1..132: mol_type = other RNA
ctgcgaatattctcgctgttctgattttgtaatagtcaggacaggctaaacattcgctatattaagaccatgcatgtgtccccaaacctagttctttccctaggtctggtttcataaatgctggtgataaac

SEQ ID NO: 603 - RNA, 133 nt, synthetic construct
  source 1..133: mol_type = other RNA
gcatggccgaatactgtgtttttatcagtagtttacacagccagacaccatgcaaaagcagtcttccctttagaatgactgatggtatgctaaggtttttcatagcatatcattattaaaggtgaatacaaat

SEQ ID NO: 604 - RNA, 139 nt, synthetic construct
  source 1..139: mol_type = other RNA
tgcactgcatggtatctgcactcagcagtttacacctgctagggtgttcaaaggtcagtgctatagaaattcagtatctggcatcgttggttttcttggctttgtgcttgttaaacctggtatttctactgatacagta

SEQ ID NO: 605 - RNA, 134 nt, synthetic construct
  source 1..134: mol_type = other RNA
tggtccatcctaatccctgccggtccatctgtggcctgccaggtttcgcttgtggaccagagcaccctagaagcctcacccgaggagtgagcagggctccagtgggctcacgtcatgggcacttctagacactc

SEQ ID NO: 606 - RNA, 136 nt, synthetic construct
  source 1..136: mol_type = other RNA
cacctgcattcaaaaatgatcacgggctgcctgtgctctggtcatcaataacgcagggagaggaattgctgaaagccgtttcccgtgtttggagggttcacacctgtccctttcaaatgctggcgctttcacacac

SEQ ID NO: 607 - RNA, 134 nt, synthetic construct
  source 1..134: mol_type = other RNA
tgcattcttaaaccctcttggtggcttccctgtaaatgcttccaagatatgagcgaatgctatagaaattgcaggaaagtccaaagggctgcgcgtctcctgtggctcagtcttatttcatacctgcaacatct

SEQ ID NO: 608 - RNA, 133 nt, synthetic construct
  source 1..133: mol_type = other RNA
attgcacctaaacccaagaatcactgtttcttatagcggtggtttaaacagaggtgcaaacagcaagcggatcttgtcgcctttggggggctgtggccgtgcccctcaaagtgaatttggaggttccacaact

SEQ ID NO: 609 - RNA, 132 nt, synthetic construct
  source 1..132: mol_type = other RNA
cttcccatttatttgctgcttgtagtctcacagtgatacgagcagttatacgcatgggataaaataacattgggccactgtaaattgagatgaagtaaccattttcatctcttctgcagggactagacattg

SEQ ID NO: 610 - RNA, 135 nt, synthetic construct
  source 1..135: mol_type = other RNA
ctgcattcttaaaccctcttggtagcttcgttctaagtgcttccaagatatgagtgaatgctatagaaattgcaggggagtccaaagggctgcgcttctcccgtggctcagtcttatttcatacctgcgacatct

SEQ ID NO: 611 - RNA, 131 nt, synthetic construct
  source 1..131: mol_type = other RNA
aggcaggatctagttacattgtagctgtgaagtgctgcattgtctttgccccctgctcaaaataaaactgttacctttcaagccctgtctgccatggtgctgtagcagcagggatgtttggtctcatacat

SEQ ID NO: 612 - RNA, 123 nt, synthetic construct
  source 1..123: mol_type = other RNA
actggaggactaaggaggctgggtctgatgaggcaagattttgctgatacattgctcctagaaaaaagggttggcaagagcagccctggagactcacacggctgactgttctacccaacactc

SEQ ID NO: 613 - RNA, 129 nt, synthetic construct
  source 1..129: mol_type = other RNA
aagcaggattcagactacaatatagctgttaagtgctgtattgtcattccccctgctcaaattaaagttgtttcttaactatacccatctgctattctgtagcagccagggatgcttggtcacatacat

SEQ ID NO: 614 - RNA, 132 nt, synthetic construct
  source 1..132: mol_type = other RNA
ggcttcctagtacttaccatggtctgtgttcttacgctgactgtatagaaacaggaggcagagtaaaccgaccccacatatacctcagcccaggccctgtgctgcgtctgtattgtgaatcaggagacatgg

SEQ ID NO: 615 - RNA, 139 nt, synthetic construct
  source 1..139: mol_type = other RNA
tggctcgatttcctggggggtggtctcagcccactccacctcccctcagccgagcctagagtagaggggccaggcatcctccccaggggaggggcgttgaagcaaggagcctctcctgggctgtcctagcctcacattt

SEQ ID NO: 616 - RNA, 132 nt, synthetic construct
  source 1..132: mol_type = other RNA
gttgaggtctatcccgataggtcttttcctgtagcctgcacgttgttggaaatgcctcatagagtaactctgtgattttactttacttacaggactattgttacatctgtgggaaggaaccacaagacagtt

SEQ ID NO: 617 - RNA, 133 nt, synthetic construct
  source 1..133: mol_type = other RNA
ttctcacctaaacccaagaatcactgtttcttatagcggtggtttaaacagaggtgcaaacagcaagtgaatctcgtcgcctttgcggggctgtggccatgcccctcaaaggaaatttggaggctctacagcc

SEQ ID NO: 618 - RNA, 128 nt, synthetic construct
  source 1..128: mol_type = other RNA
aggtcatttcaaagaggtcttgtgaggctgtgaaaccaagagctcttaacactgcgaccaaagatggaagttctctataggatgccatggcatttgatggtgctatgttttcttgaggagatataaga

SEQ ID NO: 619 - RNA, 131 nt, synthetic construct
  source 1..131: mol_type = other RNA
ccctcctacaaaggcatgtctatagttccttgtctttggacatgtaagaattggaggcaaagaaatgtggacttggagaaatctggggccagcttgctctccgcaggctcaagatcaaccatcccacatag

SEQ ID NO: 620 - RNA, 125 nt, synthetic construct
  source 1..125: mol_type = other RNA
gcagactcactatgcacctgactgtacttccaggcaggtgctttttctgtctgccagagaaacattccagggtgctgtggctgcctcacctatccagggcgatgcagctccctggggacacaggt

SEQ ID NO: 621 - RNA, 125 nt, synthetic construct
  source 1..125: mol_type = other RNA
gcagactcactctgcatctgactatacttccaggcgggtgctttttctgtctgccagataaacattccagggtgctgtggccgcctcacgtatccagagtgatgcagctccctggggacacaggt

SEQ ID NO: 622 - RNA, 148 nt, synthetic construct
  source 1..148: mol_type = other RNA
tgatggctgttcctctcactgcttgaagccttaggcagtgggattttgatccatcatatatcaaaaatggcttatcttcactcagggcaccatgaggatgggctggctgtccgttagtgccttctgatttttgcggagtcaaacaatt

SEQ ID NO: 623 - RNA, 127 nt, synthetic construct
  source 1..127: mol_type = other RNA
ctggggaattcaaacttgtgttaagaaaatgtgtcccagtgtgcaatggctgcaaacagcagcttccttggtagtgtatgcagcctgtttgttgtacgggttgctctaaagggccttggagacagtc

SEQ ID NO: 624 - RNA, 126 nt, synthetic construct
  source 1..126: mol_type = other RNA
aggtcacttcaaagagggcttgtggggctgtgaaaccaagaggtcttaacagtatgaccaaaaactgaagttctctataggatgctgtagcactcaatggtgctatgttttcctcaggagatatga

SEQ ID NO: 625 - RNA, 139 nt, synthetic construct
  source 1..139: mol_type = other RNA
ctgtccgttgctggcttcacaagtactagtataatttttaaaatgttttattattttgaaaataatgttgtaattcatgccagggactgacaaaagacttgagacaggatggttattcttgtcagctaaggtcacattg

SEQ ID NO: 626 - RNA, 113 nt, synthetic construct
  source 1..113: mol_type = other RNA
tccaaacagacactgatggcaccttctgccatttaggaatttgttttaaaacagacatttgtctagatatttcctttgtggcctcctccccatcaaaagtcaatcaaacatcg

SEQ ID NO: 627 - RNA, 143 nt, synthetic construct
  source 1..143: mol_type = other RNA
gccctatgttaaaattttaattctgcacttactaactatcttgggaaccttgggcaagtaaccaacctcttgtgctttggtttcctcattggtaaaatggggataacagtacttacctcacagagttgttgagaggaacaaat

SEQ ID NO: 628 - RNA, 125 nt, synthetic construct
  source 1..125: mol_type = other RNA
atgcaggtactgttacaatacaactgatgtgttttgttgtcgttccccctgcttaaagcacttgatgcataactctgtctaccttcattccgtagtaagacagagacgcttggcttcagacattt

SEQ ID NO: 629 - RNA, 147 nt, synthetic construct
  source 1..147: mol_type = other RNA
tcactgccctgctcacccttcctgagtccggcggcaagggtaactctgggagcatcgtagagggcagagaagaagaaaccctgaggtcccattatgtcagccccttctatcacacgggaggagactgaggacagaaagggaacagag

SEQ ID NO: 630 - RNA, 147 nt, synthetic construct
  source 1..147: mol_type = other RNA
cttttctcagtggtgcaagaagattaagccacattctggctttagagaggcatttctgagagagatgaaggacacttcgttccccagccccaacctaagcatgtgactgtactcaccttgtcagatgctgttggaacctggctgaca

SEQ ID NO: 631 - RNA, 122 nt, synthetic construct
  source 1..122: mol_type = other RNA
ctggaggactaaggaggctgggtctgatgaggcaagattttgctgatacattgctcctagaaaaaagggttggcaagagcagccctggagactcacacggctgactgttctacccaacactc

SEQ ID NO: 632 - RNA, 118 nt, synthetic construct
  source 1..118: mol_type = other RNA
tagtgtggaactgtctactcctcattcctgtggaagcaggaatacattcataacatgctccattaaaaaaggagttctaggccaggcagcgtgcctcatgcctggaatcccagcactt

SEQ ID NO: 633 - RNA, 126 nt, synthetic construct
  source 1..126: mol_type = other RNA
atacctaaacccaagaatcactttcttatagtgatgatttaaacagatgcaaacagcgagcacatcttgtcacctttgcgggactgtggctgtgcccctcgcagtaaatttggaggttctacatcc

SEQ ID NO: 634 - RNA, 133 nt, synthetic construct
  source 1..133: mol_type = other RNA
aggcaggatctagttacattgtagctgtgaagtgctgcattgtctttgccccctgctcaaaataaaactgttacctttcaagccctgtctgccatggtgctgtagcagcagggatgtttggtctcatacatgt

SEQ ID NO: 635 - RNA, 81 nt, synthetic construct
  source 1..81: mol_type = other RNA
cactagaagacagaattcacagaagtagcatttcaccttttgcctttacagaagtatatttggctgttttgtgagacattc

SEQ ID NO: 636 - RNA, 112 nt, synthetic construct
  source 1..112: mol_type = other RNA
acttttacaggtagaatagtaaagcacagtgttgattgcccaagatttattttactttgaaaaaattagaaatttattactatagcaaatgtctagaactttggaaacaagt

SEQ ID NO: 637 - RNA, 134 nt, synthetic construct
  source 1..134: mol_type = other RNA
atgcatctatttgacagacctggagcagttgctatctgctgctatggtttccaccacagatgcaagaagaacatgtccttgcgctttccgtctgtctaattgtggcagctgagattgaatagaggaatacagga

SEQ ID NO: 638 - RNA, 136 nt, synthetic construct
  source 1..136: mol_type = other RNA
aagcactgcctttgaacctgatgtgtcttgtttgtagcttcacgggccaagcaacagtgctagagcataacgacttgttataactggggctcttcagctctcaactgaactgctcttttaaaaacaaggtacattt

SEQ ID NO: 639 - RNA, 136 nt, synthetic construct; identical to SEQ ID NO: 638
  source 1..136: mol_type = other RNA

SEQ ID NO: 640 - RNA, 133 nt, synthetic construct
  source 1..133: mol_type = other RNA
agcctttgtgttgcccattcactttggaaactagtgaatgtggtgtcaaaaaaggcgtaaattaaacgctttgcagccttttcctgcccttaaatttgatacctttggtgtaggagctgcataagtaacagtt

SEQ ID NO: 641 - RNA, 132 nt, synthetic construct
  source 1..132: mol_type = other RNA
ttccaaagtgttgagttcagtccagggcagcttccctgttctgttaattaaactttgggacattaaaatgggctaagggagatgattgggtagaaagtattattctattcatttgcctcccagcctacaaaa

SEQ ID NO: 642 - RNA, 126 nt, synthetic construct
  source 1..126: mol_type = other RNA
aagcaacactctgtggcagatgatcaaaactgtctgacacaatttgagcttgctatagcaagaaagtctaacctattccggtgttctctctcccatgagacaagccgttatatagacttaaacagt

SEQ ID NO: 643 - RNA, 126 nt, synthetic construct; identical to SEQ ID NO: 642
  source 1..126: mol_type = other RNA

SEQ ID NO: 644 - RNA, 132 nt, synthetic construct
  source 1..132: mol_type = other RNA
ttccacagctactggtctgcagctgttcttatggtagcagttgtggcattcctctgtgggaaagaaactgttaacacaaacacctctttcttagcaaaacagaaagtgggtatatatgtgtgacagacacaa

SEQ ID NO: 645 - RNA, 134 nt, synthetic construct
  source 1..134: mol_type = other RNA
tgcagccgtgtcaaattcagtacctgtcctatgcatggtaggcactggcccagaaggctgccacagaaacactgtgactcatgggccctgttcctgtgtcccaggctcagggataaatttggttacagacatca

SEQ ID NO: 646 - RNA, 139 nt, synthetic construct; identical to SEQ ID NO: 604
  source 1..139: mol_type = other RNA

SEQ ID NO: 647 - RNA, 139 nt, synthetic construct; identical to SEQ ID NO: 604
  source 1..139: mol_type = other RNA

SEQ ID NO: 648 - RNA, 133 nt, synthetic construct
  source 1..133: mol_type = other RNA
tgcagccgtgtcaattcagtacctgtcctatgcatggtaggcactggcccagaaggctgccacagaaacactgtgactcatgggccctgttcctgtgtcccaggctcagggataaatttggttacagacatca

SEQ ID NO: 649 - RNA, 132 nt, synthetic construct; identical to SEQ ID NO: 641
  source 1..132: mol_type = other RNA

SEQ ID NO: 650 - RNA, 134 nt, synthetic construct
  source 1..134: mol_type = other RNA
tggtaatggatttatggtgggtccttctctgtgggcctctcatagtgtacccatgccatagcaaatggcagcctcgaaccattgcccagtccccttacctgtgggctgtgagcactgaagggggttgcacagtg

SEQ ID NO: 651 - RNA, 134 nt, synthetic construct; identical to SEQ ID NO: 650
  source 1..134: mol_type = other RNA

SEQ ID NO: 652 - RNA, 132 nt, synthetic construct
  source 1..132: mol_type = other RNA
cagcatgtttccaagggctgtggctggtcatagccatgggatctccaactgcatgcaagagcaacctggaaagactttgacagcgcaggtcagtacaatacctgcaagctgccactcagctttcctataatg

SEQ ID NO: 653 - RNA, 132 nt, synthetic construct; identical to SEQ ID NO: 652
  source 1..132: mol_type = other RNA

SEQ ID NO: 654 - RNA, 133 nt, synthetic construct
  source 1..133: mol_type = other RNA
ggtctctcagctccgcttaaccacacgggtccagtgtgtgcttggcgtgttttcagggaggcagagaaaggctctcctaatgcacgacagacccgcccagaatggcctctctgttcctaggagtgcgacaatt

SEQ ID NO: 655 - RNA, 135 nt, synthetic construct
  source 1..135: mol_type = other RNA
agcactatatttaaacctgtggatgggaatattccccattcttggttacgctgtagtgcaaaagaattcctggctctctgttgcacagctgacttgtgccattctgctgttgctgtatagagttaaggaacatgg

SEQ ID NO: 656 - RNA, 133 nt, synthetic construct; identical to SEQ ID NO: 654
  source 1..133: mol_type = other RNA

SEQ ID NO: 657 - RNA, 130 nt, synthetic construct
  source 1..130: mol_type = other RNA
tgcctcattctagagaatgggcactgttgatcatggtgtccaaaaatagttaatgtggctaaattgagacaggttatgcttccatcacagtatgcatattgcagtggtgacaatgagacctgtaacattt

SEQ ID NO: 658 - RNA, 135 nt, synthetic construct
  source 1..135: mol_type = other RNA
taggccctgaatcaagaccaatggtttgctgtagctgttggtttcaaacaggagctaagagtgatgtcttccttgtggtctgttggctattcagtattccagtgcgaattgccaattcagttggaagaaacatag

SEQ ID NO: 659 - RNA, 135 nt, synthetic construct; identical to SEQ ID NO: 658
  source 1..135: mol_type = other RNA

SEQ ID NO: 660 - RNA, 130 nt, synthetic construct
  source 1..130: mol_type = other RNA
atcgaggctagagtcacgcttgggtatcggctattgcctgagtgtgctagagtcctcgaagagtaactgctgaccttattcactggctgtgggccttatggcacagtcagtcaccaggttagagacatgc

SEQ ID NO: 661 - RNA, 130 nt, synthetic construct; identical to SEQ ID NO: 660
  source 1..130: mol_type = other RNA

SEQ ID NO: 662 - RNA, 149 nt, synthetic construct
  source 1..149: mol_type = other RNA
tgcacactattaaagctcagggtggaggccagtcttggctcatgaacttctgagtgtcggaagtgtgctatatcaatggcaggattttcgctaacaccagtagagcttgcctctatgactggagtttggtagtactcgctgccacatag

SEQ ID NO: 663 - RNA, 139 nt, synthetic construct
  source 1..139: mol_type = other RNA
gacctcctgggatcgcatctggagagtgcctagtattctgccagcttcggaaagggagggaaagcaagcctggcagaggcacccattccattcccagcttgctccgtagctggcgattggaagacactctgcgacagtg

SEQ ID NO: 664 - RNA, 139 nt, synthetic construct; identical to SEQ ID NO: 663
  source 1..139: mol_type = other RNA

SEQ ID NO: 665 - RNA, 133 nt, synthetic construct
  source 1..133: mol_type = other RNA
tagcaagcctccagcgtgcttgggtctgcggtgaccctatgcattccttcagtgcttgctagaacagttttgaaacggtttgaggccttgccctgctccatccagagcaaggttatagaaatttcagacaatg

SEQ ID NO: 666 - RNA, 133 nt, synthetic construct; identical to SEQ ID NO: 665
  source 1..133: mol_type = other RNA

SEQ ID NO: 667 - RNA, 134 nt, synthetic construct
  source 1..134: mol_type = other RNA
ttggcccttatcgaagctgcagctgcttccgcatagctgctgtggtcaaaaaggagcccagagtgacagttttccttgacggtcgccgttctgtttgttgtaactgatctgcaacattttgggaaaatacagtt

SEQ ID NO: 668 - DNA, 31 bp, synthetic construct
  source 1..31: mol_type = other DNA
acacaggcactggccactgaaatttttggag

SEQ ID NO: 669 - DNA, 47 bp, synthetic construct
  source 1..47: mol_type = other DNA
taggctttctggcttttcaccggaaagcccctaaatgattaaattaa

SEQ ID NO: 670 - DNA, 31 bp, synthetic construct
  source 1..31: mol_type = other DNA
gattaggcacacacaggcacaatttttggag

SEQ ID NO: 671 - DNA, 47 bp, synthetic construct; identical to SEQ ID NO: 669
  source 1..47: mol_type = other DNA

SEQ ID NO: 672 - DNA, 31 bp, synthetic construct
  source 1..31: mol_type = other DNA
cttccttgacgattaggcacaatttttggag

SEQ ID NO: 673 - DNA, 47 bp, synthetic construct; identical to SEQ ID NO: 669
  source 1..47: mol_type = other DNA

SEQ ID NO: 674 - DNA, 31 bp, synthetic construct
  source 1..31: mol_type = other DNA
accttcttccttgacgattaaatttttggag

SEQ ID NO: 675 - DNA, 47 bp, synthetic construct; identical to SEQ ID NO: 669
  source 1..47: mol_type = other DNA

SEQ ID NO: 676 - DNA, 31 bp, synthetic construct
  source 1..31: mol_type = other DNA
acacaggcactggccactgatgcgtgtcatt

SEQ ID NO: 677 - DNA, 47 bp, synthetic construct; identical to SEQ ID NO: 669
  source 1..47: mol_type = other DNA

SEQ ID NO: 678 - DNA, 31 bp, synthetic construct
  source 1..31: mol_type = other DNA
ggcacacacaggcactggcctgcgtgtcatt

SEQ ID NO: 679 - DNA, 47 bp, synthetic construct; identical to SEQ ID NO: 669
  source 1..47: mol_type = other DNA

SEQ ID NO: 680 - DNA, 31 bp, synthetic construct
  source 1..31: mol_type = other DNA
acgagctcagcctatgcgagaatttttggag

SEQ ID NO: 681 - DNA, 47 bp, synthetic construct; identical to SEQ ID NO: 669
  source 1..47: mol_type = other DNA

SEQ ID NO: 682 - DNA, 31 bp, synthetic construct
  source 1..31: mol_type = other DNA
ccacctaccctatcgtgcggaatttttggag

SEQ ID NO: 683 - protein, 47 aa, synthetic construct
TAGGCTTTCTGGCTTTTCACCGGAAAGCCCCTAAATGATTAAATTAA

SEQ ID NO: 684 - DNA, 83 bp, synthetic construct
  source 1..83: mol_type = other DNA
ggtcaatgatgtgttggcatgtatcaggtattcctgtggctgatgtgtaataacactggatgaagggacacacactgagacct

SEQ ID NO: 685 - DNA, 103 bp, synthetic construct
  source 1..103: mol_type = other DNA
tatgatagggacttagggtgggtcaatgatgtgttggcatgtatcaggtattcctgtggctgatgtgtaataacactggatgaagggacacacactgagacct

SEQ ID NO: 686 - DNA, 103 bp, synthetic construct
  source 1..103: mol_type = other DNA
ggtcaatgatgtgttggcatgtatcaggtattcctgtggctgatgtgtaataacactggatgaagggacacacactgagaccttatgatagggacttagggtg

SEQ ID NO: 687 - DNA, 83 bp, synthetic construct
  source 1..83: mol_type = other DNA
ggtcaatgatgtgttggcatgtatggtacaggtattcctctgatgtgtaataacactctgtggatgaagggacactgagacct

SEQ ID NO: 688 - DNA, 103 bp, synthetic construct
  source 1..103: mol_type = other DNA
tatgatagggacttagggtgggtcaatgatgtgttggcatgtatggtacaggtattcctctgatgtgtaataacactctgtggatgaagggacactgagacct

SEQ ID NO: 689 - DNA, 103 bp, synthetic construct
  source 1..103: mol_type = other DNA
ggtcaatgatgtgttggcatgtatggtacaggtattcctctgatgtgtaataacactctgtggatgaagggacactgagaccttatgatagggacttagggtg

SEQ ID NO: 690 - DNA, 135 bp, synthetic construct
  source 1..135: mol_type = other DNA
agcactataaagggacctgtggatgggaatattccccattcttggtacacacatagtgcaaaagaattcctggctctctgttgcacagctgacttgtgccattctgctgttgctgtatagagttaaggaacatgg

SEQ ID NO: 691 - DNA, 155 bp, synthetic construct
  source 1..155: mol_type = other DNA
tatgatagggacttagggtgagcactataaagggacctgtggatgggaatattccccattcttggtacacacatagtgcaaaagaattcctggctctctgttgcacagctgacttgtgccattctgctgttgctgtatagagttaaggaacatgg

SEQ ID NO: 692 - DNA, 155 bp, synthetic construct
  source 1..155: mol_type = other DNA
agcactataaagggacctgtggatgggaatattccccattcttggtacacacatagtgcaaaagaattcctggctctctgttgcacagctgacttgtgccattctgctgttgctgtatagagttaaggaacatggtatgatagggacttagggtg

SEQ ID NO: 693 - DNA, 132 bp, synthetic construct
  source 1..132: mol_type = other DNA
cagcatgtttccaagggctgtggctggtcatagccatgggatctccaactgcatgcaagagcaacctggaaagacacacacagcgcaggtcagtacaatacctgcaagctgcatgccagctttcctataatg

SEQ ID NO: 694 - DNA, 152 bp, synthetic construct
  source 1..152: mol_type = other DNA
tatgatagggacttagggtgcagcatgtttccaagggctgtggctggtcatagccatgggatctccaactgcatgcaagagcaacctggaaagacacacacagcgcaggtcagtacaatacctgcaagctgcatgccagctttcctataatg

SEQ ID NO: 695 - DNA, 152 bp, synthetic construct
  source 1..152: mol_type = other DNA
cagcatgtttccaagggctgtggctggtcatagccatgggatctccaactgcatgcaagagcaacctggaaagacacacacagcgcaggtcagtacaatacctgcaagctgcatgccagctttcctataatgtatgatagggacttagggtg

SEQ ID NO: 696 - DNA, 247 bp, synthetic construct
  source 1..247: mol_type = other DNA
tacttacctggcaggggagataccatgatcacgaaggtggtttttcccagggcgaggcttatccattgcactccggatgtgctgacccctgcgatttccccaaatgtgggaaactcgactgcataatttgtggtagtgggggactgcgttcgcgctttcccctgtcactagtctgtggtgtgatatccatggcggcctacttatcctgtcccttttttttccacagnnnnnnnnnnnnnnnnnnnnn

SEQ ID NO: 697 - DNA, 242 bp, synthetic construct
  source 1..242: mol_type = other DNA
acctggcaggggagataccatgatcacgaaggtggttttcccagggcgaggcttatccattgcactccggatgtgctgacccctgcgatttccccaaatgtgggaaactcgactgcataattttgtggtagtgggggactgcgttcgcgctttcccctgtcactagtctgtggtgtgatatccatggcggcctacttatcctgtcccttttttttccacagnnnnnnnnnnnnnnnnnnnnn

SEQ ID NO: 698 - DNA, 237 bp, synthetic construct
  source 1..237: mol_type = other DNA
ggcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgctttcccctgtcactagtctgtggtgtgatatccatggcggcctacttatcctgtcccttttttttccacagnnnnnnnnnnnnnnnnnnnnn

SEQ ID NO: 699 - DNA, 216 bp, synthetic construct
  source 1..216: mol_type = other DNA
aaaaagggcttctgtcgtgagtggcacacgtagggcaactcgattgctctgcgtgcggaatcgacatcaagagatttcggaagcataattttttggtatttgggcagctggtgatcgttcgtcccggcgccctttcactagtctgtggtgtgctatccatggcggcctacttatcctgtcccttttttttccacagnnnnnnnnnnnnnnnnnnnn

SEQ ID NO: 700 - DNA, 213 bp, synthetic construct
  source 1..213: mol_type = other DNA
agggcttctgtcgtgagtggcacacgtagggcaactcgattgctctgcgtgcggaatcgacatcaagagatttcggaagcataattttttggtatttgggcagctggtgatcgttcgtcccggcgccctttcactagtctgtggtgtgactatccatggcggcctacttatcctgtcccttttttttccacagnnnnnnnnnnnnnnnnnnnn

SEQ ID NO: 701 - DNA, 208 bp, synthetic construct
  source 1..208: mol_type = other DNA
gcttctgtcgtgagtggcacacgtagggcaactgattgctctgcgtgcggaatcgacatcaagagatttcggaagcataattttttggtatttgggcagctggtgatcgttcgtcccggcgccctttcactagtctgtggtgtgatatccatggcggcctacttatcctgtcccttttttttccacagnnnnnnnnnnnnnnnnnnnn

SEQ ID NO: 702 - DNA, 127 bp, synthetic construct
  source 1..127: mol_type = other DNA
aatttttggagcaggttttccagtgttcactgaaatttgtctcttcactagtctgtggtgtgatatcccatggcggcctacttatcctgtcccttttttttccacagnnnnnnnnnnnnnnnnnnnn

SEQ ID NO: 703 - DNA, 124 bp, synthetic construct
  source 1..124: mol_type = other DNA
aatttttggagaaggttttctggtctttatcagaaagcctcctcactagtctgtggtgtgatatccatggcggcctacttatcctgtcccttttttttccacagnnnnnnnnnnnnnnnnnnnn

SEQ ID NO: 704 - DNA, 125 bp, synthetic construct
  source 1..125: mol_type = other DNA
aatttttggagcaggttttctggtttcactgcaaaaccccatcactagtctgtggtgtgatatccatggcggcctactggatcctgtccctttttttttccacagnnnnnnnnnnnnnnnnnnnn

SEQ ID NO: 705 - DNA, 138 bp, synthetic construct
  source 1..138: mol_type = other DNA
aatttttggagcacgttttccggttgtcactcccaggtaggctggggaagaggcattcactagtctgtggtgtgatatccatggcggcctacttatcctgtcccttttttttccacagnnnnnnnnnnnnnnnnnnnn

SEQ ID NO: 706 - DNA, 101 bp, synthetic construct
  source 1..101: mol_type = other DNA
aattttttggagcgtctcttcactagtctgtggtgtgatatccatggcggcctacttatcctgtcccttttttttccacagnnnnnnnnnnnnnnnnnnnn

SEQ ID NO: 707 - DNA, 98 bp, synthetic construct
  source 1..98: mol_type = other DNA
aatttttggagaatcctcactagtctgtggtgtgatatccatggcggcctacttatcctgtcccttttttttccacagnnnnnnnnnnnnnnnnnnnn

SEQ ID NO: 708 - DNA, 102 bp, synthetic construct
  source 1..102: mol_type = other DNA
aatttttggagcagcccatcactagtctgtggtgtgatatccatggcgtgcctacttatcctgtccctttttttttccacagnnnnnnnnnnnnnnnnnnnn

SEQ ID NO: 709 - DNA, 109 bp, synthetic construct
  source 1..109: mol_type = other DNA
aatttttggagcacgttttccggttattcactagtctgtggtgtgatatccatggcggcctacttatcctgtcccttttttttccacagnnnnnnnnnnnnnnnnnnnn

SEQ ID NO: 710 - DNA, 195 bp, synthetic construct
  source 1..195: mol_type = other DNA
aggtcagagggcttgtggggctgtgaaaccaagaggtctttgaccaaaaactgaagttctctataggatgctgtagcactcaatggtgctatgttttcctcaggagatatgatcactagtctgtggtgtgatatccatggcggcctacttatcctgtccctttttttttccacagnnnnnnnnnnnnnnnnnnnn

SEQ ID NO: 711 - DNA, 186 bp, synthetic construct
  source 1..186: mol_type = other DNA
ctggagggctgggtctgatgaggcaagattttgctctcctagaaaaaagggttggcaagagcagccctggagactcacacggctgactgttctacccaacactctcactagtctgtggtgtgatatccatggcggcctacttatcctgtcccttttttttccacagnnnnnnnnnnnnnnnnnnnn

SEQ ID NO: 712 - DNA, 209 bp, synthetic construct
  source 1..209: mol_type = other DNA
tggctcgggggggtggtctcagcccactccacctccccgagcctagagtagaggggccaggcatcctccccaggggaggggcgttgaagcaaggagcctctcctgggctgtcctagcctcacattttcactagtctgtggtgtgatatccatggcggcctacttatcctgtccctttttttttccacagnnnnnnnnnnnnnnnnnnnn

SEQ ID NO: 713 - DNA, 214 bp, synthetic construct
  source 1..214: mol_type = other DNA
tcactgccctcttcctgagtccggcggcaagggtaactctgggagagggcagagaagaagaaaccctgaggtcccattatgtcagccccttctatcacacgggaggagactgaggacagaaagggaacagagtcactagtctgtggtgtgatatccatggcggcctacttatcctgtcccttttttttccacagnnnnnnnnnnnnnnnnnnnn